(19) United States
(12) Patent Application Publication
(10) Pub. No.: US 2006/0116826 A1
(43) Pub. Date: Jun. 1, 2006

(54) Crystals of cytochrome P450 2C9, structures thereof and their use

(75) Inventors: Pamela Ann Williams, Cambridge (GB); Jose Marie Cosme, Cambridge (GB); Dijana Matak Vinkovic, Cambridge (GB); Christopher William Murray, Cambridge (GB); Harren Jhoti, Cambridge (GB)

Correspondence Address: NIXON & VANDERHYE, PC, 901 NORTH GLEBE ROAD, 11TH FLOOR, ARLINGTON, VA, 22203, US

(73) Assignee: ASTEX THERAPEUTICS LIMITED, Cambridge (GB)

(21) Appl. No.: 11/258,403

(22) Filed: Oct. 26, 2005

Related U.S. Application Data
(63) Continuation of application No. PCT/GB04/01864, filed on Apr. 30, 2004.
(63) Continuation-in-part of application No. 10/426,058, filed on Apr. 30, 2003.
Application No. 10/426,058 is a continuation-in-part of application No. 10/280,137, filed on Oct. 25, 2002.
(60) Provisional application No. 60/330,585, filed on Oct. 25, 2001.
(60) Provisional application No. 60/339,421, filed on Dec. 14, 2001.
(60) Provisional application No. 60/341,267, filed on Dec. 20, 2001.
(60) Provisional application No. 60/396,588, filed on Jul. 18, 2002.

(51) Int. Cl. G06F 19/00; C12N 9/02

(52) U.S. Cl. 702/19; 435/189

(57) ABSTRACT

The present invention provides co-crystals of cytochrome P450 2C9 proteins and a ligand such as warfarin which has been crystallised to provide a high resolution structure. The structure may be used for homology modelling of other cytochrome P450 structures such as 2C8, 2C18 and 2C19, and for analysis of the interaction of ligands with P450.

This application is a continuation of PCT/GB2004/001864, which designated the U.S. and was filed Apr. 30, 2004 (pending); the present application is also a continuation-in-part of U.S. application Ser. No. 10/426,058, filed Apr. 30, 2003 (pending), the present application is also a continuation-in-part of U.S. application Ser. No. 10/280,137, filed Oct. 25, 2002 (pending), and U.S. application Ser. No. 10/280,137 claims benefit of priority of U.S. Provisional Application No. 60/330,585, filed Oct. 25, 2001; U.S. Provisional Application No. 60/339,421, filed Dec. 14, 2001; U.S. Provisional Application No. 60/341,267, filed Dec. 20, 2001; and U.S. Provisional Application No. 60/396,588, filed Jul. 18, 2002; the entire contents of each of the above-identified applications being incorporated herein by reference.

### FIELD OF THE INVENTION

The present invention relates to a co-crystals of human cytochrome P450 protein 2C9 and a ligand such as S-warfarin, methods of production of co-crystals of 2C9, and the uses of such crystals and their structures, in particular the structures of 2C9 binding regions.

### BACKGROUND TO THE INVENTION

Cytochrome P450s (CYP450) form a very large and complex gene superfamily of haemproteins that metabolise physiologically important compounds in many species of microorganisms, plants and animals. Cytochrome P450s are important in the oxidative, peroxidative and reductive metabolism of numerous and diverse endogenous compounds such as steroids, bile, fatty acids, prostaglandins, leukotrienes, retinoids and lipids. Many of these enzymes also metabolise a wide range of xenobiotics including drugs, environmental compounds and pollutants. Their involvement in drug metabolism is extensive, it is estimated that 50% of all known drugs are affected in some way by the action of CYP450 enzymes. Significant resource is employed by the pharmaceutical industry to optimise drug candidates in order to avoid their detrimental interactions with the CYP450 enzymes. Another level of complication results from the fact that these enzymes exhibit different tissue distributions and polymorphisms between individuals and ethnic populations

Most mammalian P450s are located in the liver, but other organs and tissues have high concentrations of certain cytochrome P450s, including the intestinal wall, lung, kidney, adrenal cortex and nasal epithelium. Mammals have about 50 unique CYP450 genes and each family member is 45-55 KDa in size and contains a haem moiety that catalyses a two-electron activation of oxygen. The source of electrons may be used to classify CYP450s. Those that receive electrons in a three protein chain in which electrons flow from a flavin adenine dinucleotide (FAD) containing reductase, to an iron-sulphur protein, and then to P450 belong to the group of class I P450s, and include most of the bacterial enzymes. Class II P450s receive electrons from a reductase containing both FAD and flavin mononucleotide (FMN), and comprise the microsomal P450s that are the main culprits of drug metabolism. The mammalian microsomal cytochrome P450s are integral membrane proteins anchored by an N-terminal transmembrane spanning α-helix. They are inserted in the membrane of the endoplasmic reticulum by a short, highly hydrophobic N-terminal segment that acts as a non-cleavable signal sequence for insertion into the membrane. The remainder of the mammalian cytochrome P450 protein is a globular structure that protrudes into the cytoplasmic space. Hence, the bulk of the enzyme faces the cytoplasmic surface of the lipid bilayer. P450s require other membranous enzymatic components for activity including the flavoprotein NADPH-cytochrome P450 oxidoreductase and, in some cases, cytochrome b5. A single cytochrome P450 oxidoreductase supports the activity of all the mammalian microsomal enzymes by interacting directly with the P450s and transferring the required two electrons from NADPH. Cytochrome P450s are able to incorporate one of the two oxygen atoms of an O₂ molecule into a broad variety of substrates with concomitant reduction of the other oxygen atom by two electrons to H₂O. Cytochrome P450 are known to catalyse hydroxylations, epoxidation, N—, S—, and O-dealkylations, N-oxidations, sulfoxidations, dehalogenations, and other reactions.

The genes of the P450 superfamily have been categorized by Nelson et al (Pharmacogenetics, 6; 1-42, 1996) who proposed a systematic nomenclature for the family members. This nomenclature is used widely in the art, and is adopted herein. Nelson et al provide cross-references to sequence database entries for P450 sequences.

Homo sapiens has 17 cytochrome P450 gene families and 42 subfamilies that total more than 50 sequenced isoforms. Cytochrome P450s from families 1, 2 and 3 constitute the major pathways for drug metabolism. Many drugs rely on hepatic metabolism by cytochrome P450s for clearance from the circulation and for pharmacological inactivation. Conversely, some drugs have to be converted in the body to their pharmacologically active metabolites by P450s. Many promising lead compounds are terminated in the development phase due to their interaction with one or more P450s. One of the greatest problems in drug discovery is the prediction of the role of cytochrome P450s on the metabolism or modification of drug leads. Early detection of metabolic problems associated with a chemical lead series is of paramount importance for the pharmaceutical industry. Obtaining crystal structures of the main human drug metabolising cytochrome P450s would be highly valuable for drug design, as this would provide detailed information on how P450 enzymes recognize drug molecules and the mode of drug binding. This in turn would allow drug companies to develop strategies to modify metabolic clearance and decrease the attrition rates of compounds in development.

The major human CYP450 isoforms involved in drug metabolism are CYP1A2, CYP2C9, CYP2C19, CYP2D6 and CYP3A4. The level of sequence identity between these family members ranges from about 20-80%, with much of the variability within the residues involved in substrate recognition. CYP450 enzymes are also present in bacteria and much of the understanding of substrate recognition is derived from crystal structures obtained of bacterial CYP450 enzymes.

It is well-known in the art of protein chemistry, that crystallising a protein is a chancy and difficult process without any clear expectation of success. It is now evident that protein crystallisation is the main hurdle in protein structure determination. For this reason, protein crystallisation has become a research subject in and of itself, and is not simply an extension of the protein crystallographer's laboratory. There are many references which describe the difficulties associated with growing protein crystals. For example, Kierzek, A. M. and Zielenkiewicz, P., (2001), Biophysical Chemistry, 91,1-20, Models of protein crystal growth, and Wiencek, J. M. (1999) Annu. Rev. Biomed. Eng., 1,505-534, New Strategies for crystal growth.

It is commonly held that crystallisation of protein molecules from solution is the major obstacle in the process of determining protein structures. The reasons for this are many; proteins are complex molecules, and the delicate balance involving specific and non-specific interactions with other protein molecules and small molecules in solution, is difficult to predict.

Each protein crystallises under a unique set of conditions, which cannot be predicted in advance. Simply supersaturating the protein to bring it out of solution may not work, the result would, in most cases, be an amorphous precipitate. Many precipitating agents are used, common ones are different salts, and polyethylene glycols, but others are known. In addition, additives such as metals and detergents can be added to modulate the behaviour of the protein in solution. Many kits are available (e.g. from Hampton Research), which attempt to cover as many parameters in crystallisation space as possible, but in many cases these are just a starting point to optimise crystalline precipitates and crystals which are unsuitable for diffraction analysis. Successful crystallisation is aided by a knowledge of the proteins behaviour in terms of solubility, dependence on metal ions for correct folding or activity, interactions with other molecules and any other information that is available. Even so, crystallisation of proteins is often regarded as a time-consuming process, whereby subsequent experiments build on observations of past trials.

In cases where protein crystals are obtained, these are not necessarily always suitable for diffraction analysis; they may be limited in resolution, and it may subsequently be difficult to improve them to the point at which they will diffract to the resolution required for analysis. Limited resolution in a crystal can be due to several things. It may be due to intrinsic mobility of the protein within the crystal, which can be difficult to overcome, even with other crystal forms. It may be due to high solvent content within the crystal, which consequently results in weak scattering. Alternatively, it could be due to defects within the crystal lattice which mean that the diffracted x-rays will not be completely in phase from unit to unit within the lattice. Any one of these or a combination of these could mean that the crystals are not suitable for structure determination.

Some proteins never crystallise, and after a reasonable attempt it is necessary to examine the protein itself and consider whether it is possible to make individual domains, different N or C-terminal truncations, or point mutations. It is often hard to predict how a protein could be re-engineered in such a manner as to improve crystallisability. Our understanding of crystallisation mechanisms are still incomplete and the factors of protein structure which are involved in crystallisation are poorly understood.

As of 2000, eight cytochrome P450 structures had been solved by X-ray crystallography and were available in the public domain. Six structures correspond to bacterial cytochrome P450s: P450cam (CYP101 Poulos et al., 1985, J. Biol. Chem., 260, 16122), the haemprotein domain of P450BM3 (CYP102, Ravichandran et al., 1993, Science, 261, 731), P450terp (CYP108, Hasemann et al., 1994, J. Mol. Biol. 236, 1169), P450eryF (CYP107A1, Cupp-Vickery and Poulos, 1995, Nature Struct. Biol. 2, 144), P450 14α-sterol demethylase (CYP51, Podust et al., 2001, Proc. Natl. Acad. Sci. USA, 98, 3068) and the crystal structure of a thermophilic cytochrome P450 (CYP119) from Archaeon sulfolobus solfataricus was solved (Yano et al., 2000, J. Biol. Chem. 275, 31086). The structure of cytochrome P450nor was obtained from the denitrifying fungus Fusarium oxysporum (Shimizu et al. 2000, J. lnorg. Biochem. 81, 191). The eighth structure is that of the rabbit 2C5 isoform, the first structure of a mammalian cytochrome P450 (Williams et al. 2000, Mol. Cell. 5, 121). Our understanding of the structural variability of these enzymes has been advanced further in recent years, with the addition of nine non-mammalian crystal structures; CYP152A1 from Bacillus subtilis (Lee et al, 2003, J. Biol. Chem, 278, 9761), CYP165B1 from Amycolatopsis orientalis (P450 OxyB) (Zerbe et al, 2002, J. Biol. Chem, 277, 47476), CYP165C1 from Amycolatopsis orientalis (P450 OxyC) (Pylypenko et al, 2002, J. Biol. Chem, 278, 46727), CYP167A1 from Polyangium cellulosum (P450 EpoK) (Nagano et al, 2003, J. Biol. Chem. 278, 44886), CYP 1 19A2 from sulfolobus tokodaii (CYP119) (Yano etal,2000, J. Biol. Chem. 31086), CYP175A1 from Thermus thermophilus strain HB27 (Yano etal,2003, J. Biol. Chem. 278, 608), CYP121 from mycobacterium tuberculosis (Leys et al, 2003, J. Biol. Chem. 278, 5141), CYP154C1 from streptomyces coelicolor (Podust et al, 2003, J. Biol. Chem. 278, 12214), and CYP154A1 from streptomyces coelicolor (Podust et al, 2004, Protein Sci., 13, 255).

In addition, another three mammalian structures have been solved, namely the rabbit CYP2B4 in the absence (Scott etal, 2003, P.N.A.S., 100, 13196) and presence of compound (Scott et al, 2004, J. Biol. Chem, April 2004; 10.1074/jbc.M403349200), the human CYP2C8 (Schoch et al, 2003, Biochemistry, 279, 9497) in the absence of compound, and human CYP2C9 has also been solved in the absence and presence of the substrate S-warfarin (Williams et al 2003, Nature, 424, 464). Two compound complexes with rabbit CYP2C5 with diclofenac and a sulfaphenazole derivative have been also been solved (Wester et al, 2003, Biochemistry, 42, 9335; Wester et al, 2003, Biochemistry, 42, 6370).

The reason why the mammalian cytochrome P450s have been particularly difficult to crystallise, compared to their bacterial counterparts, resides in the nature of these proteins. The bacterial cytochrome P450s are soluble whereas the mammalian P450s are membrane-associated proteins. Thus, structural studies on mammalian cytochrome P450s may use the combination of heterologous expression systems that allow expression of single cytochrome P450s at high concentration with modification of their sequences to improve the solubility and the behaviour of these proteins in solution.

Due to significant sequence differences from both the bacterial proteins and rabbit proteins, to fully understand the role of the human CYP450 enzymes in drug metabolism, the crystal structures of human isoforms are still required.

Ibeanu et al., (1996), J Biol Chem, Vol. 271, 12496-12501 describe the production of modified 2C9 proteins in yeast in which certain residues, including Ser 220 and Pro 221, were altered.

These altered proteins were found to exhibit 2C19-like activity for omeprazole. The proteins retained wild-type N-terminal sequence.

WO 03/035693 describes the crystallisation of a human 2C9 P450 protein molecule and provides an analysis of the protein crystal structure.

### DISCLOSURE OF THE INVENTION

The present invention provides a co-crystal of CYP450 2C9 and warfarin.

In another aspect, the invention relates to the crystal structure of human CYP450 2C9 to which warfarin is bound.

The present invention additionally relates to a method of providing a co-crystal of CYP450 2C9 and a ligand.

In general aspects, the present invention is concerned with the provision of P450 structures and their use in modelling the interaction of molecular structures, e.g. potential pharmaceutical compounds, with this structure.

The above aspects of the invention, both singly and in combination, all contribute to features of the invention which are advantageous.

### DESCRIPTION OF THE DRAWINGS

FIG. 1 sets out Table 1, providing the coordinates of a 2C9 structure and S-warfarin bound thereto.

FIG. 2 illustrates the 2C9 binding pocket containing a haem group and S-warfarin.

FIG. 3 shows the metabolism of S-warfarin by mutants of 2C9trunc. Metabolites of S-warfarin produced by the parental enzyme 2C9trunc and its mutants were quantified as described in the method section, using 100 pmol of enzyme in presence of 100 μM of S-warfarin. Data are presented as the average of the relative peak area for each metabolite measured in three independent experiments.

FIG. 4 shows the metabolism of S-warfarin by mutants of 2C9-FGloop K206E. Metabolites of S-warfarin produced by the parental enzyme 2C9trunc and its mutants were quantified as described in the method section, using 100 pmol of enzyme in presence of 100 μM of S-warfarin. Data are presented as the average of the relative peak area for each metabolite measured in three independent experiments.

FIG. 5 shows the metabolism of diclofenac by mutants of 2C9trunc. The assay was performed using 20 pmol of enzyme in presence of 100 μM of diclofenac. Results are presented as means±standard deviation of three determinations.

FIG. 6 shows the metabolism of diclofenac by mutants of 2C9-FGloop K206E. The assay was performed using 20 pmol of enzyme in presence of 100 μM of diclofenac. Results are presented as means±standard deviation of three determinations.

FIG. 7 shows an alignment of the 2C9FGloop K206E and 2C9trunc sequences with wild-type 2C9.

FIG. 8 sets out Table 2, showing the coordinates of an apo 2C9 structure.

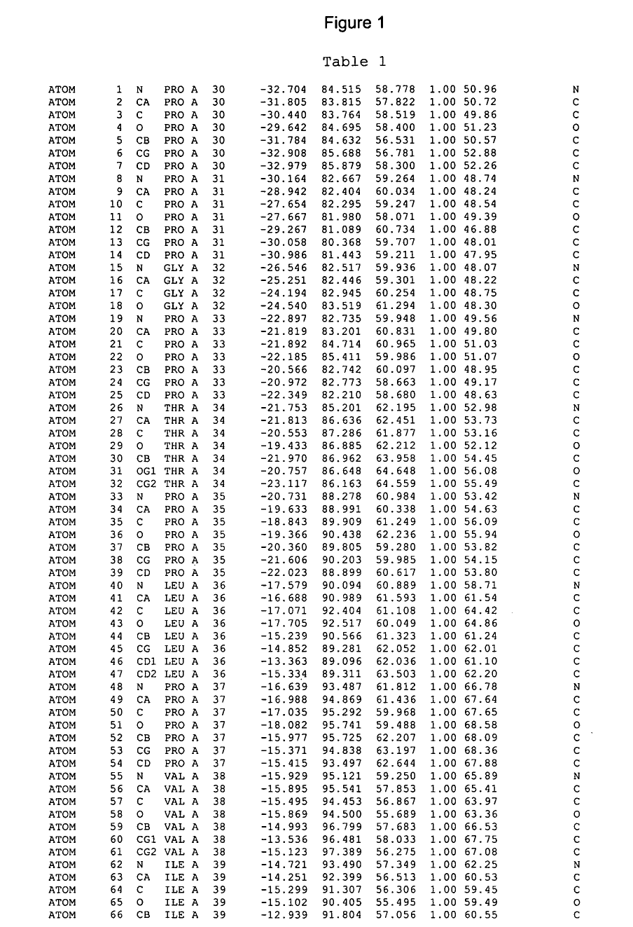

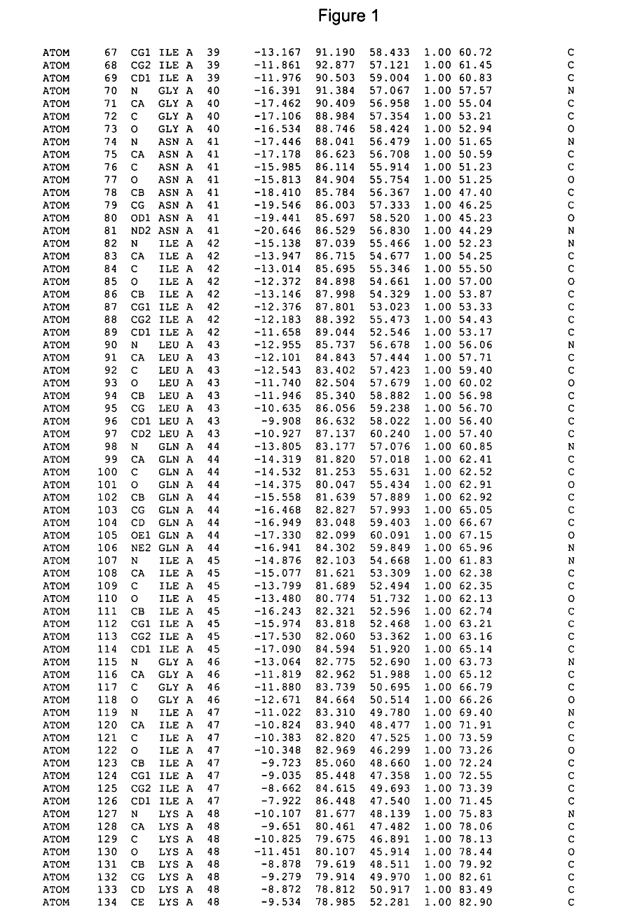

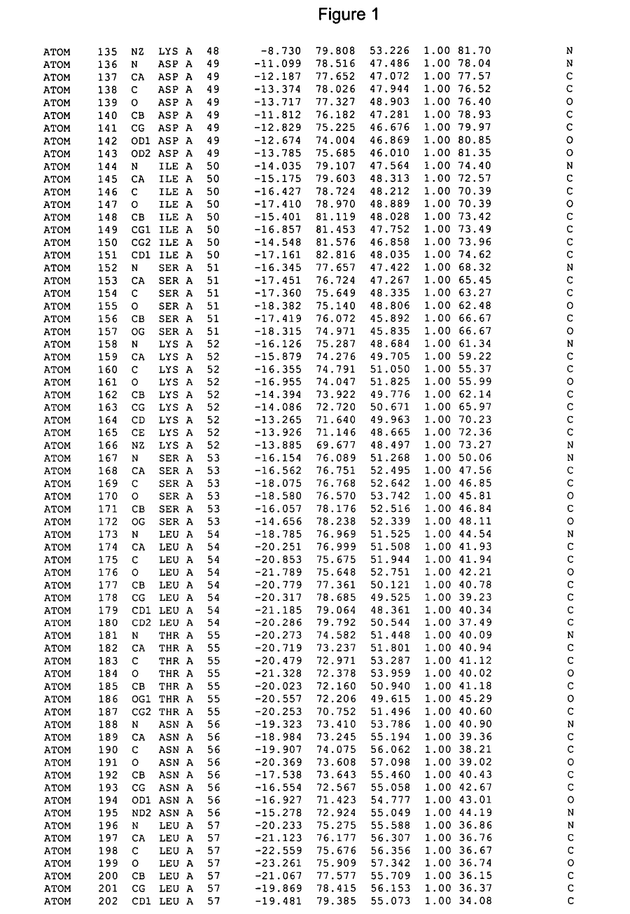

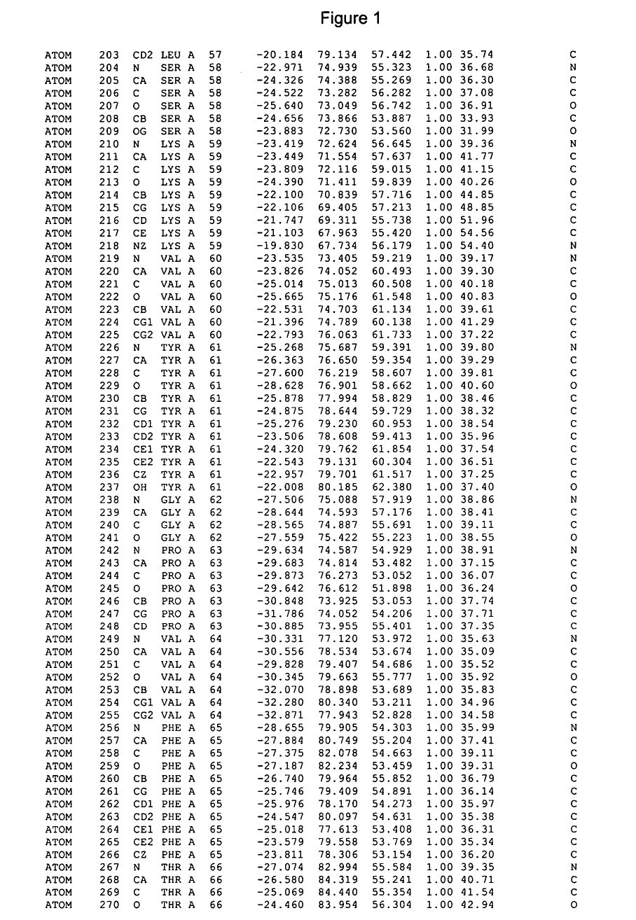

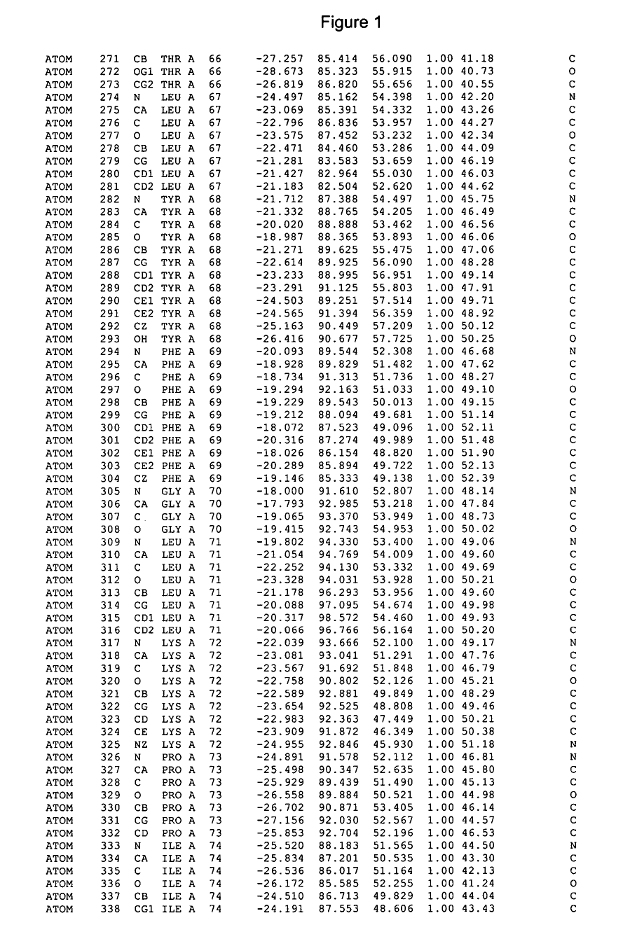

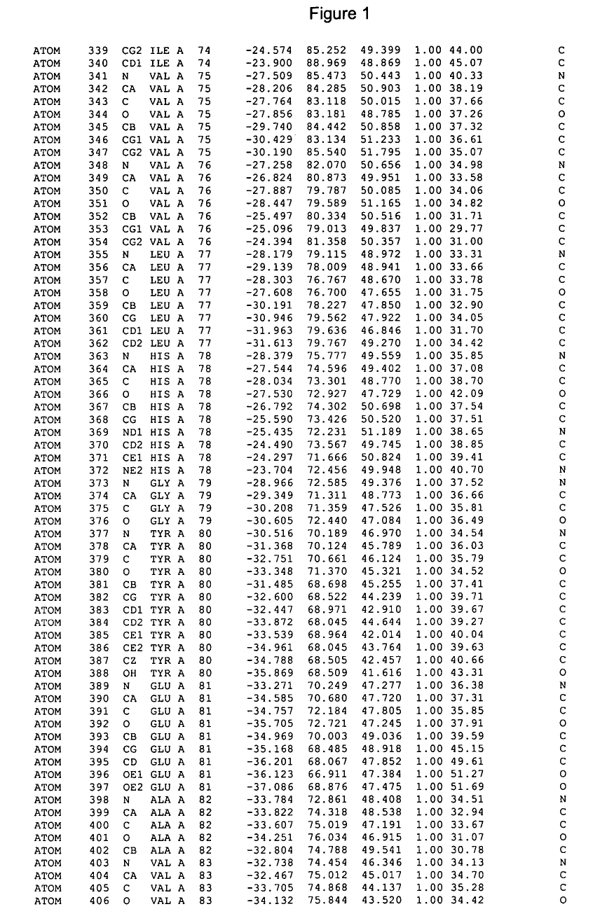

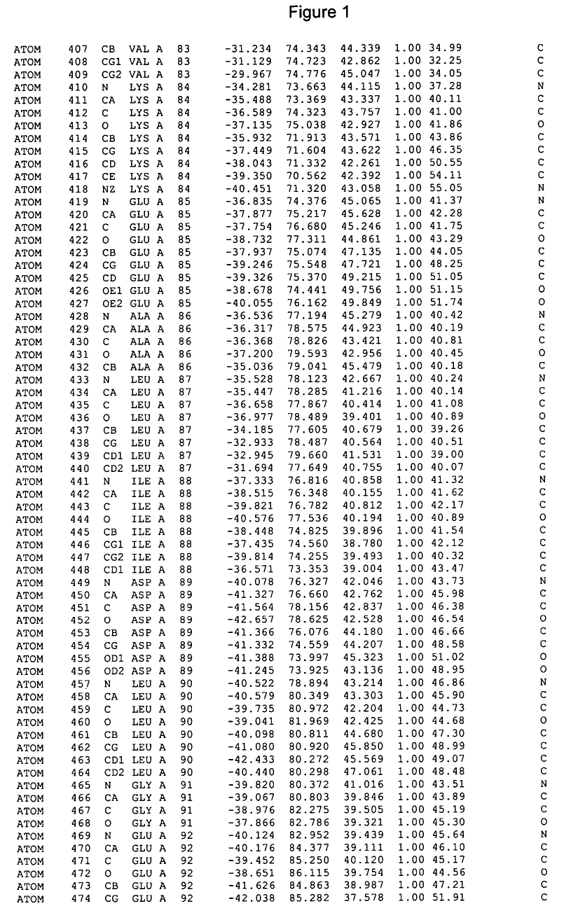

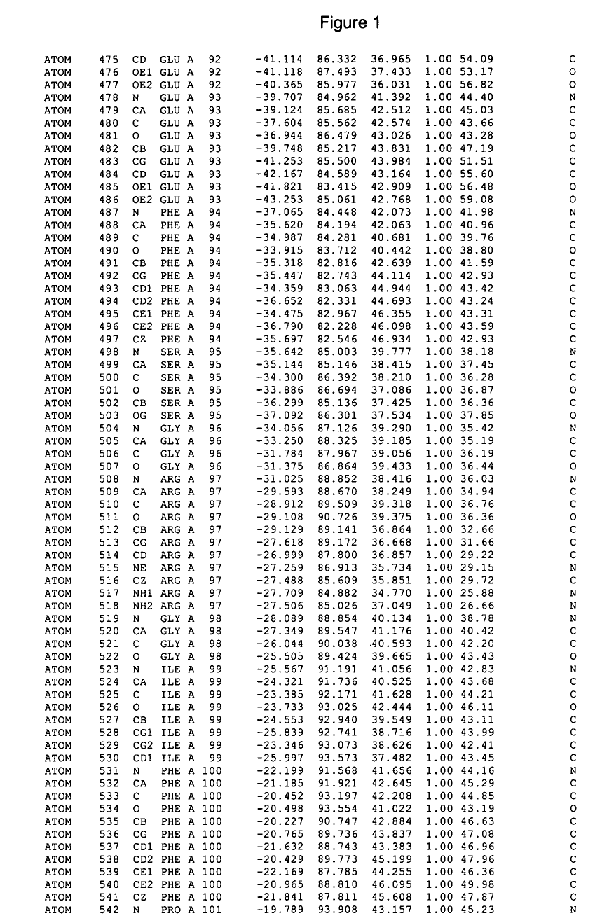

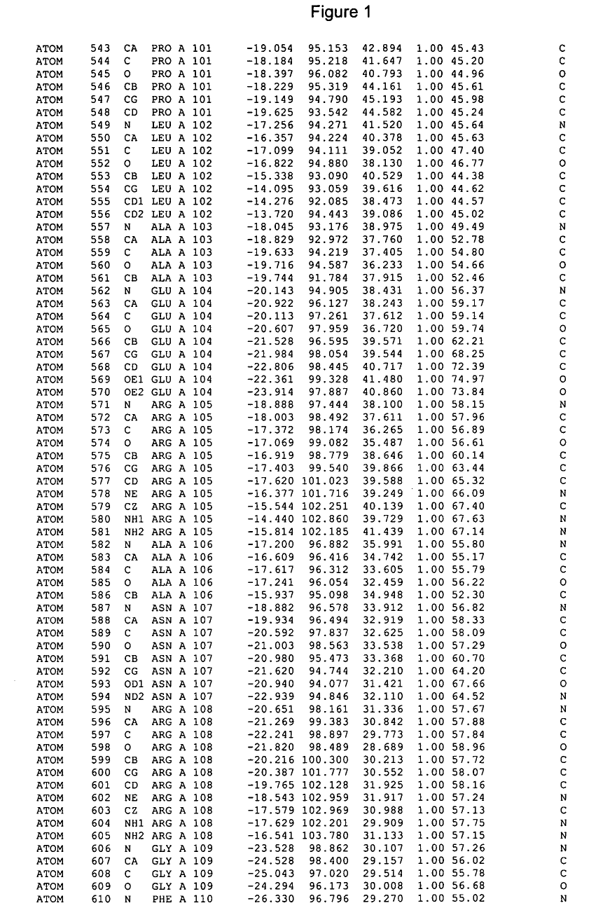

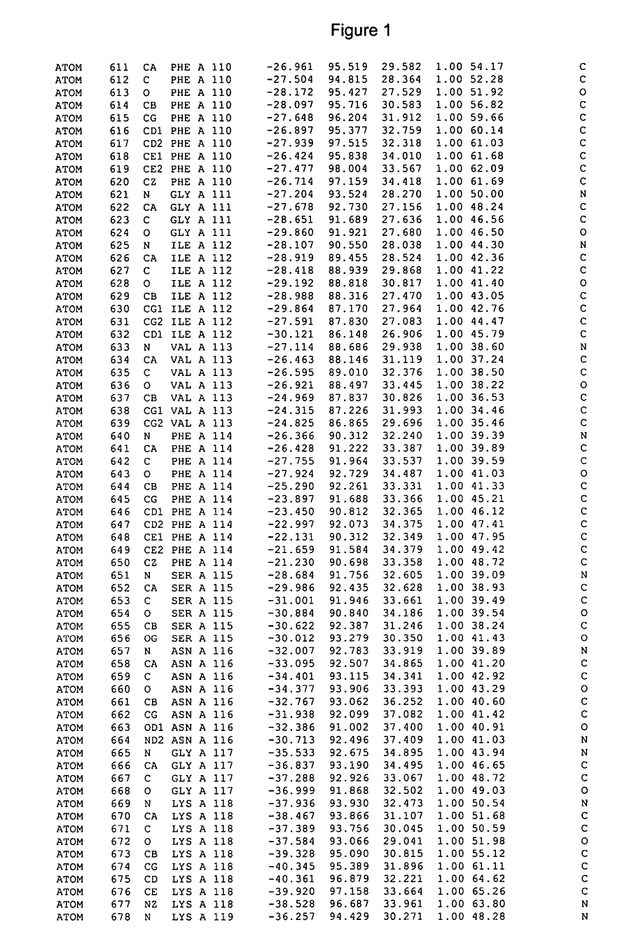

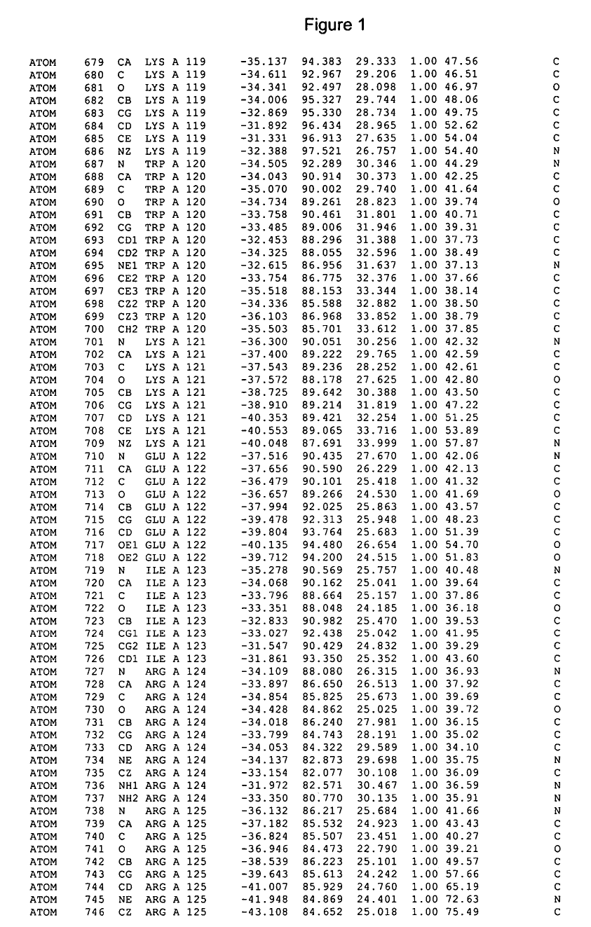

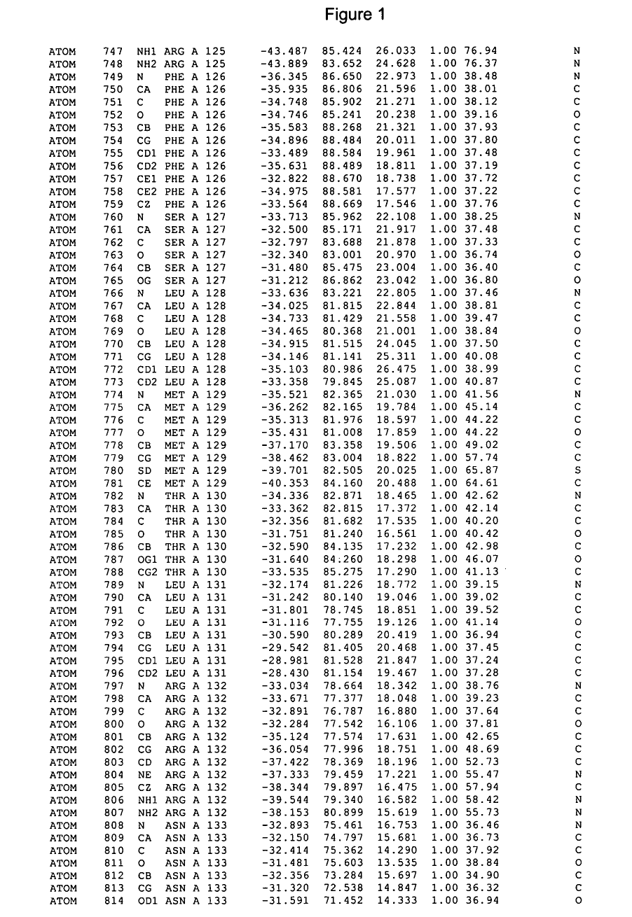

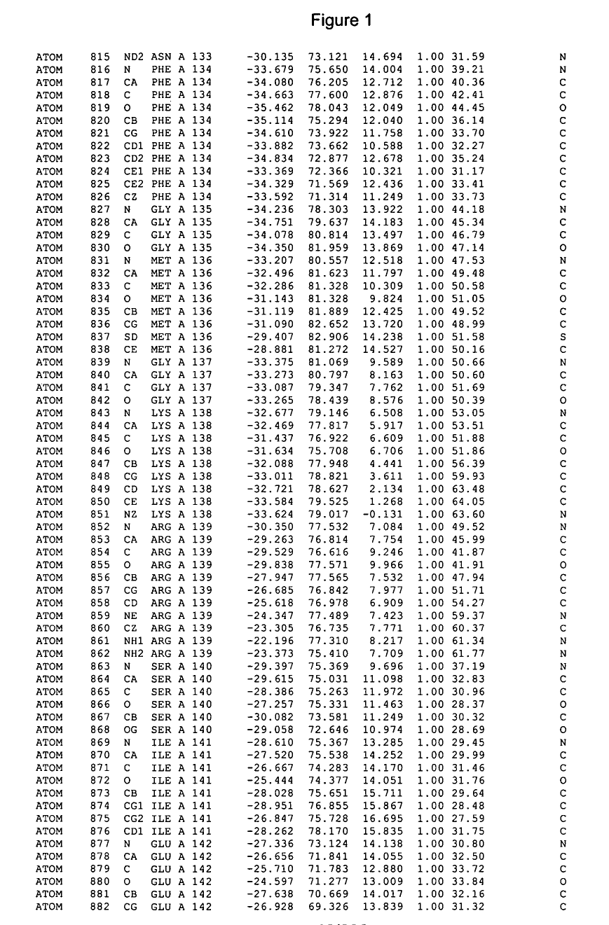

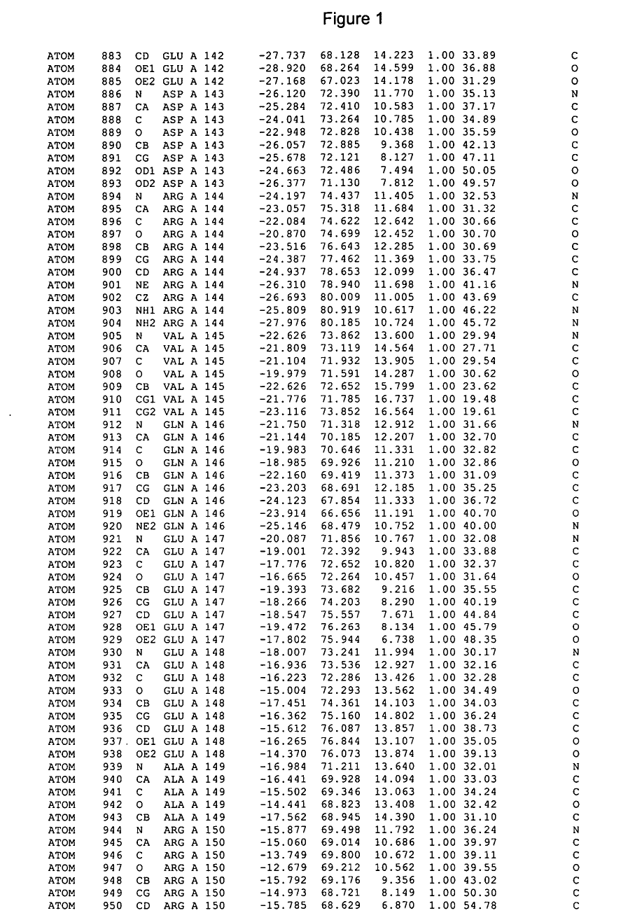

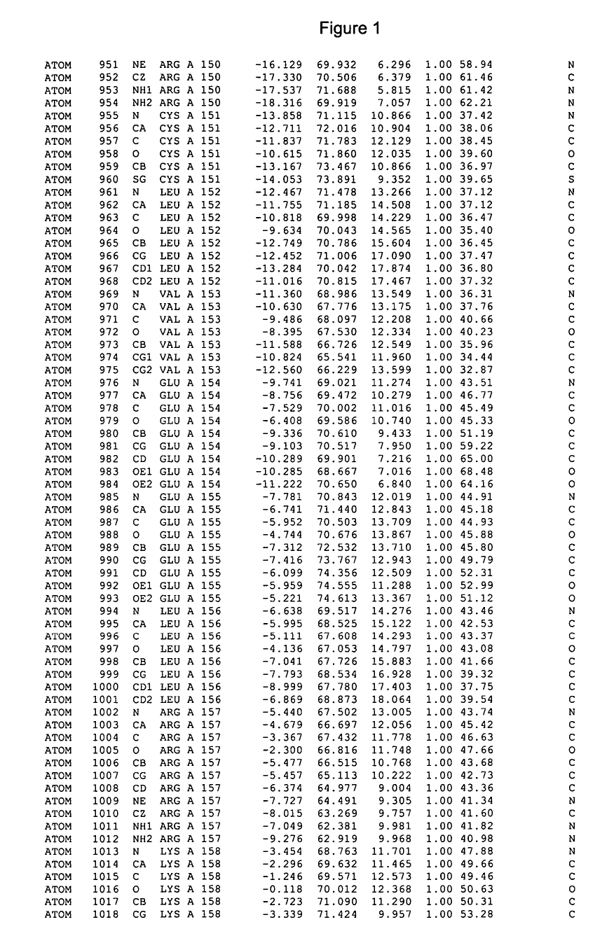

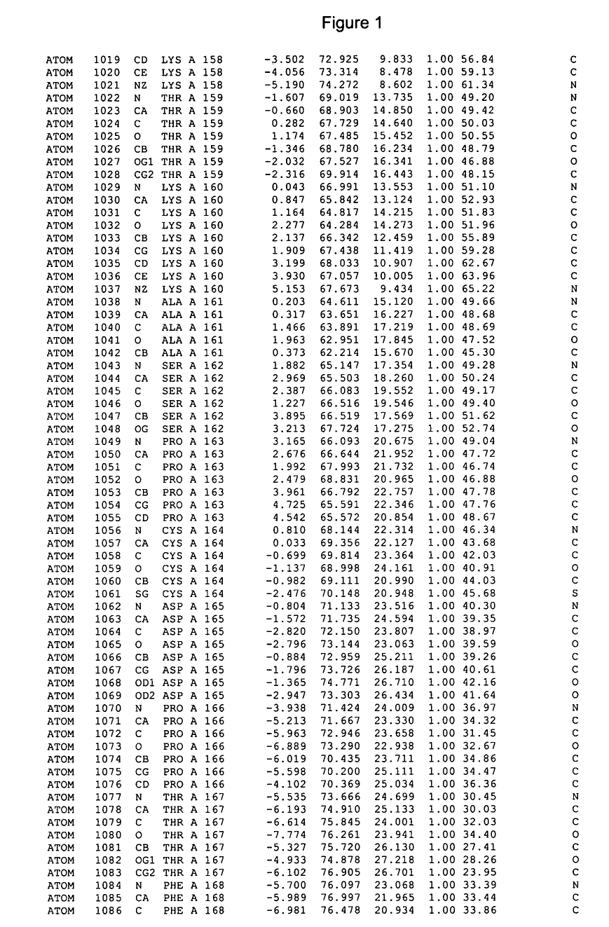

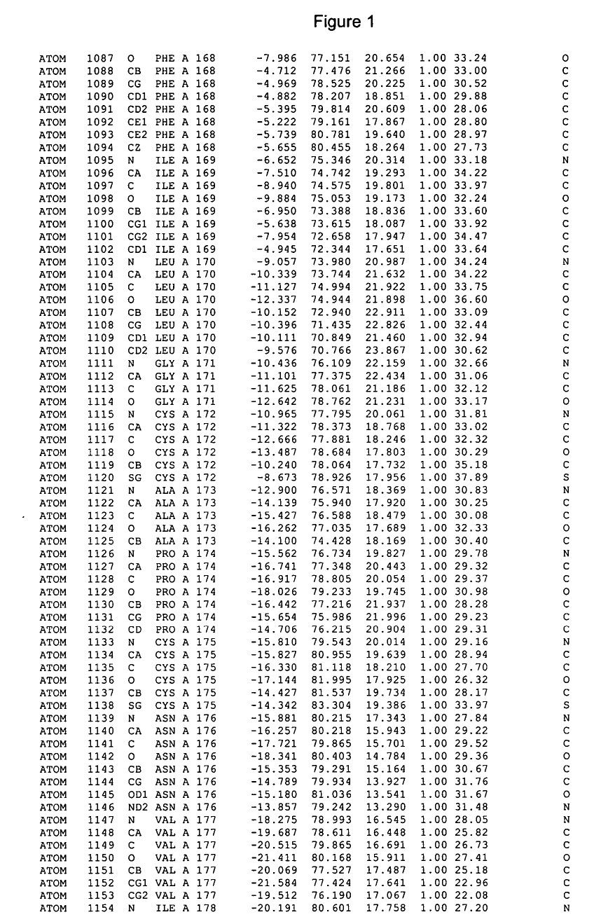

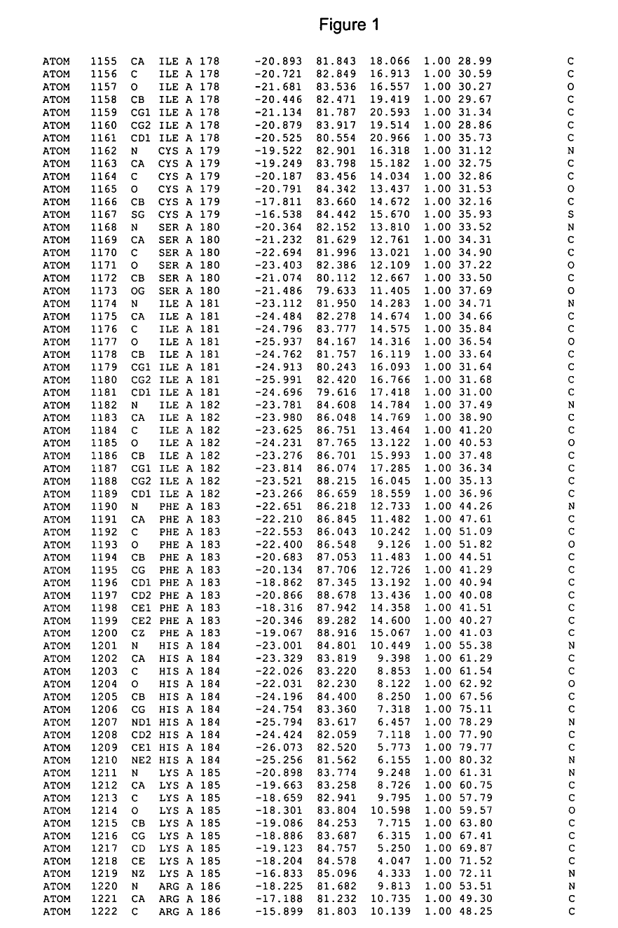

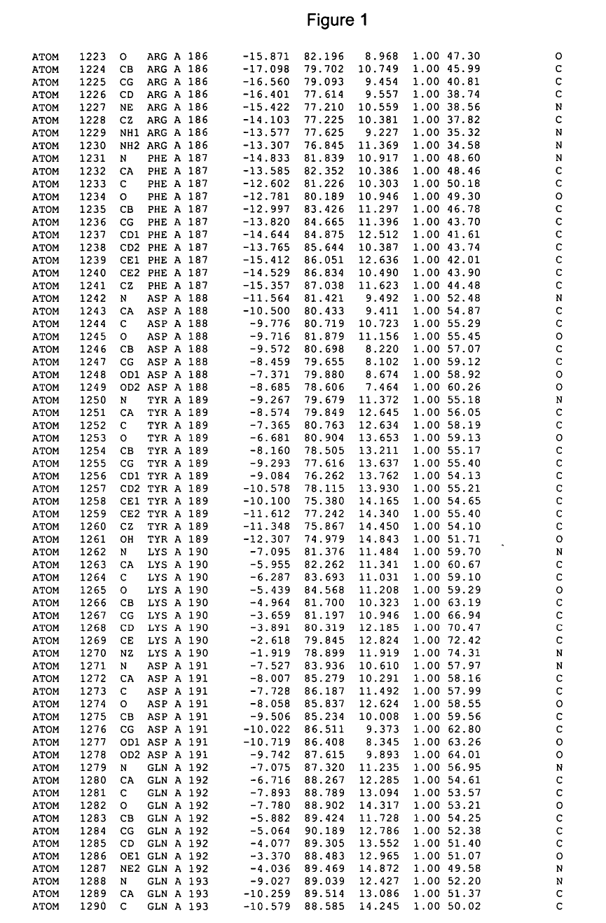

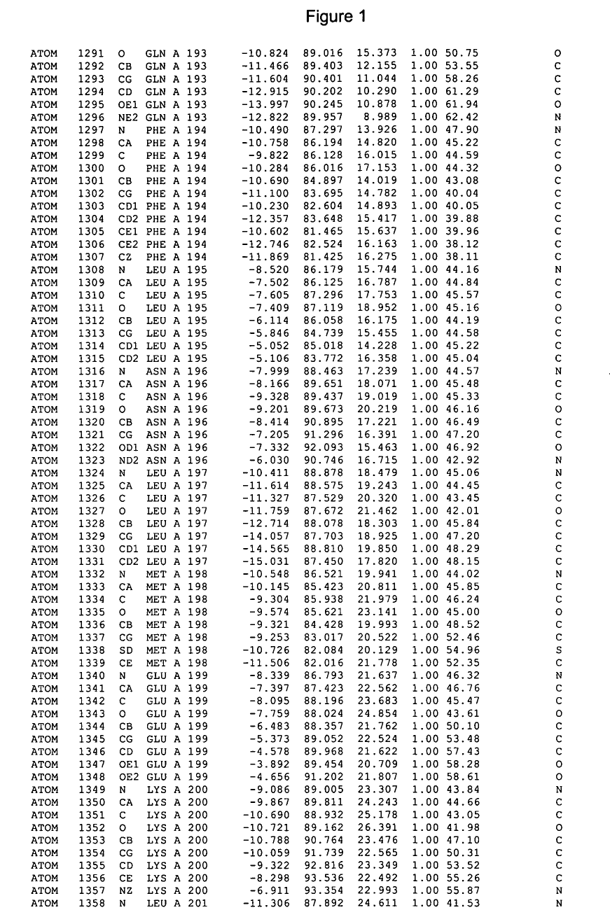

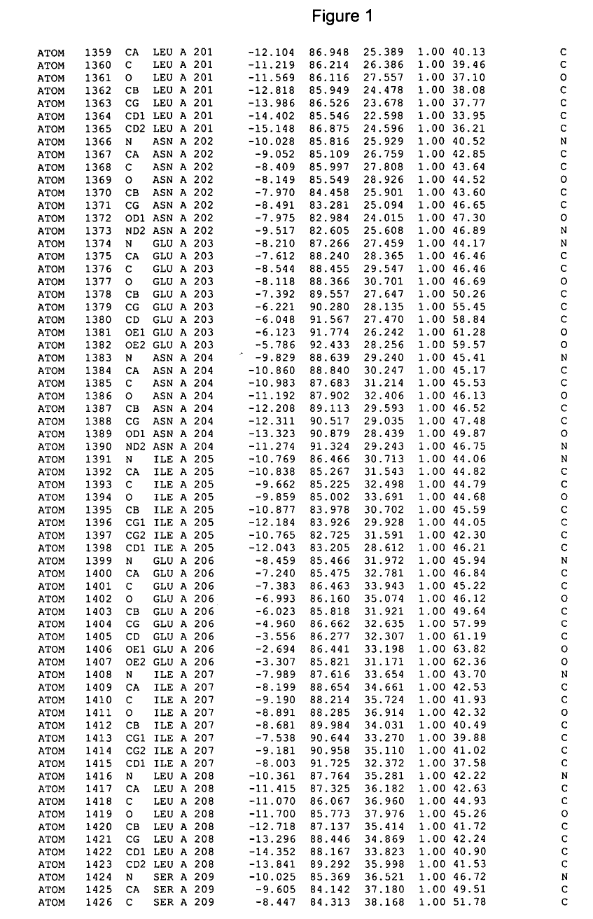

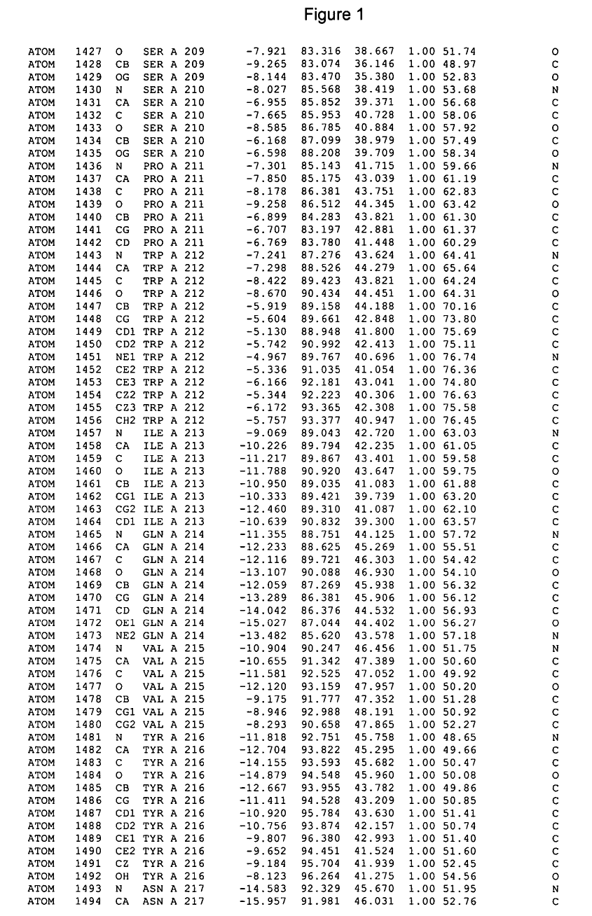

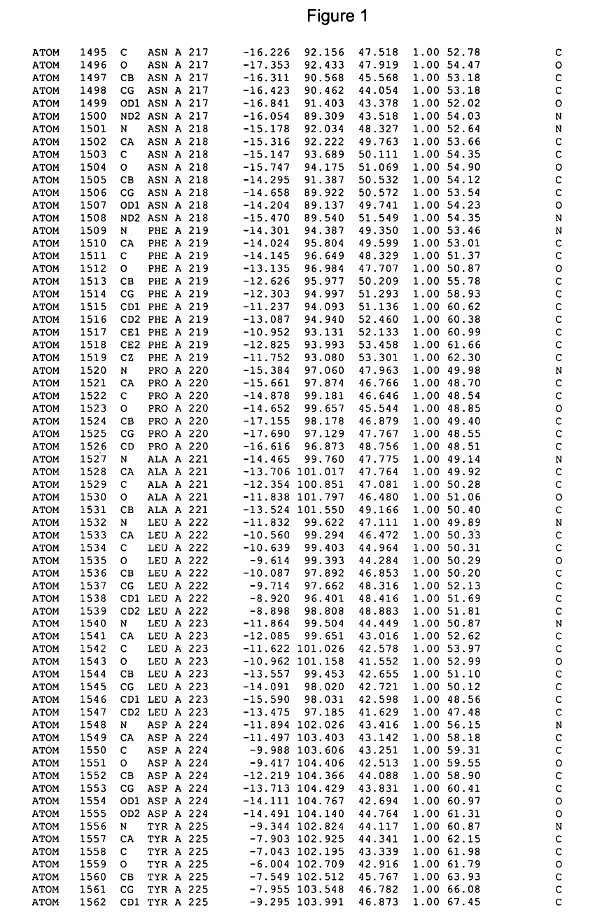

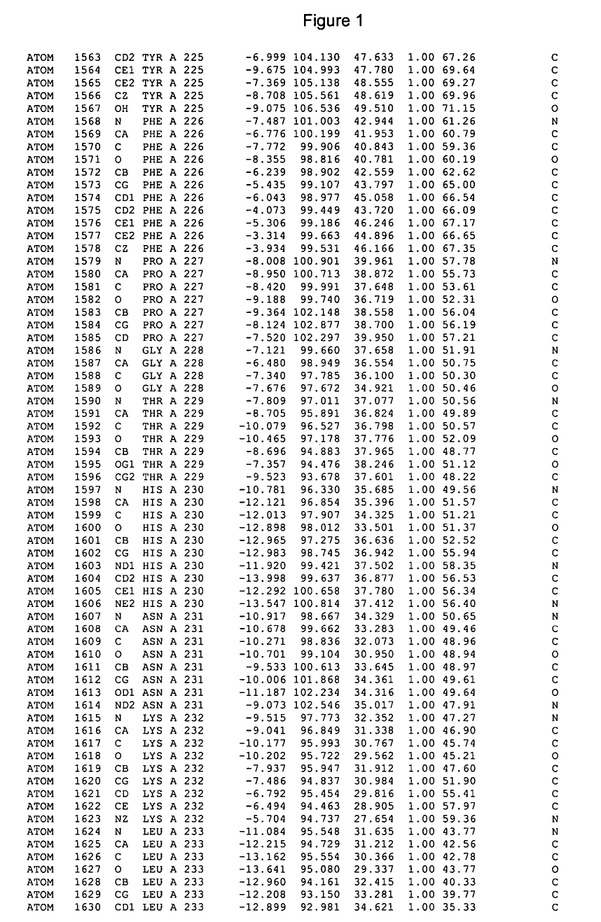

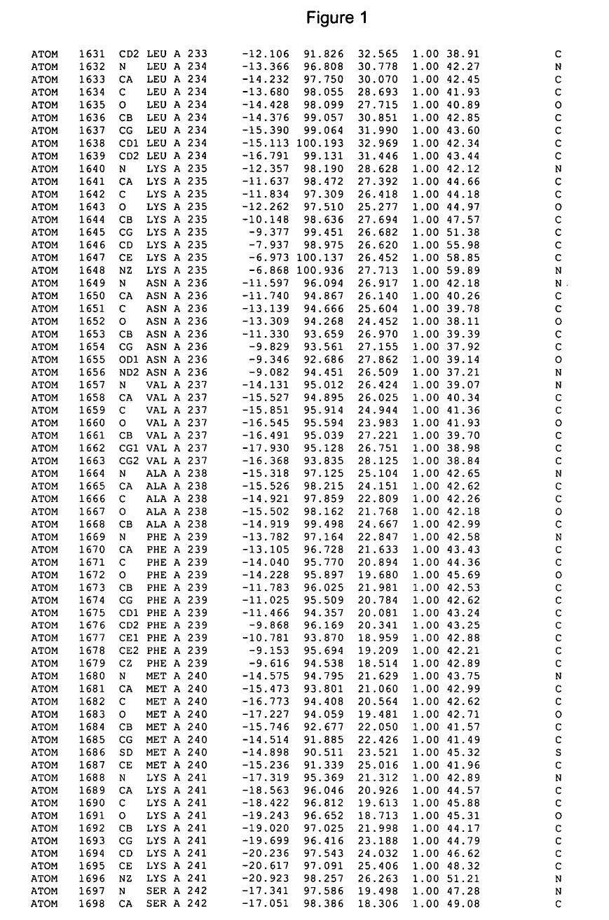

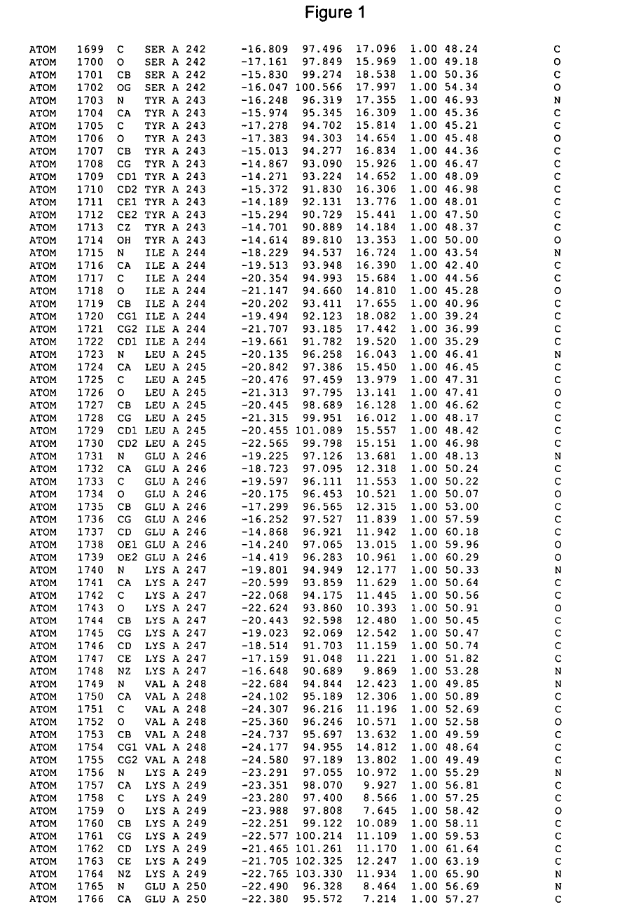

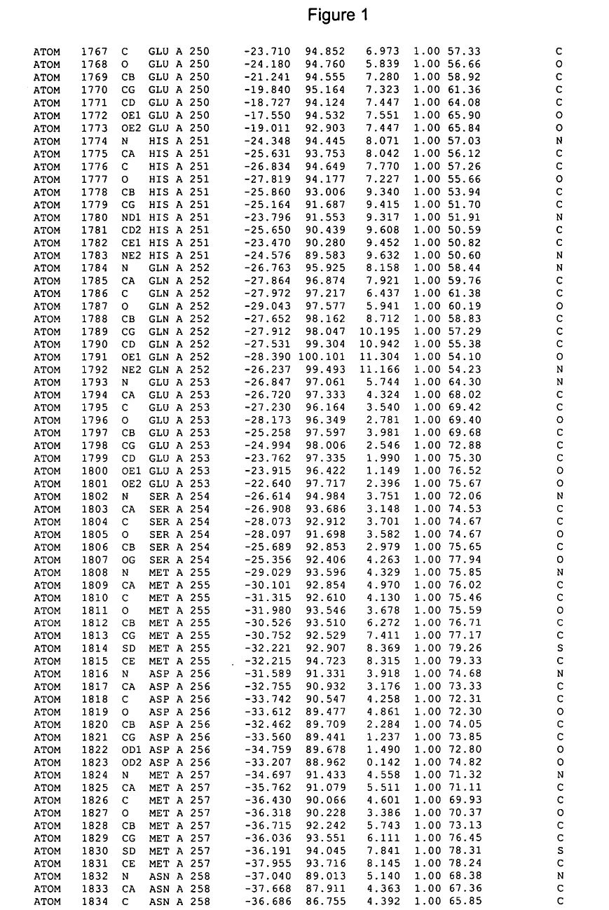

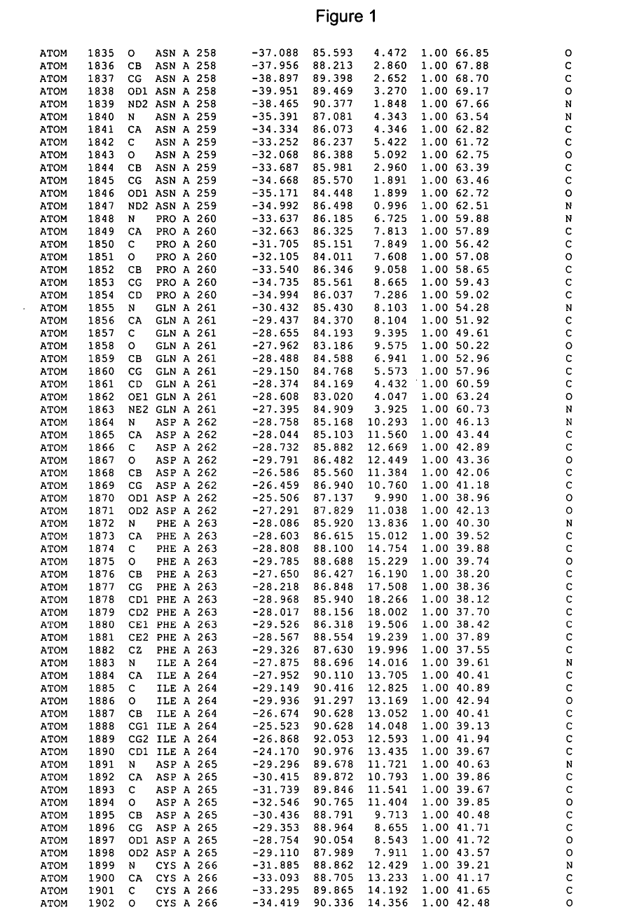

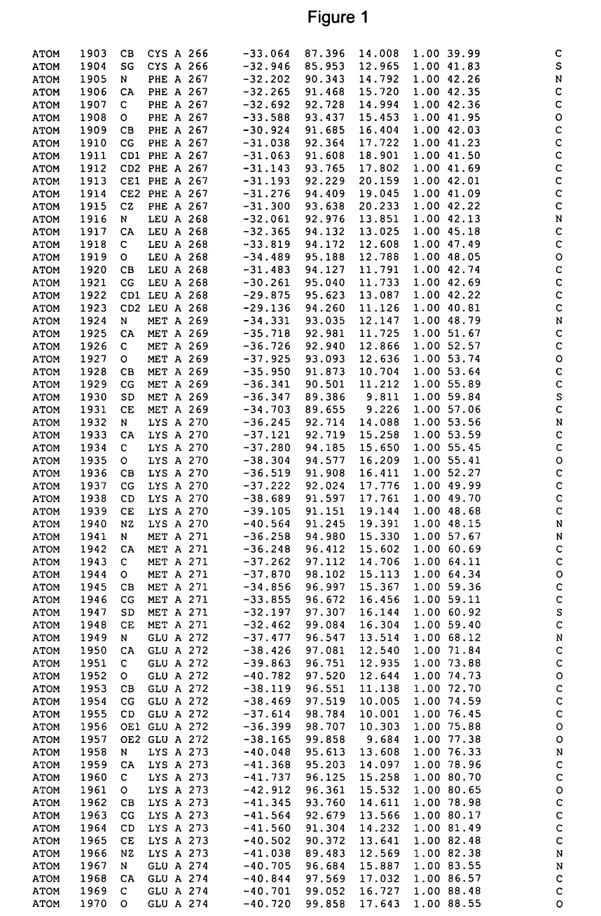

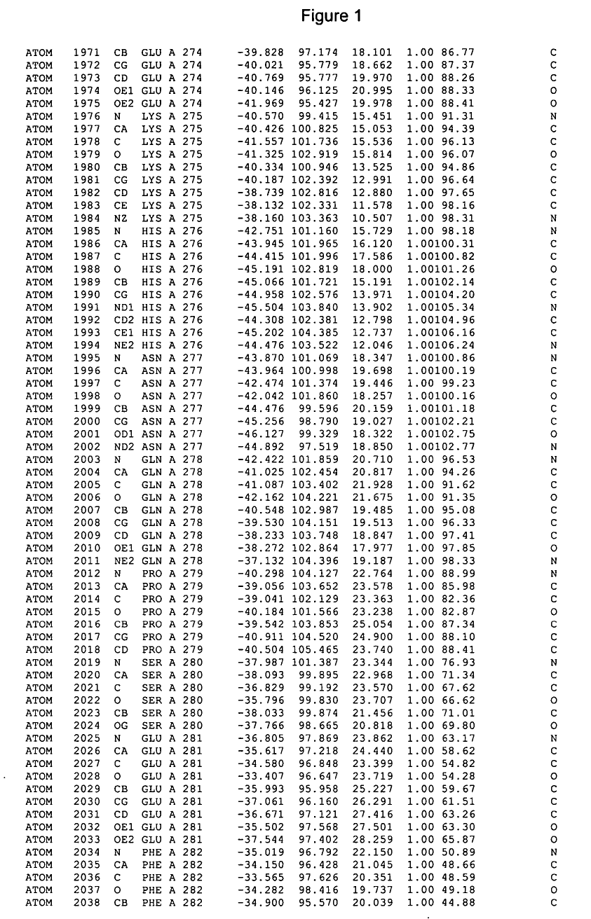

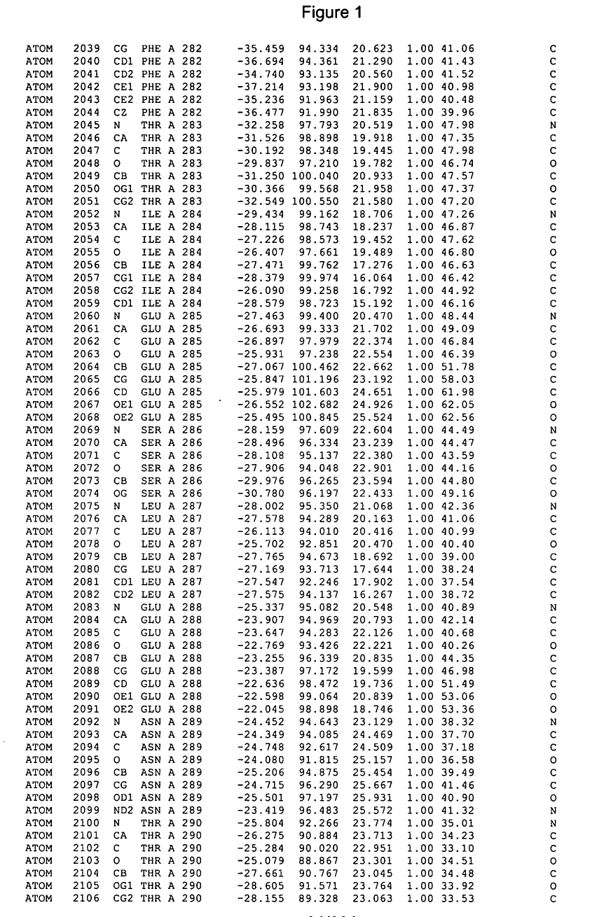

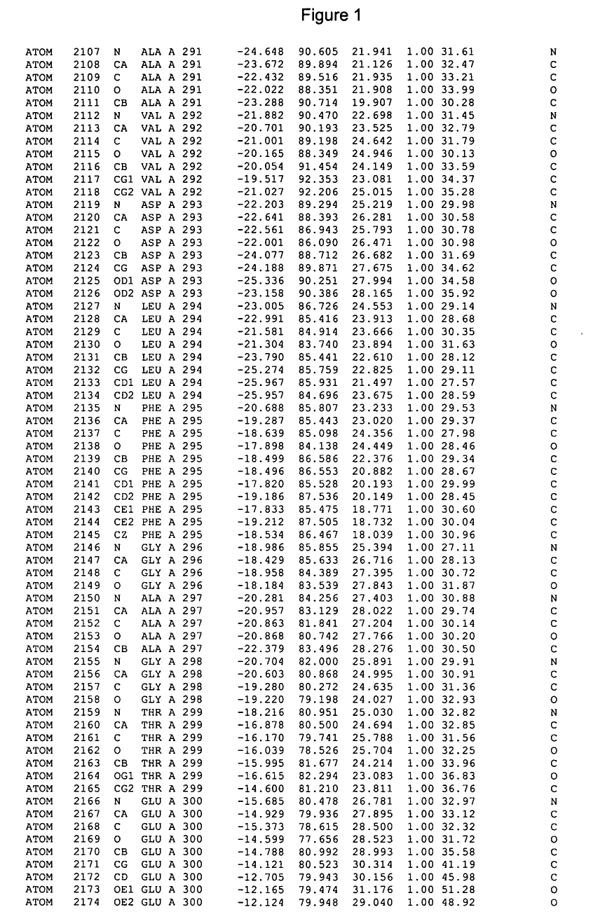

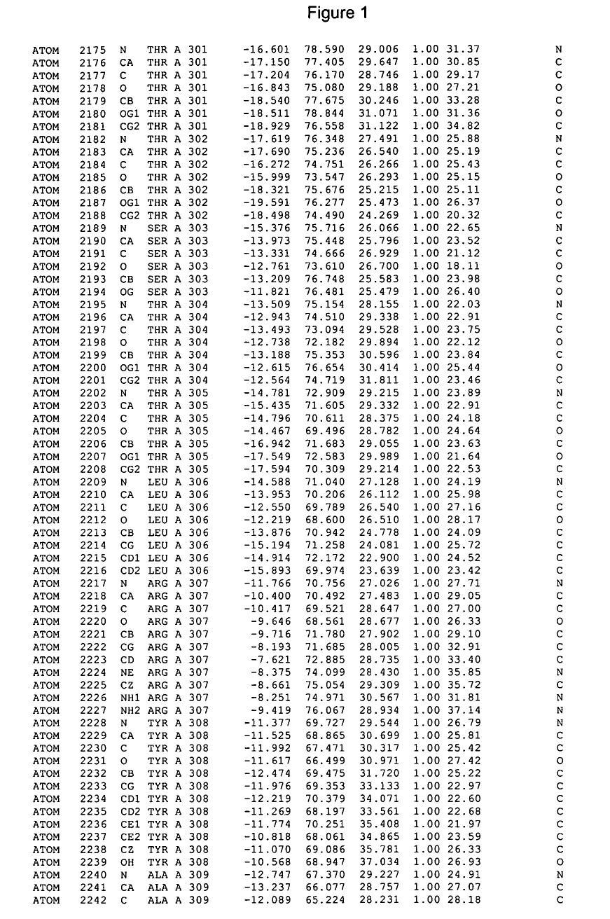

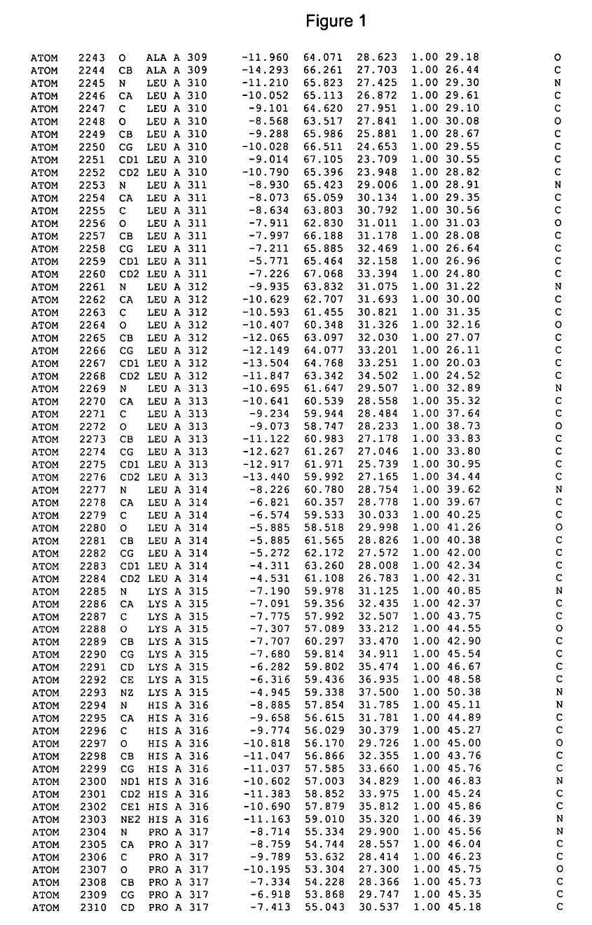

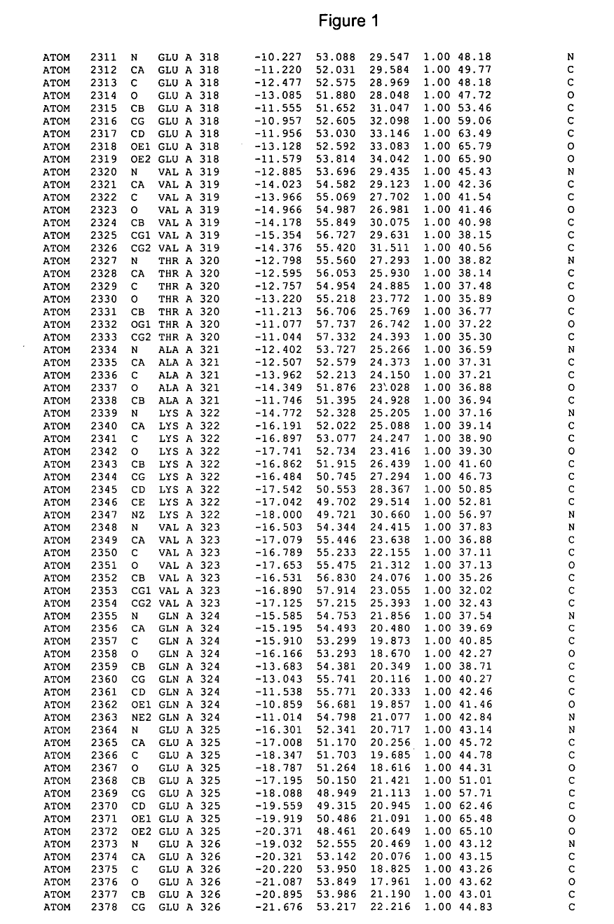

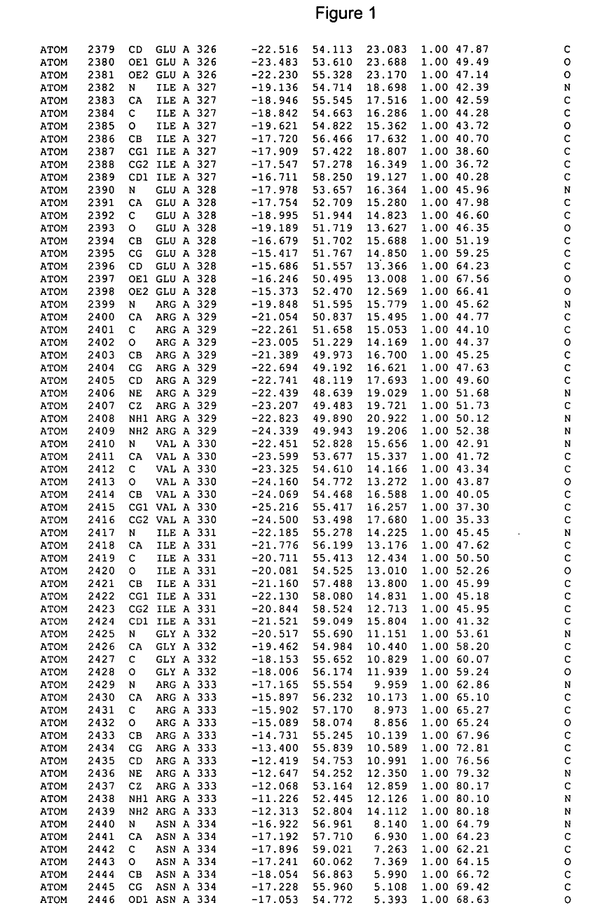

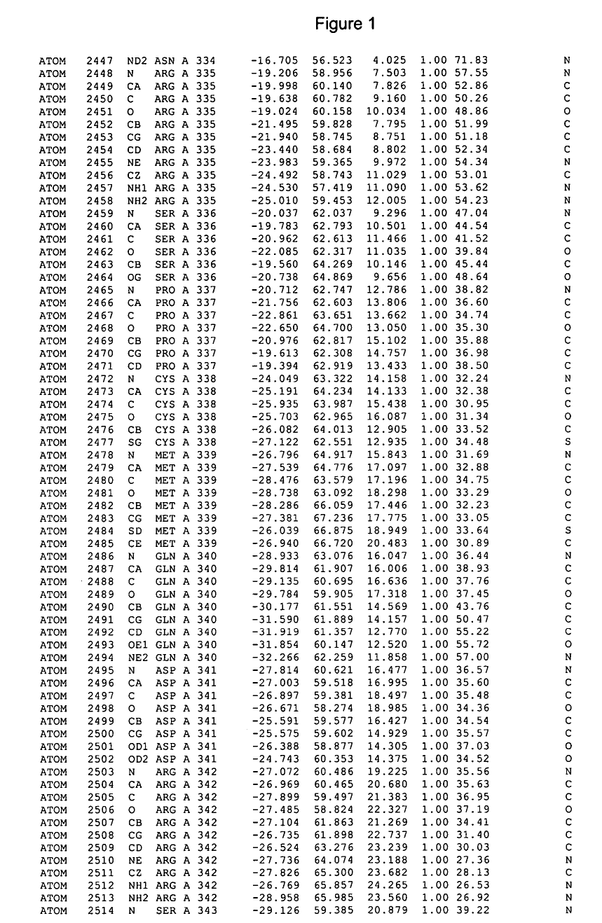

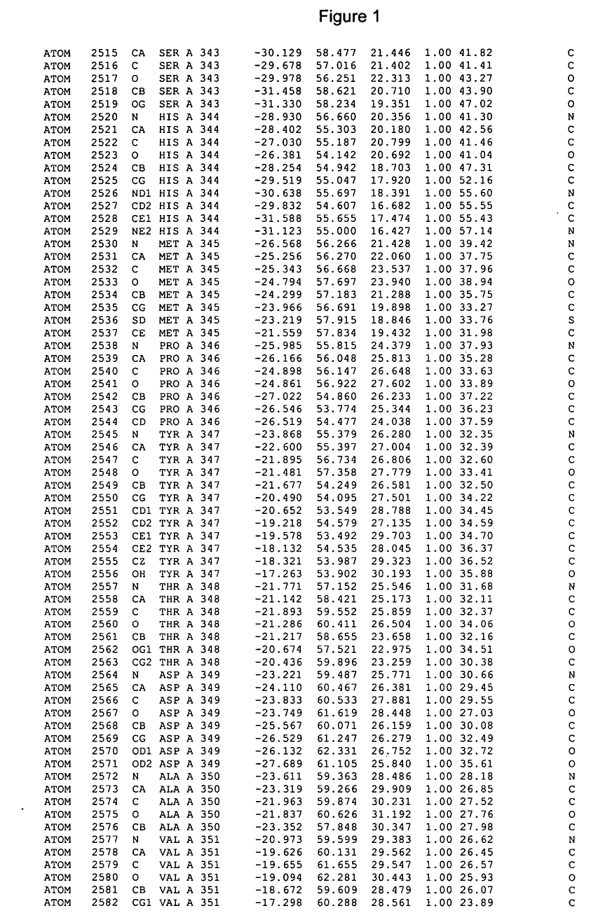

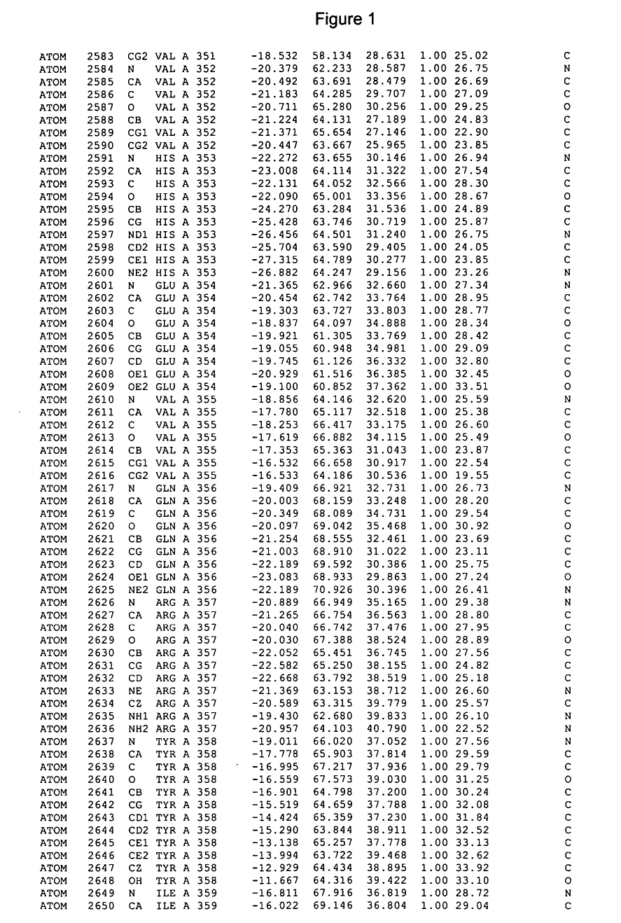

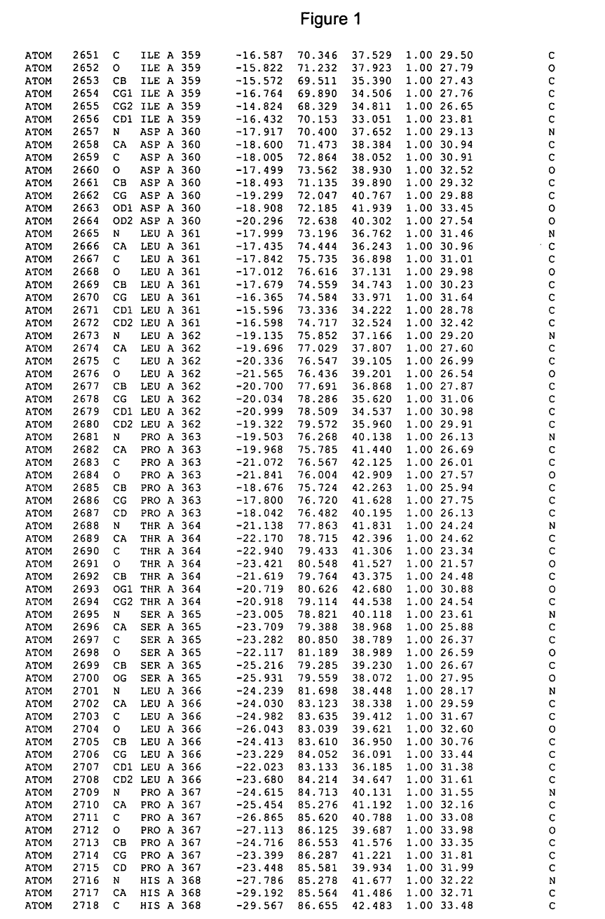

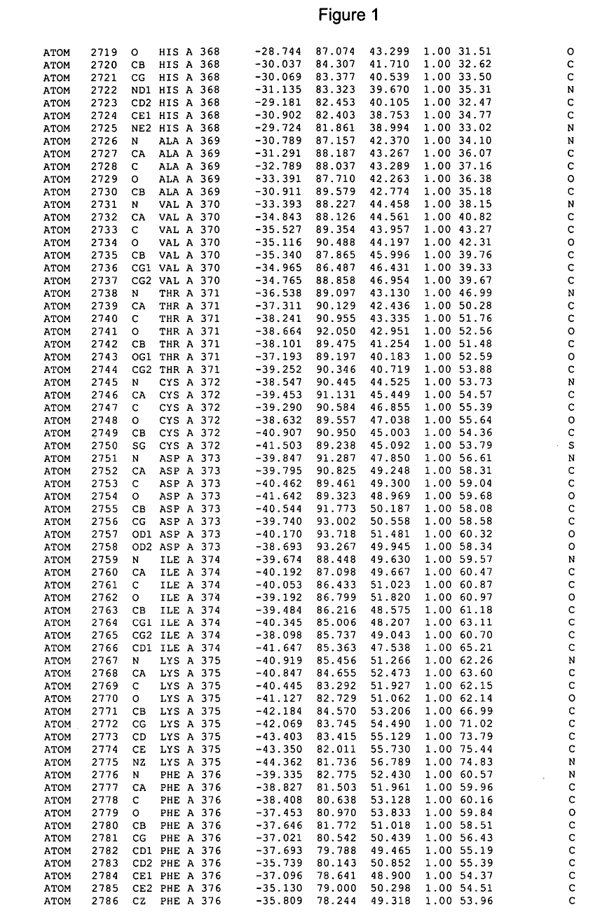

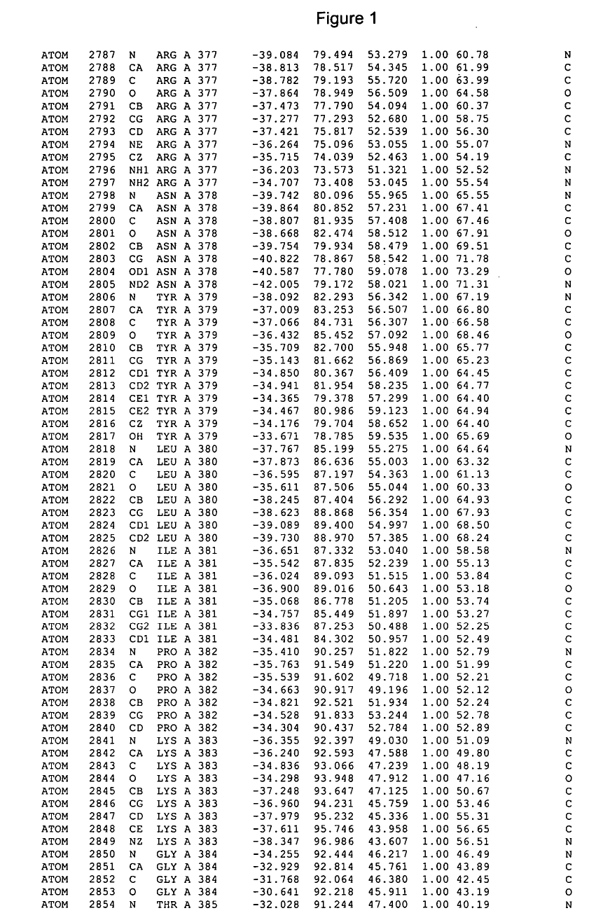

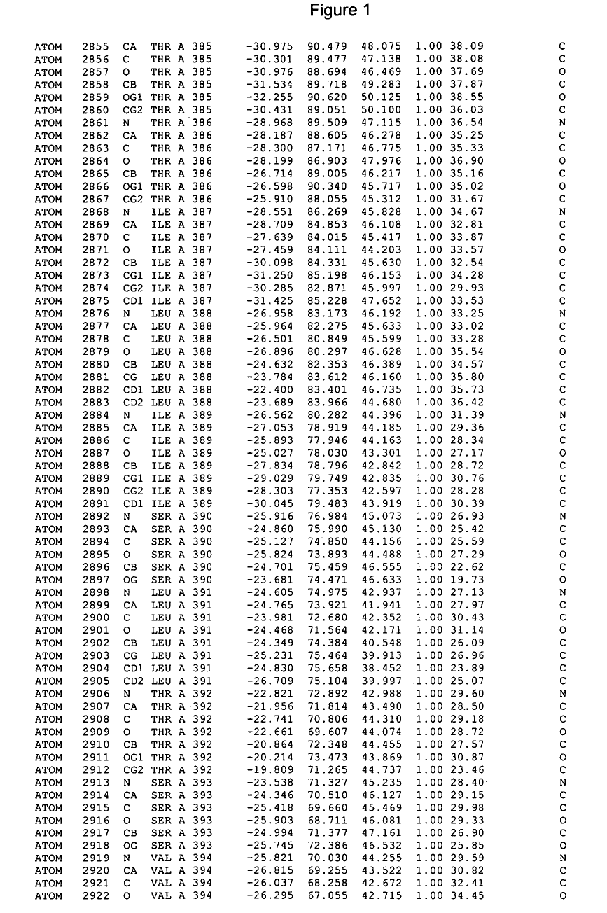

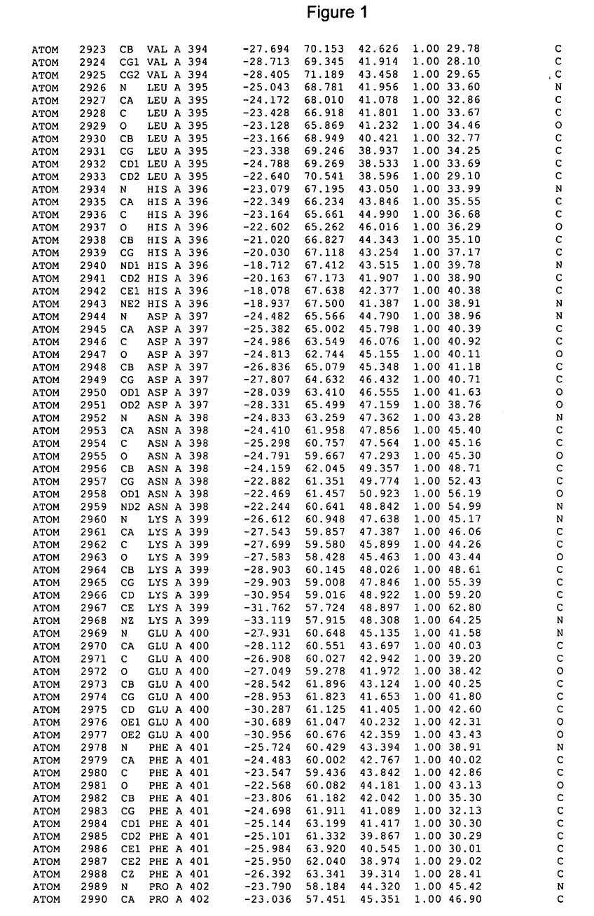

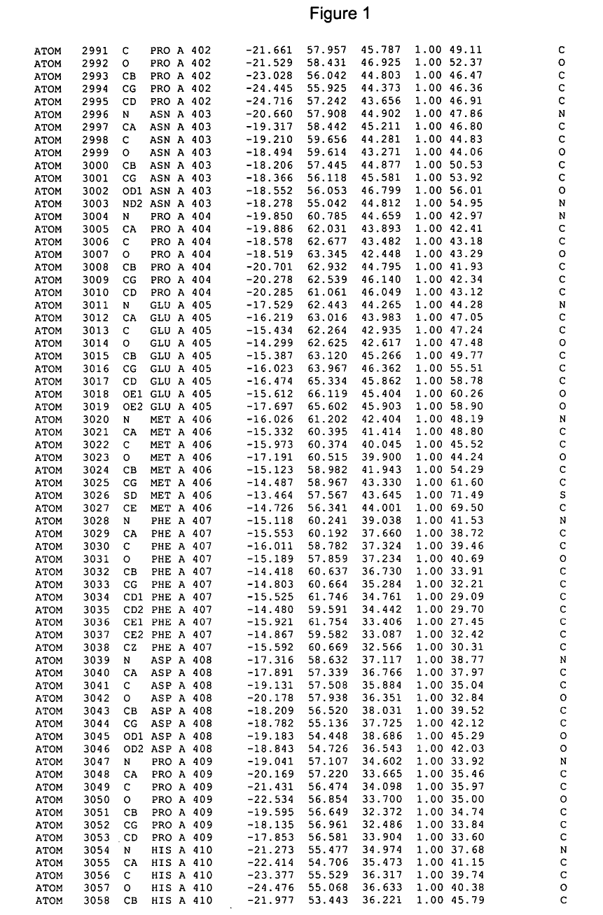

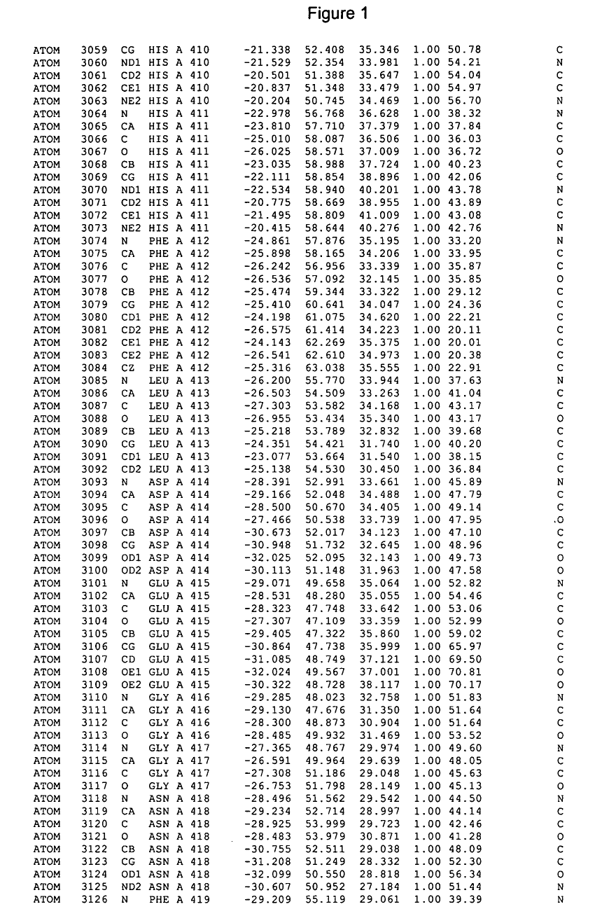

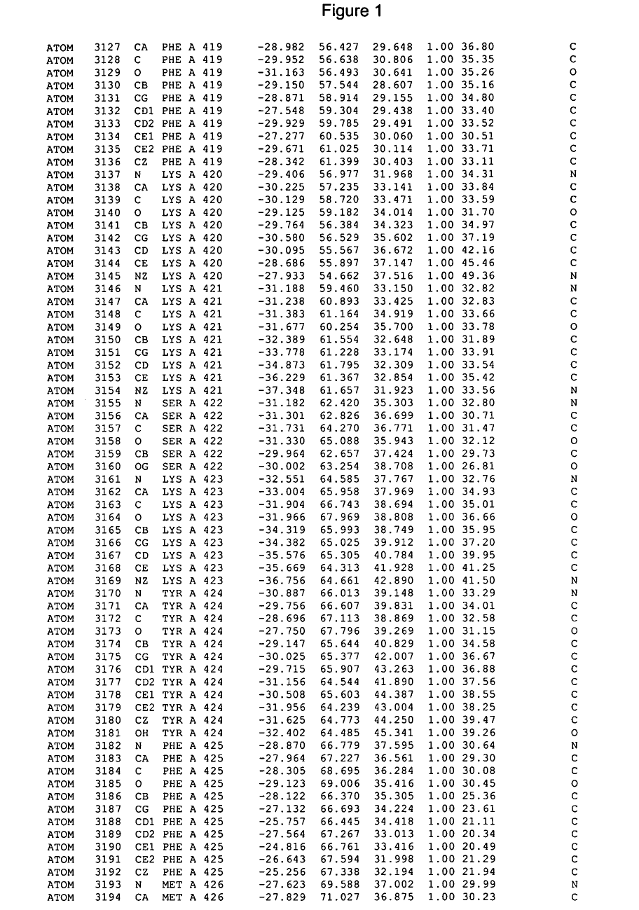

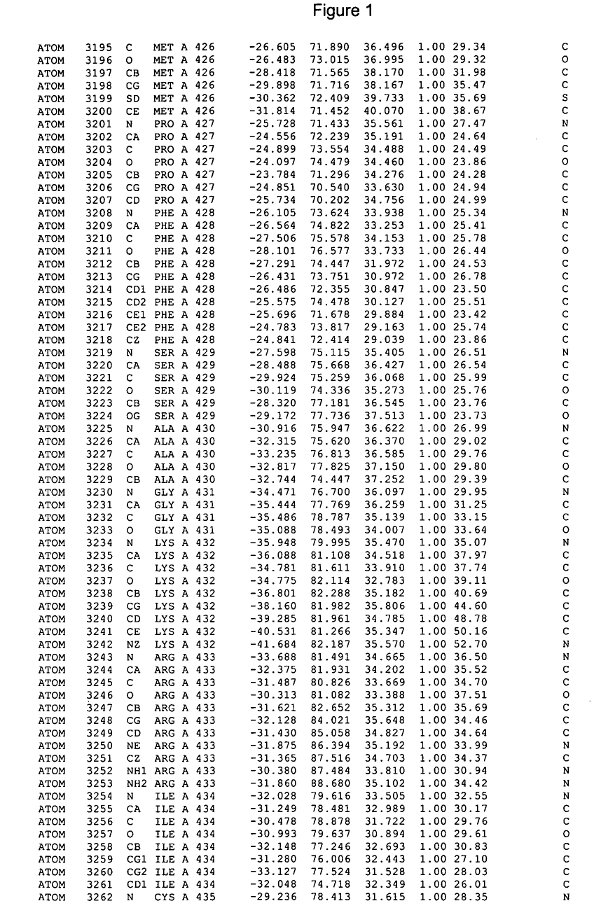

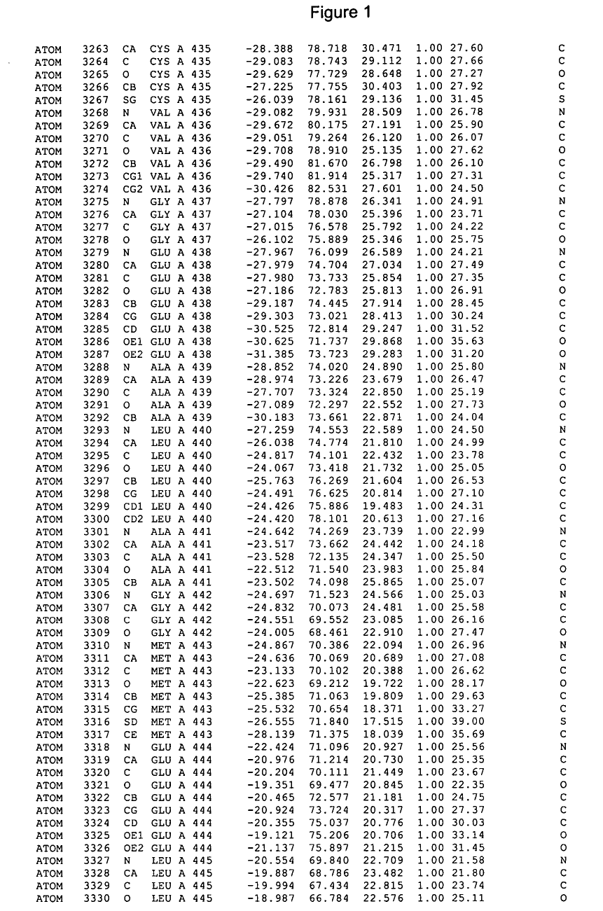

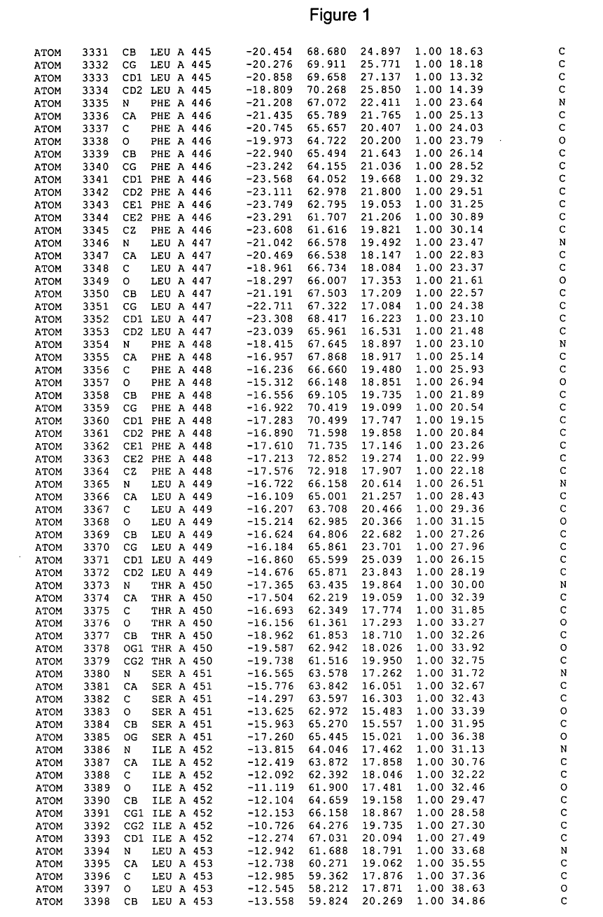

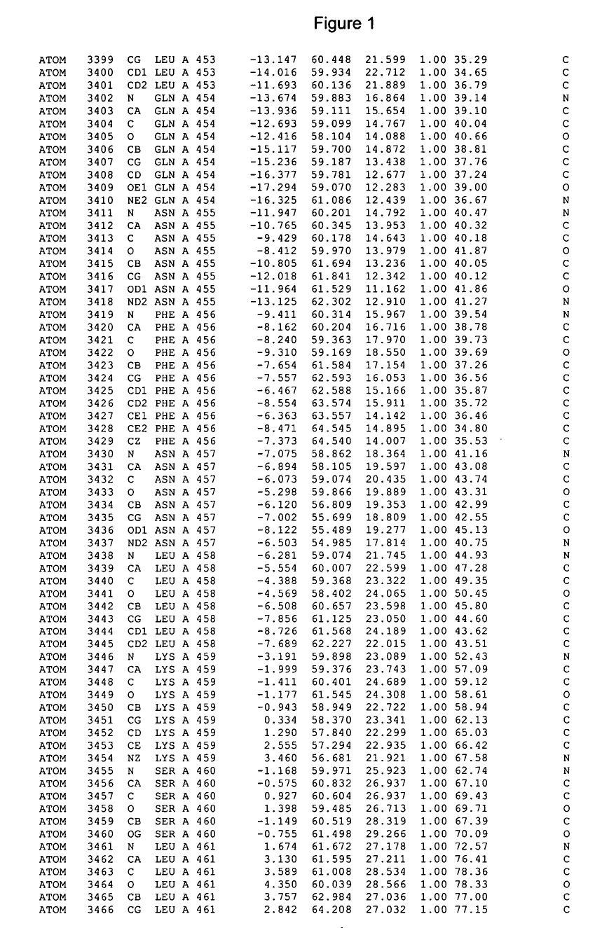

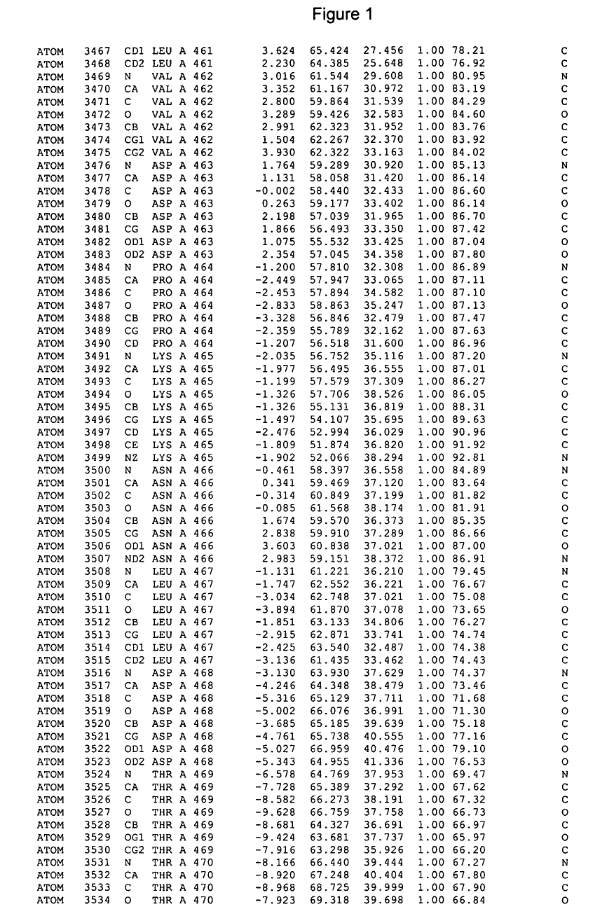

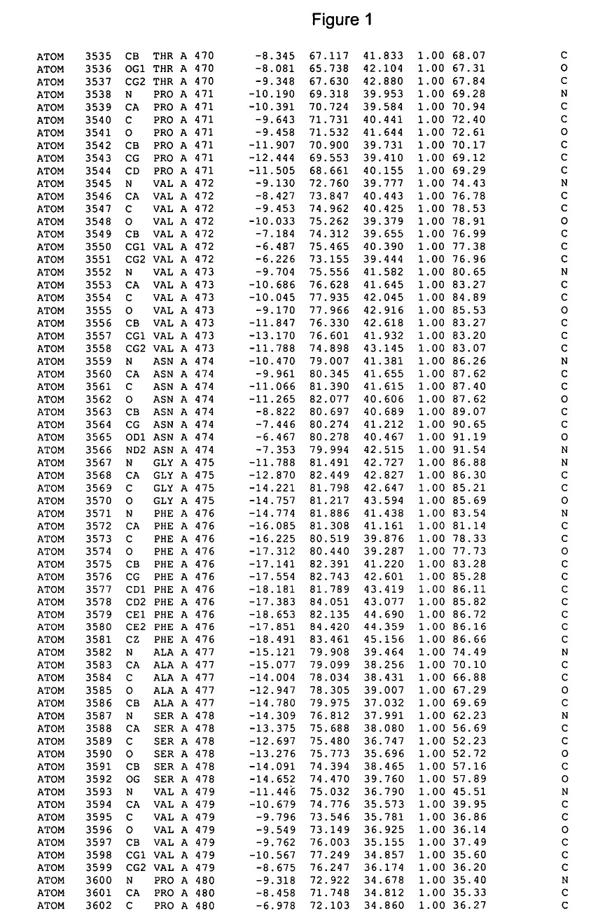

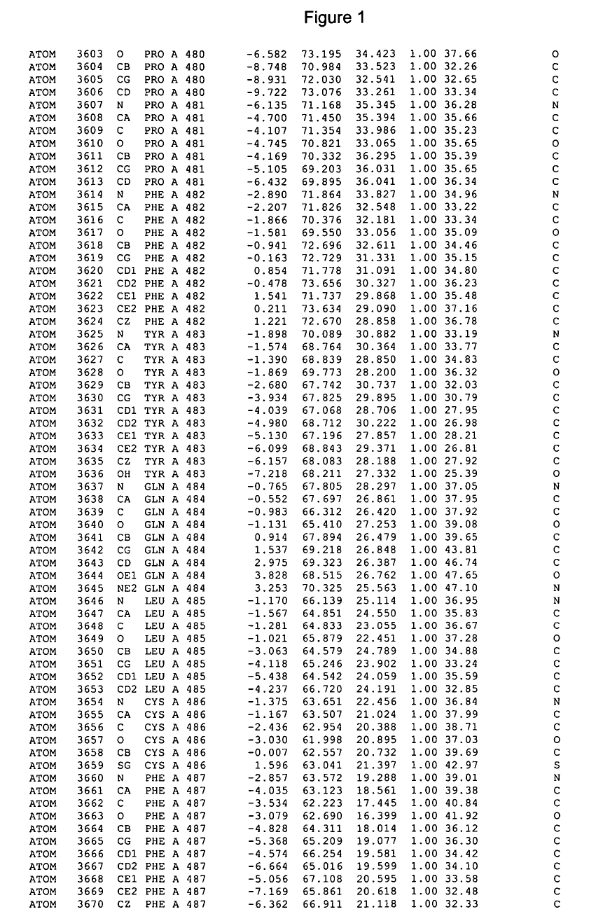

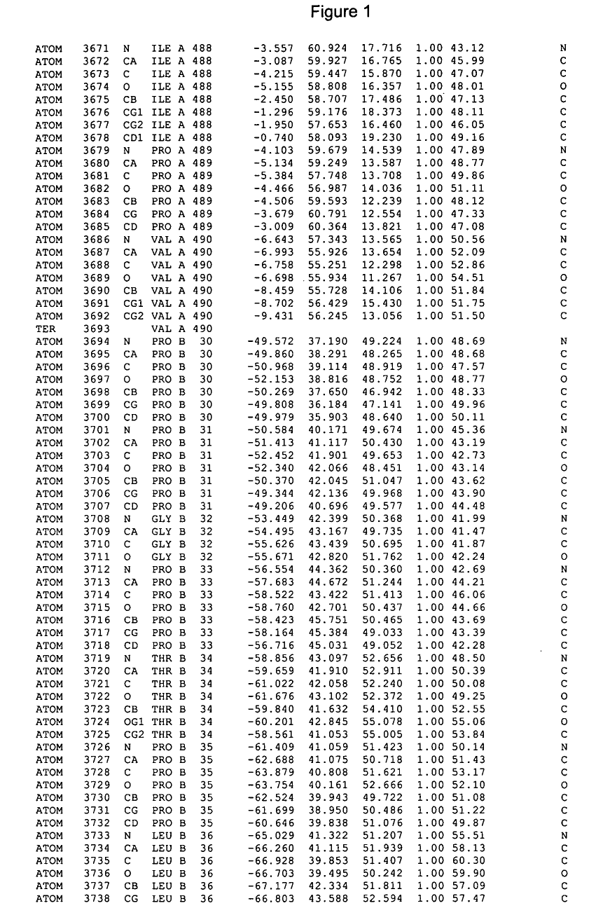

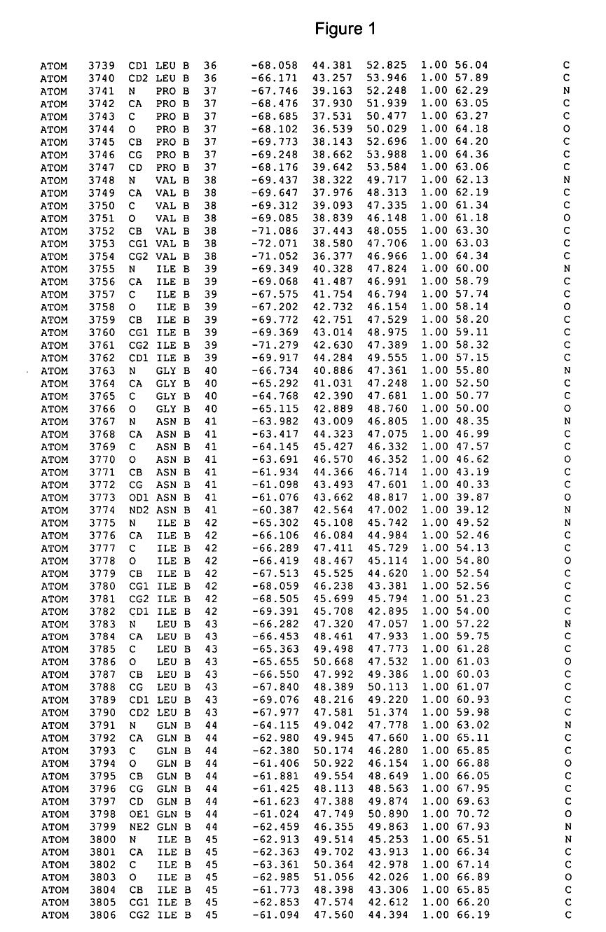

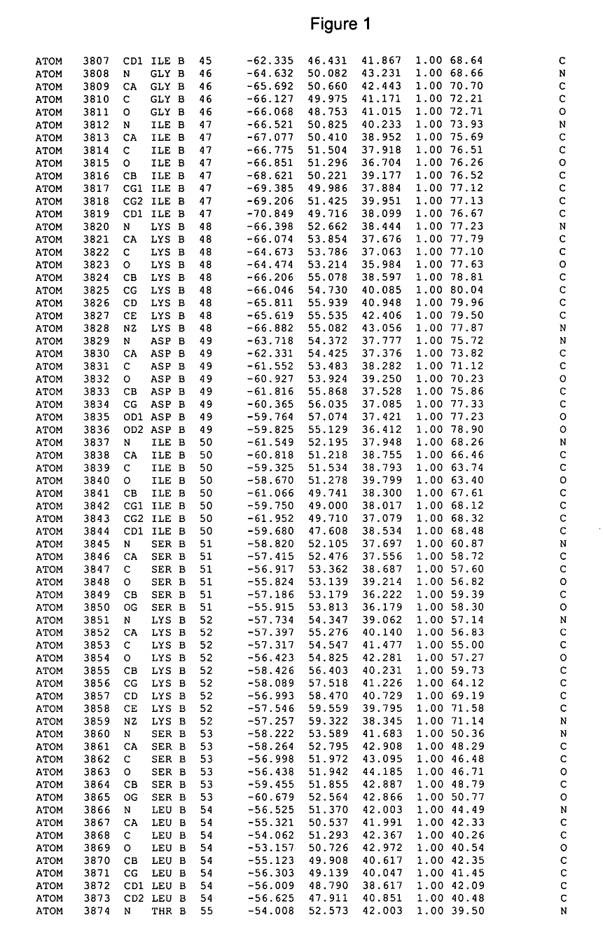

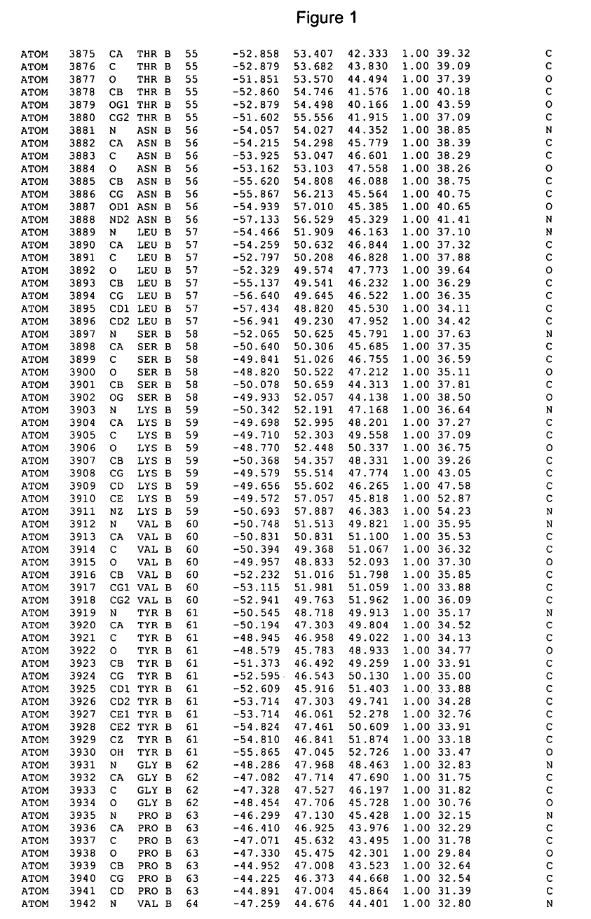

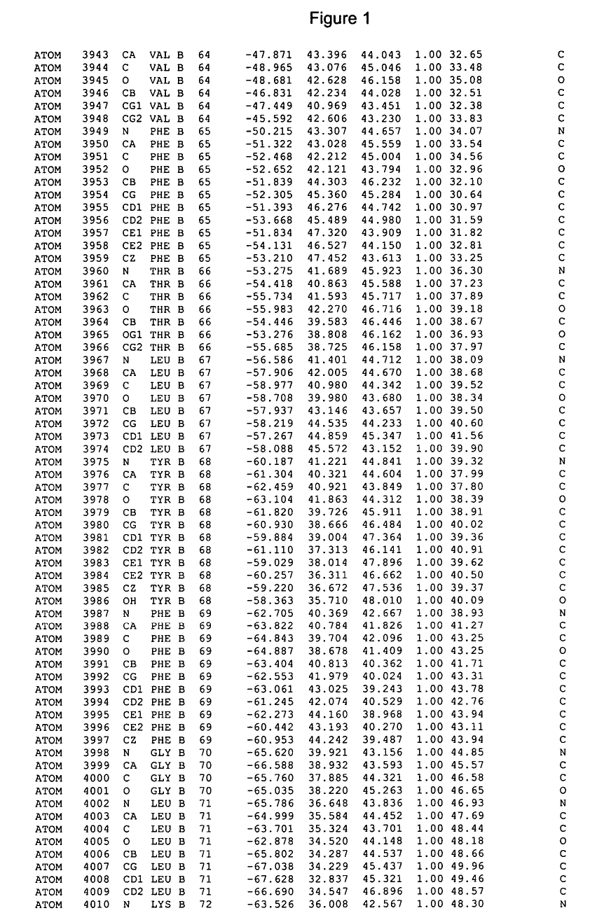

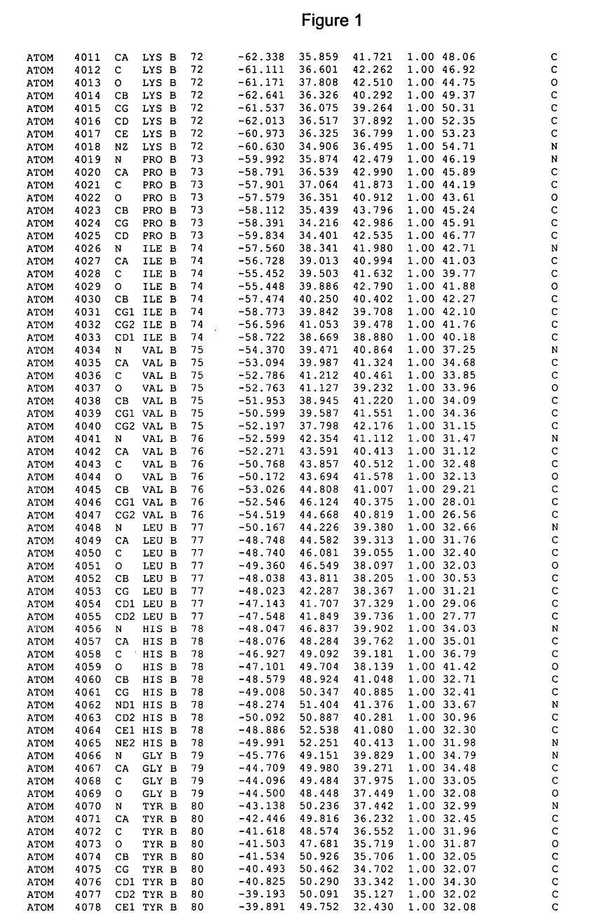

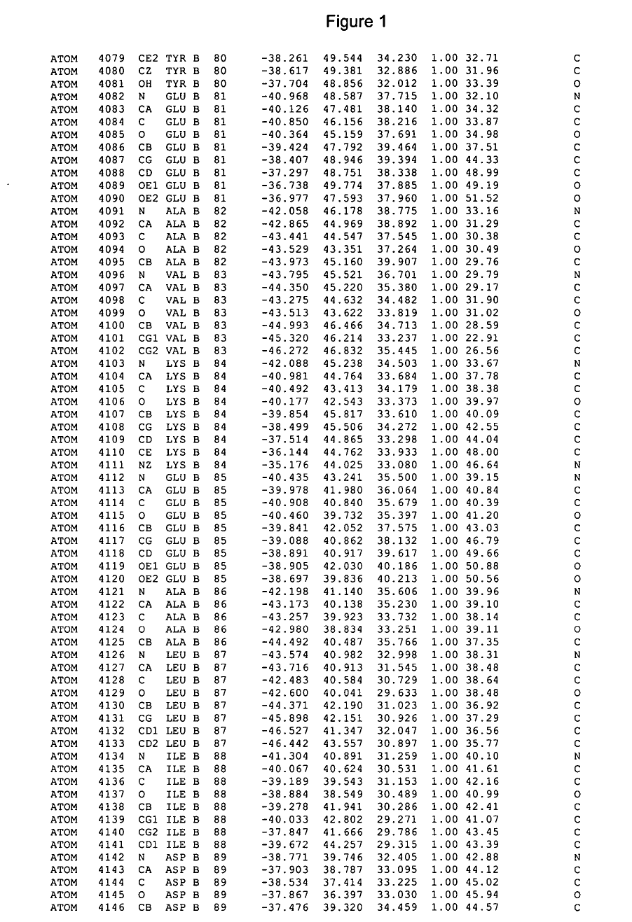

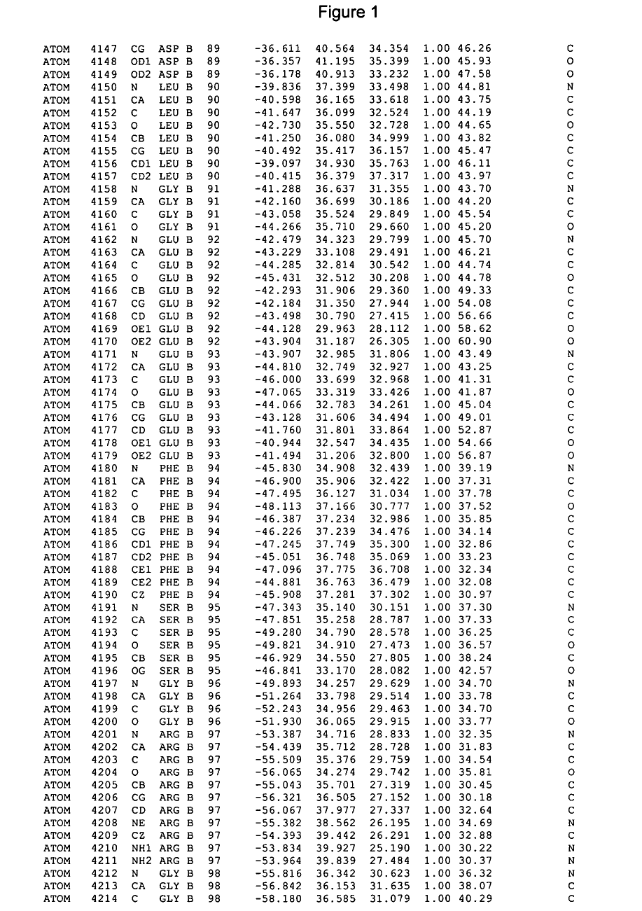

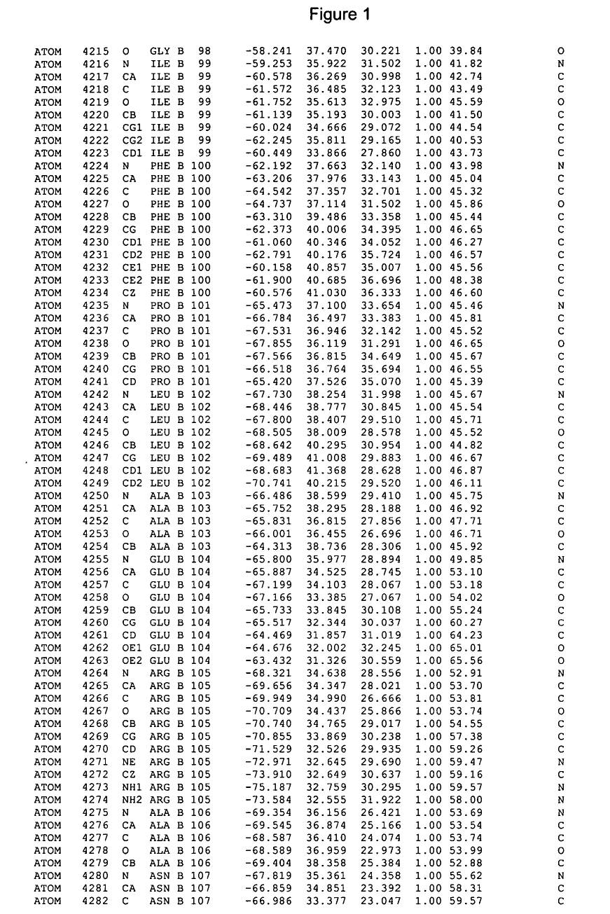

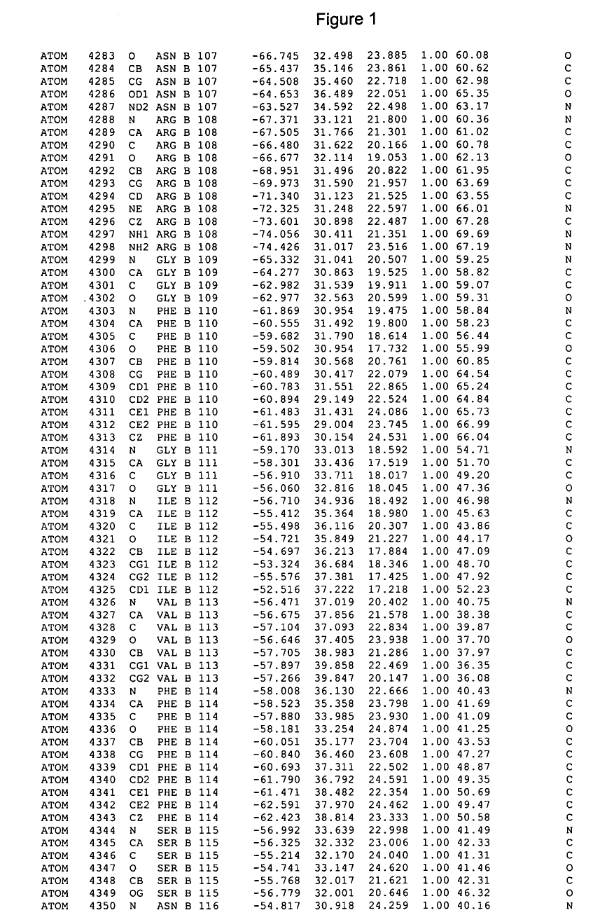

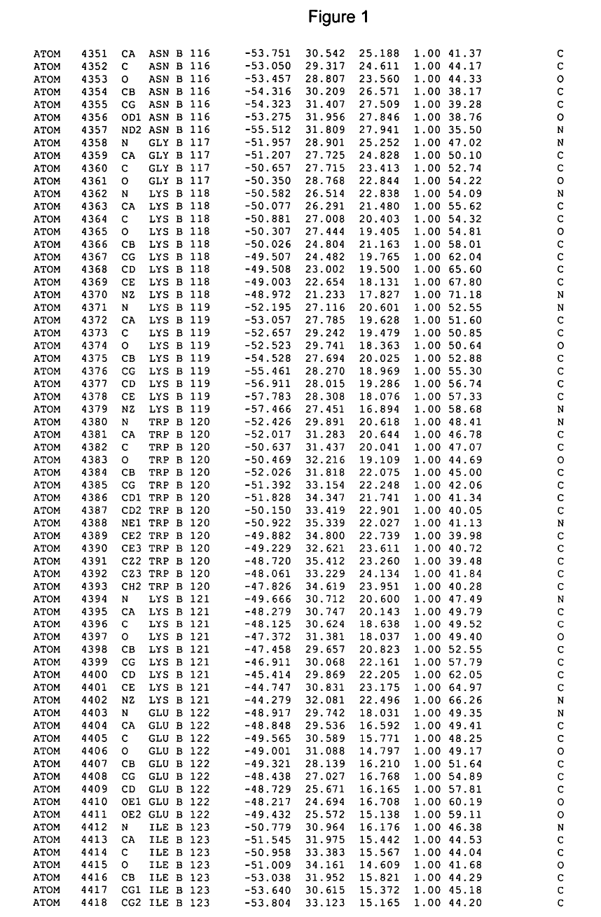

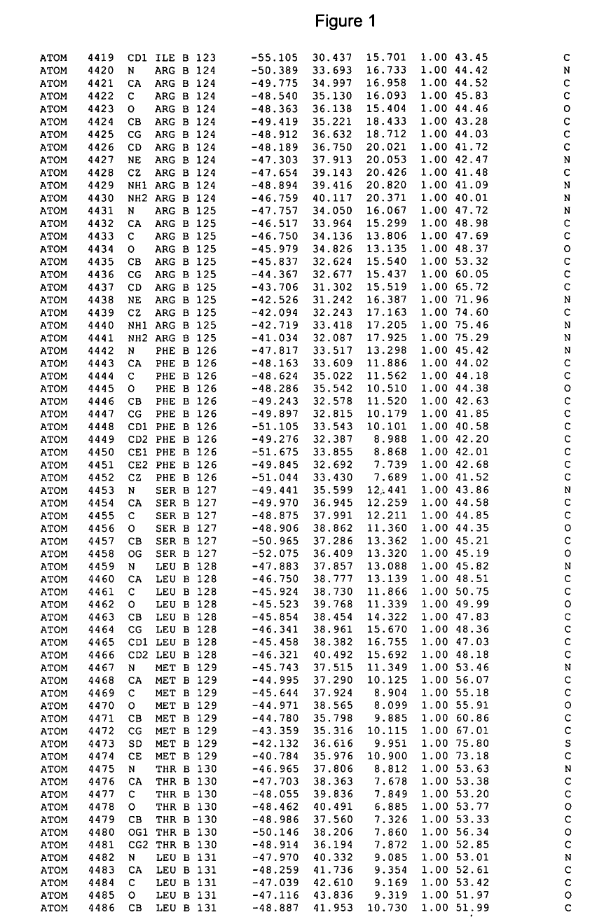

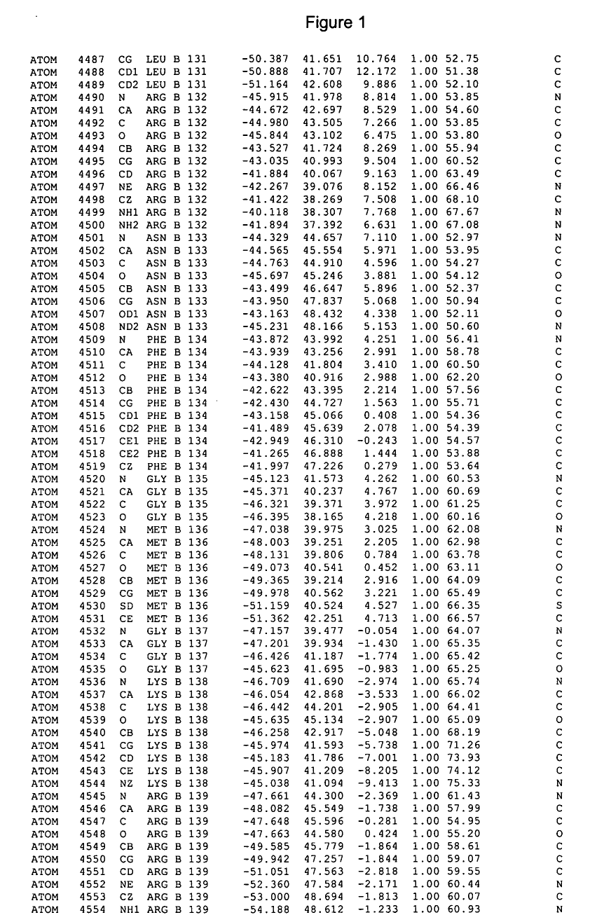

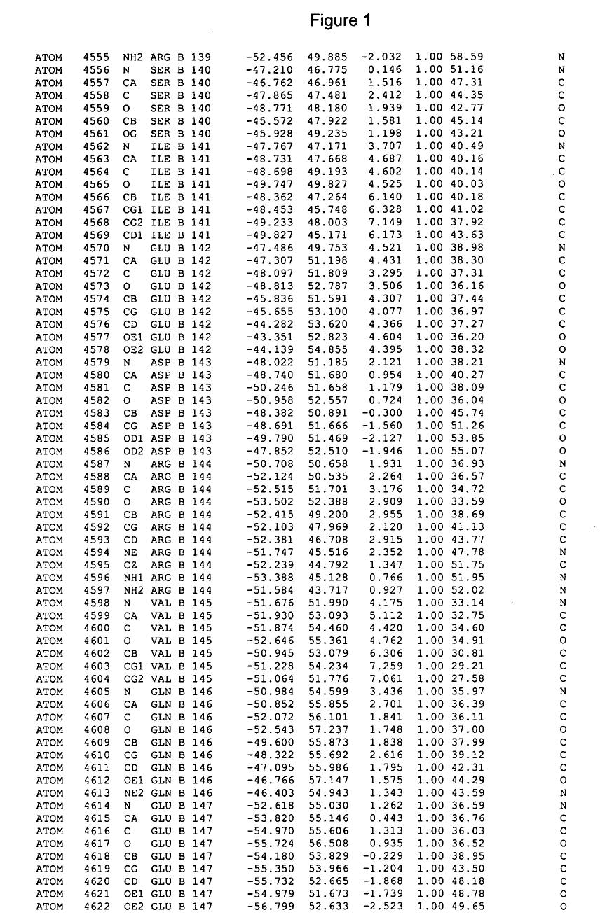

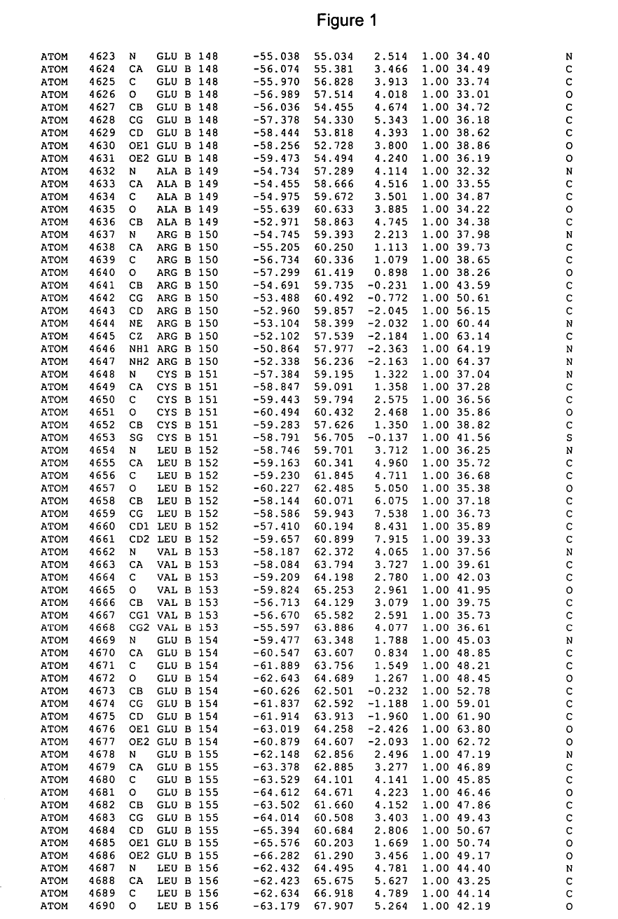

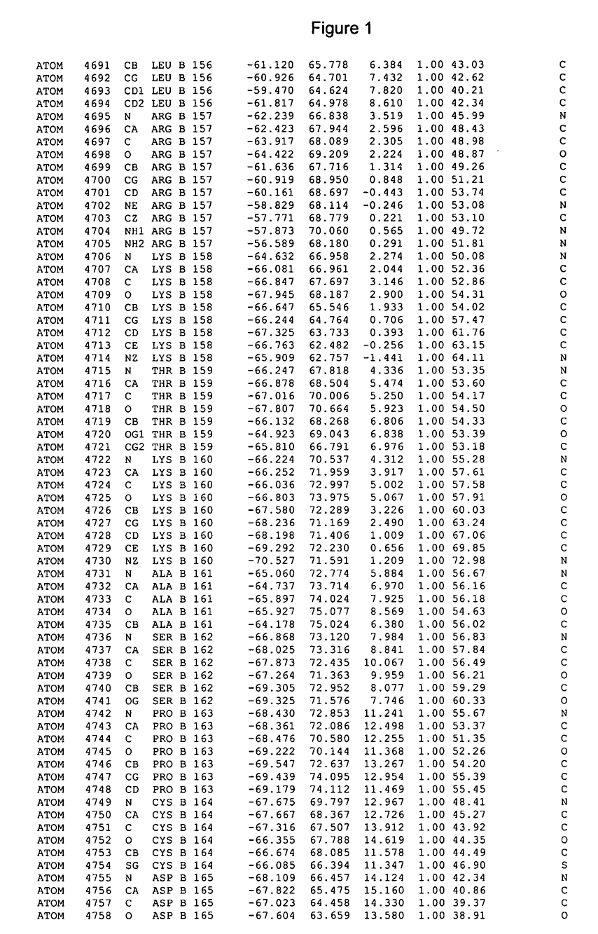

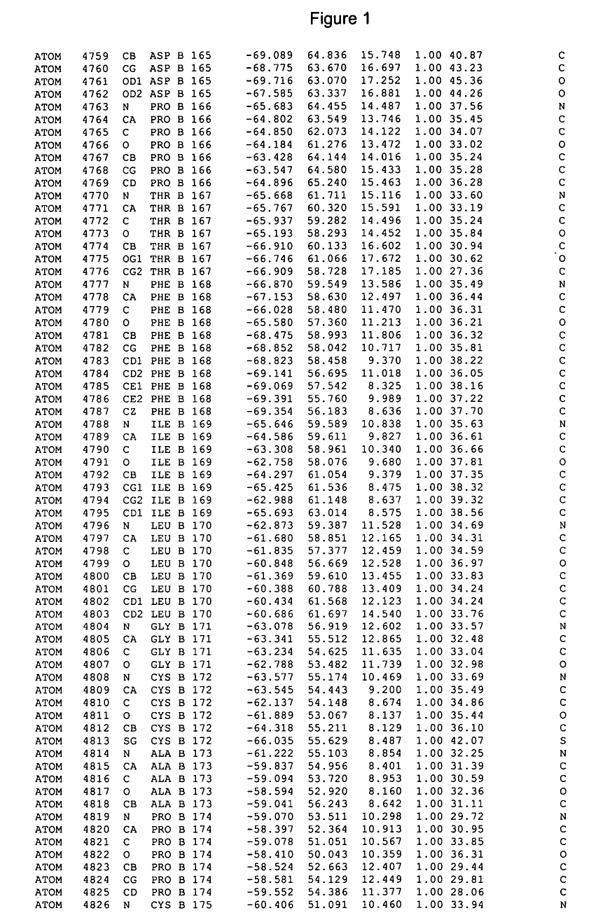

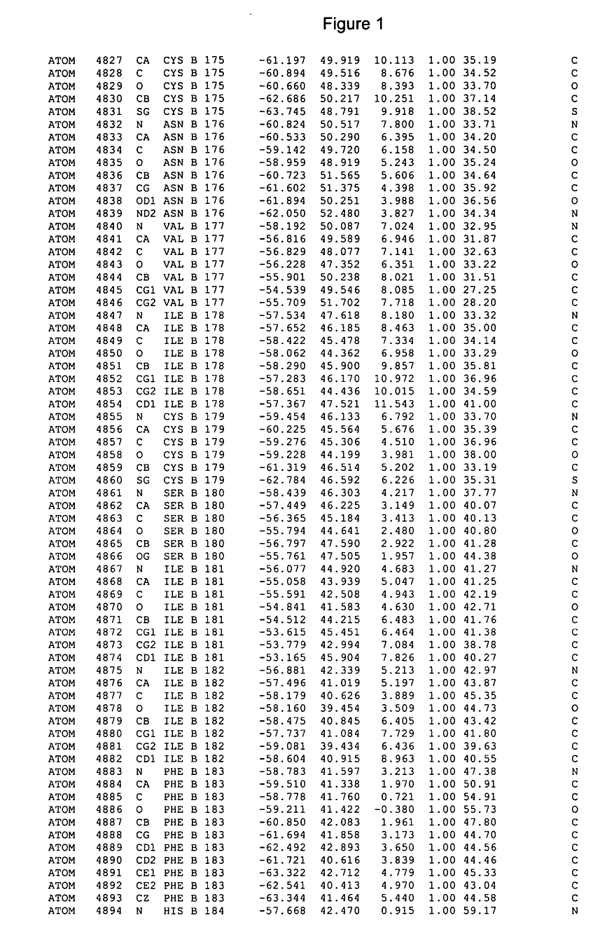

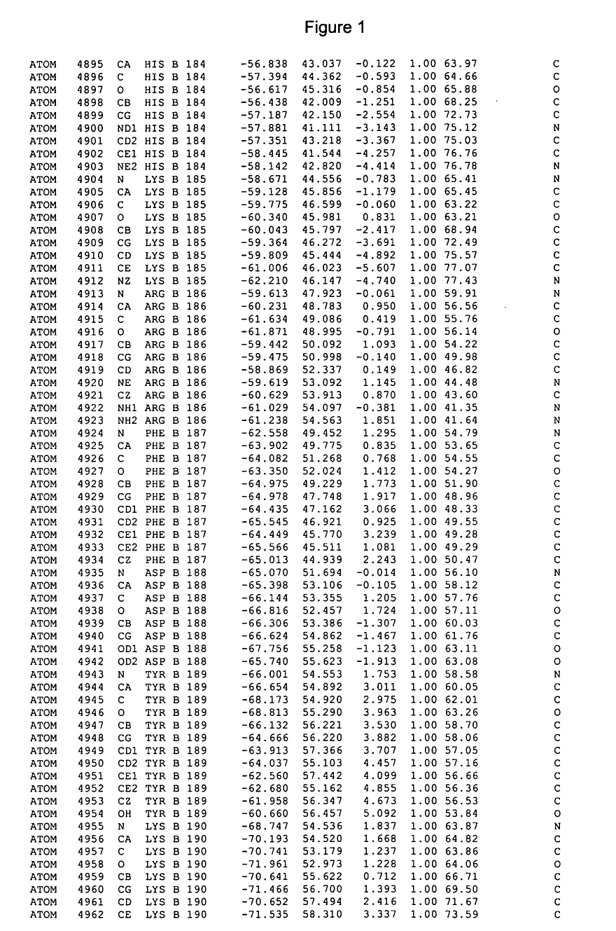

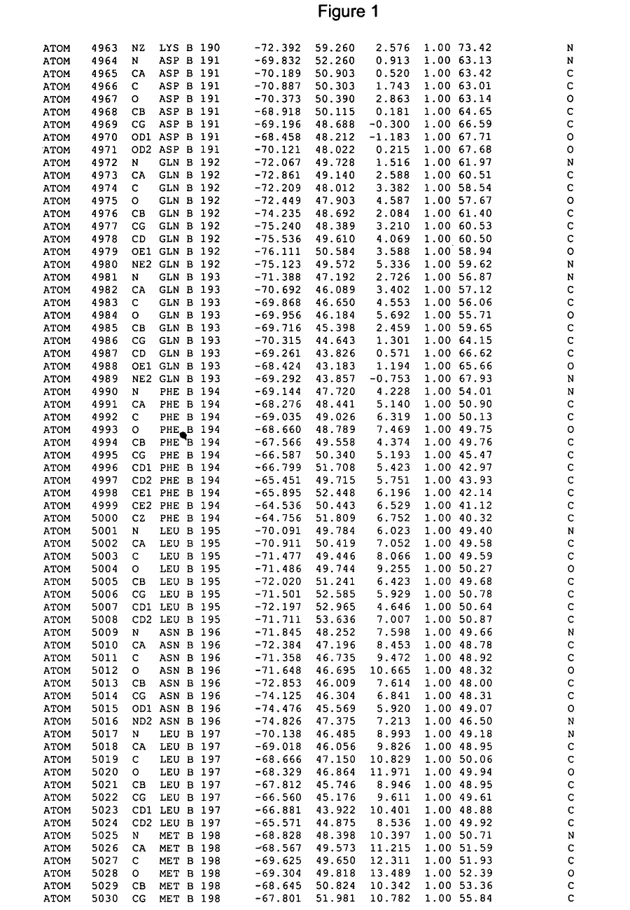

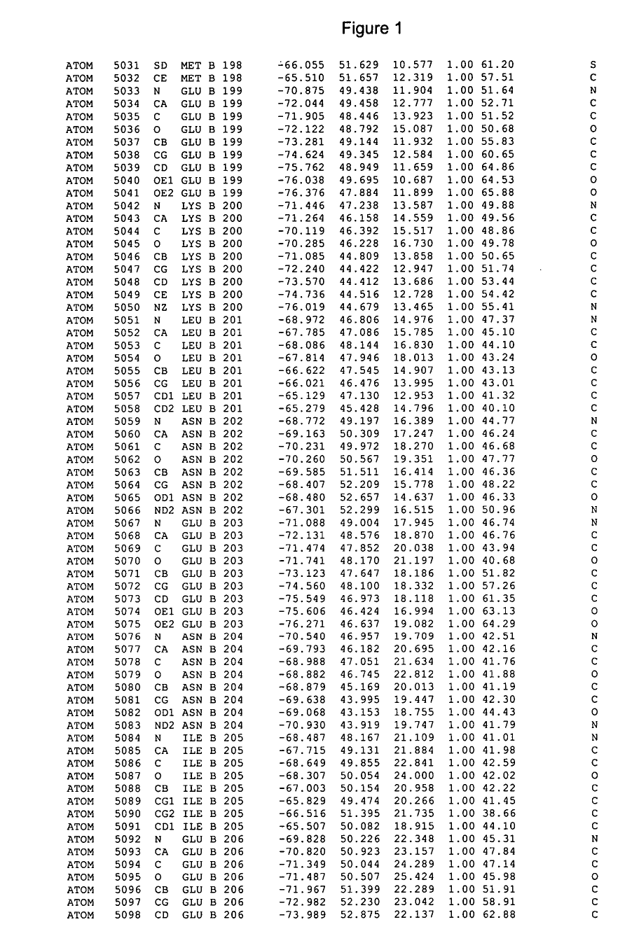

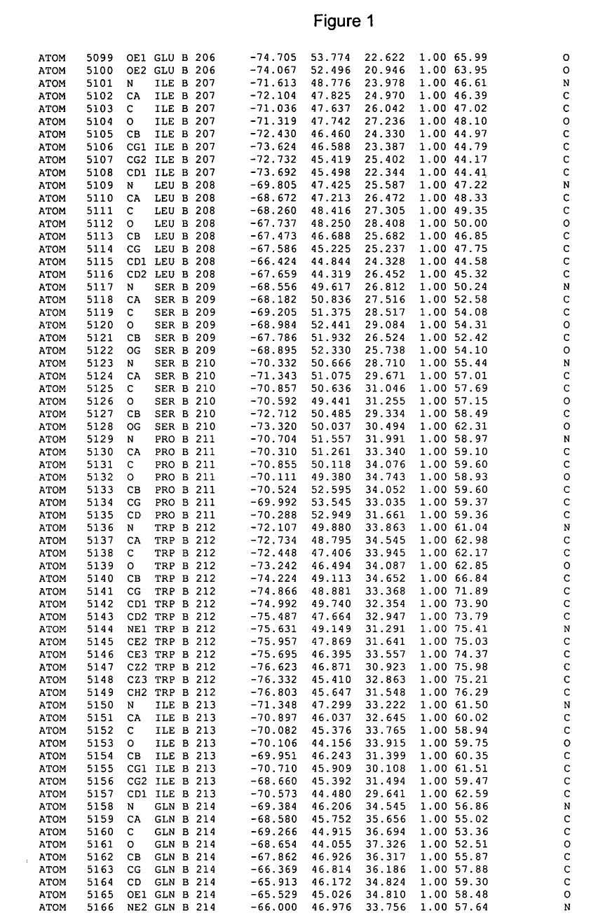

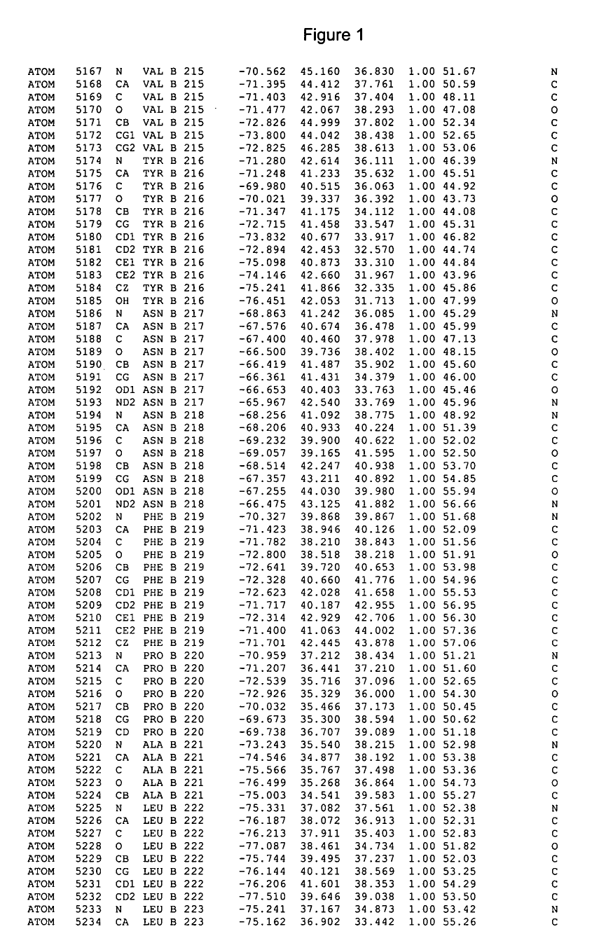

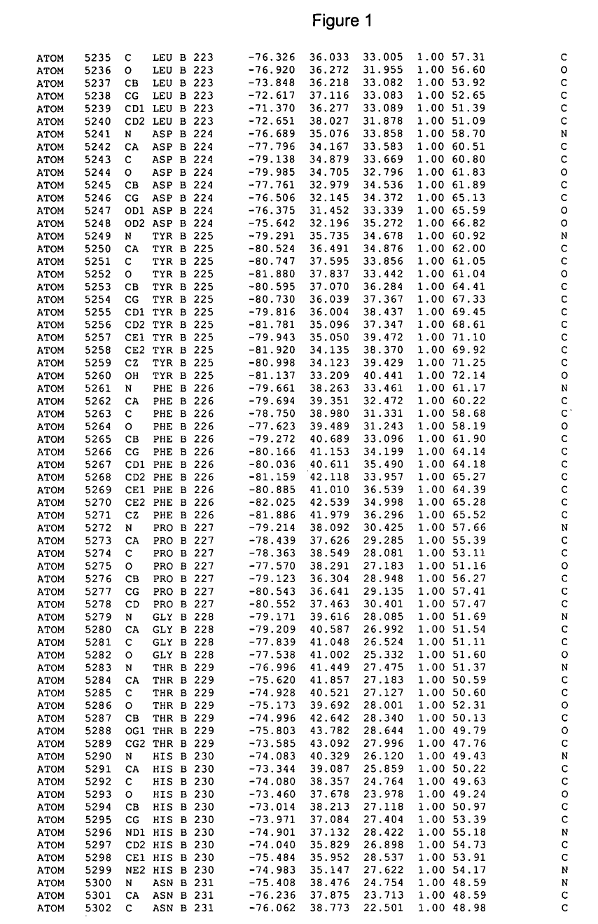

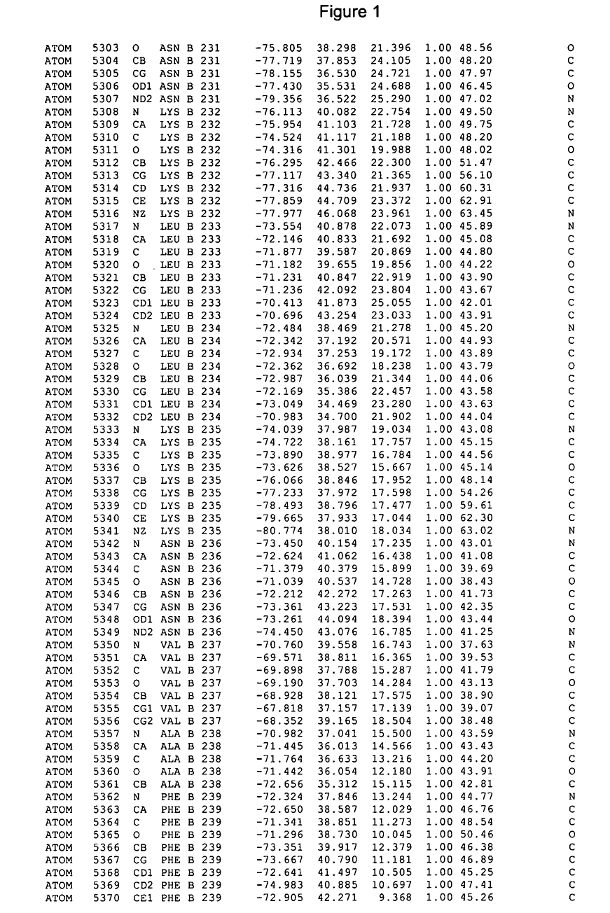

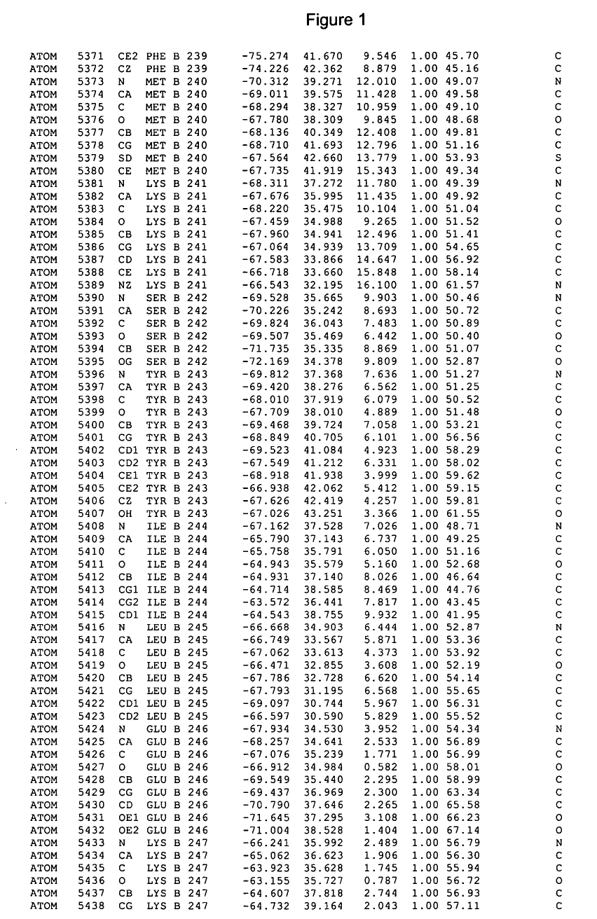

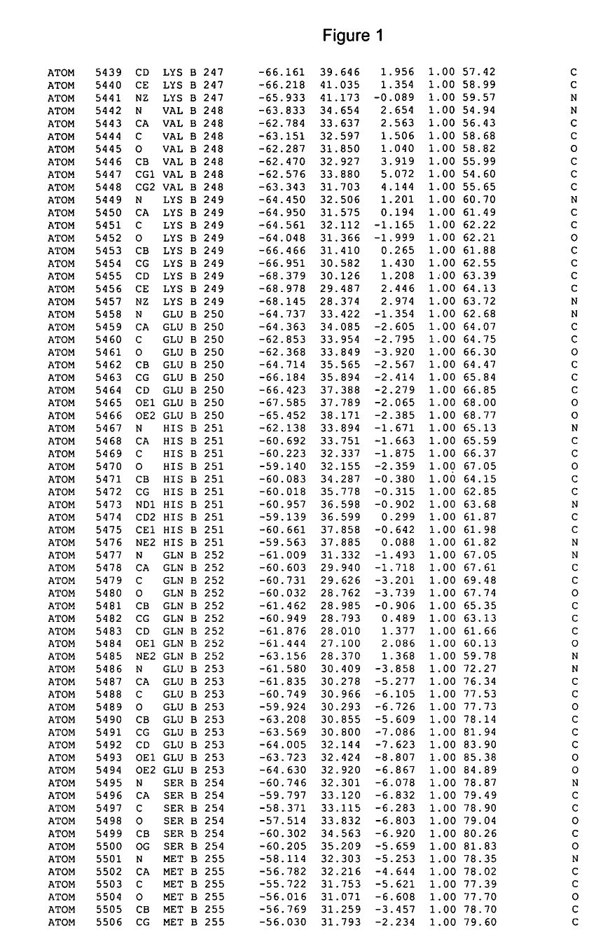

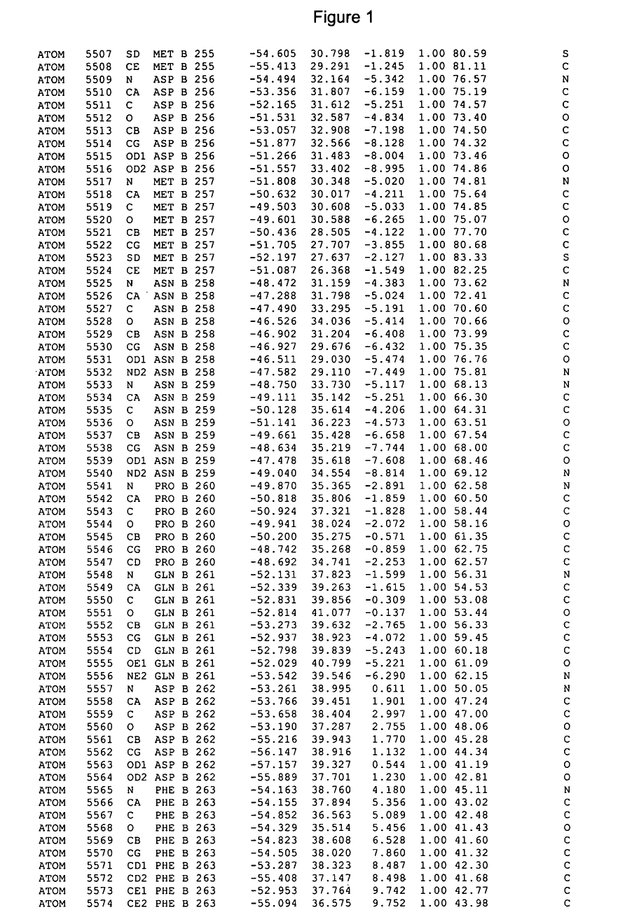

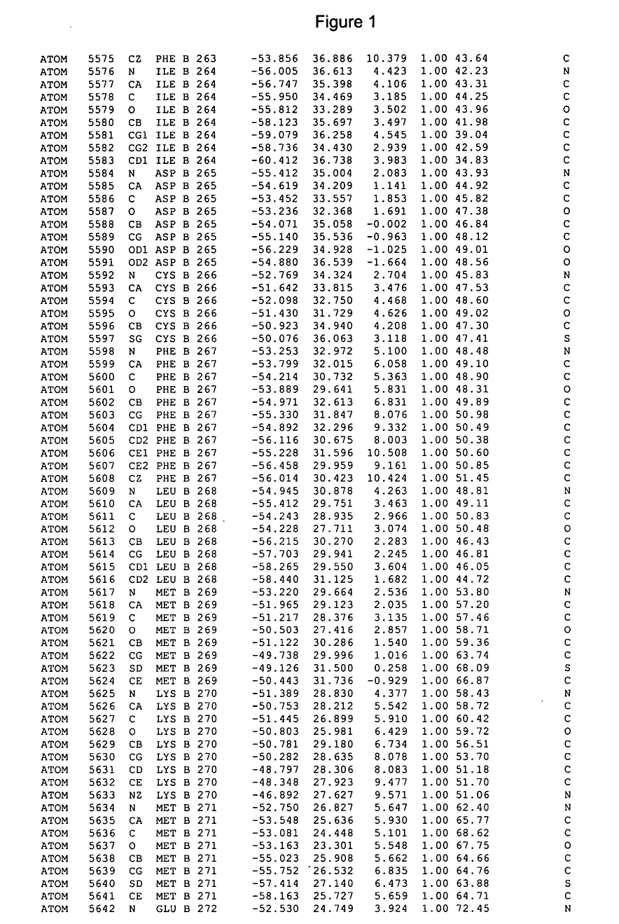

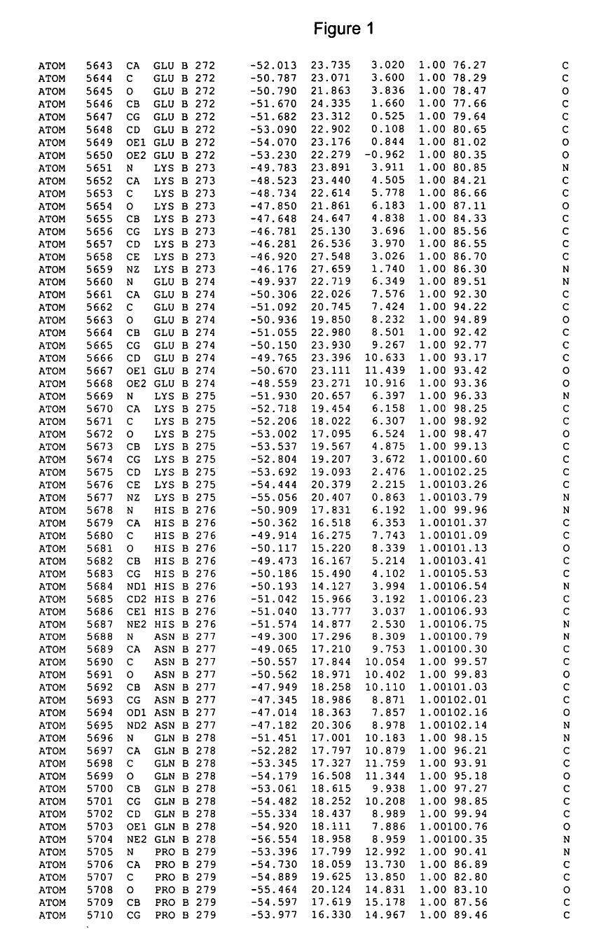

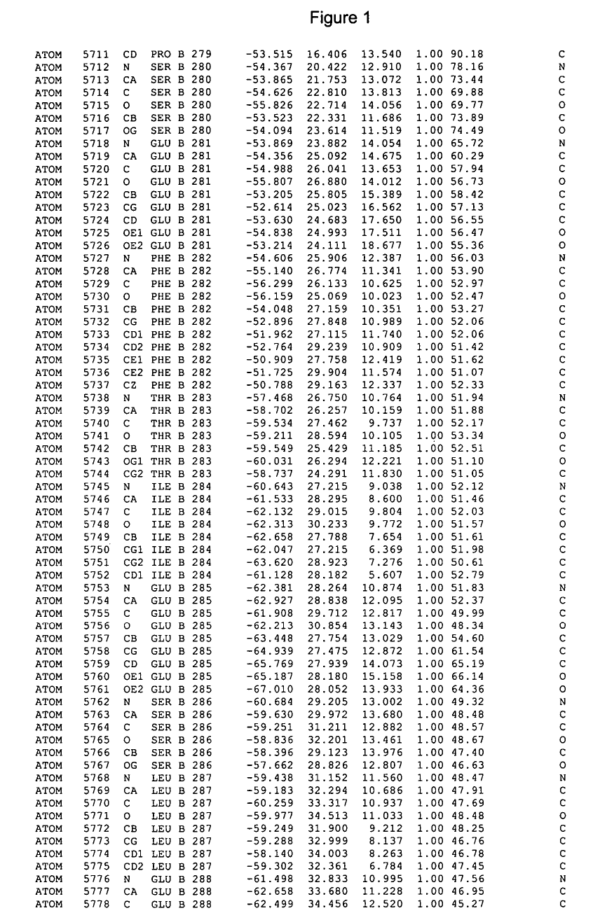

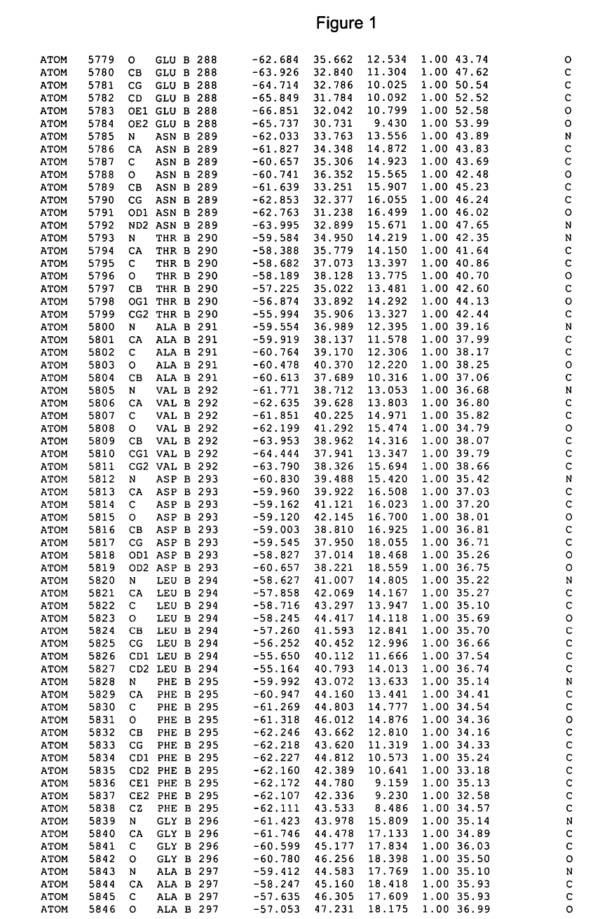

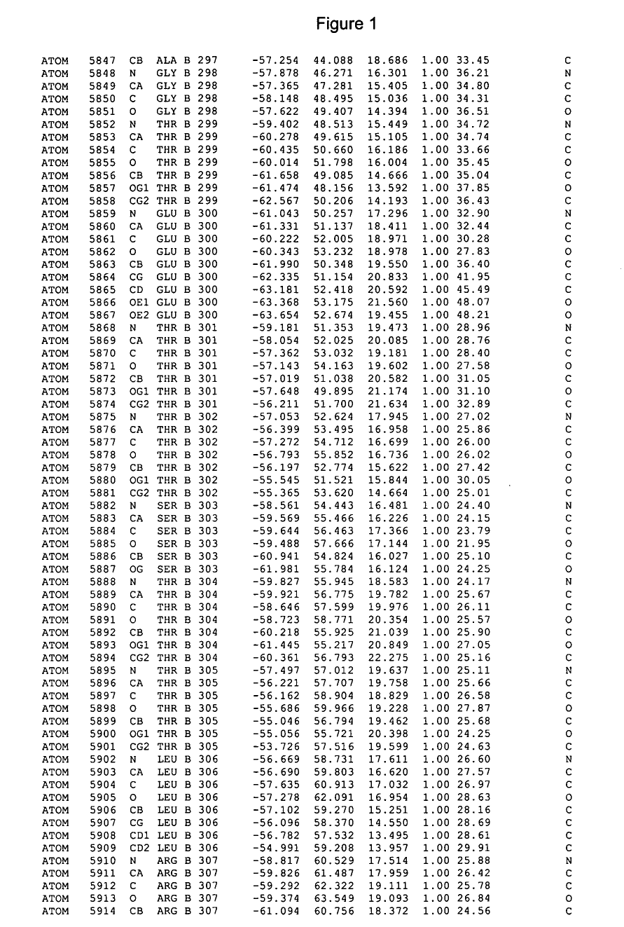

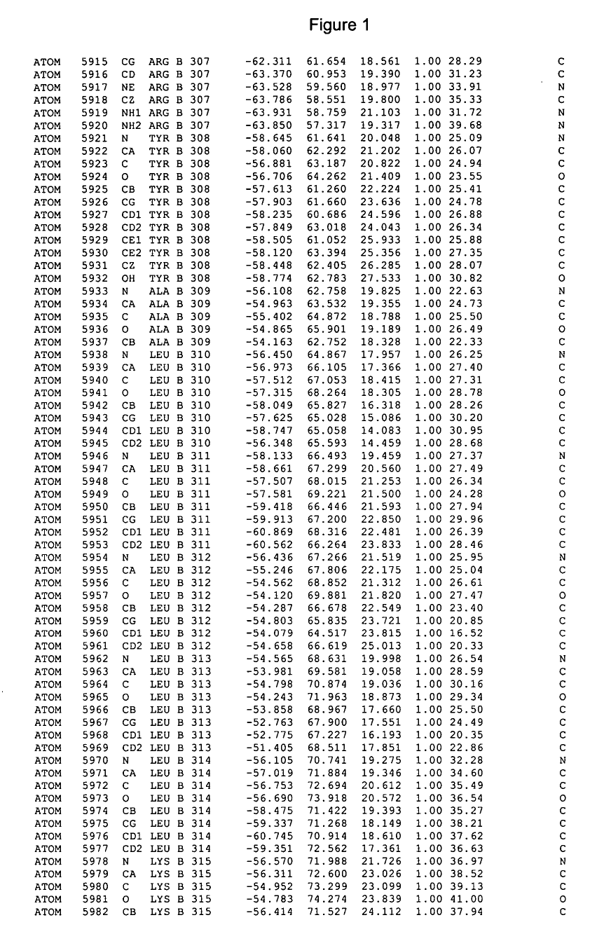

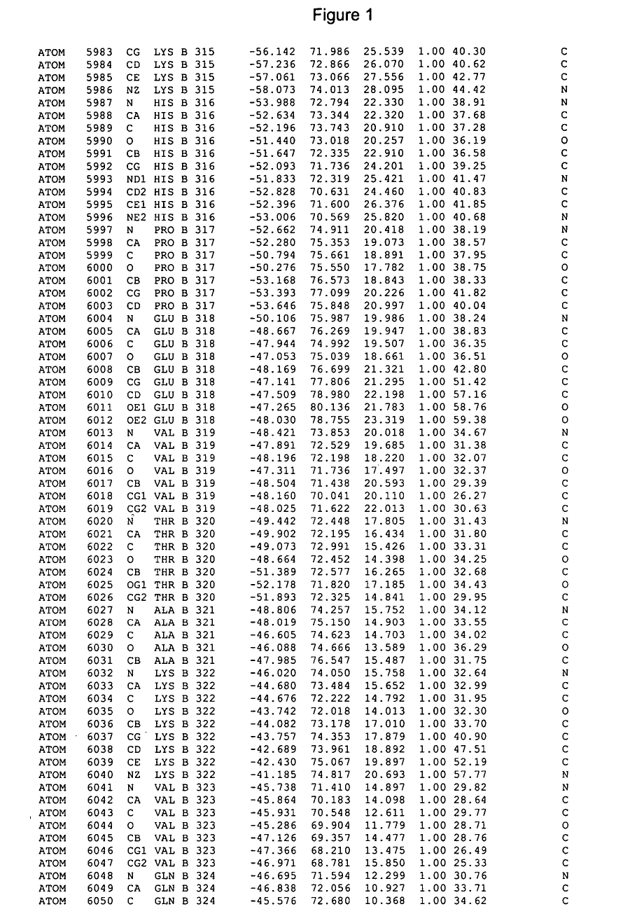

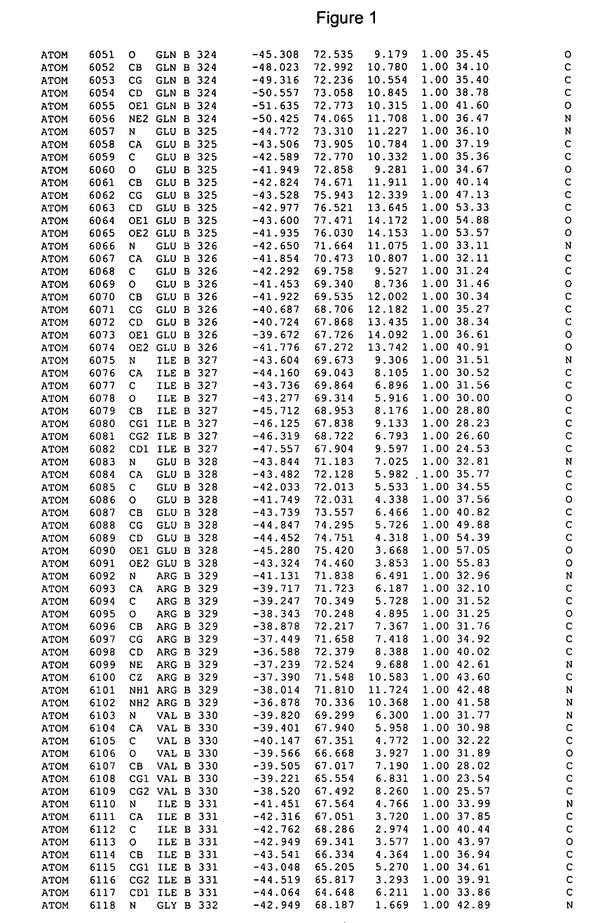

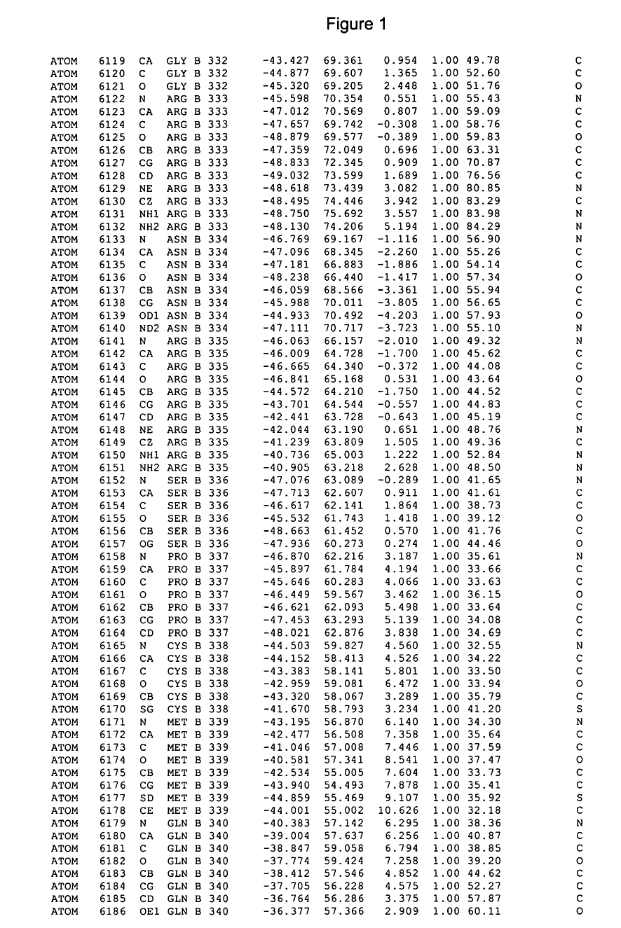

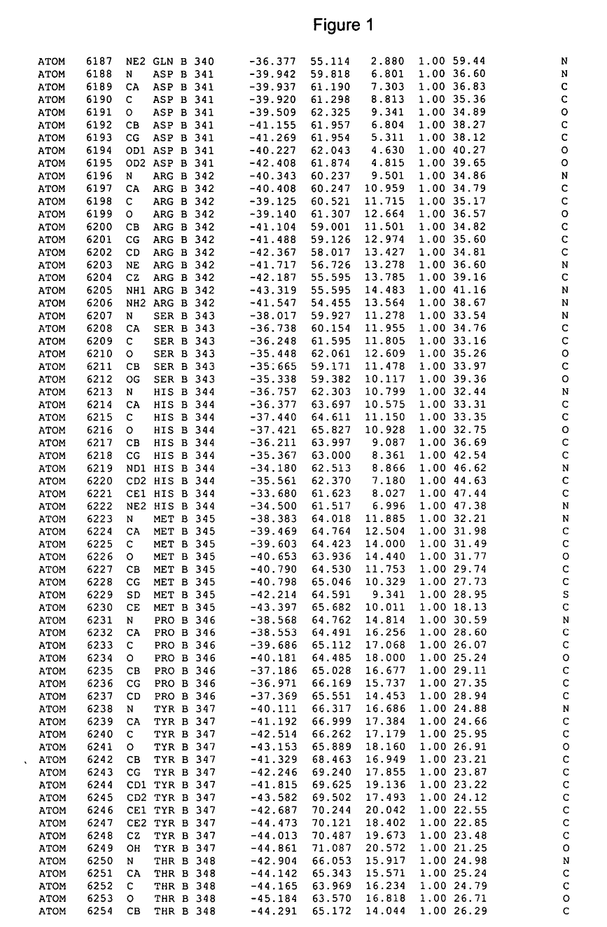

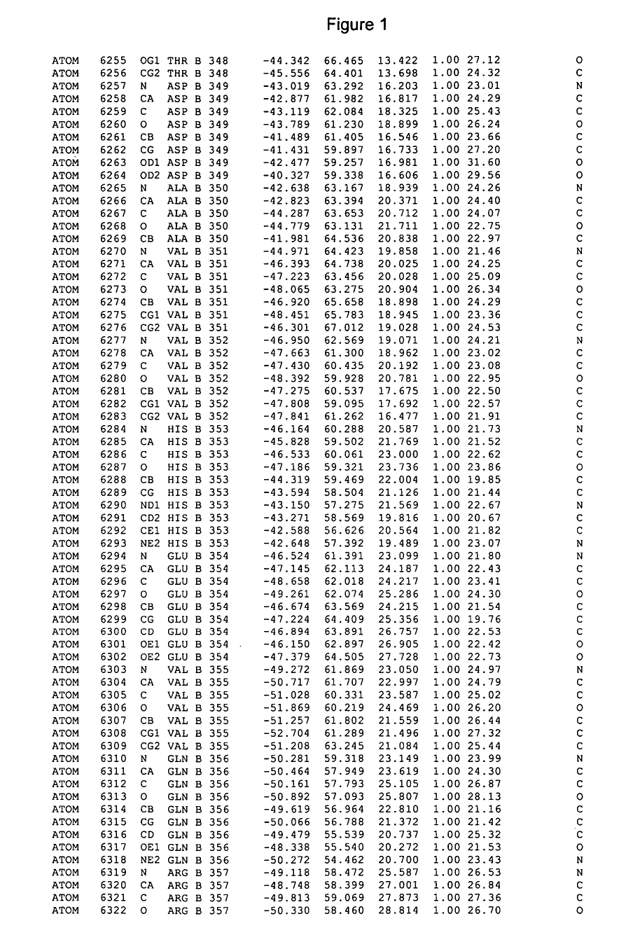

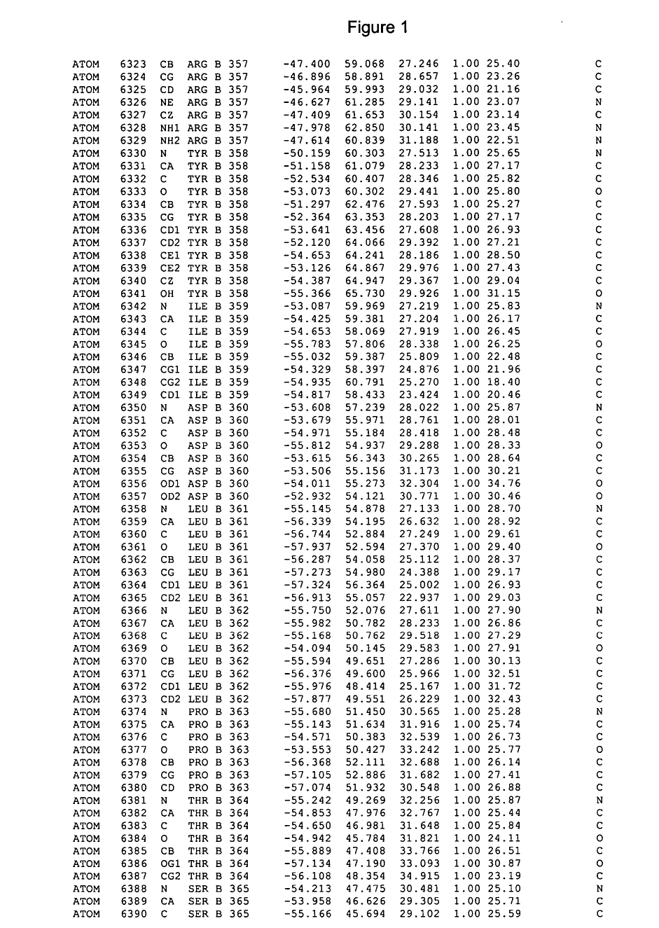

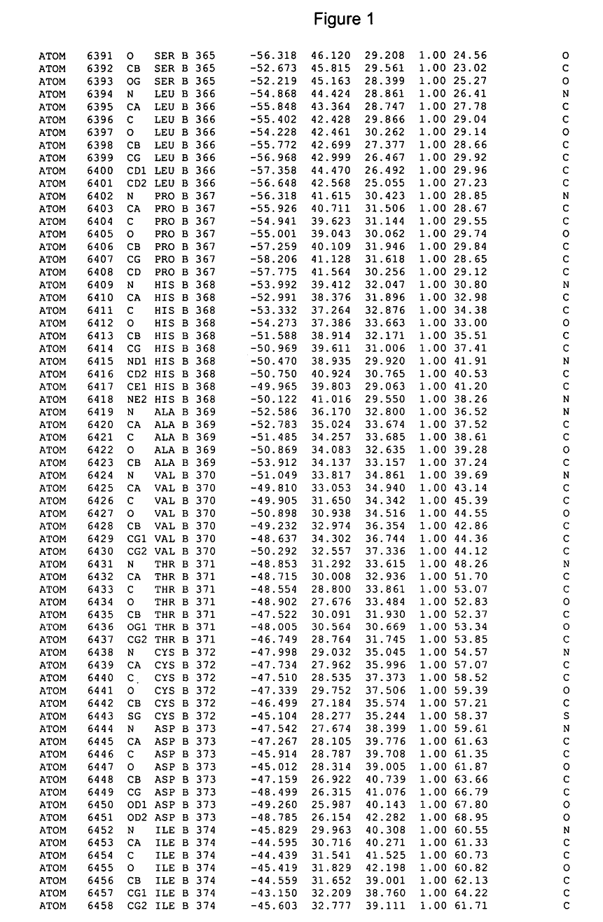

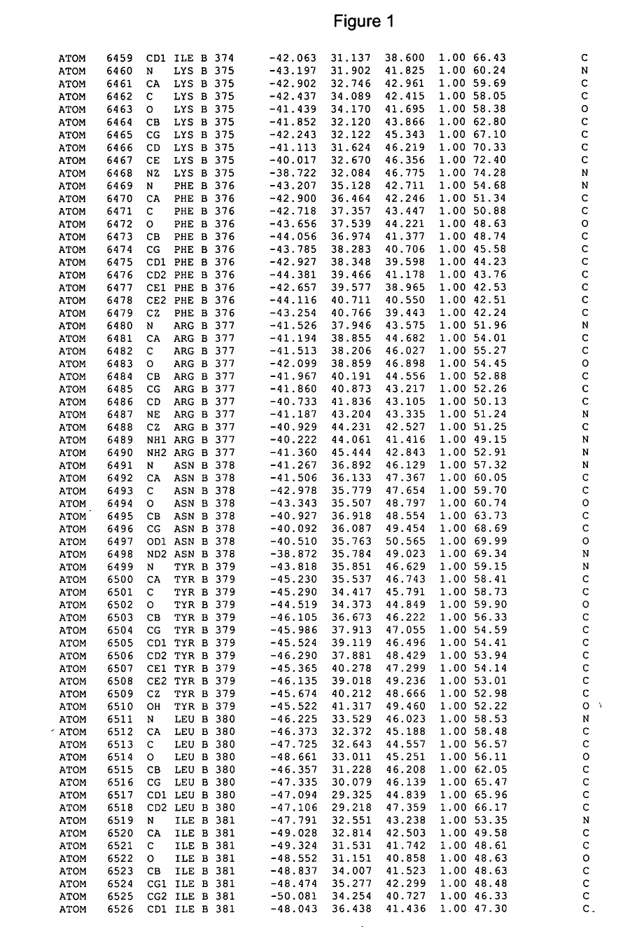

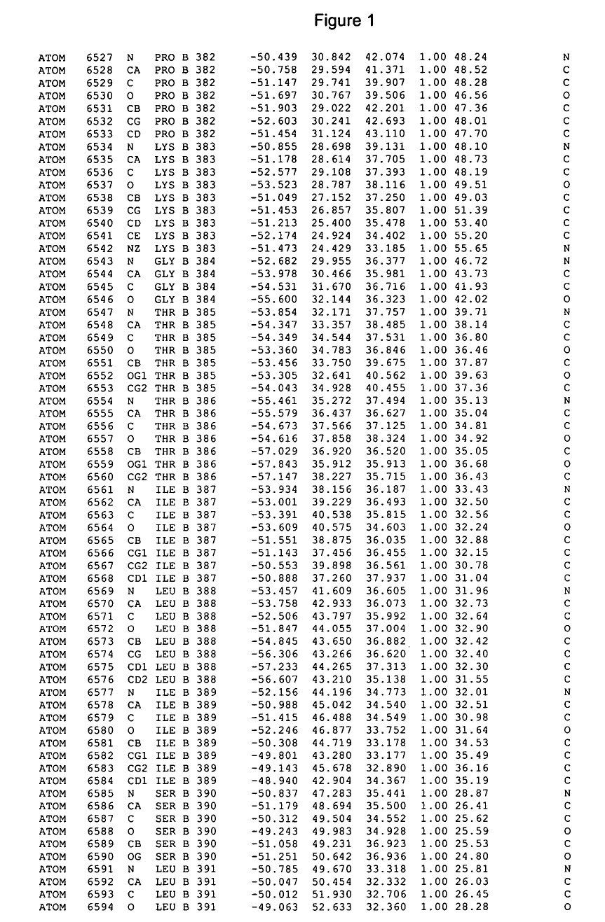

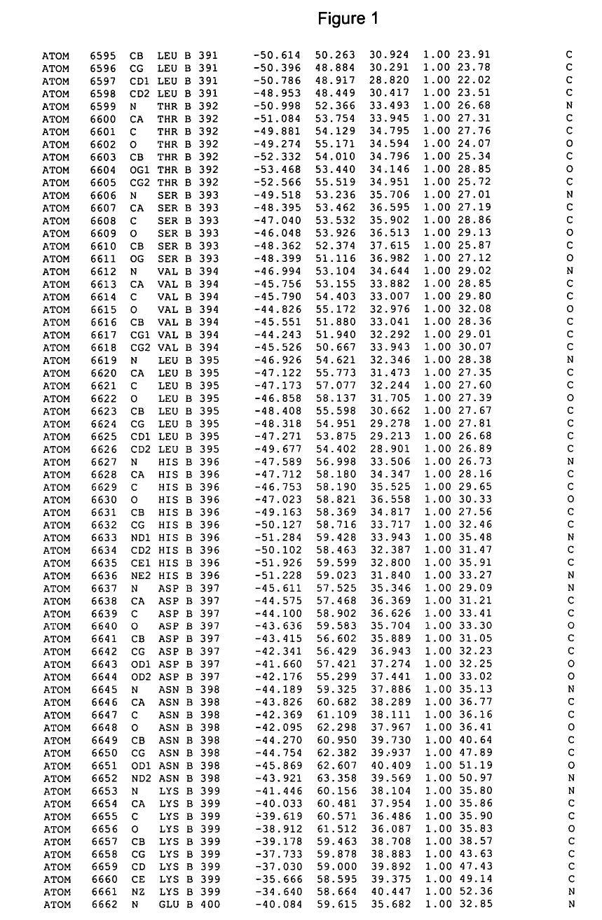

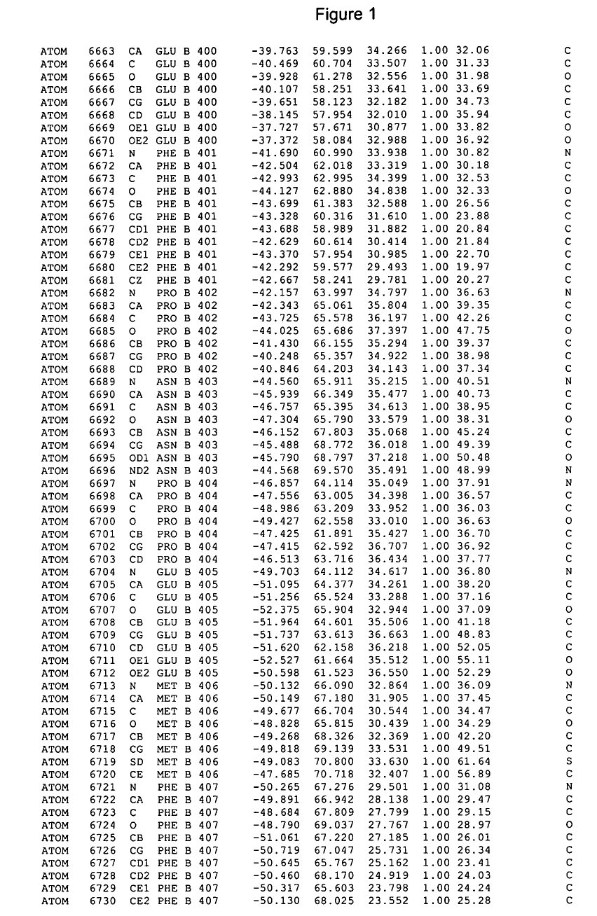

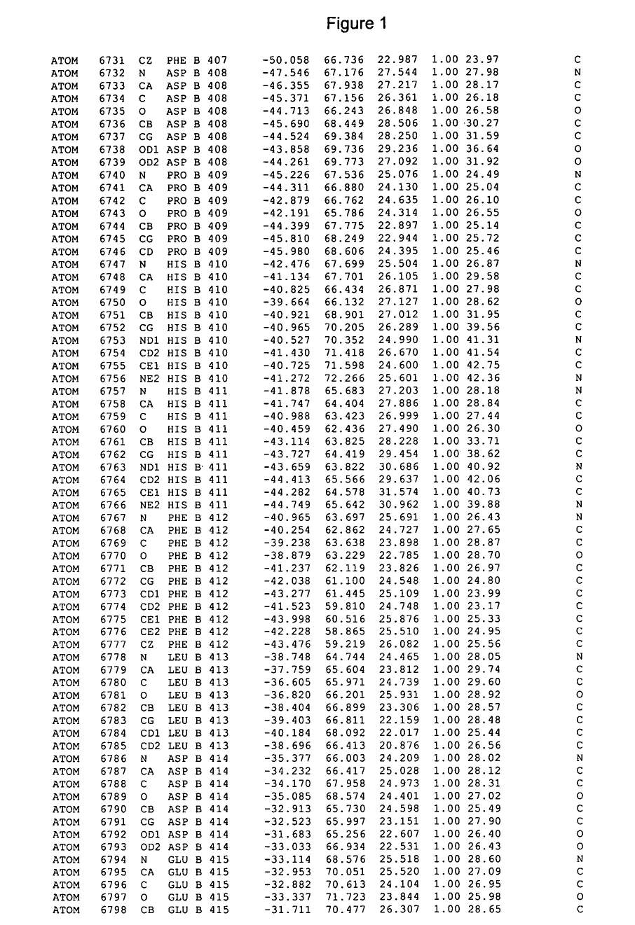

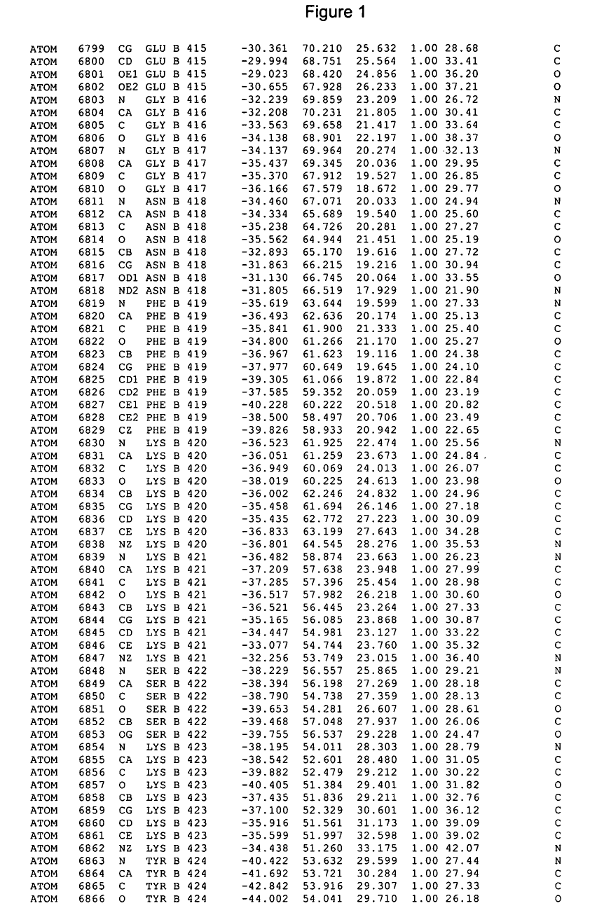

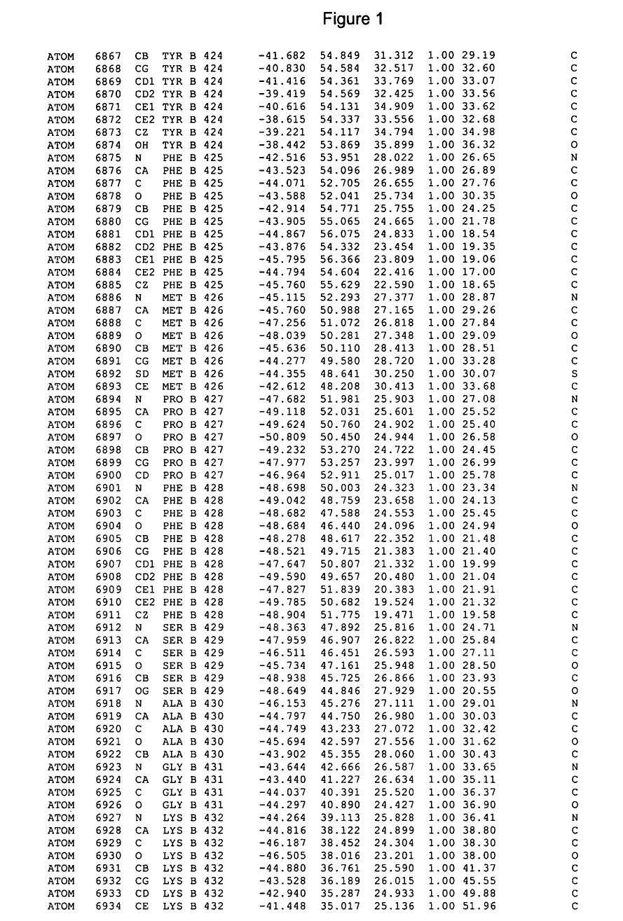

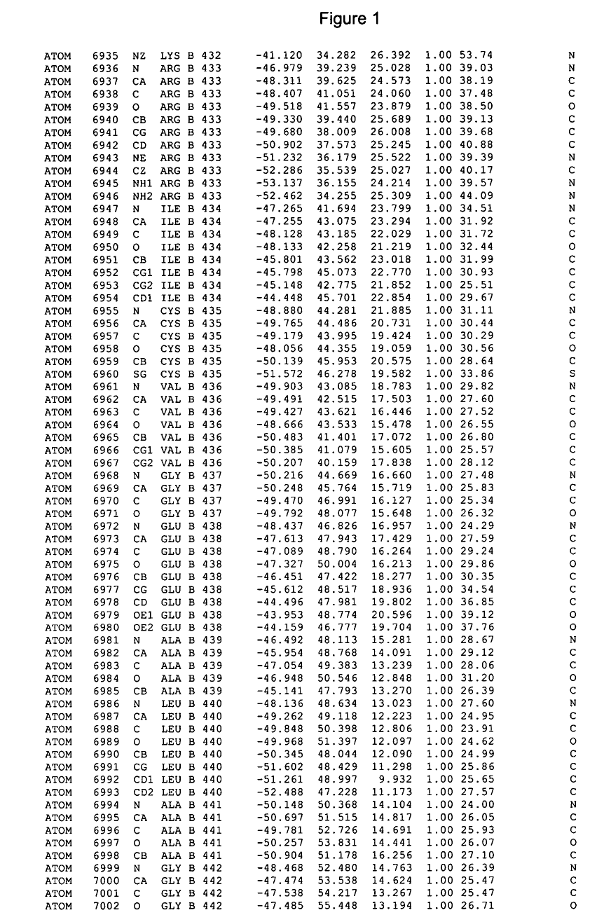

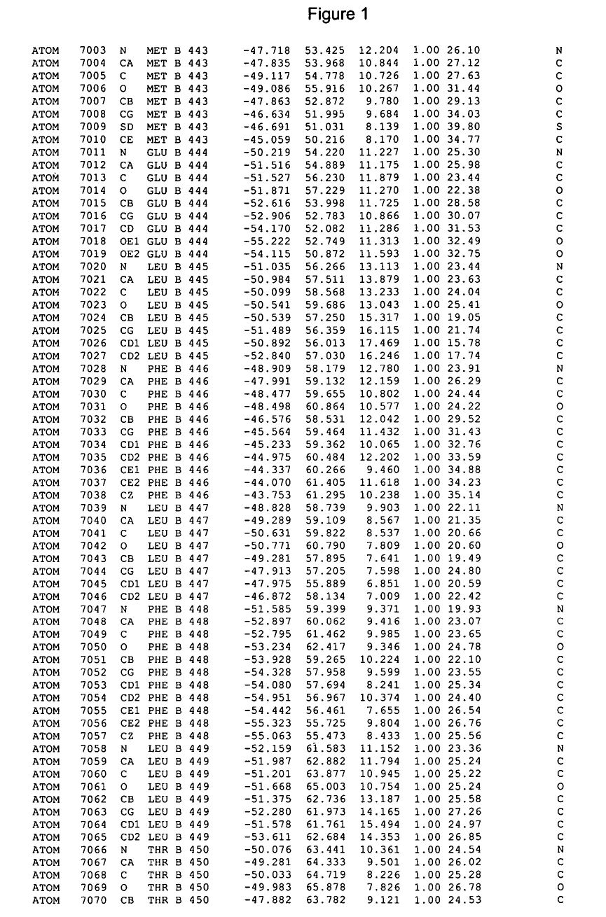

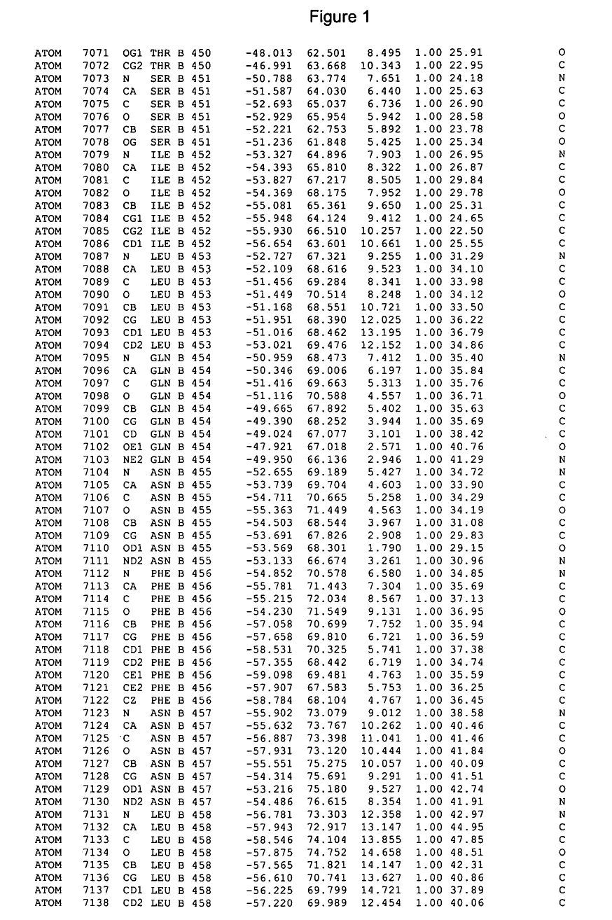

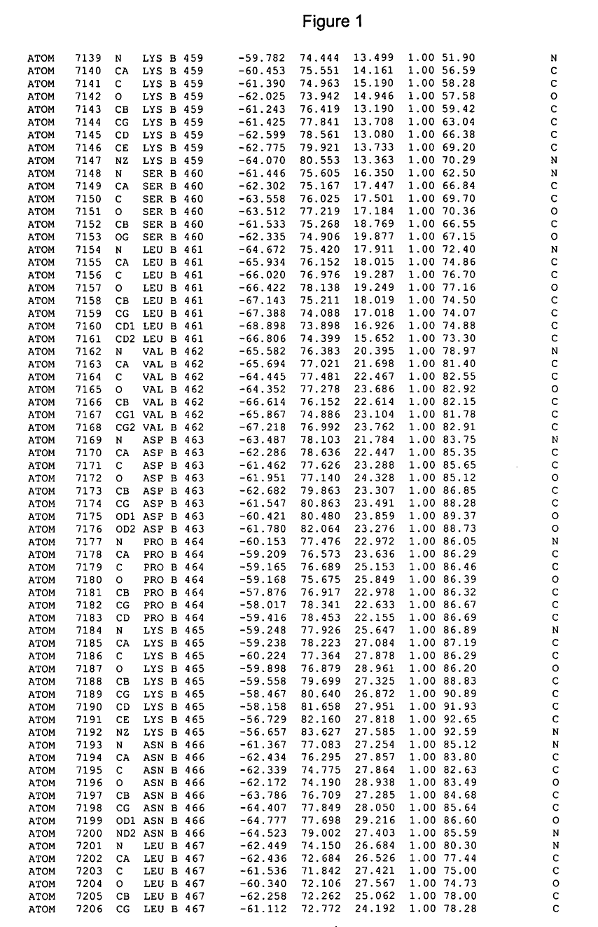

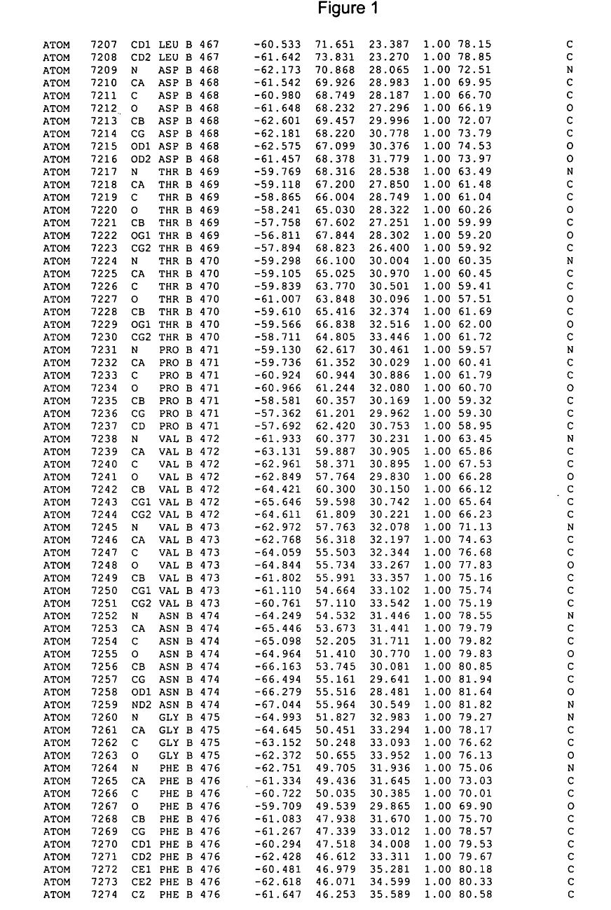

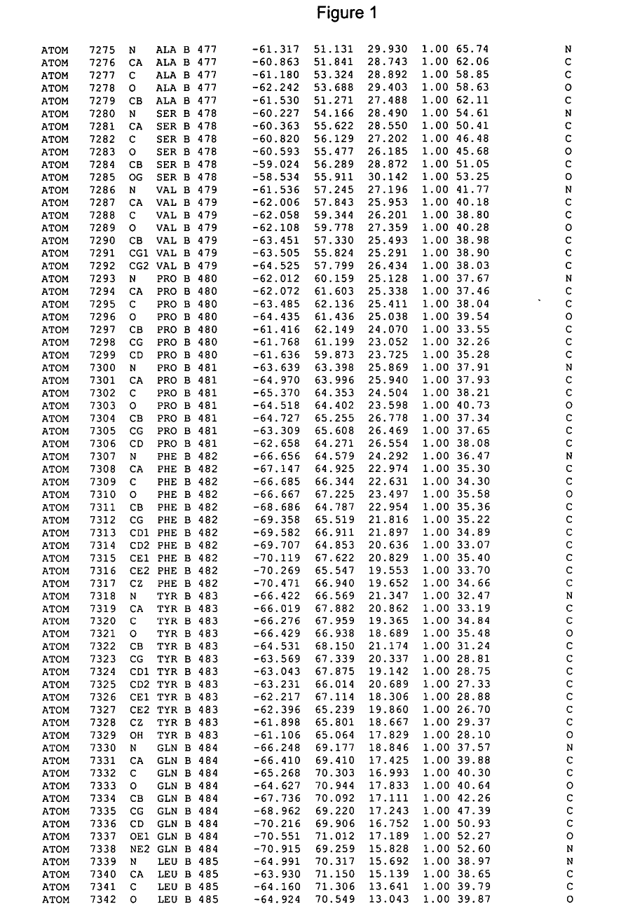

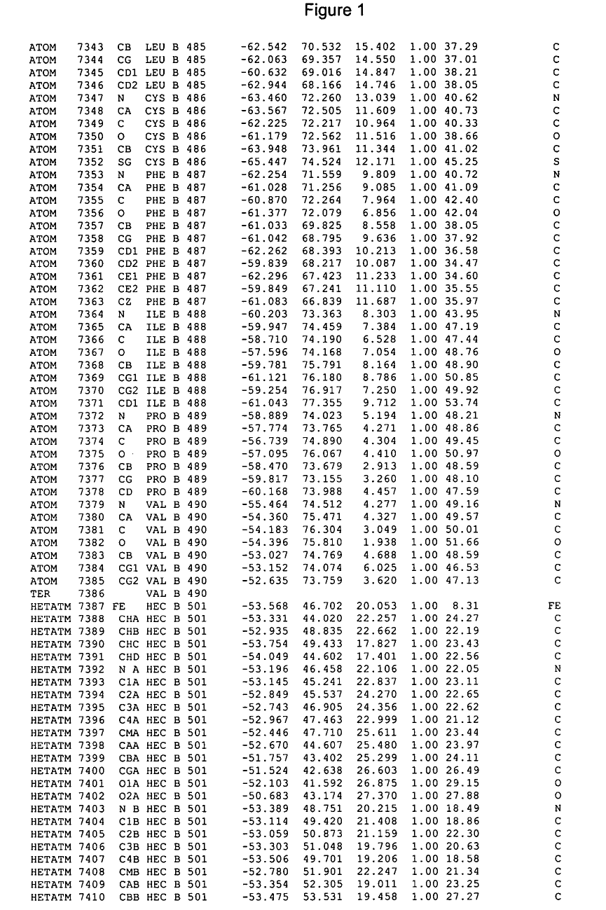

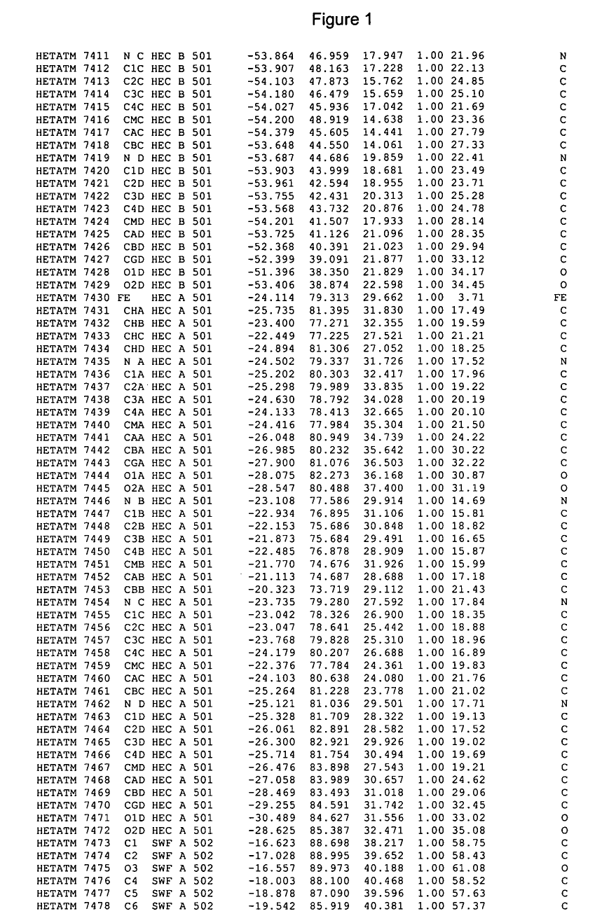

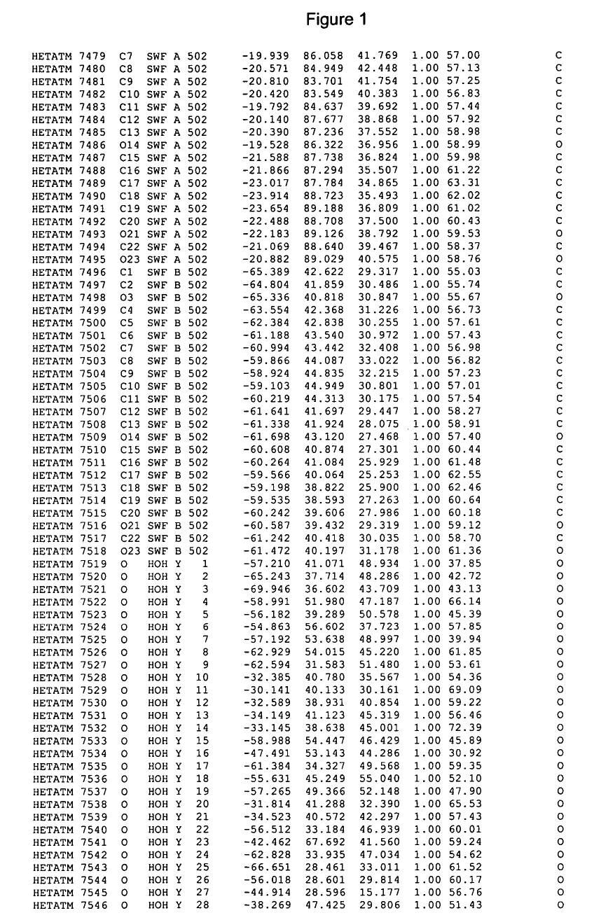

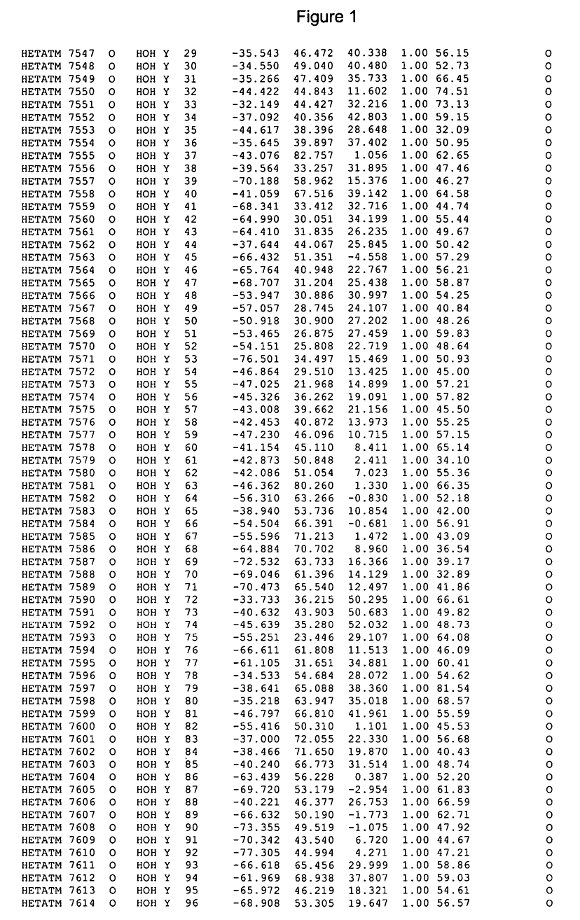

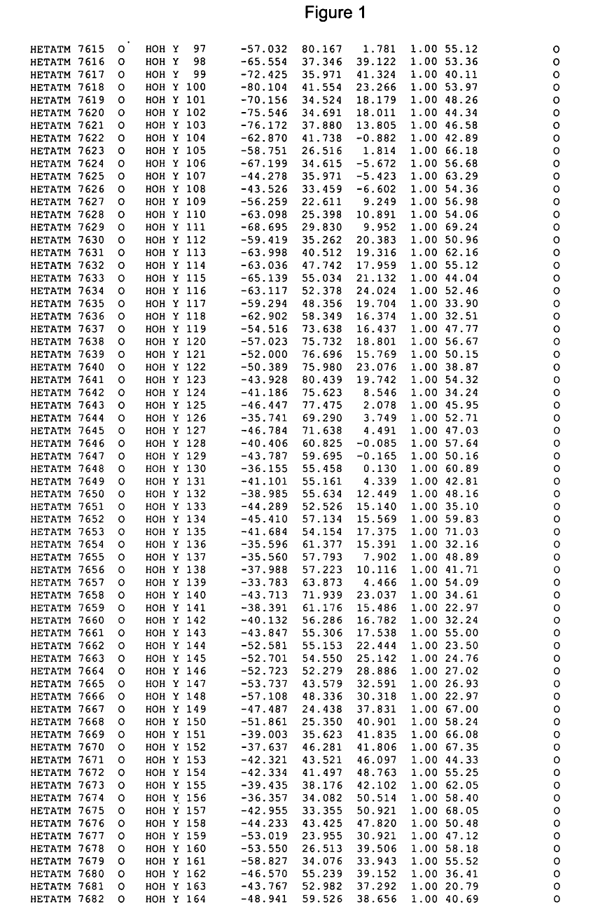

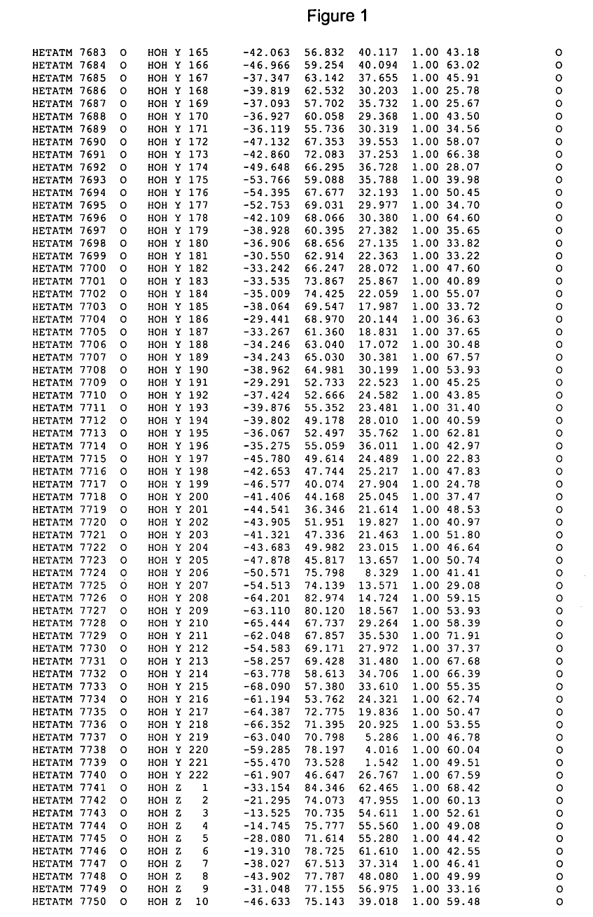

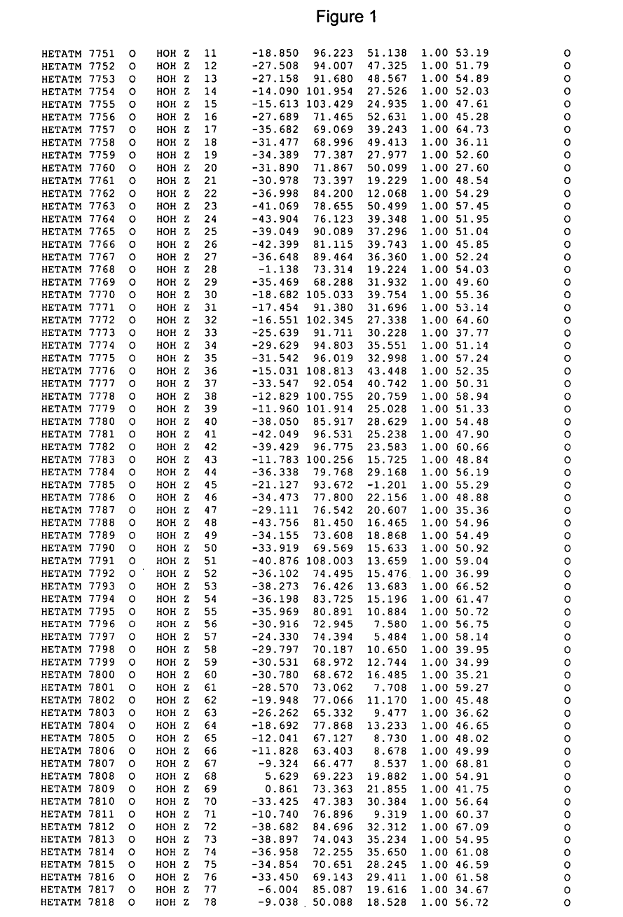

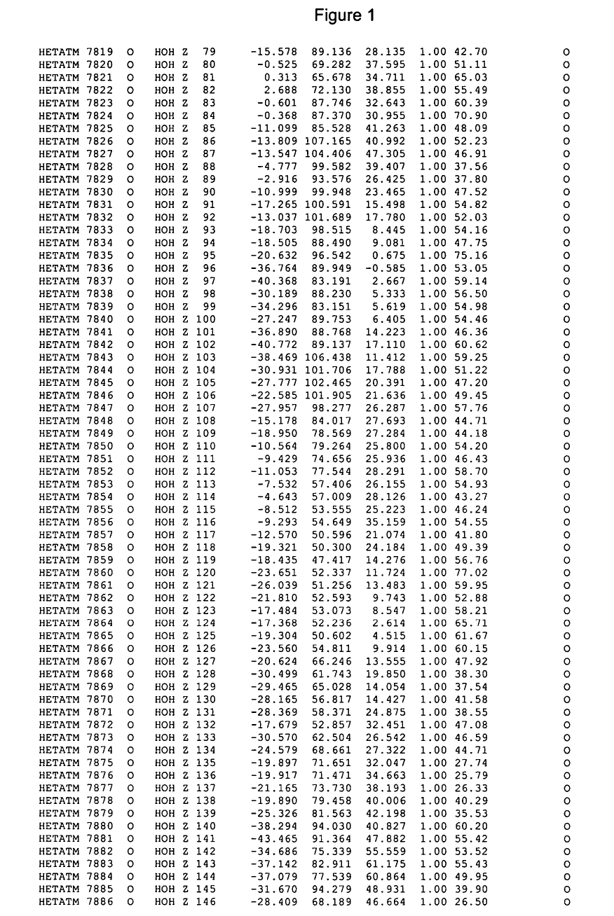

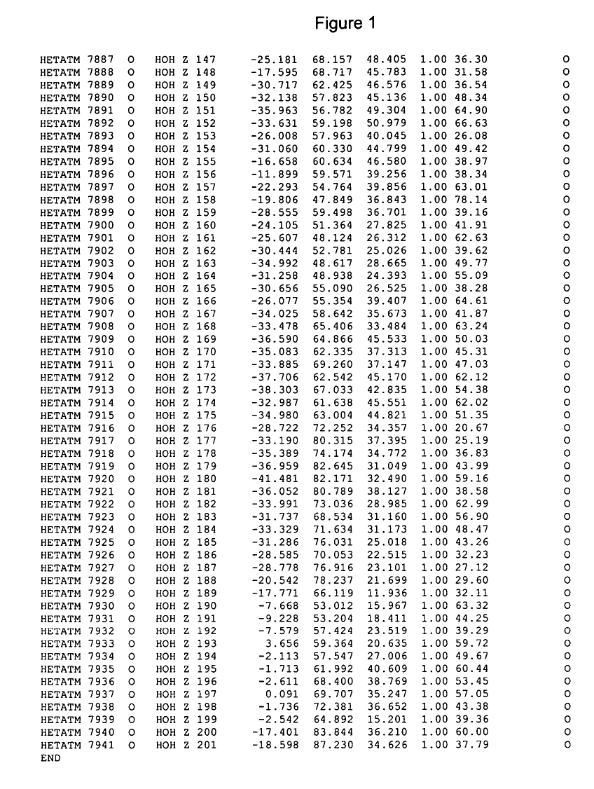

### DESCRIPTION OF TABLES

Table 1 provides the coordinates of 2C9-FGloop K206E co-crystallised with S-warfarin.

Table 2 sets out the coordinates of an apo 2C9 structure.

Table 3 sets out the residues lining the 2C9 binding pocket.

Table 4 sets out the residues newly identified as lining the 2C9 binding pocket.

Table 5 sets out residues of the 2C9 warfarin binding pocket.

Table 6 sets out oligonucleotides used to generate mutations in 2C9 proteins.

Table 7 sets out kinetic parameters of 2C9 proteins for 2C9 substrates.

### DESCRIPTION OF SEQUENCES ID NOs: 1-6

SEQ ID NO:1 is the DNA sequence encoding 2C9-FGloop K206E (also referred to as 1155).

SEQ ID NO:2 is the sequence of 2C9-FGloop K206E.

SEQ ID NO:3 is the DNA sequence encoding 2C9trunc (also referred to as 1003).

SEQ ID NO:4 is the sequence of 2C9truc.

SEQ ID NO:5 is the sequence of 2C9 wild type.

SEQ ID NO:6 is the N-terminal sequence of SEQ ID NO:2 and SEQ ID NO:4.

### DETAILED DESCRIPTION OF THE INVENTION

A. 2C9 Protein.

Co-crystals according to the invention may be produced using the 2C9 protein of SEQ ID NO:2, or similar 2C9 proteins which are described in detail in WO03/035693. The sequence of 2C9 is available in the art, for example from a number of database sources cited in Nelson et al, 1996, ibid. This includes the SwissProt database, in which 2C9 is entry number P11712.

The 2C9 P450 protein is desirably truncated in its N-terminal region to delete the hydrophobic trans-membrane domain, and the region replaced by a short (e.g. 8 to 12 amino acid sequence containing one or more (e.g. 3, 4 or 5) positively charged amino acids. For expression of the human 2C9 P450, we have used an N-terminal sequence MAKKTSSKGR (SEQ ID NO:6) in place of the N-terminal 29 amino acid residues, which increases expression of the proteins in E. coil and increases solubility.

The 2C9 P450 may optionally comprise a tag, such as a C-terminal polyhistidine tag to allow for recovery and purification of the protein.

We have found that the position of the proline residue in the F-G loop appears to play a significant role in the formation of a P450 crystal. In particular, the presence of a proline at position 220 or 222 in 2C9 appears to be important for crystallisation to occur.

In 2C9 wild type there is a proline residue at position 221. Moving it to position 220, by substituting position 220 by proline and removing the Pro221 (by substitution by any other residue, but preferably alanine or threonine) in 2C9 promotes crystallisation. Alternatively the proline may be moved to position 222, with position 221 likewise being substituted.

In 2C9 we have made the changes to positions 220 and 221 with and without other changes. Where other changes were made, these were I215V, C216Y, I222L and I223L, although it is not essential that any or all of these be made to provide for crystallisation.

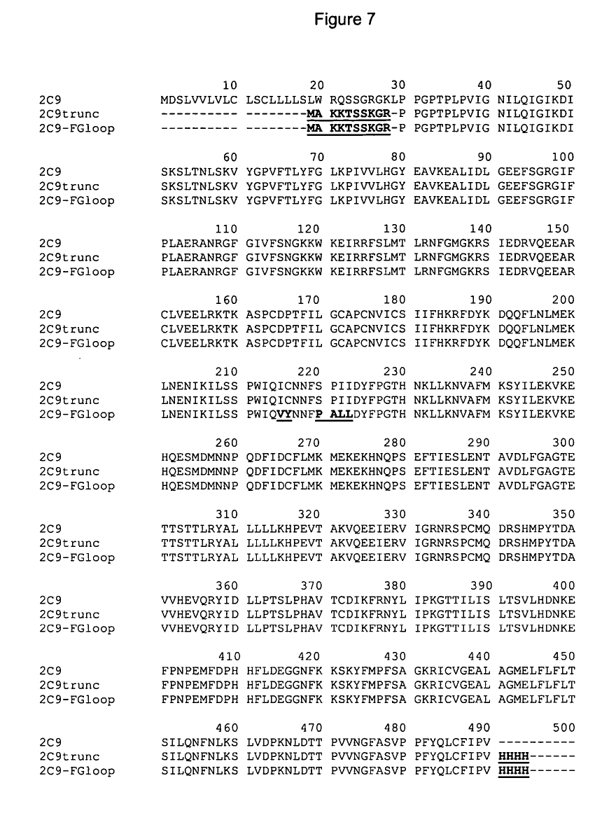

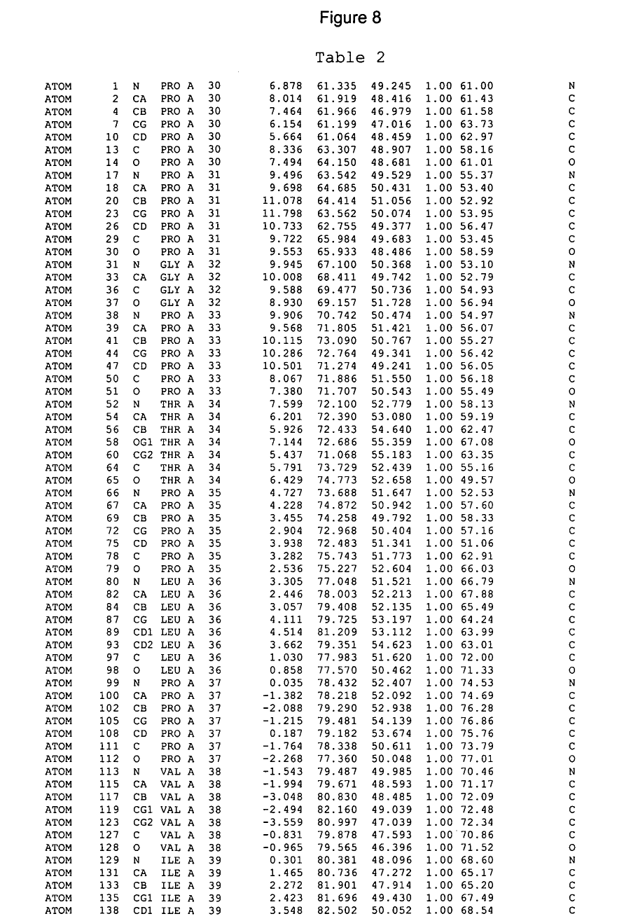

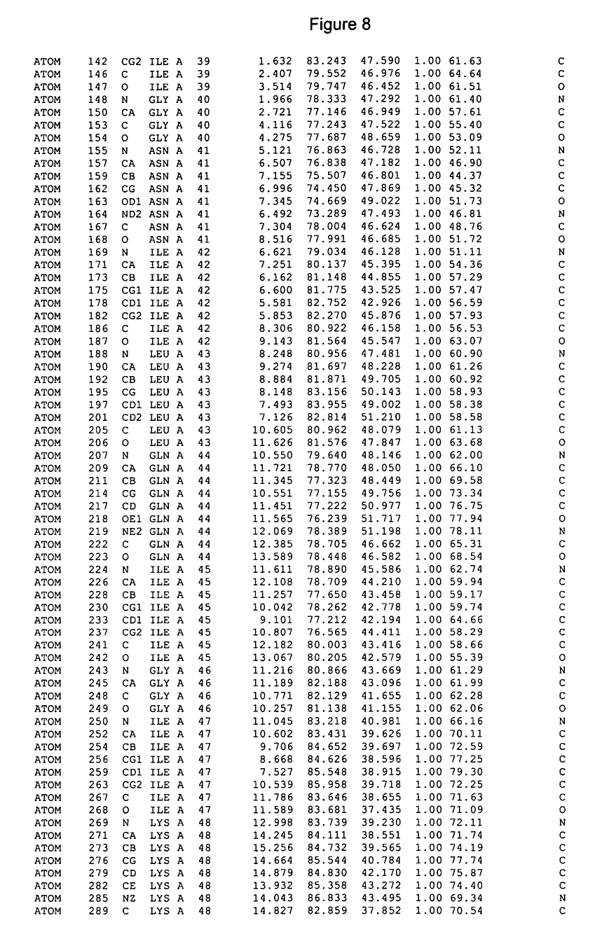

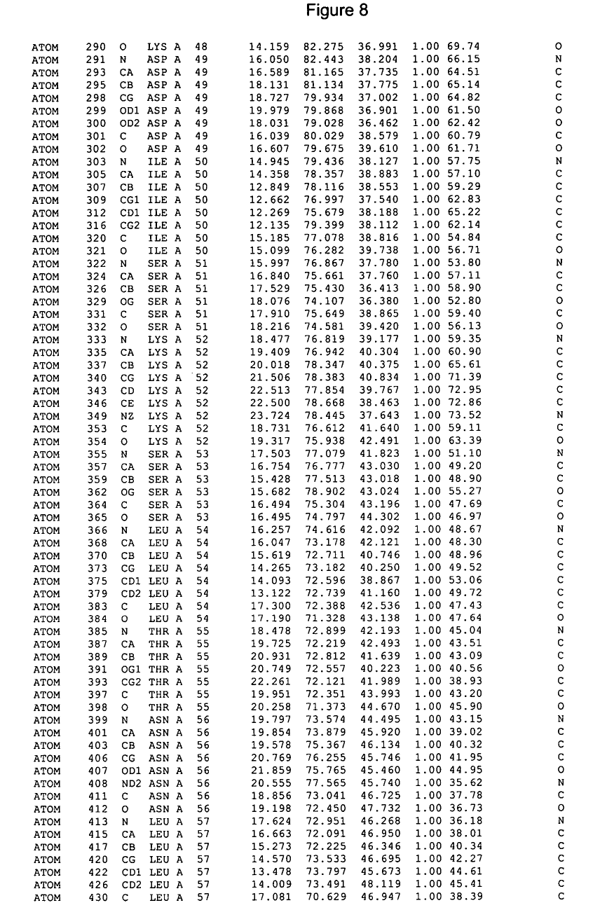

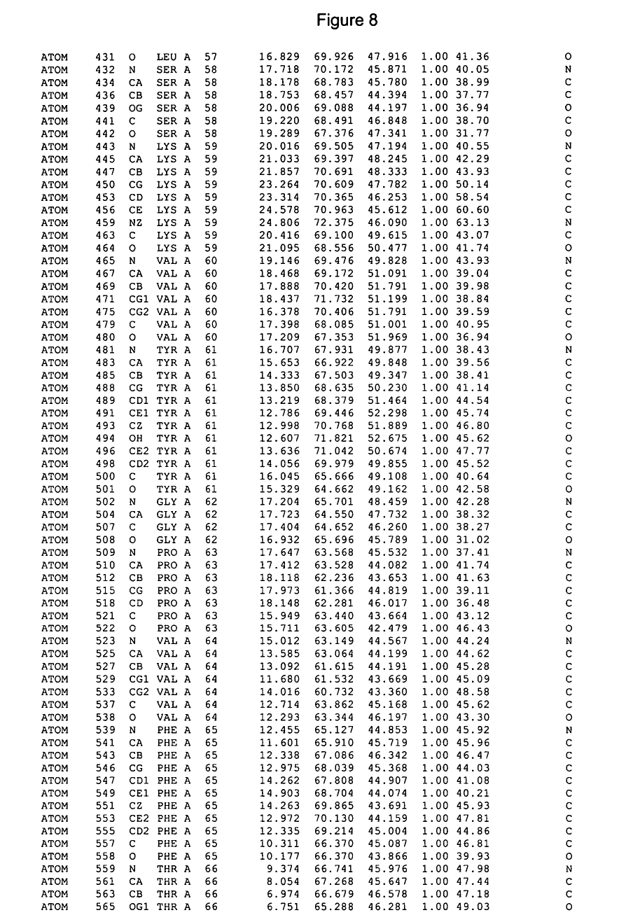

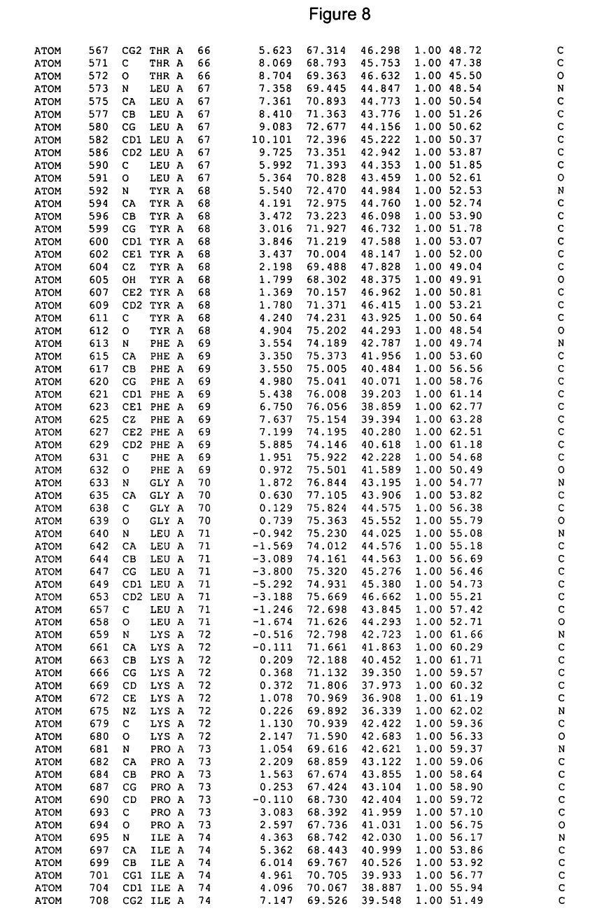

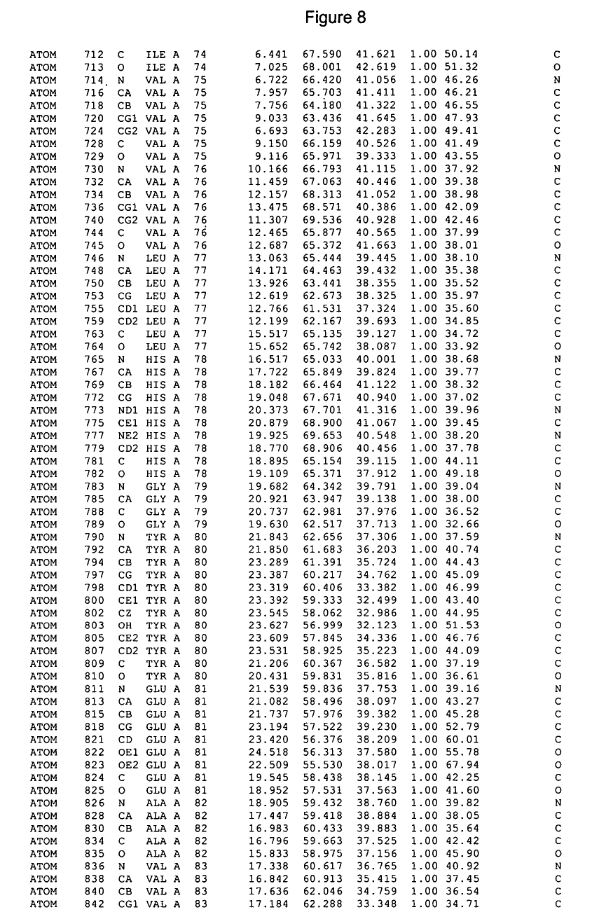

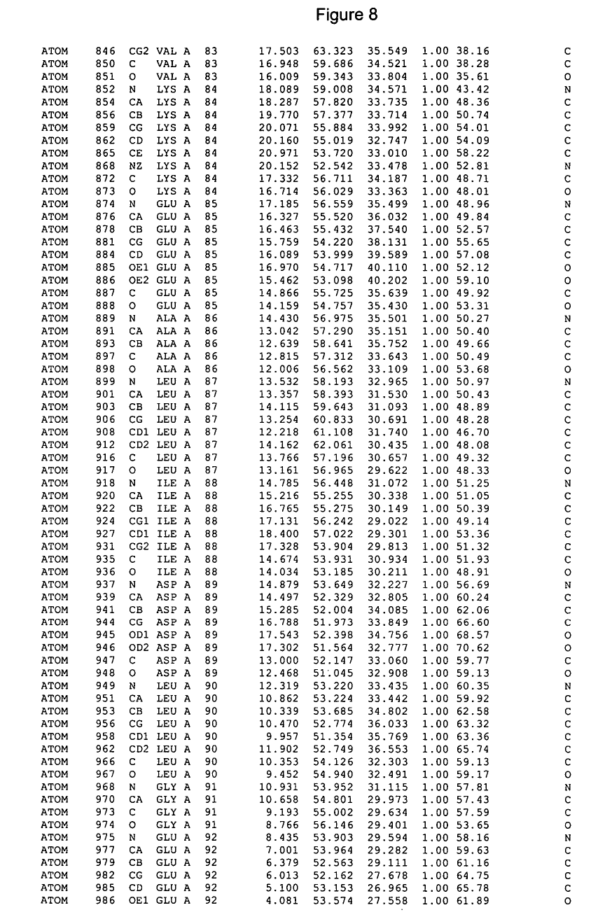

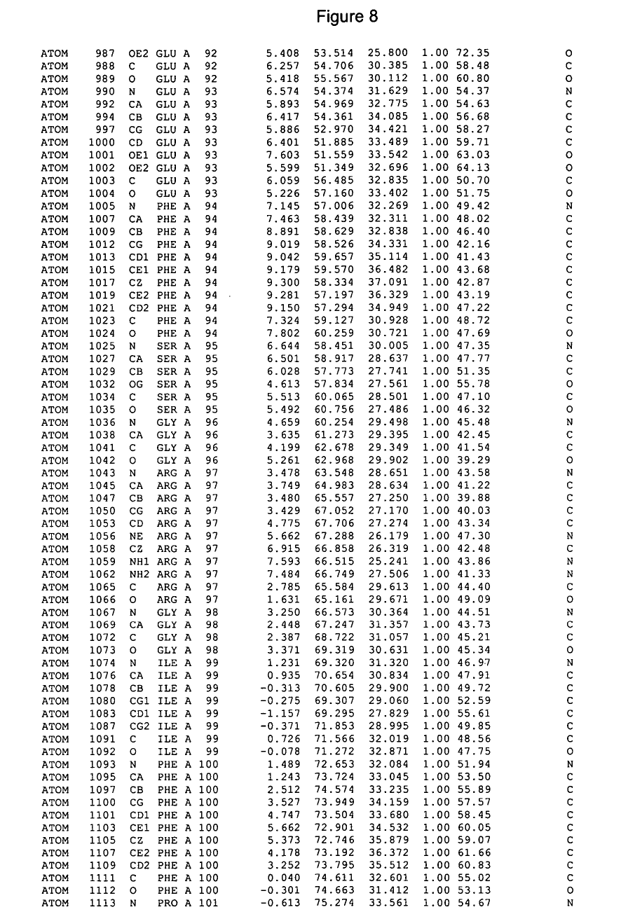

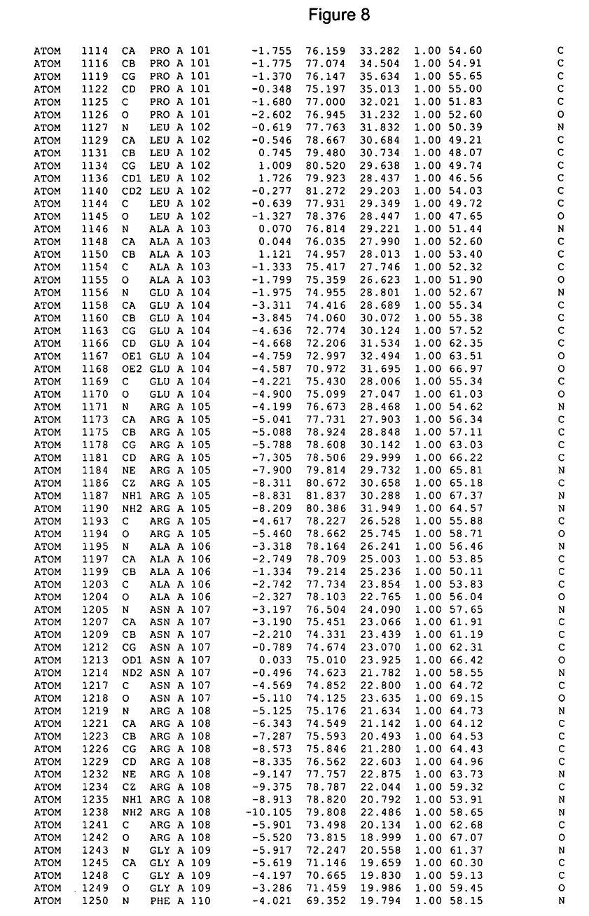

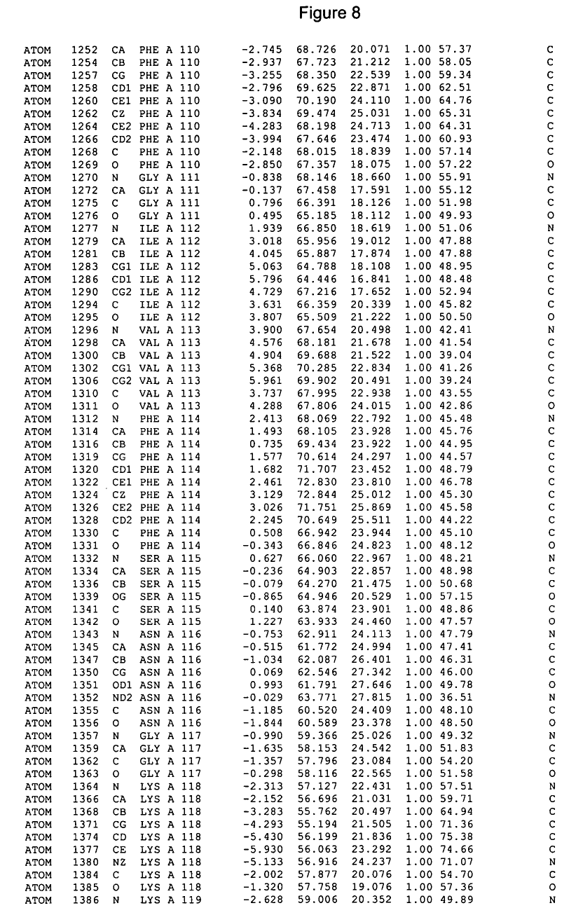

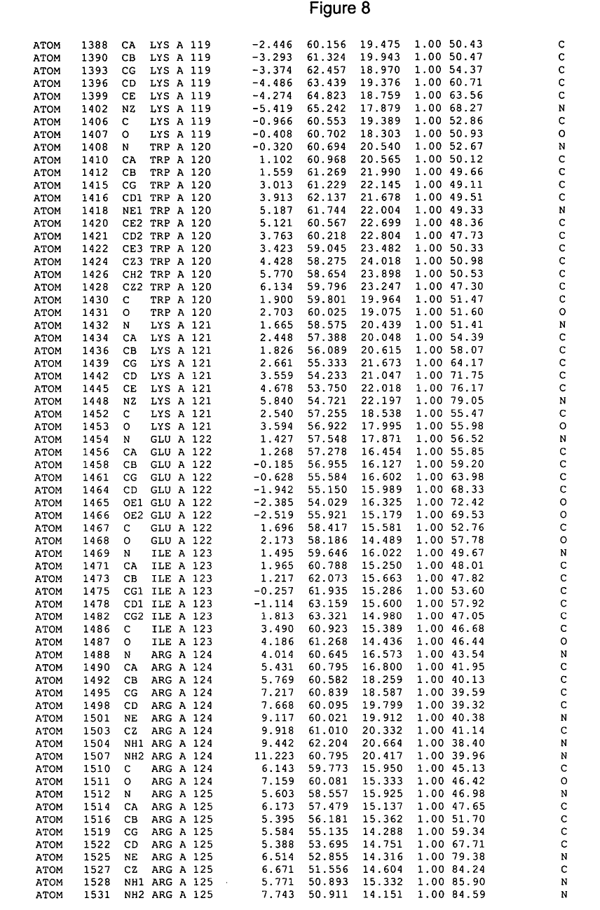

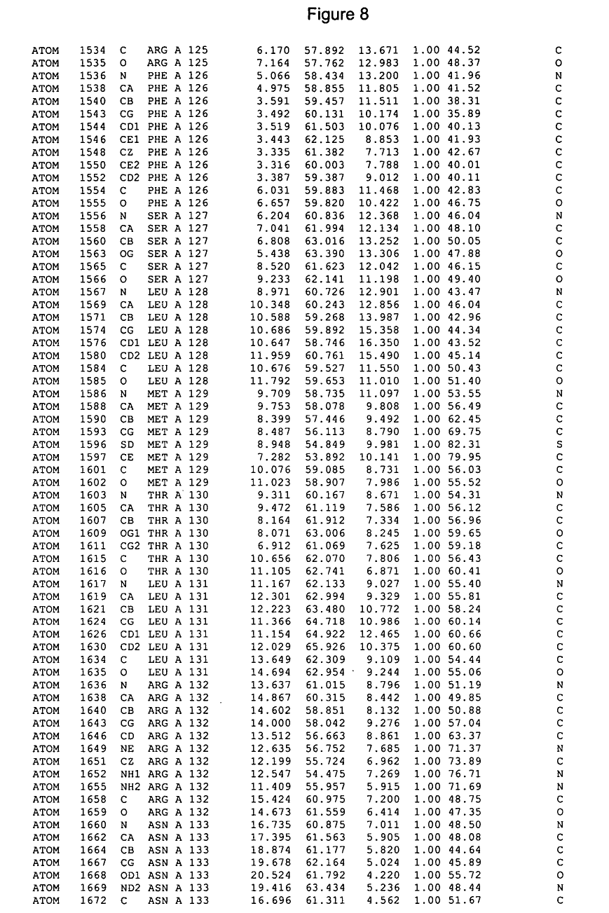

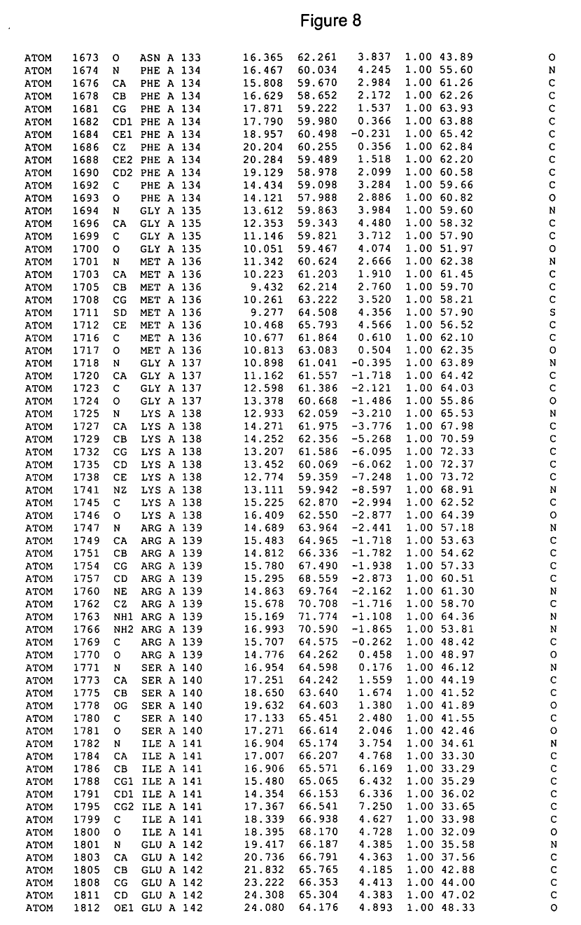

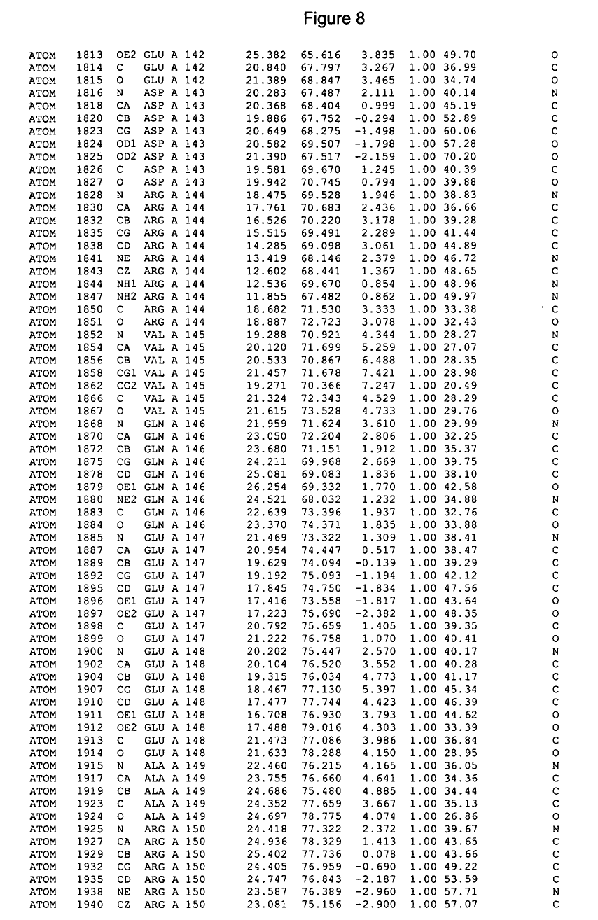

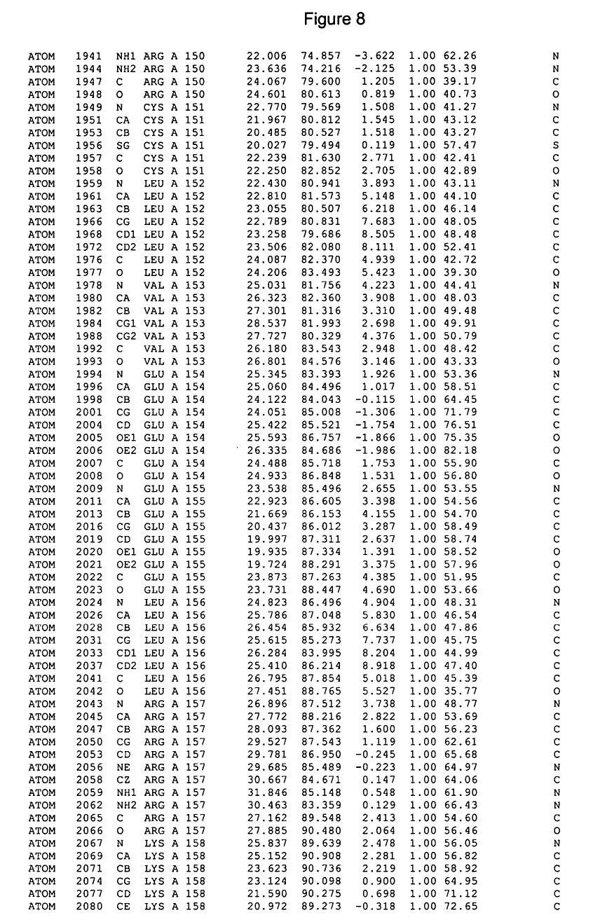

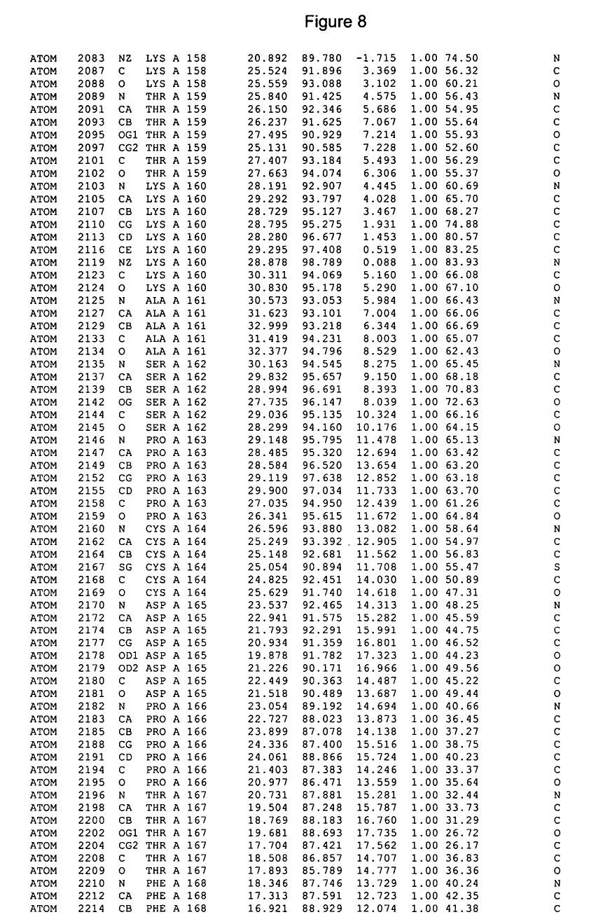

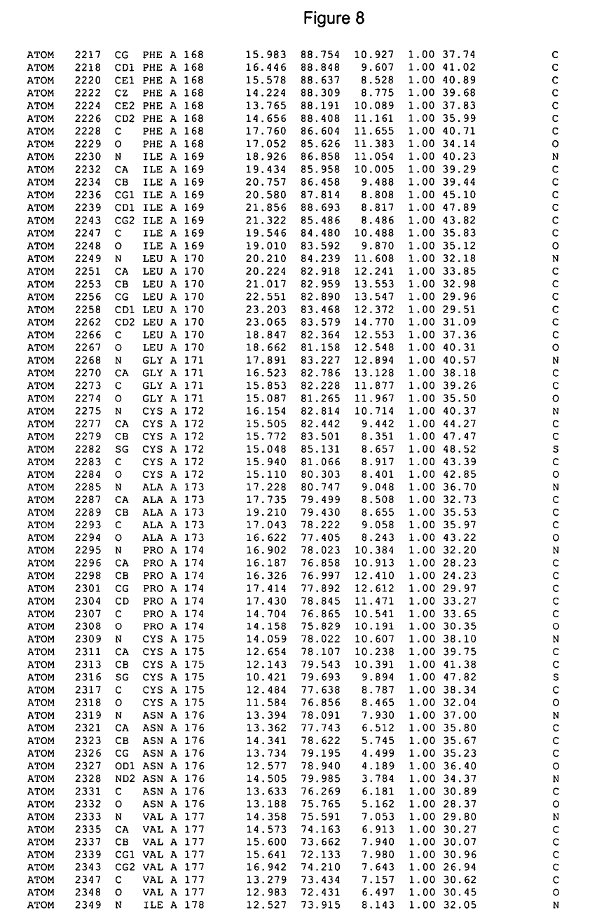

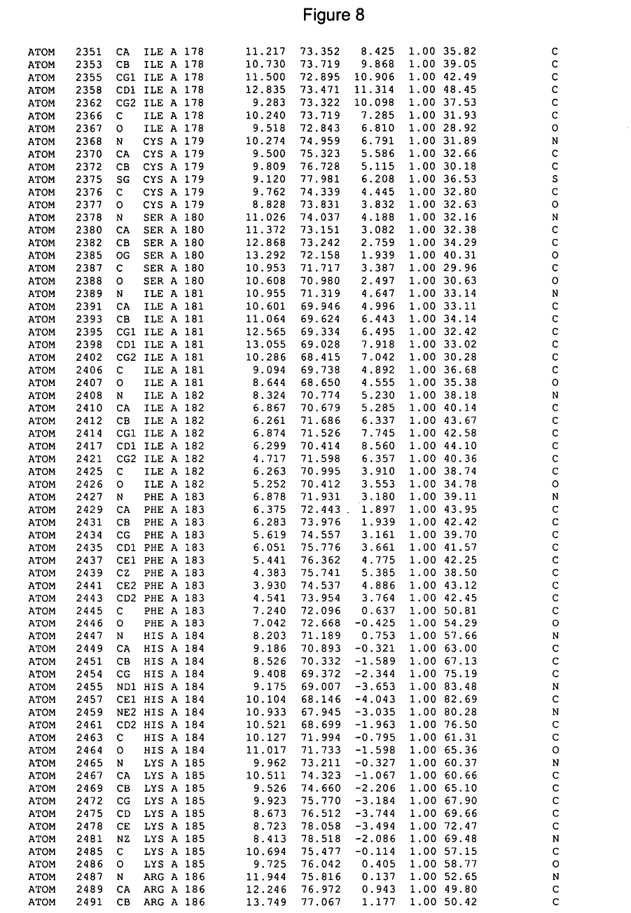

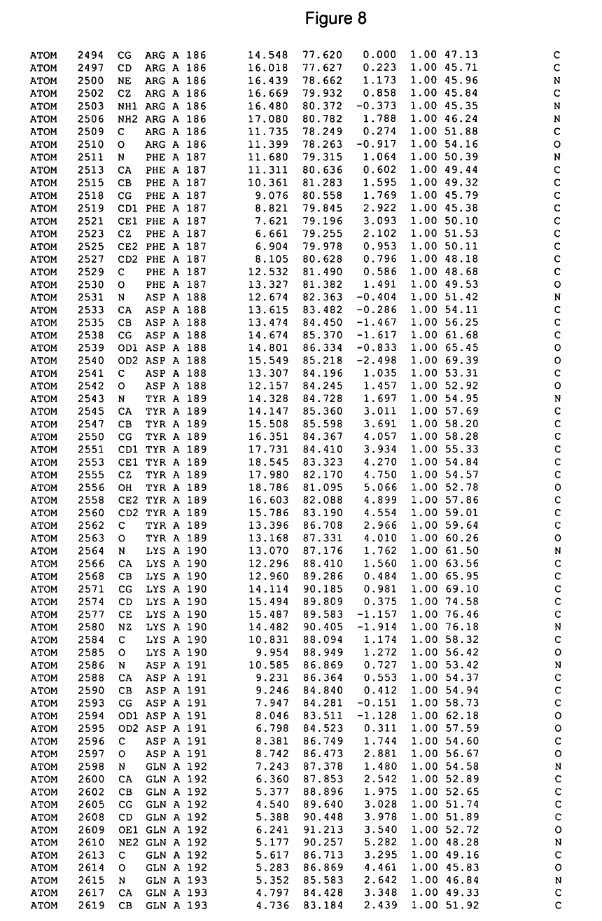

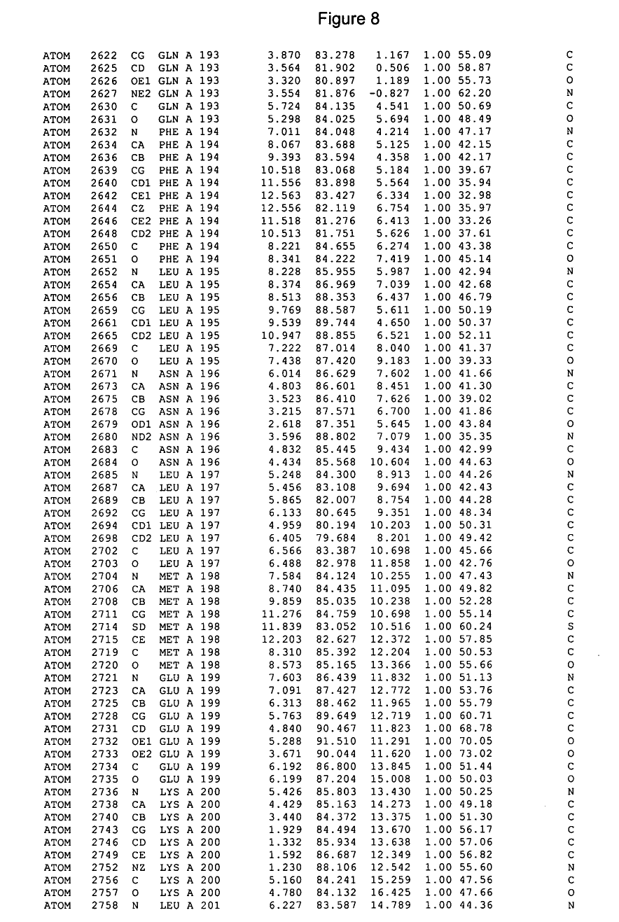

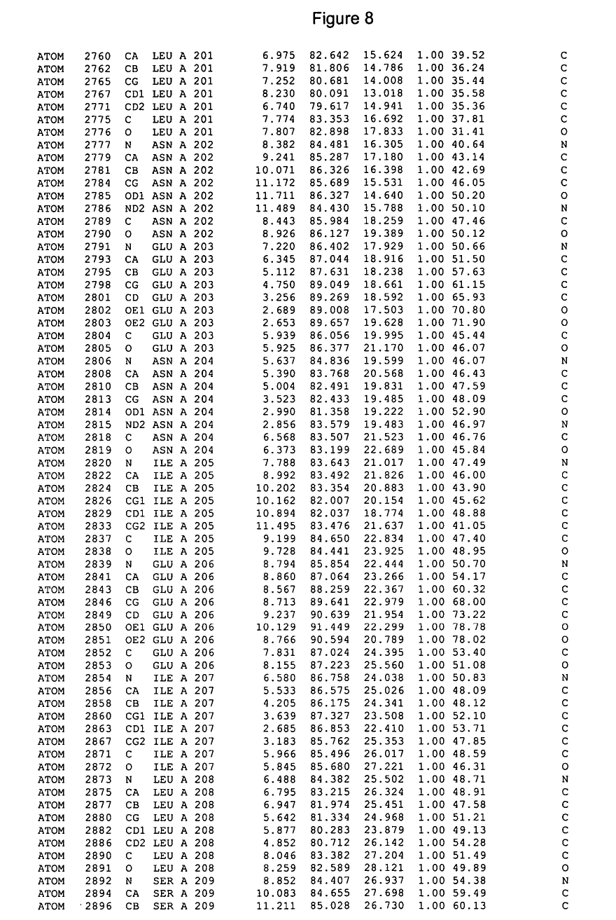

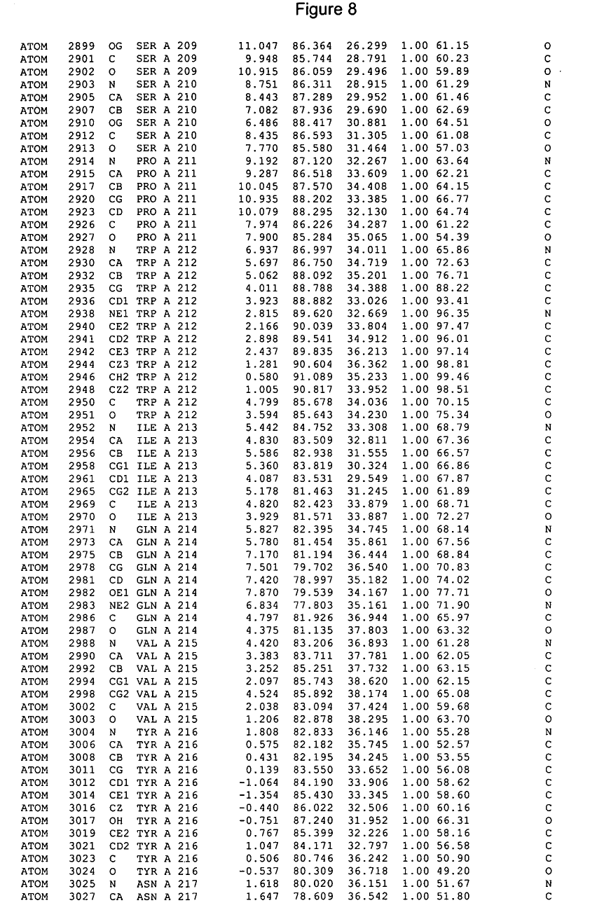

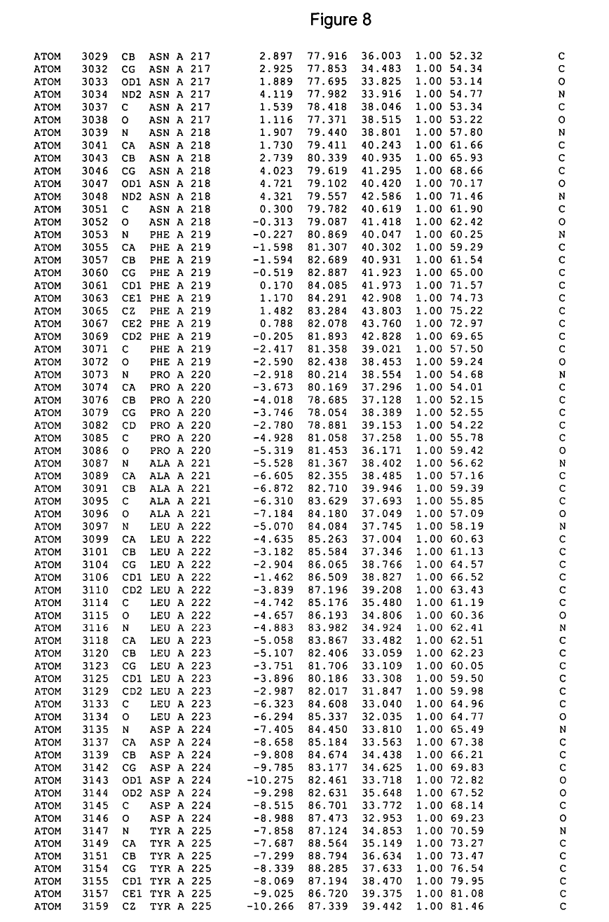

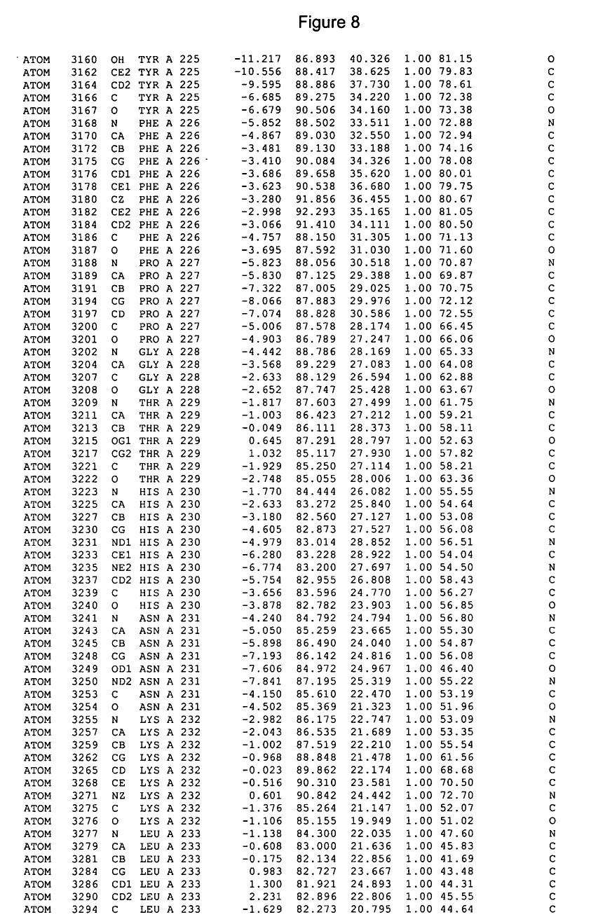

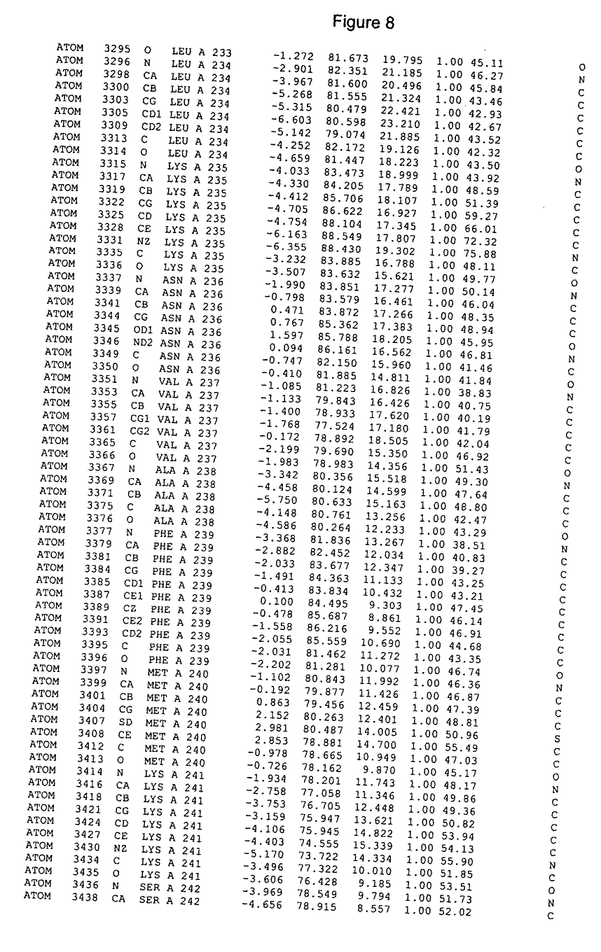

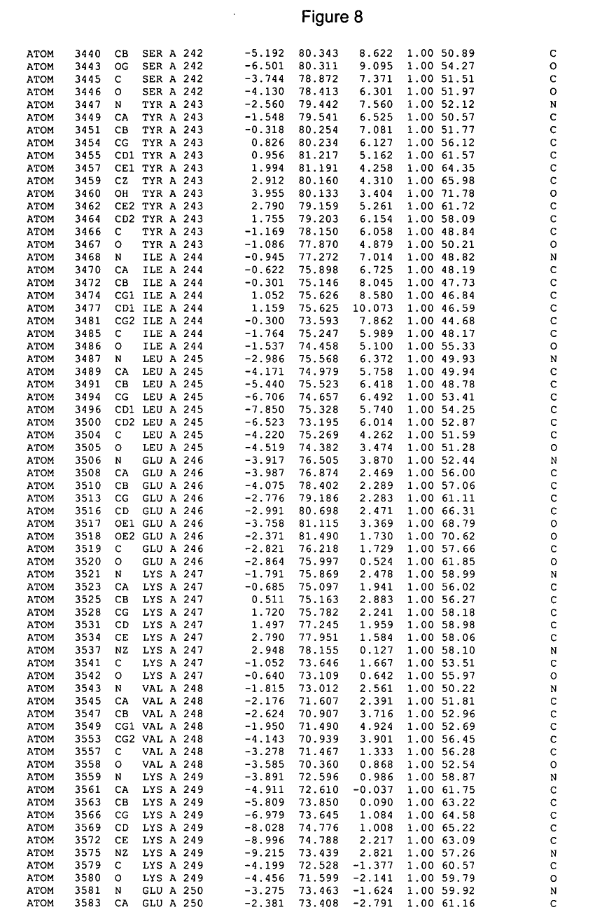

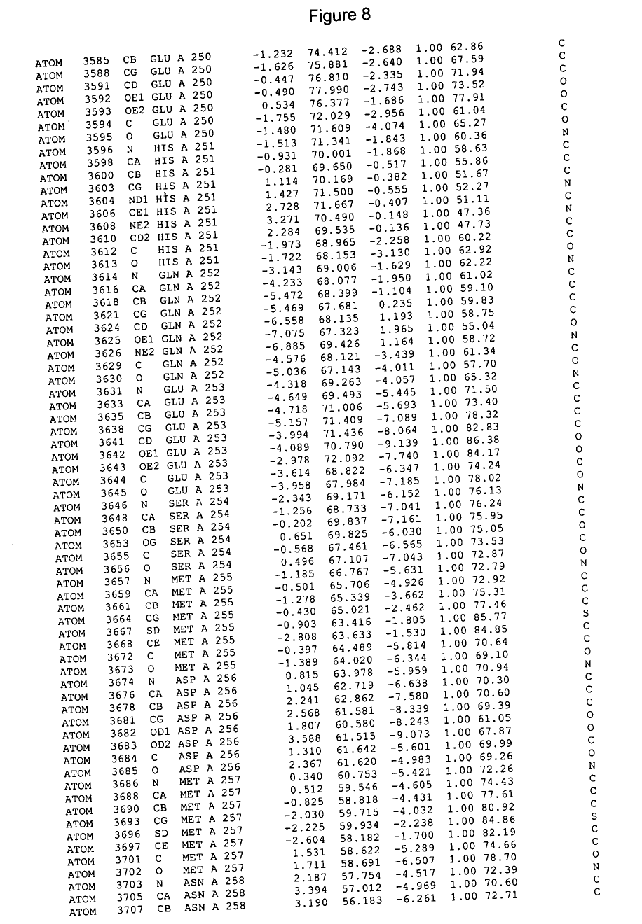

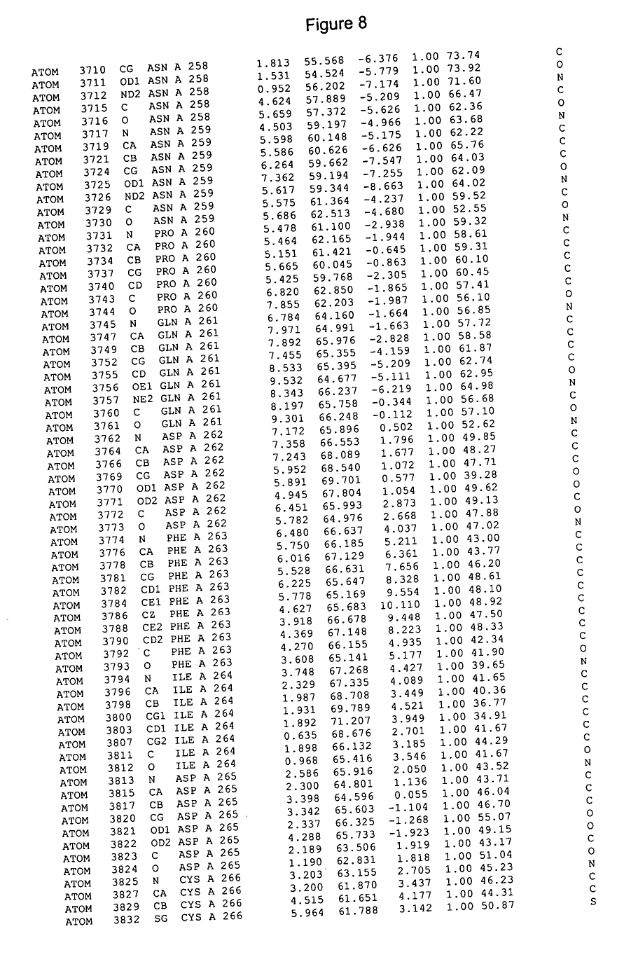

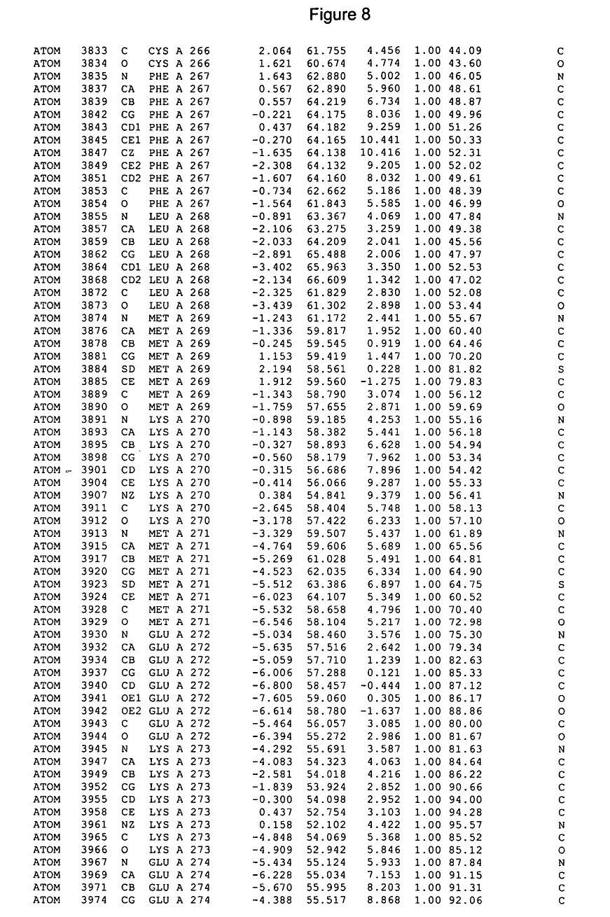

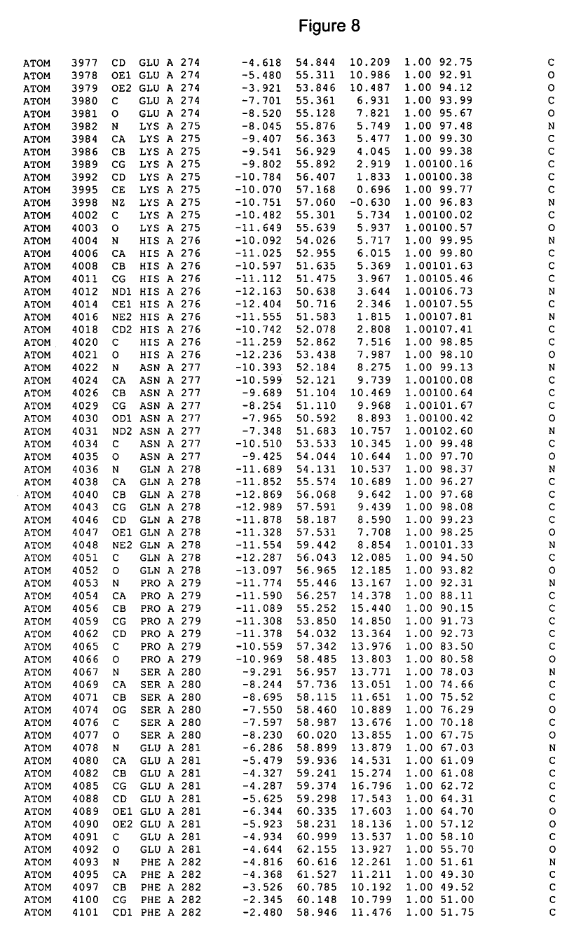

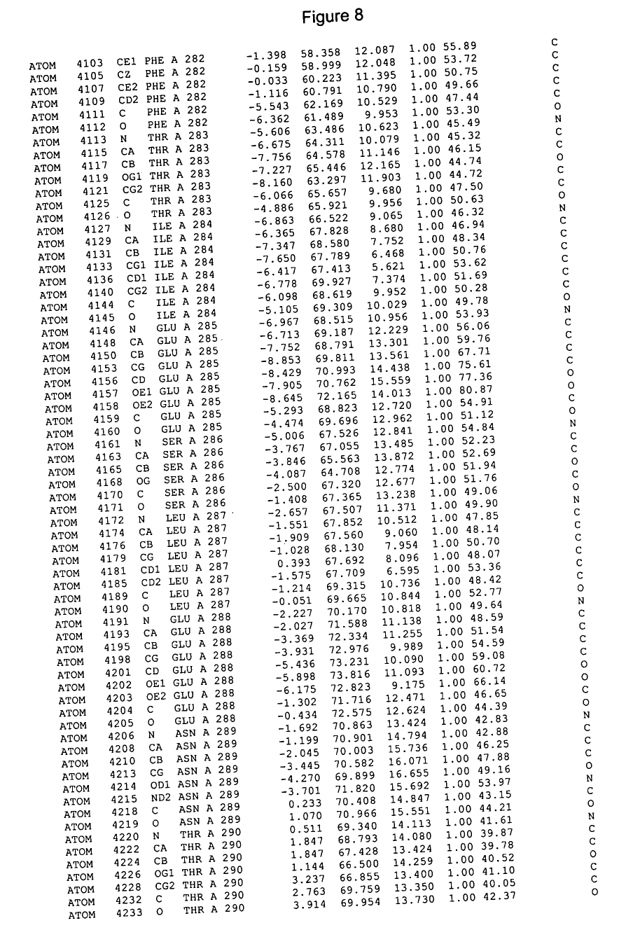

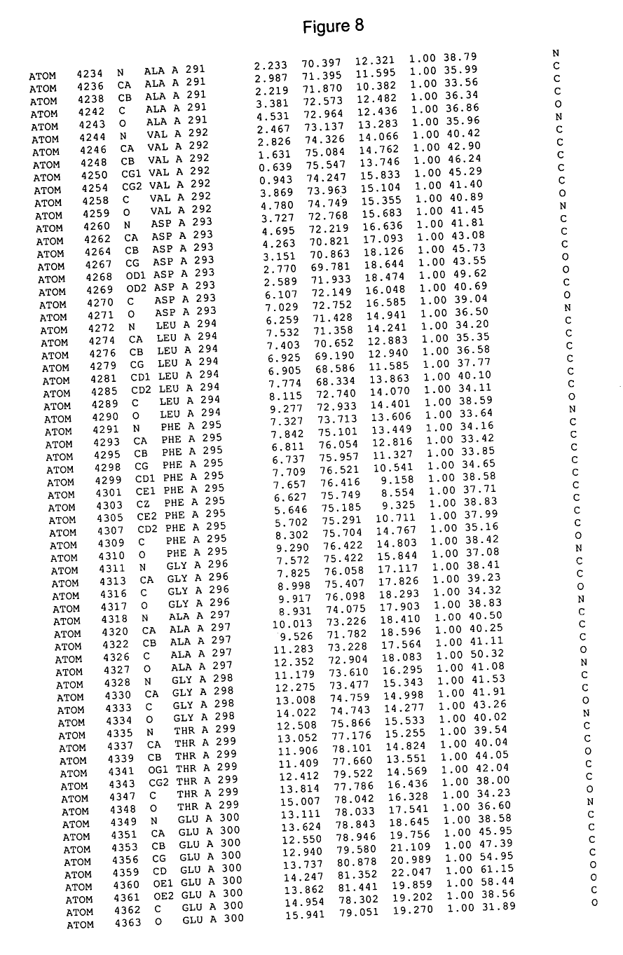

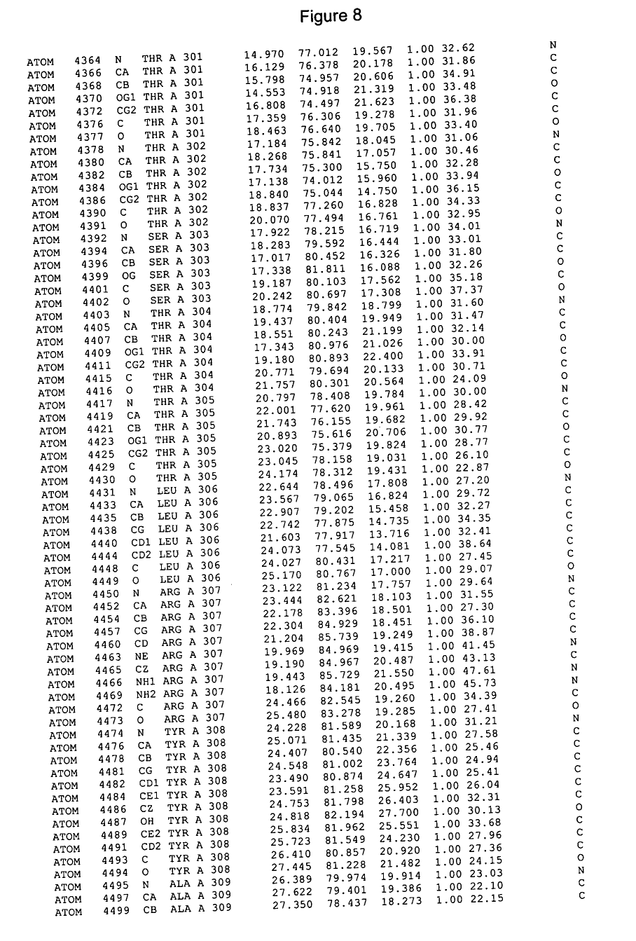

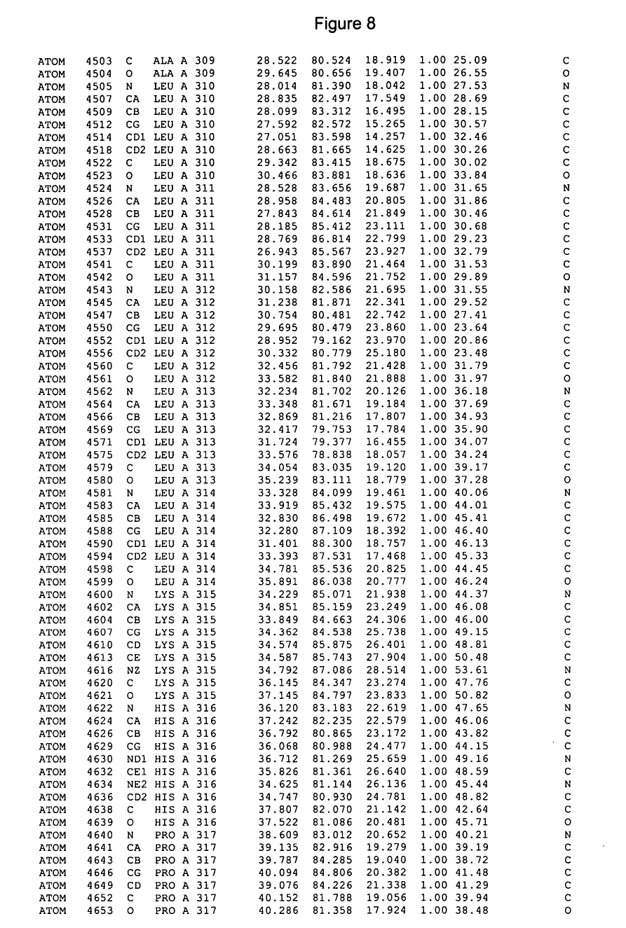

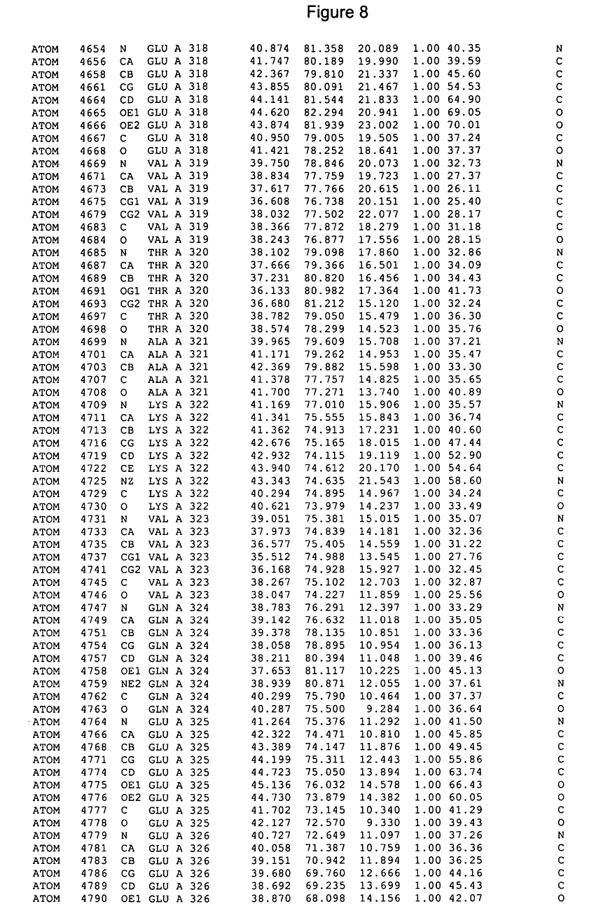

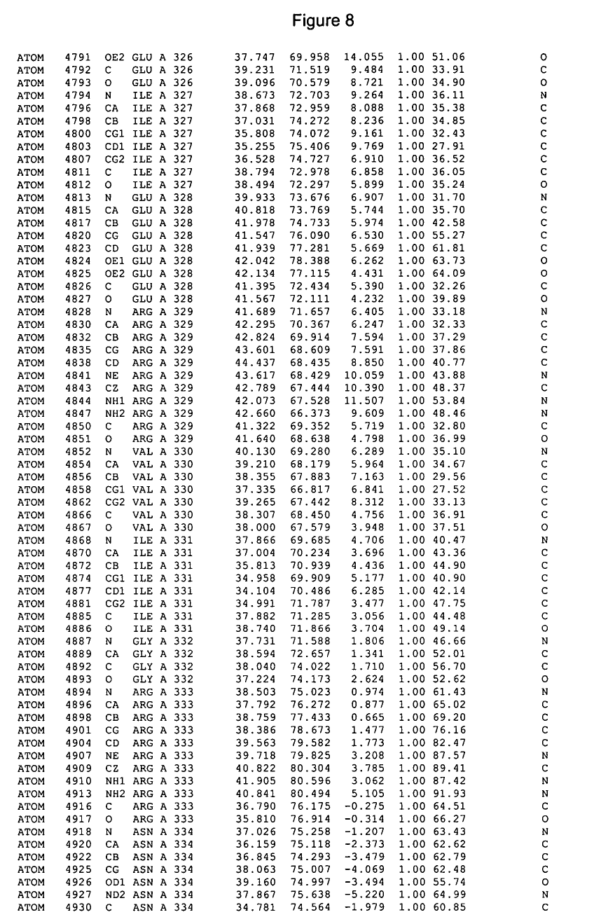

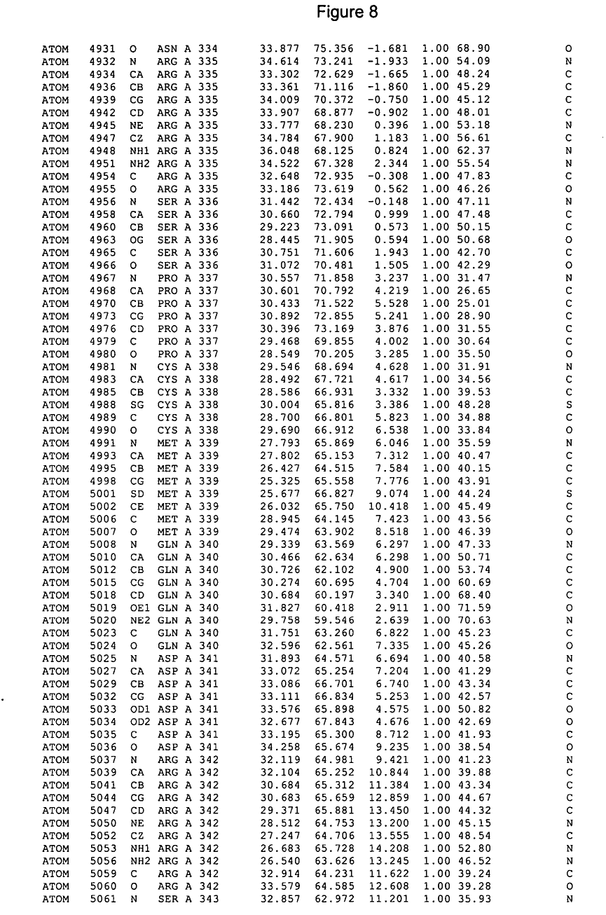

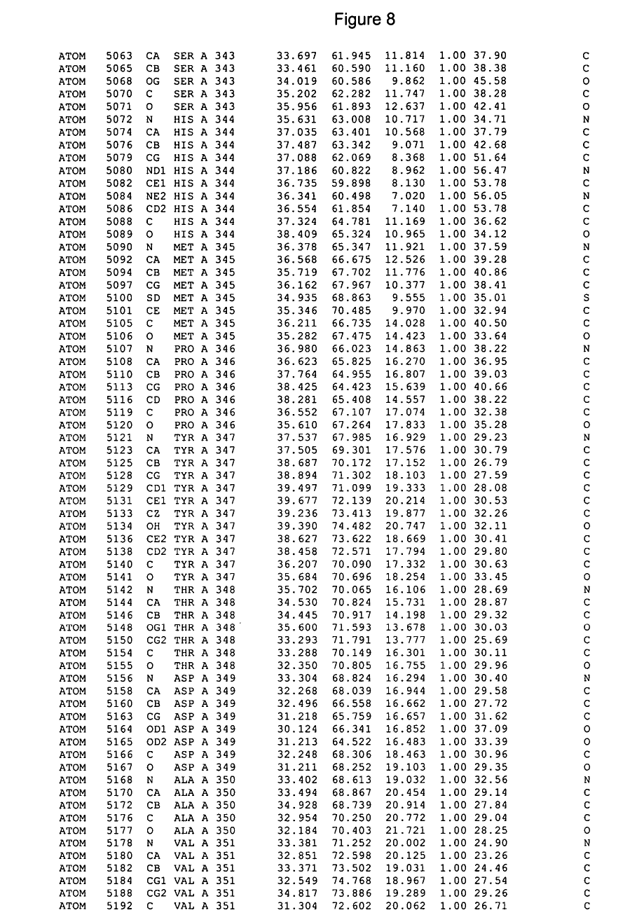

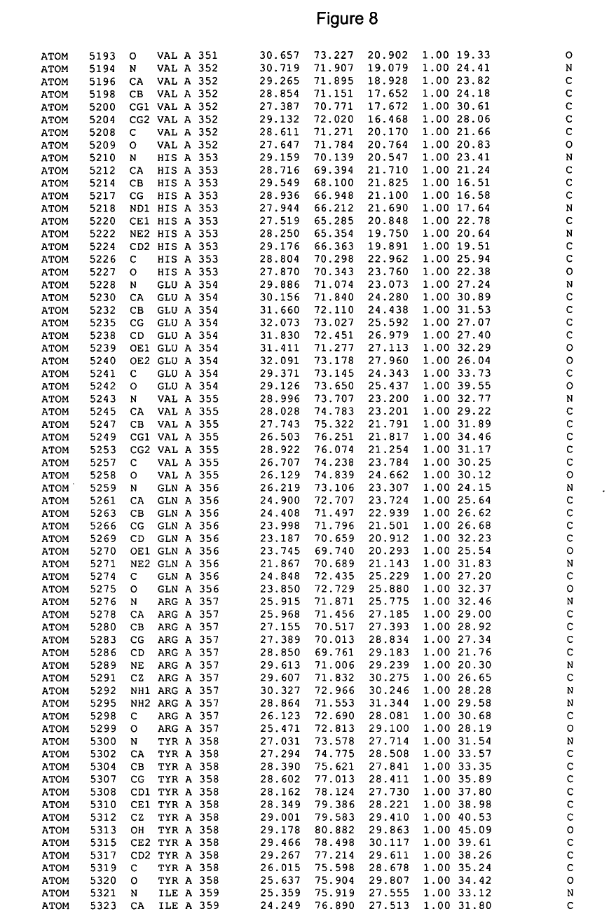

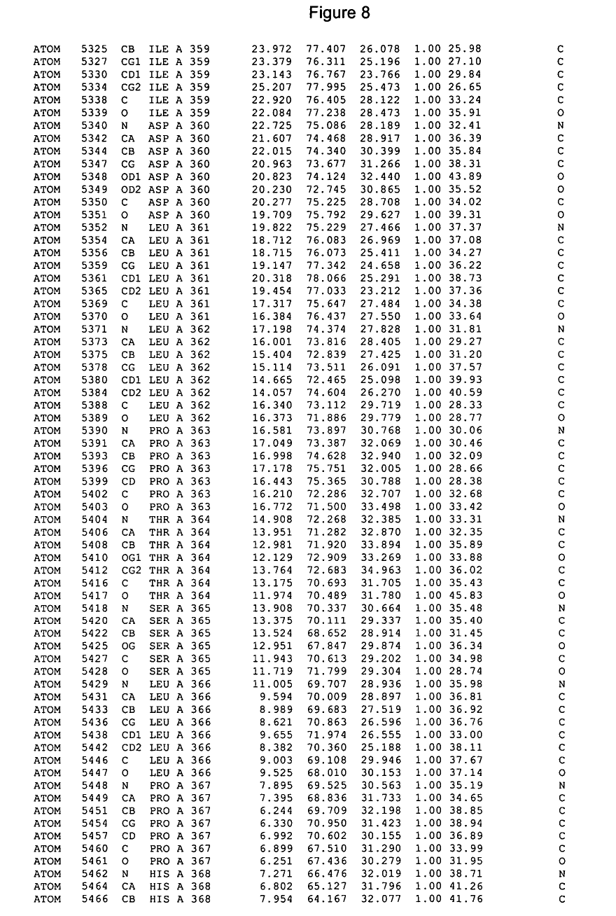

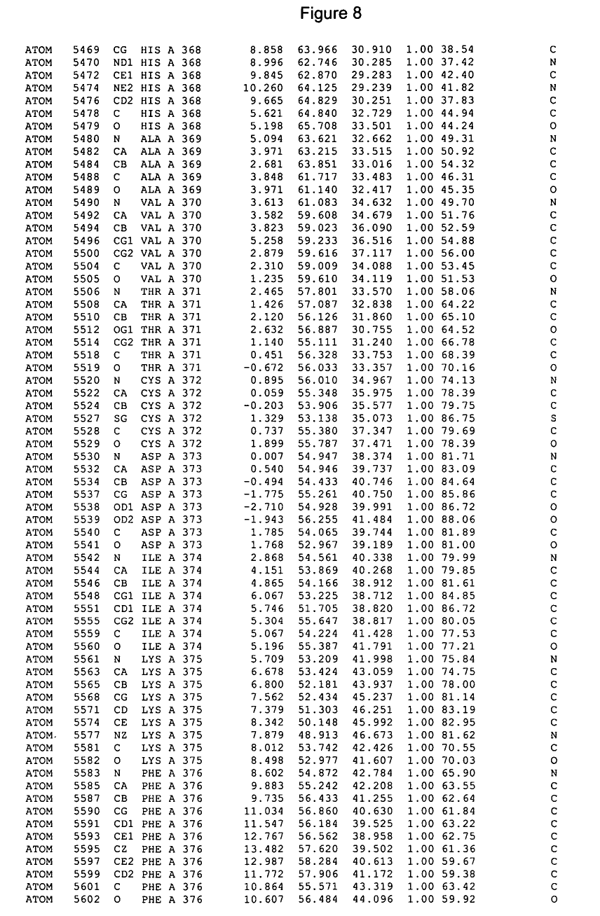

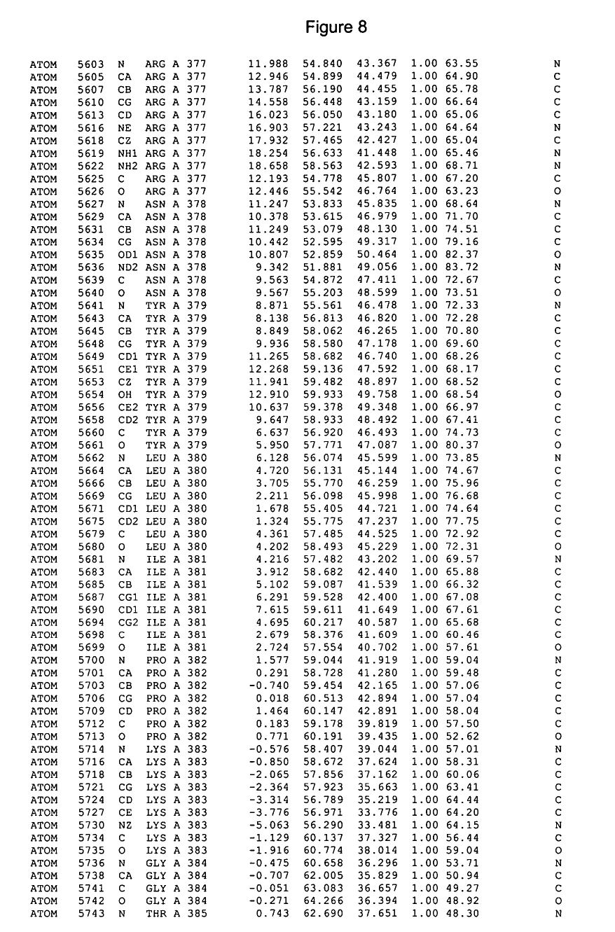

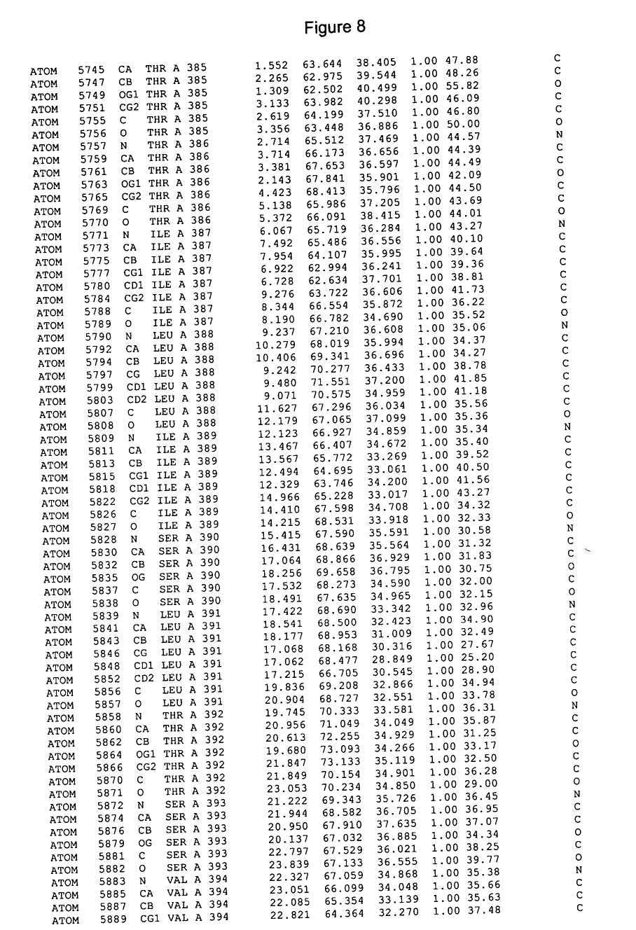

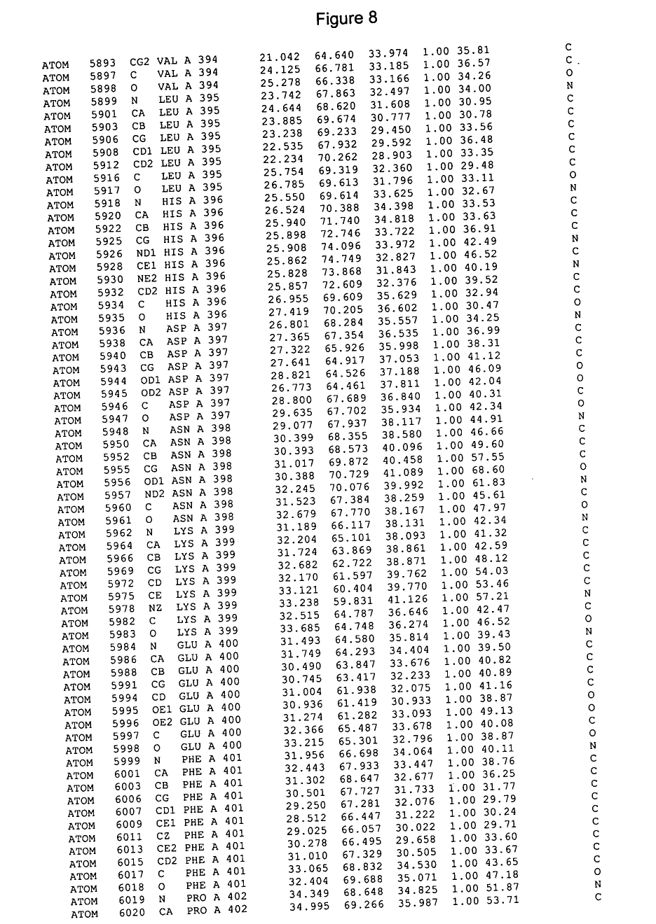

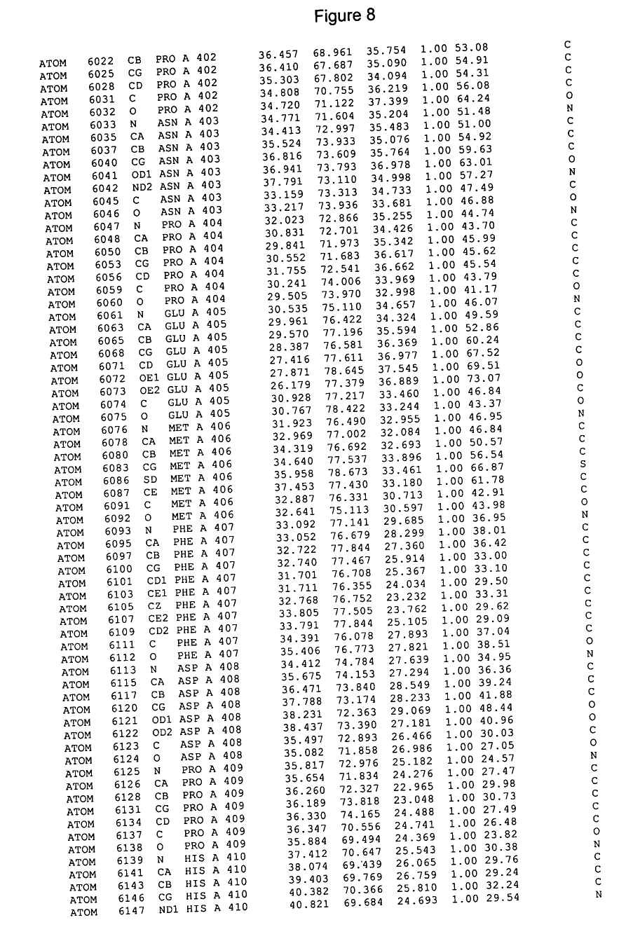

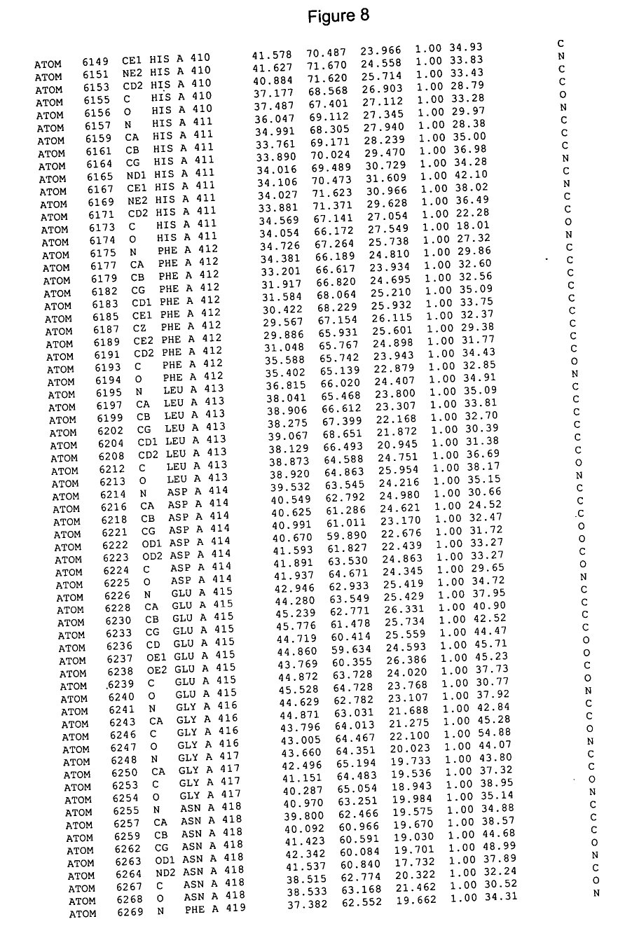

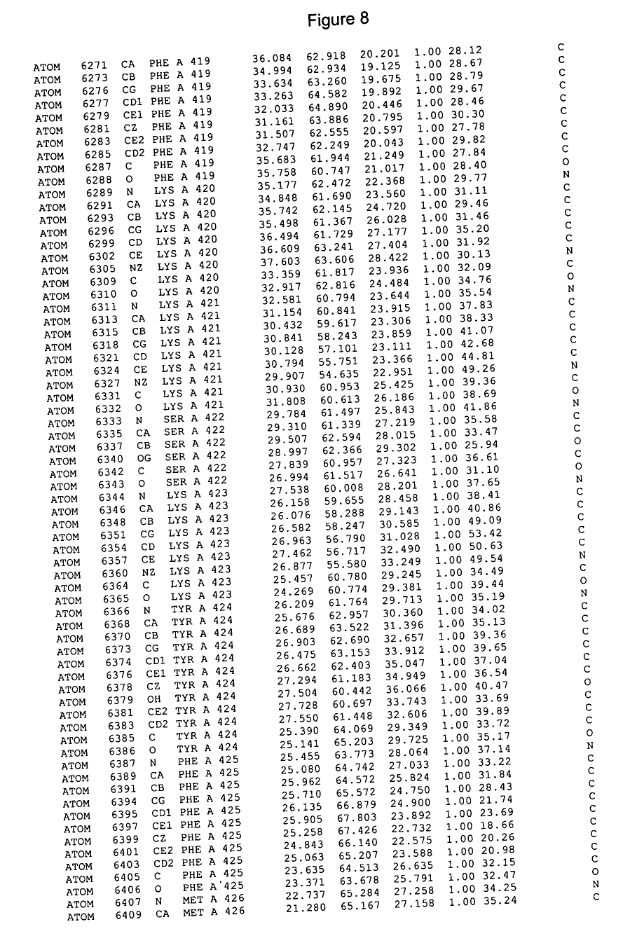

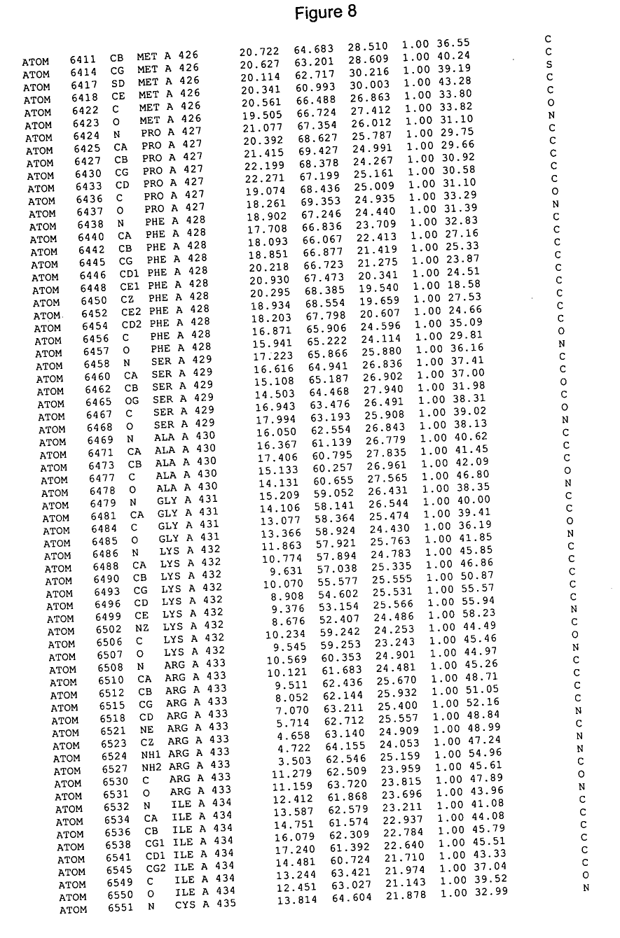

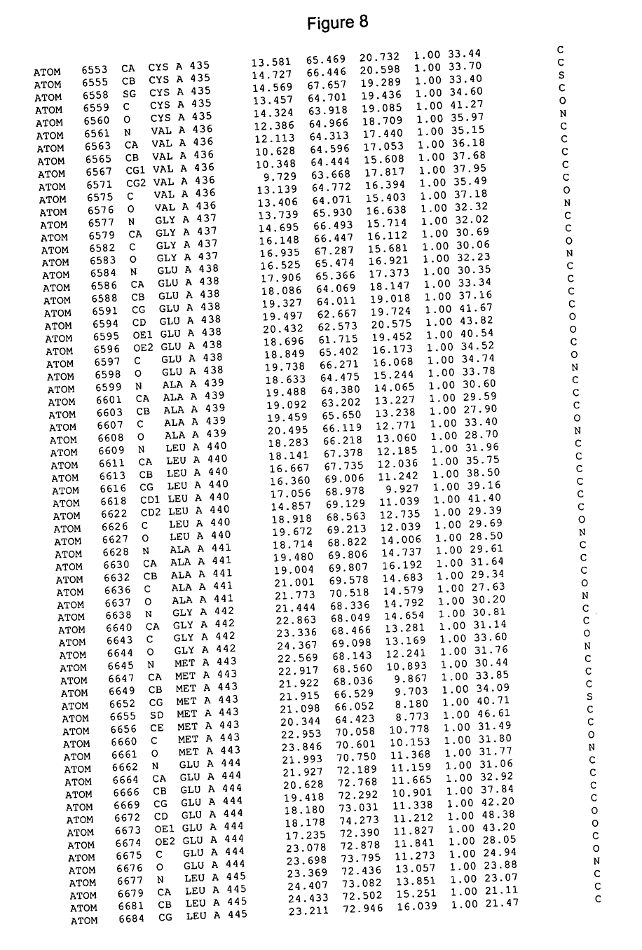

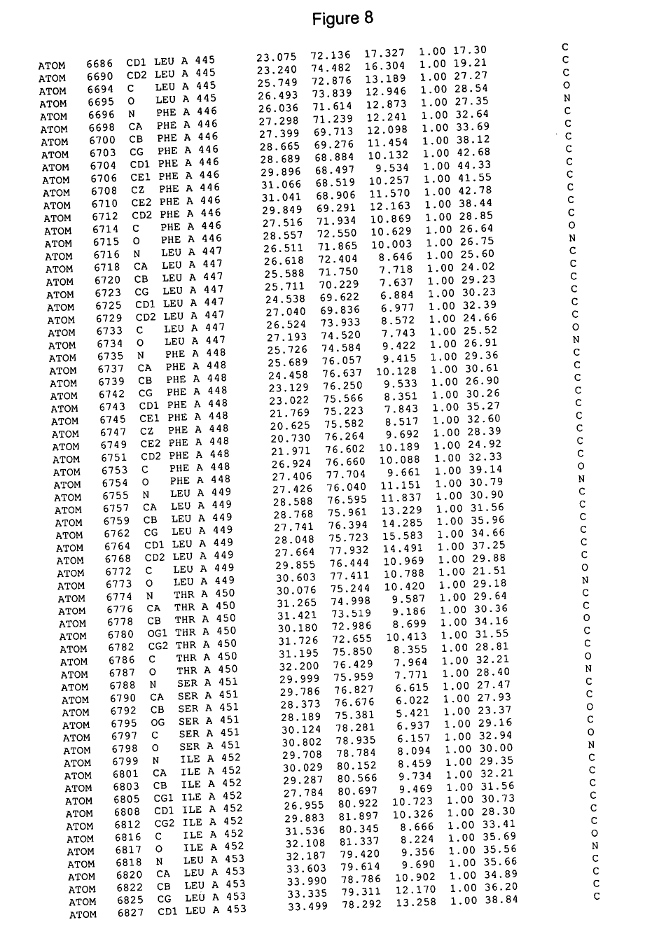

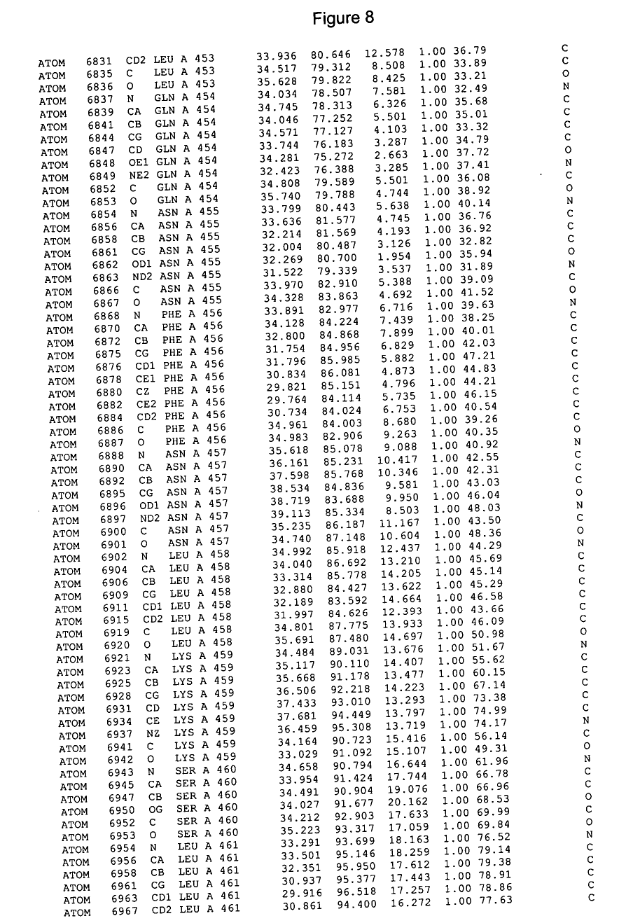

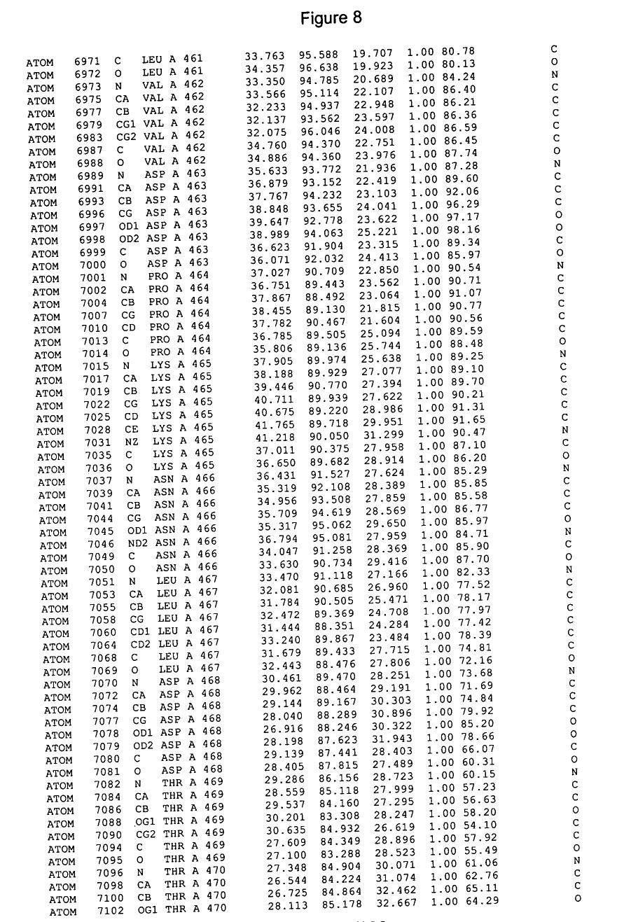

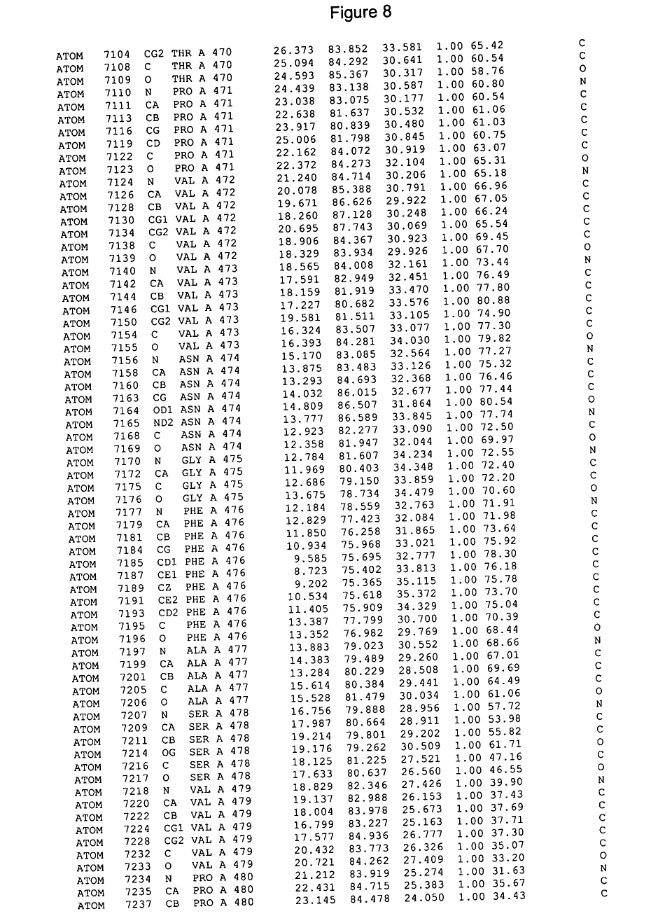

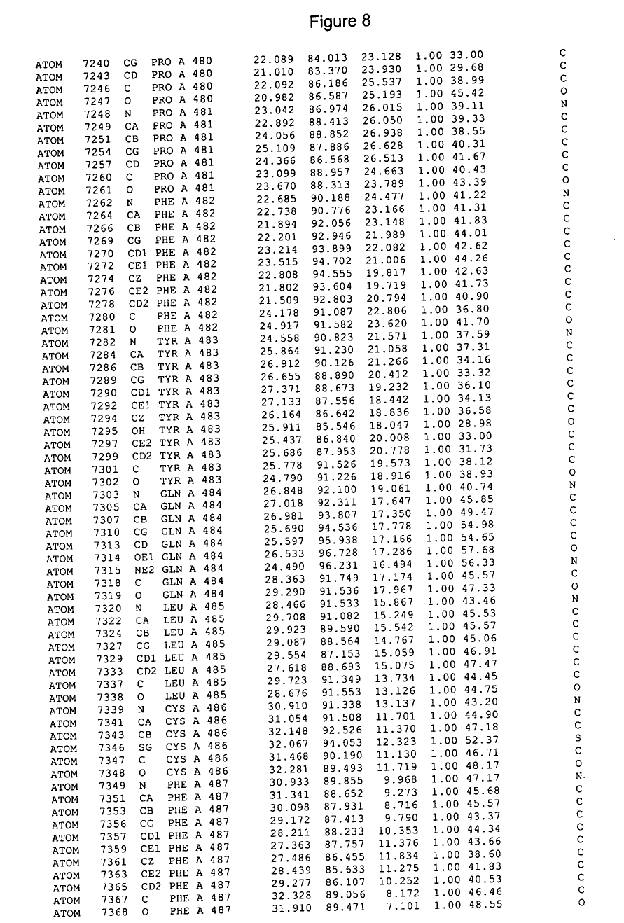

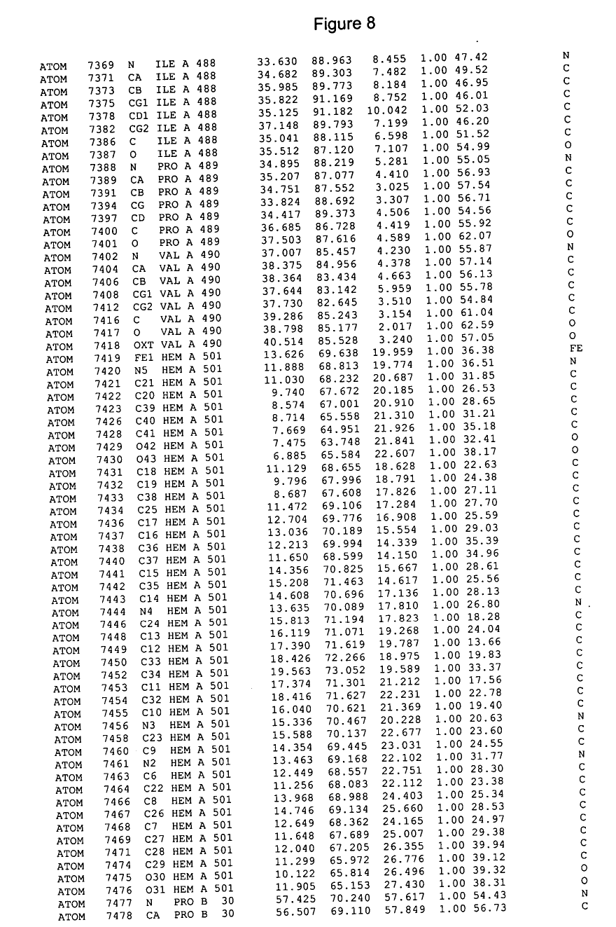

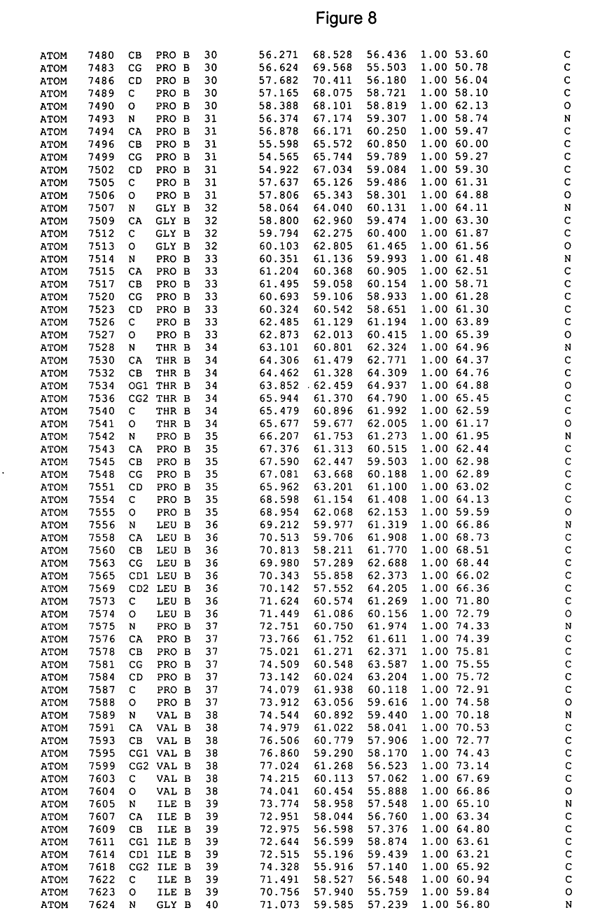

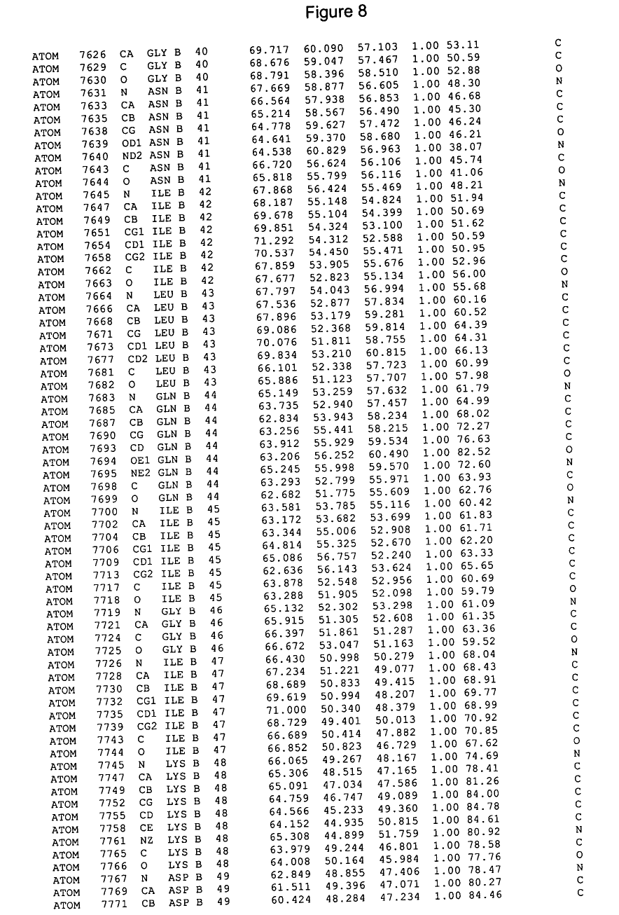

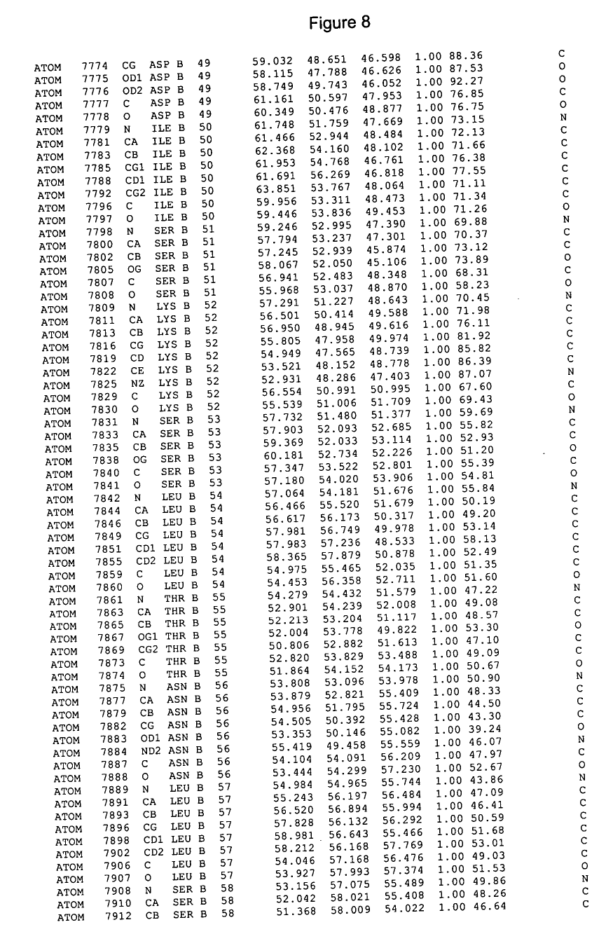

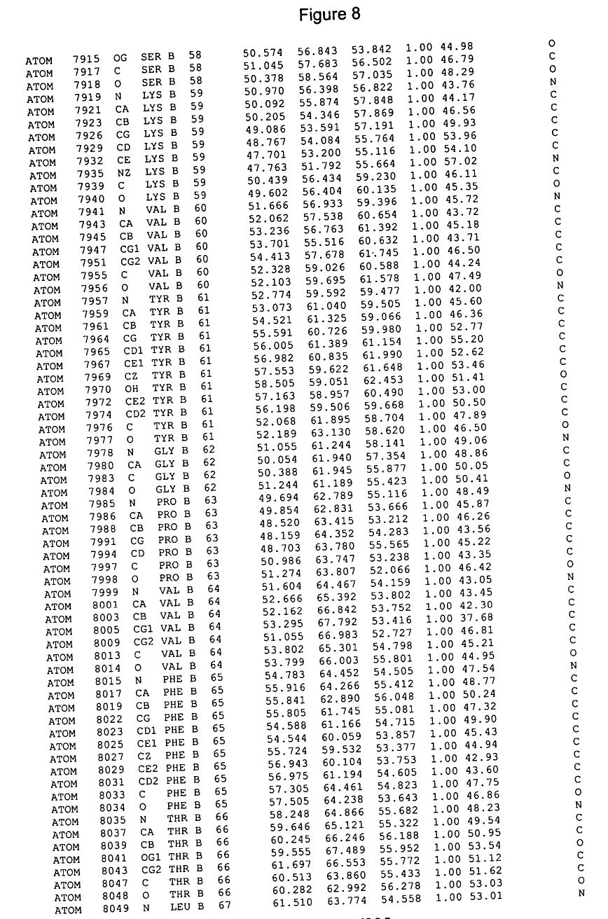

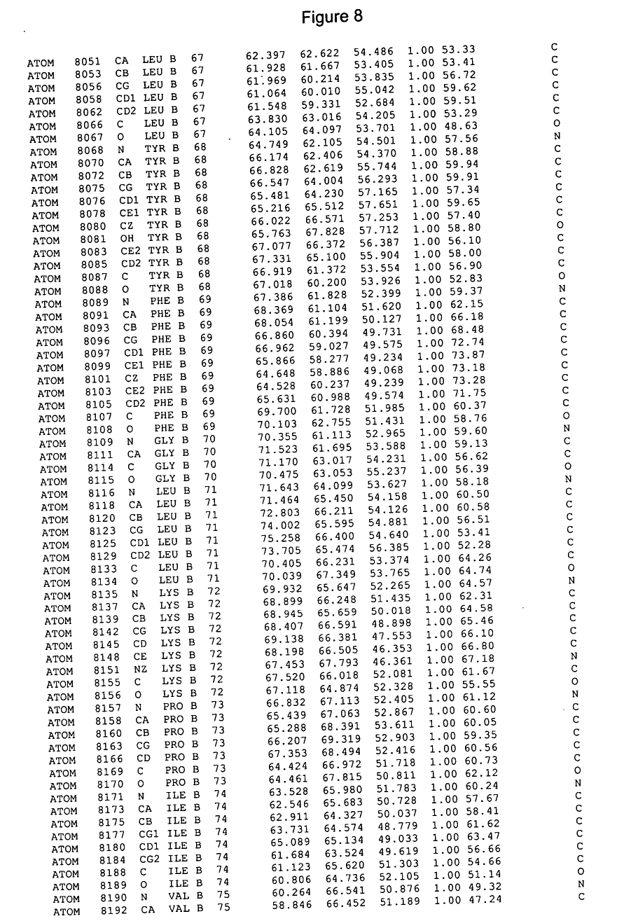

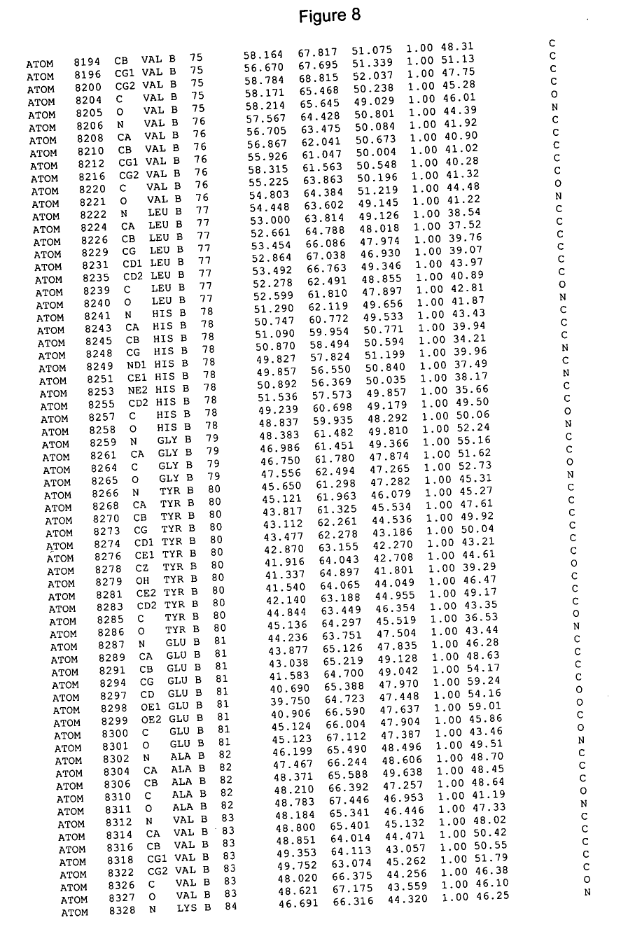

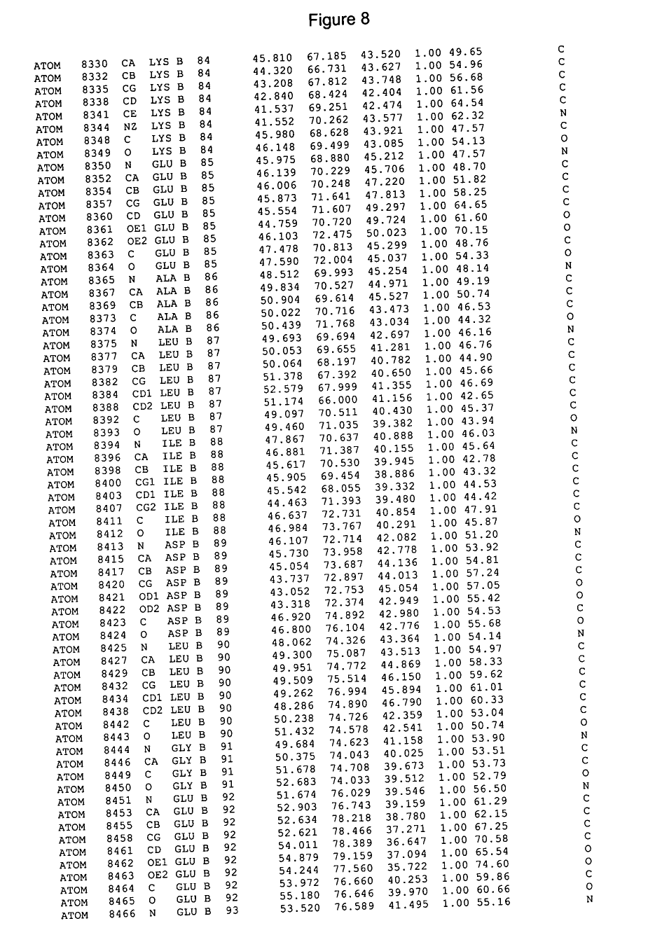

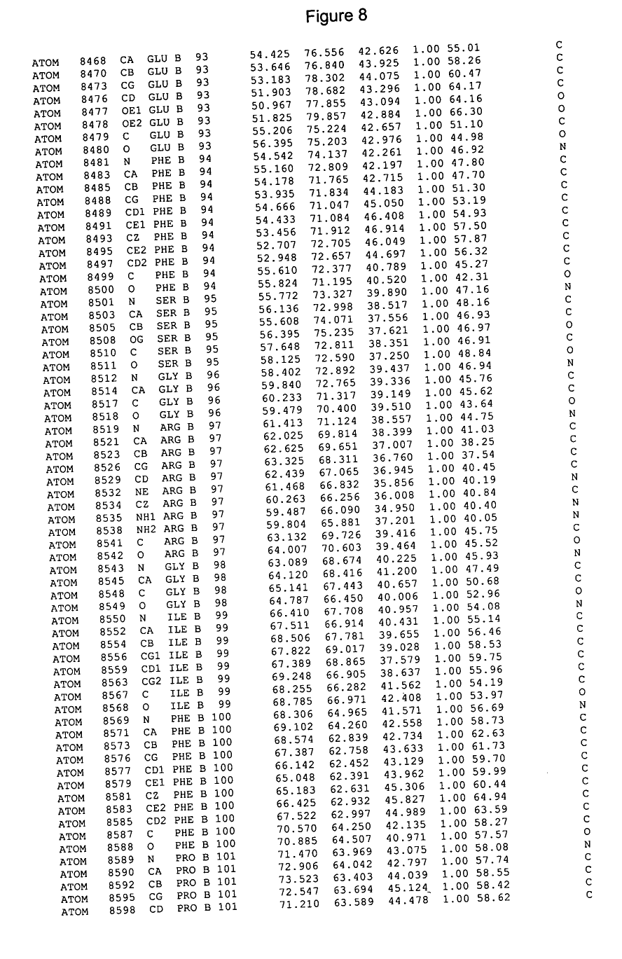

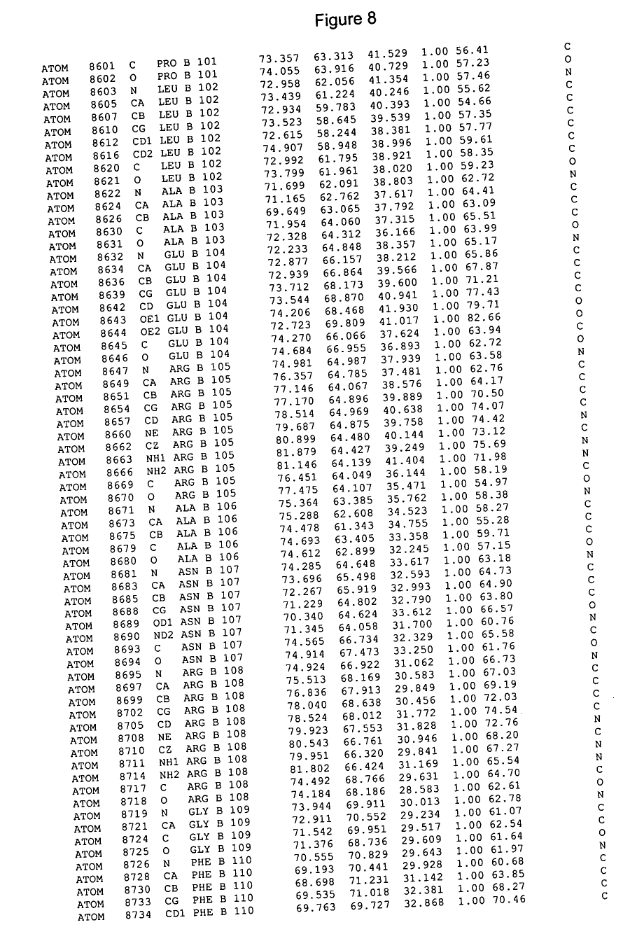

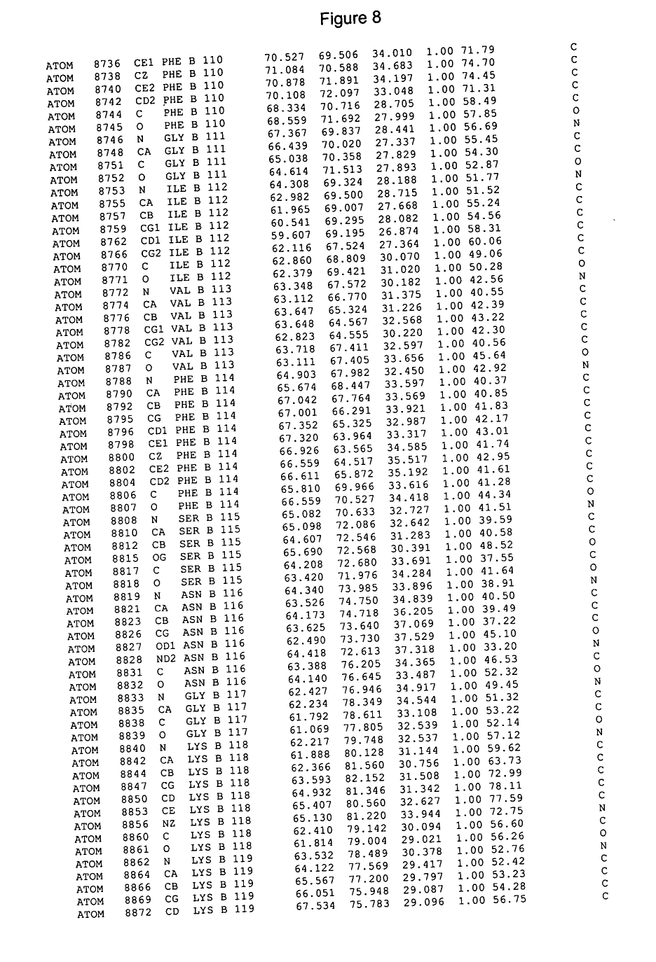

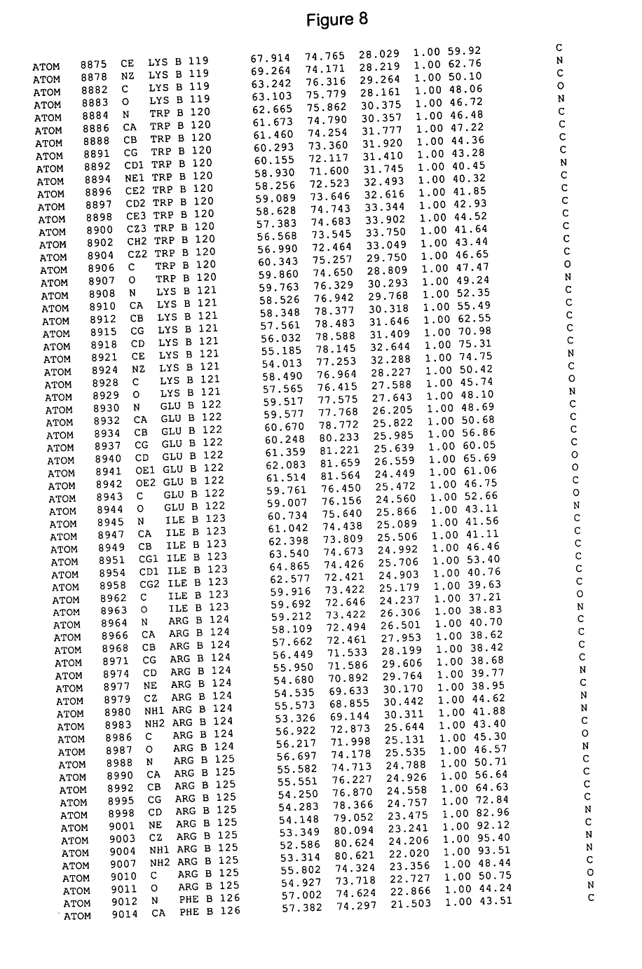

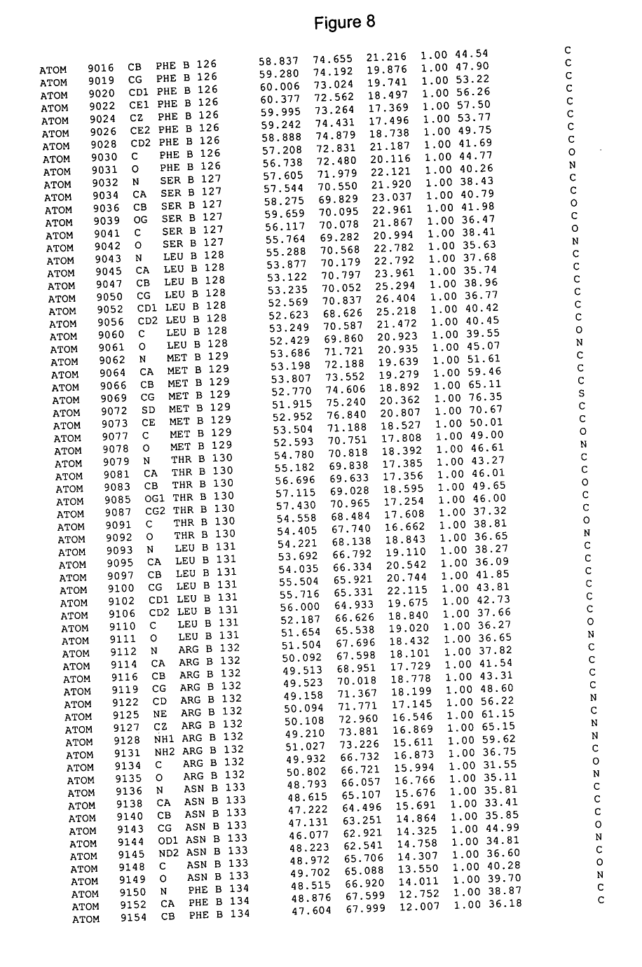

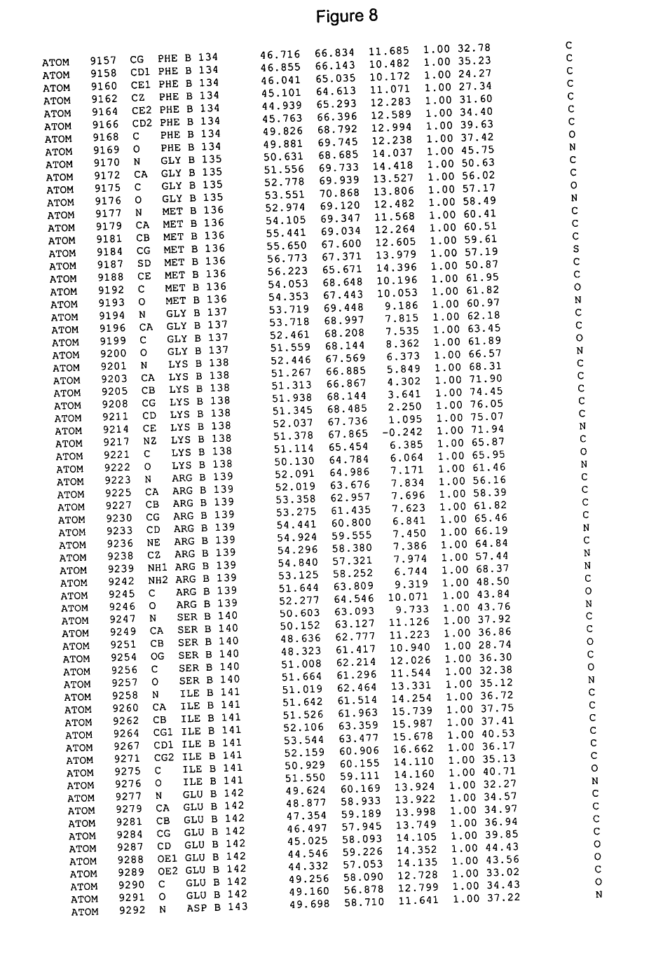

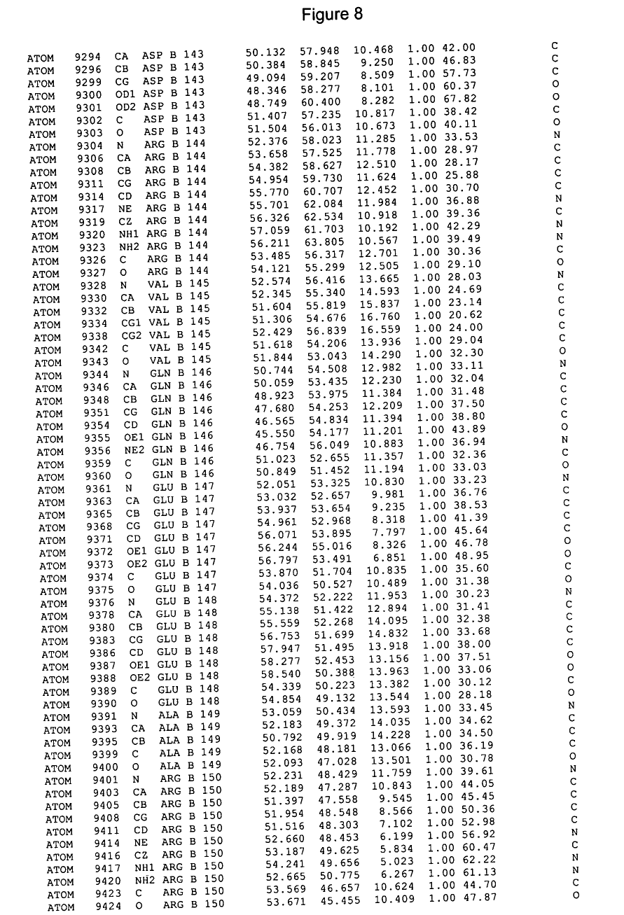

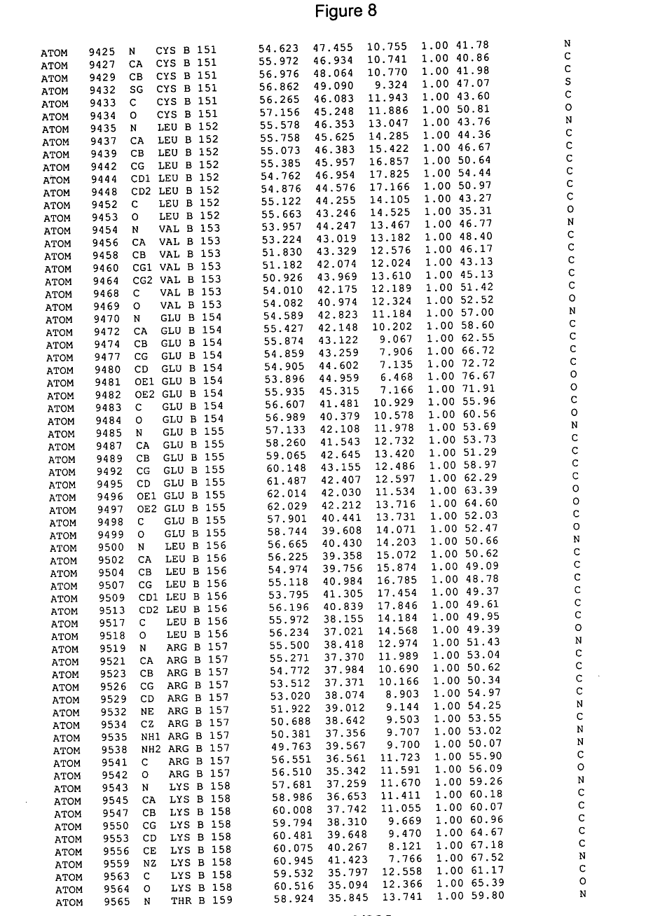

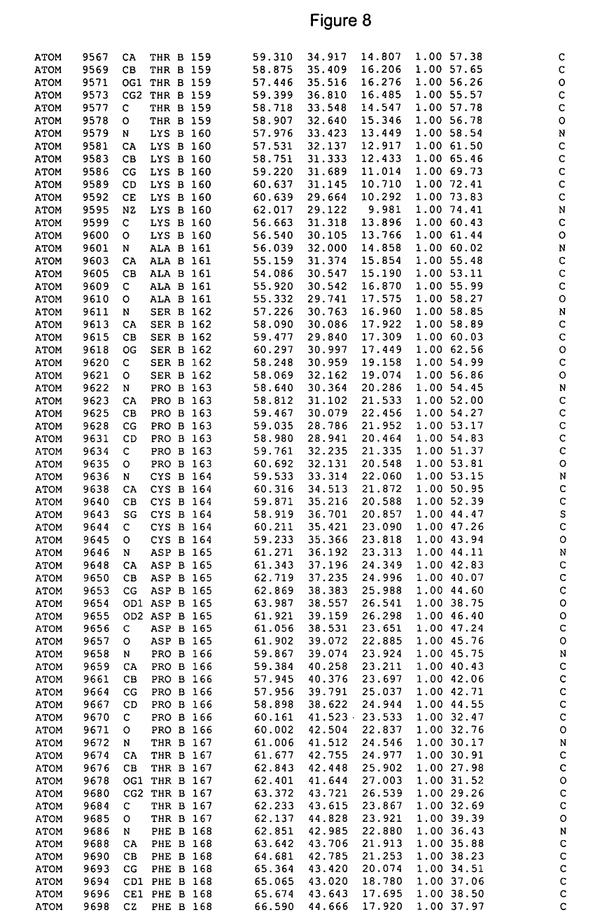

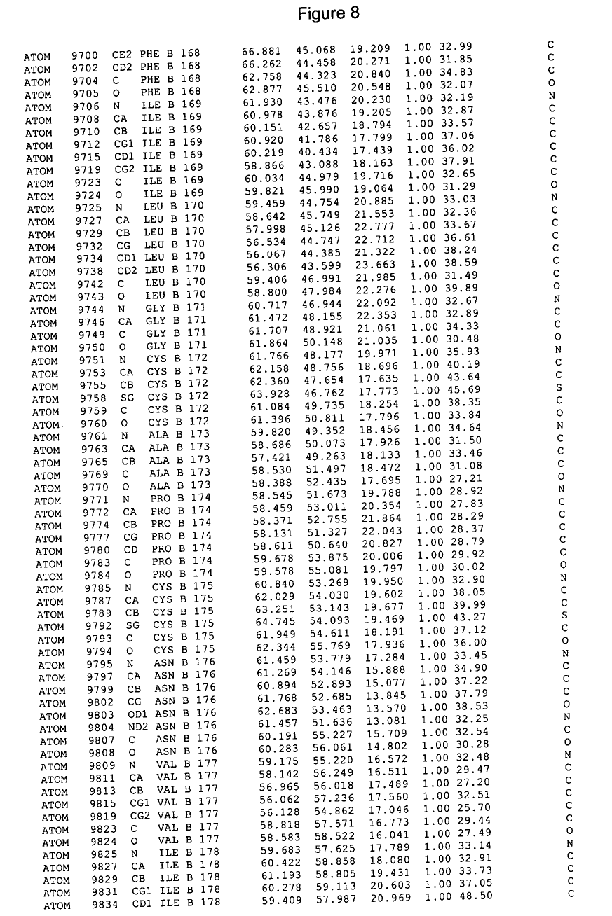

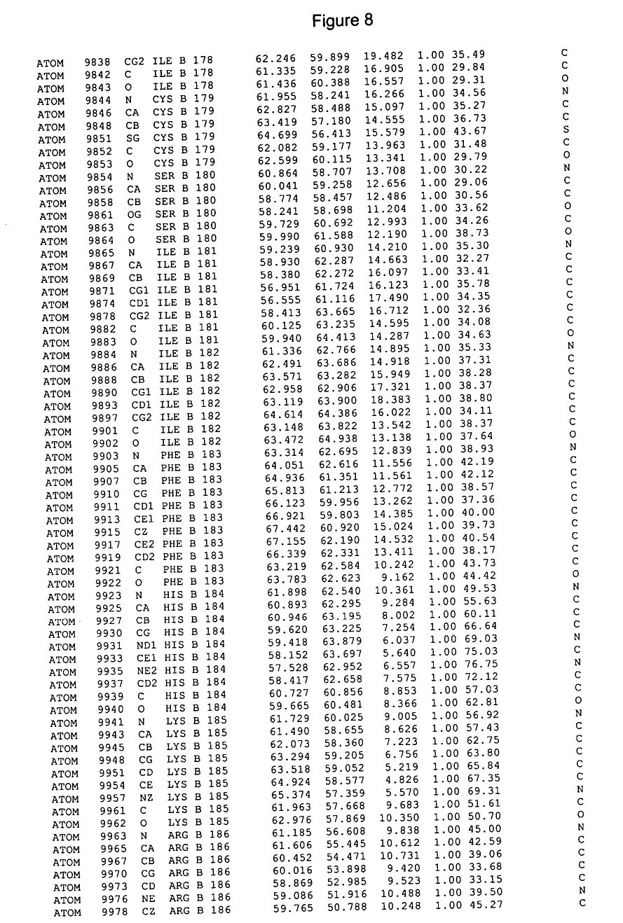

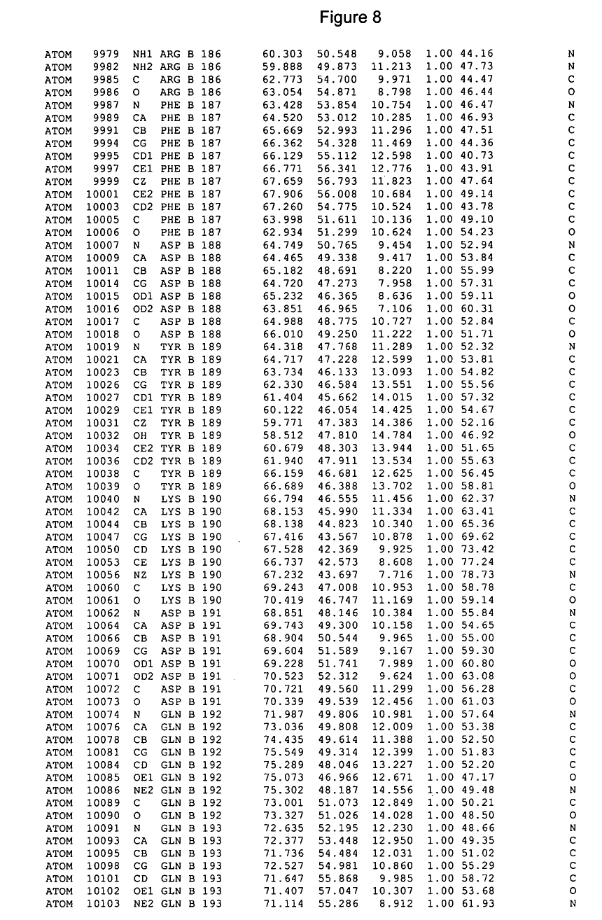

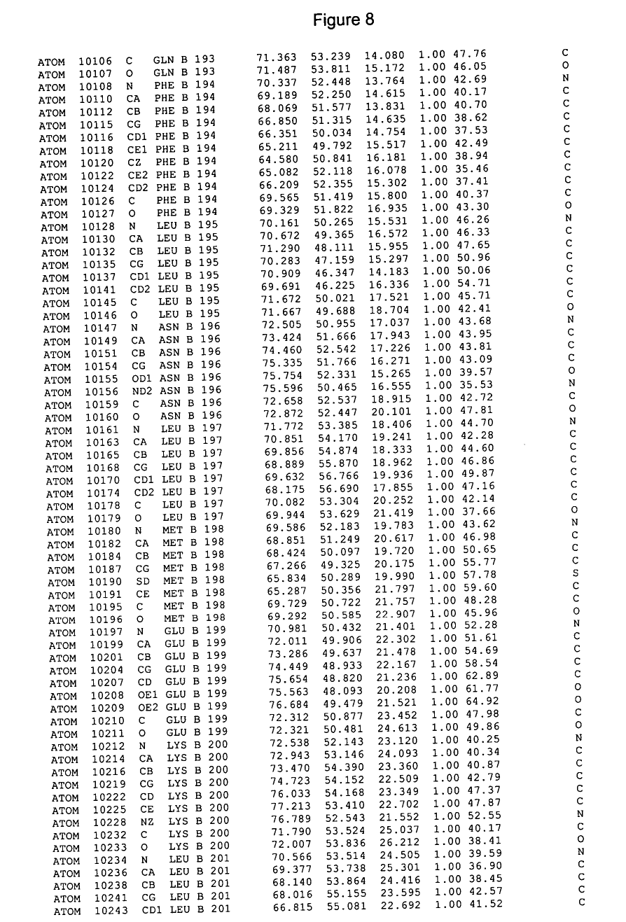

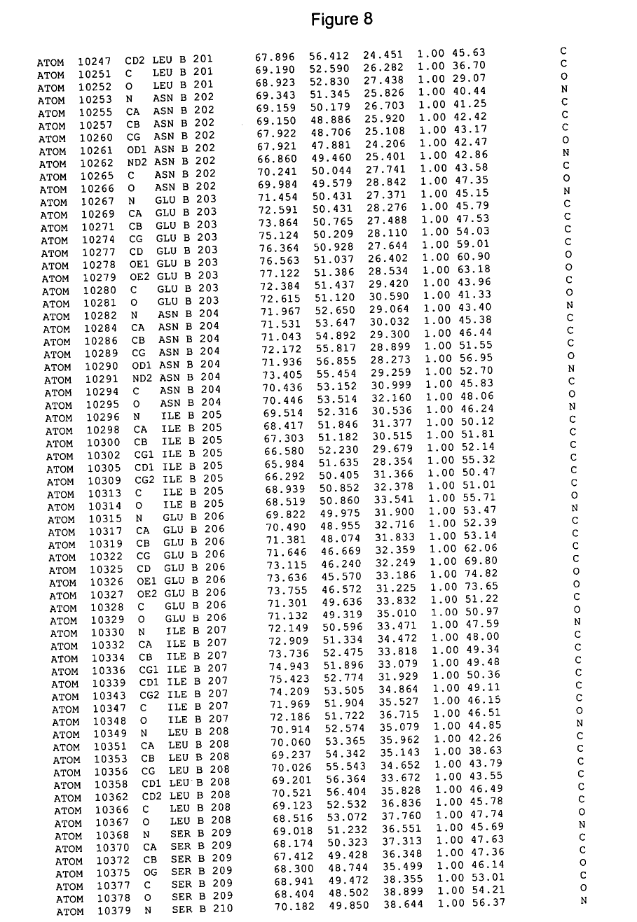

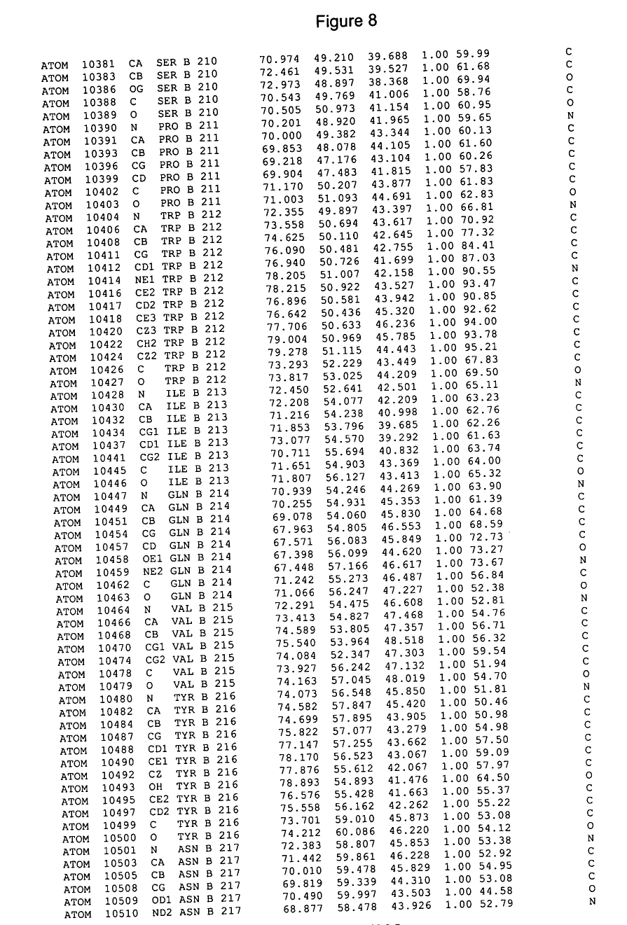

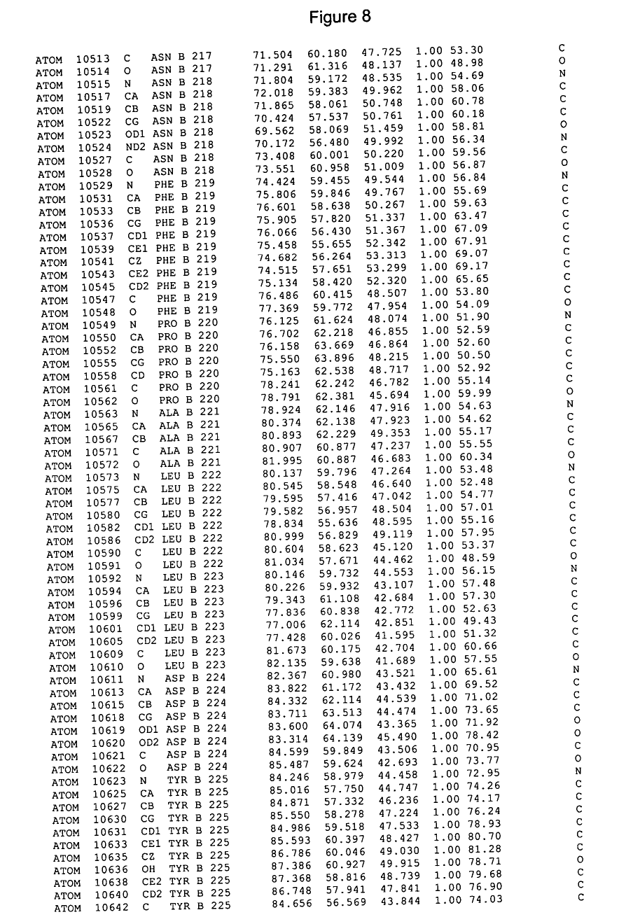

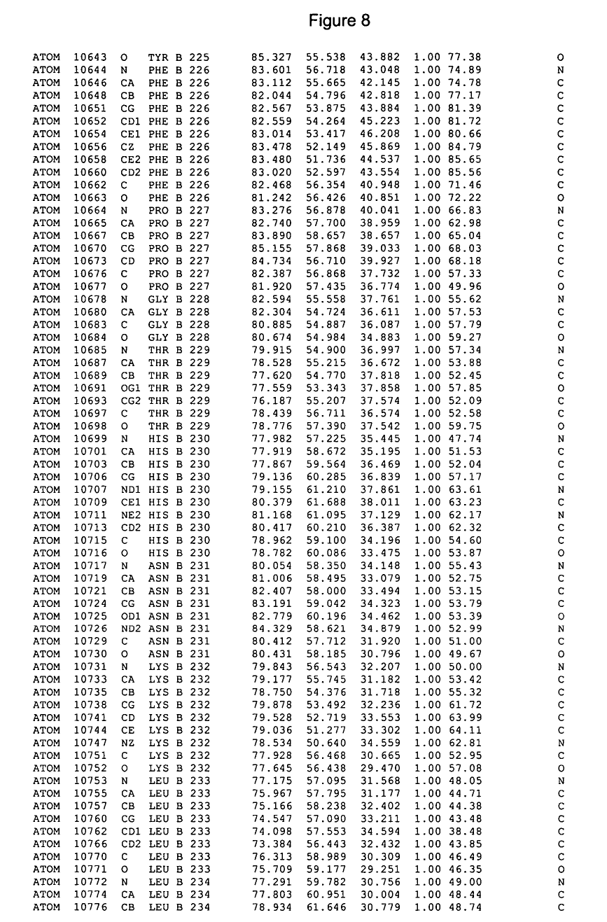

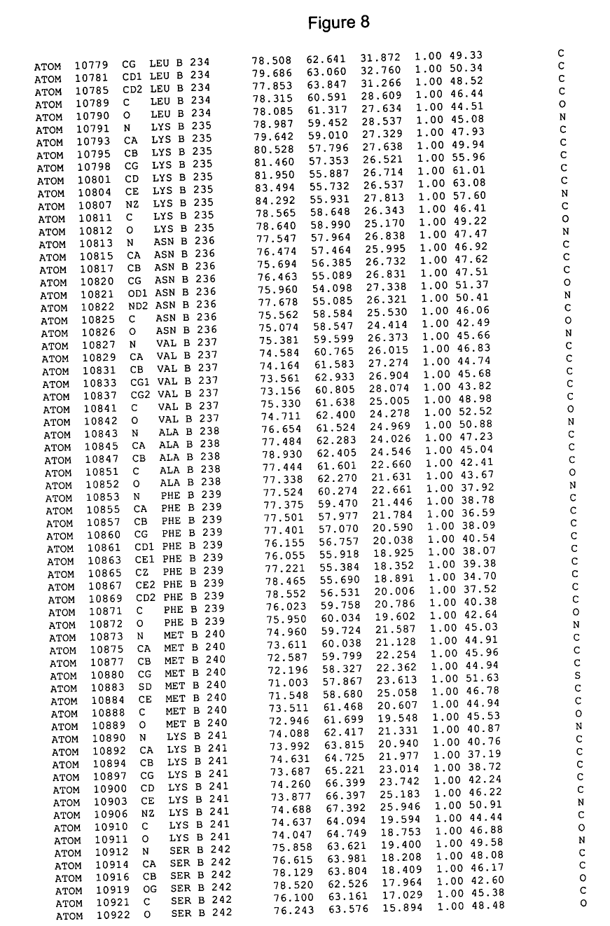

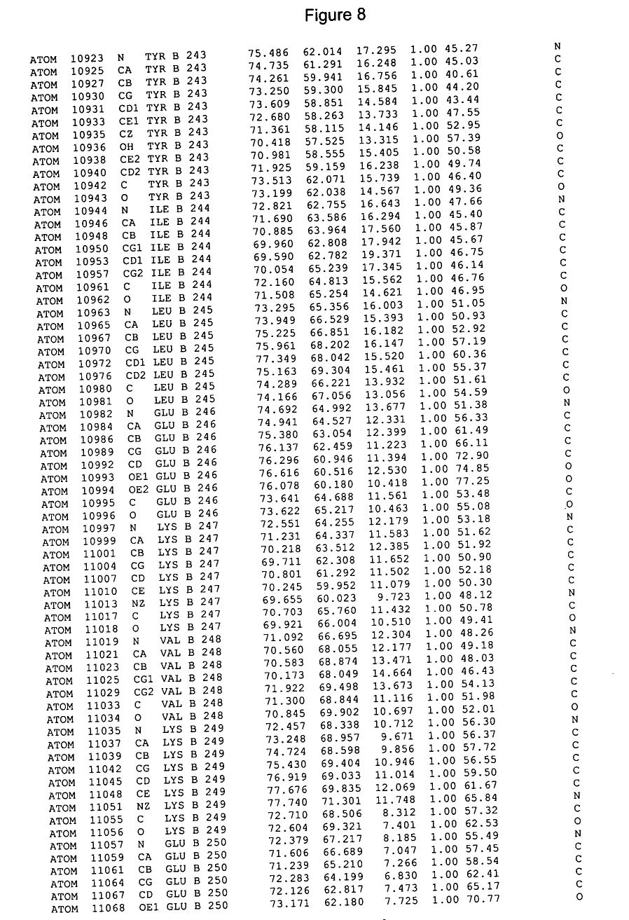

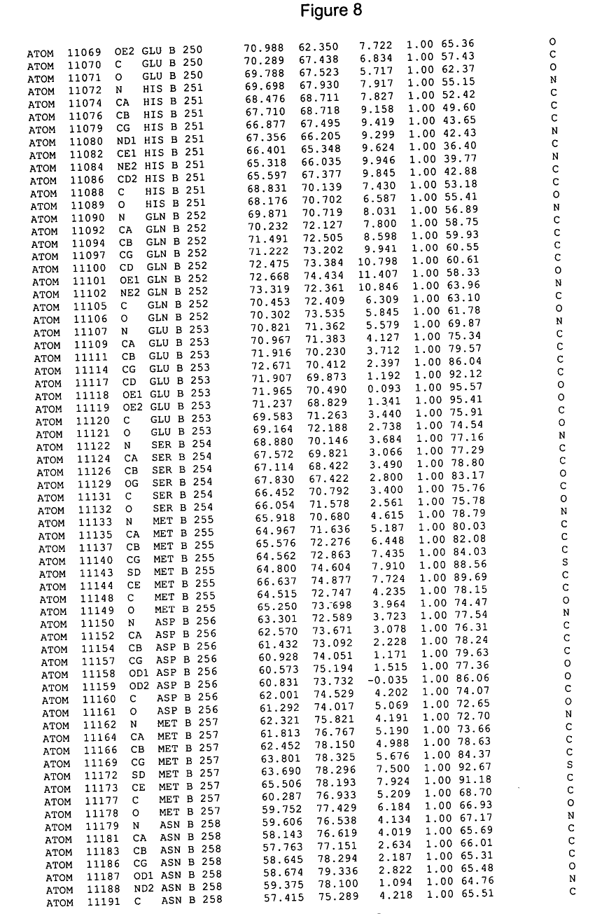

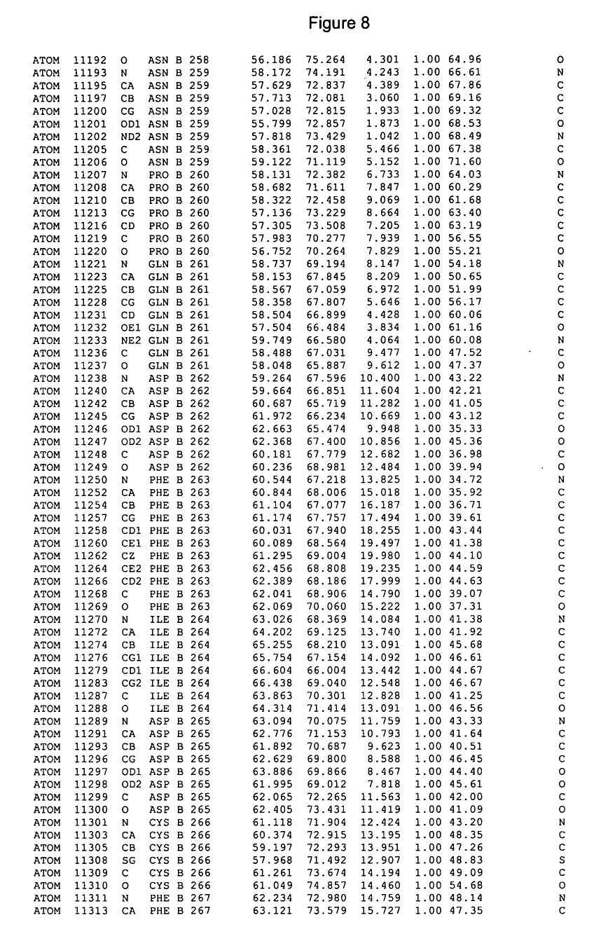

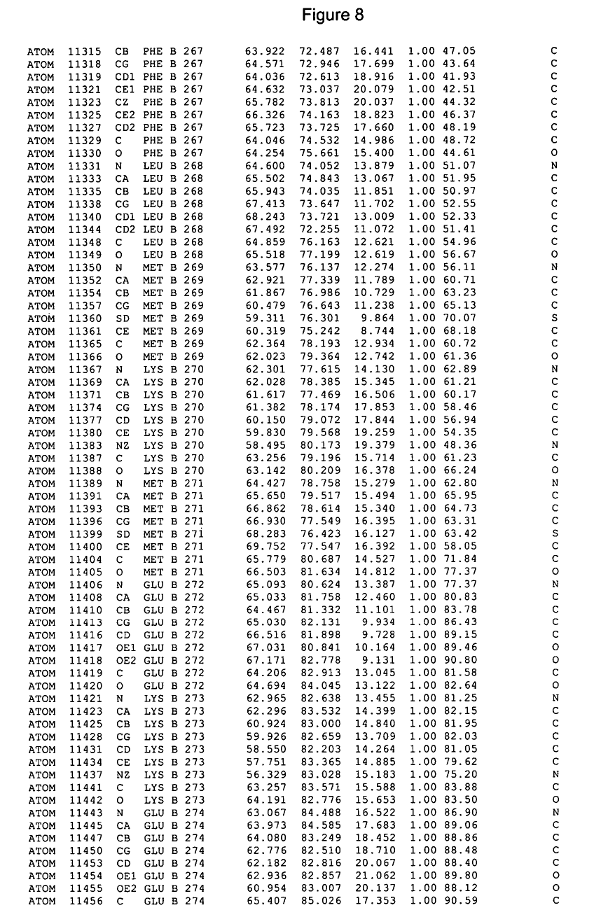

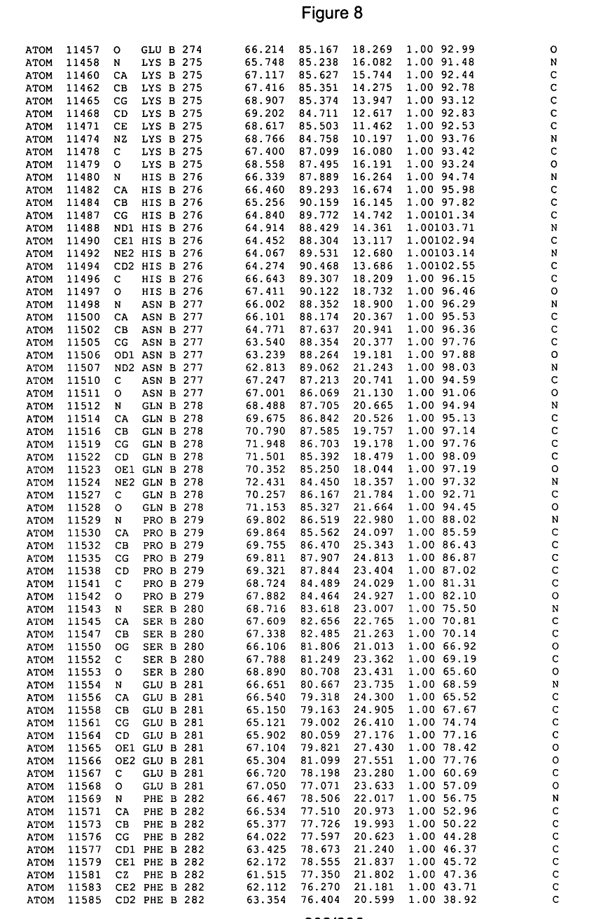

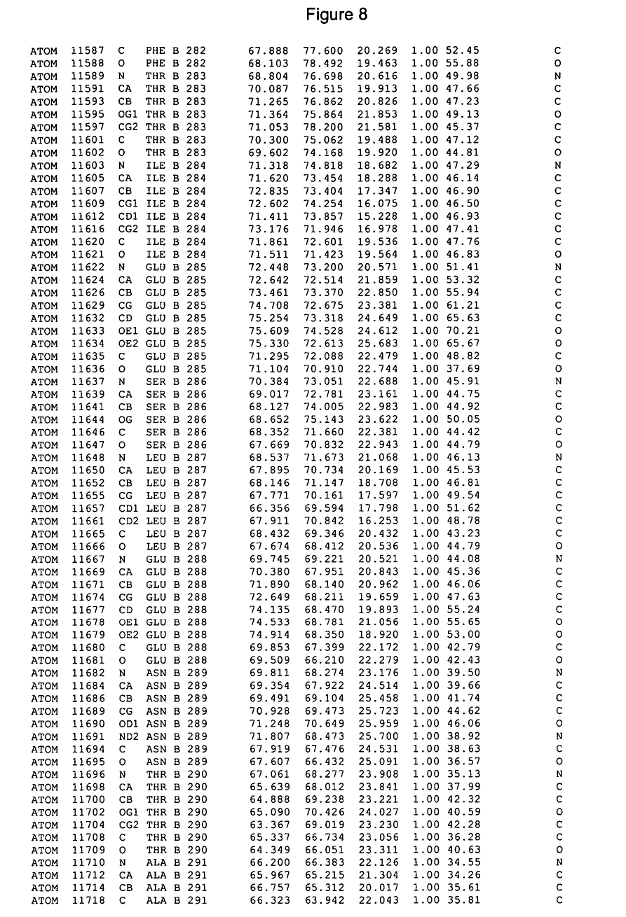

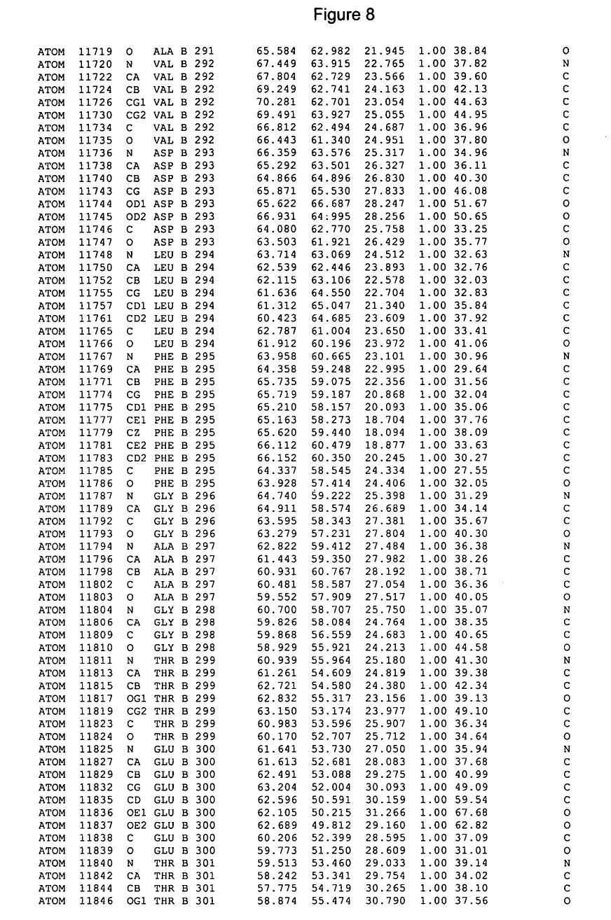

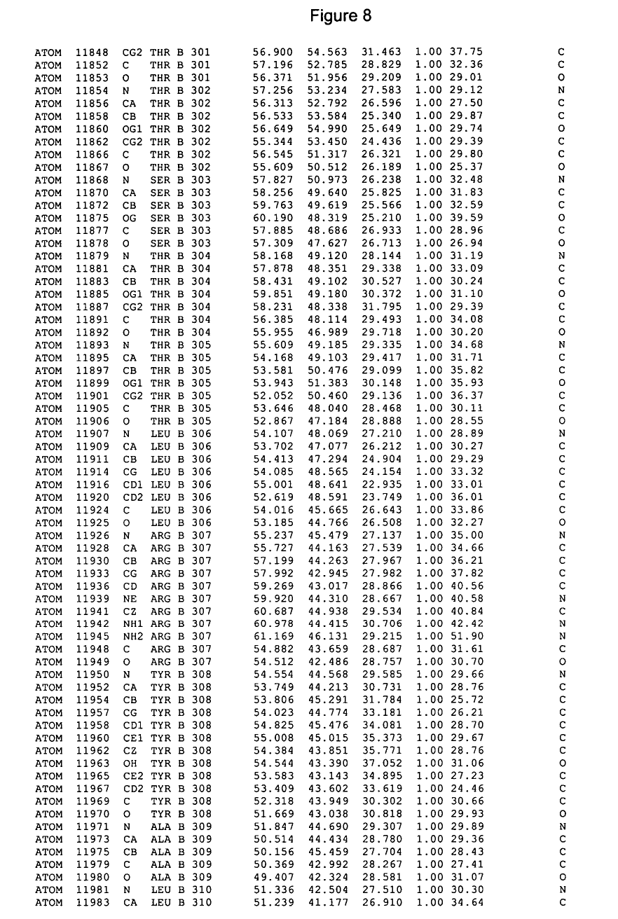

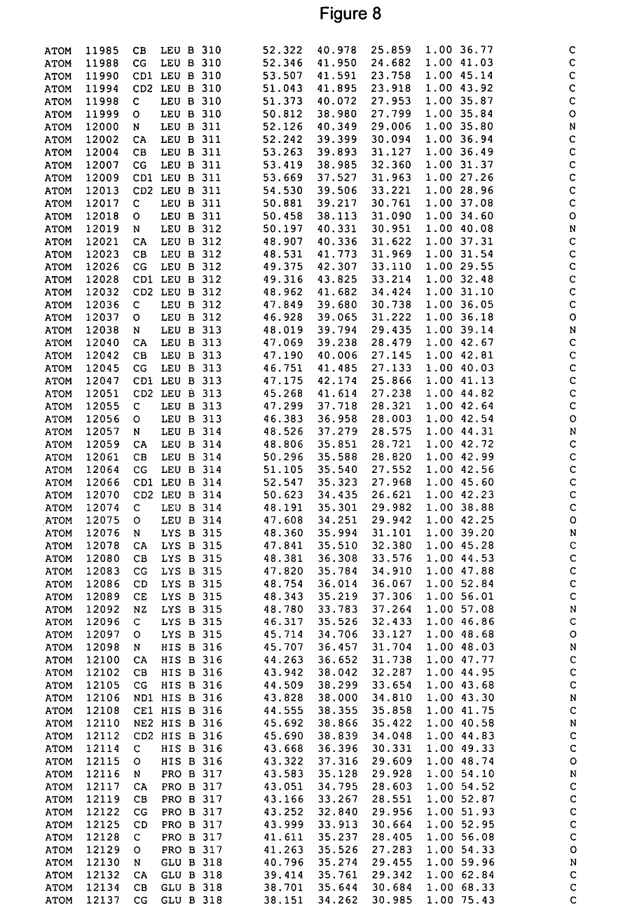

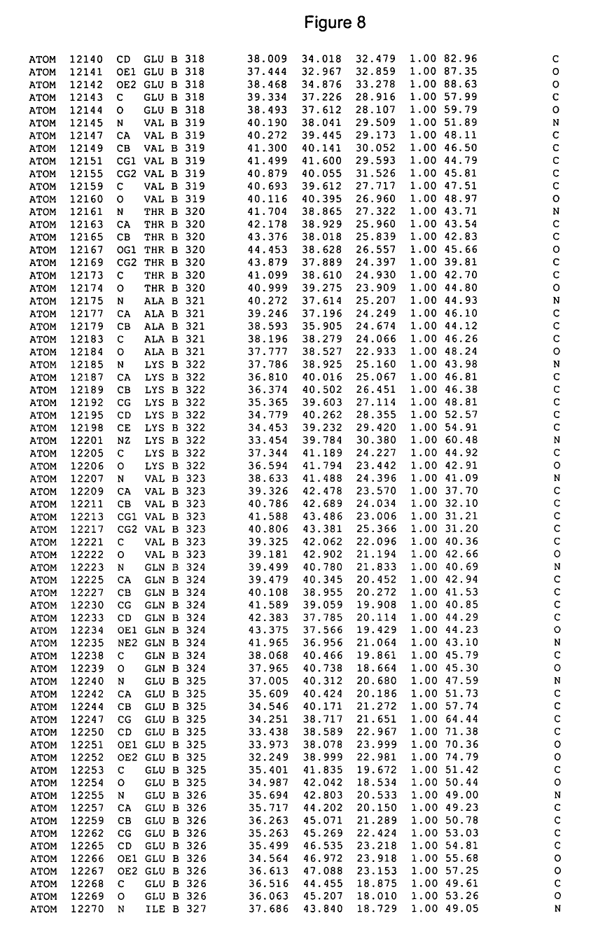

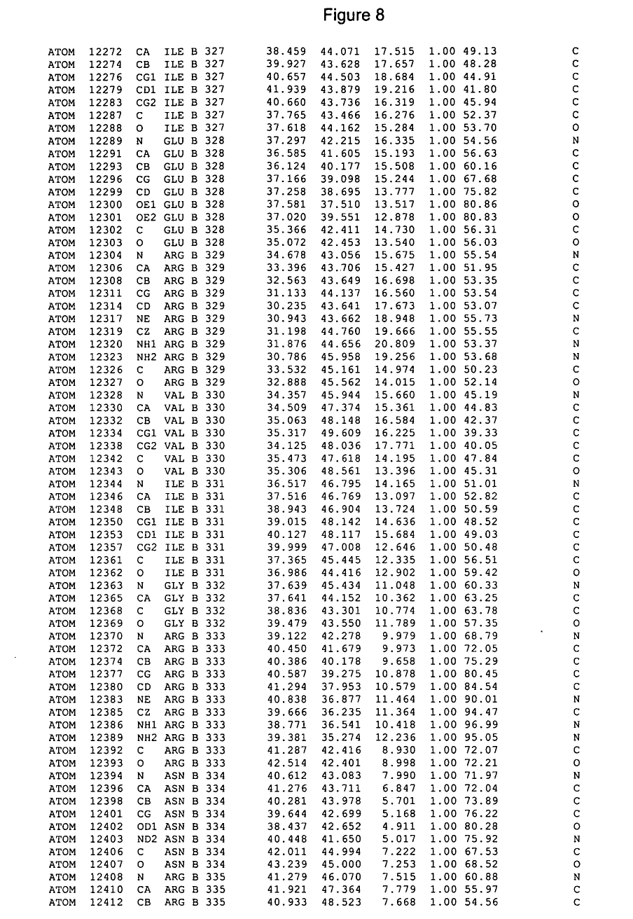

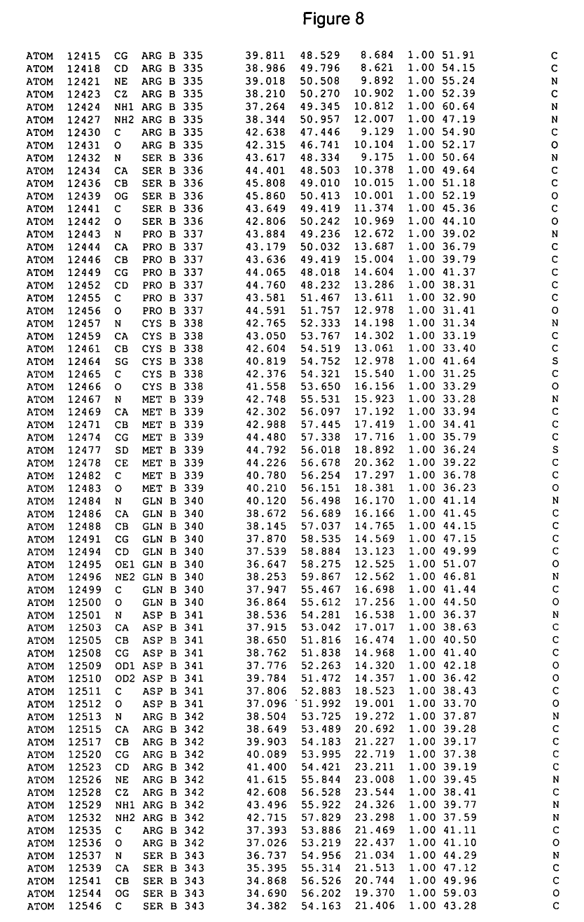

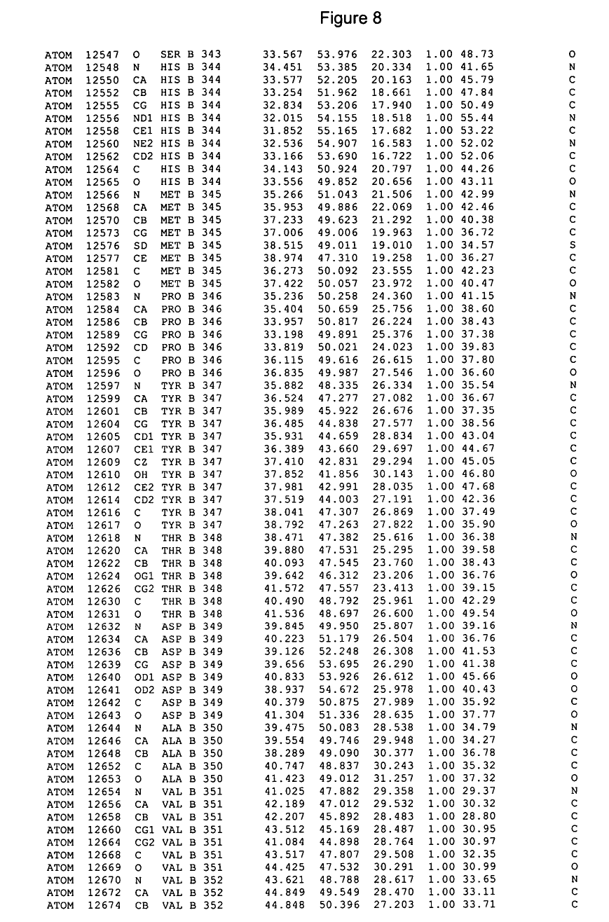

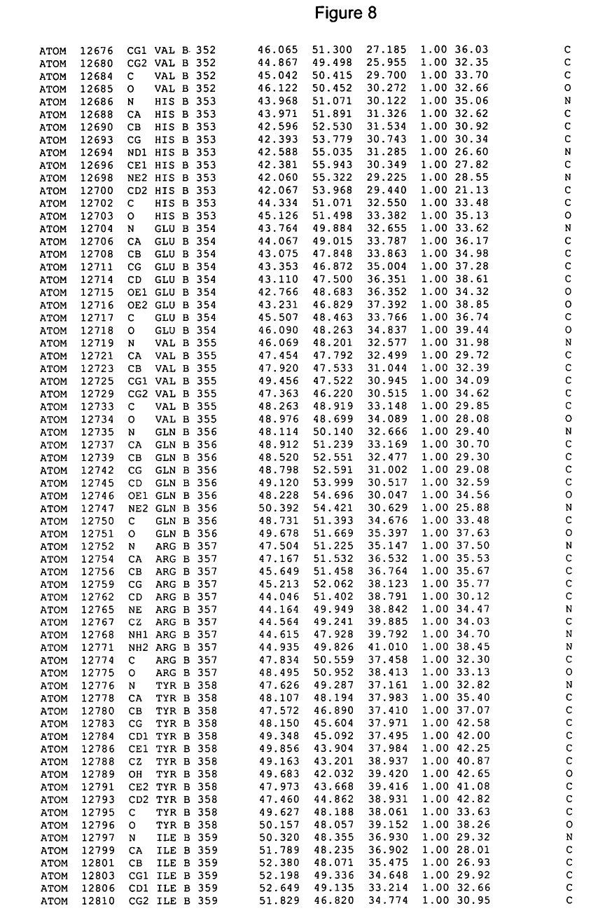

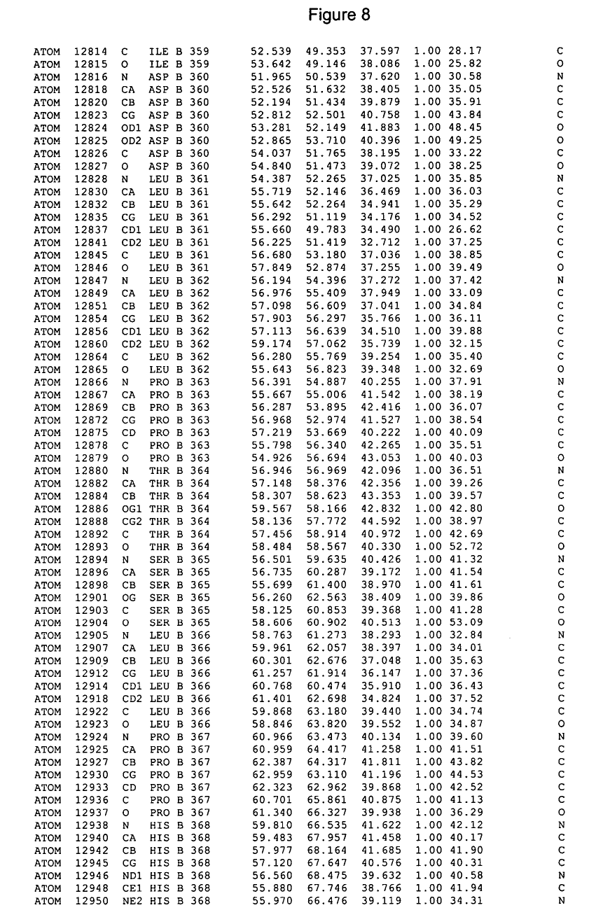

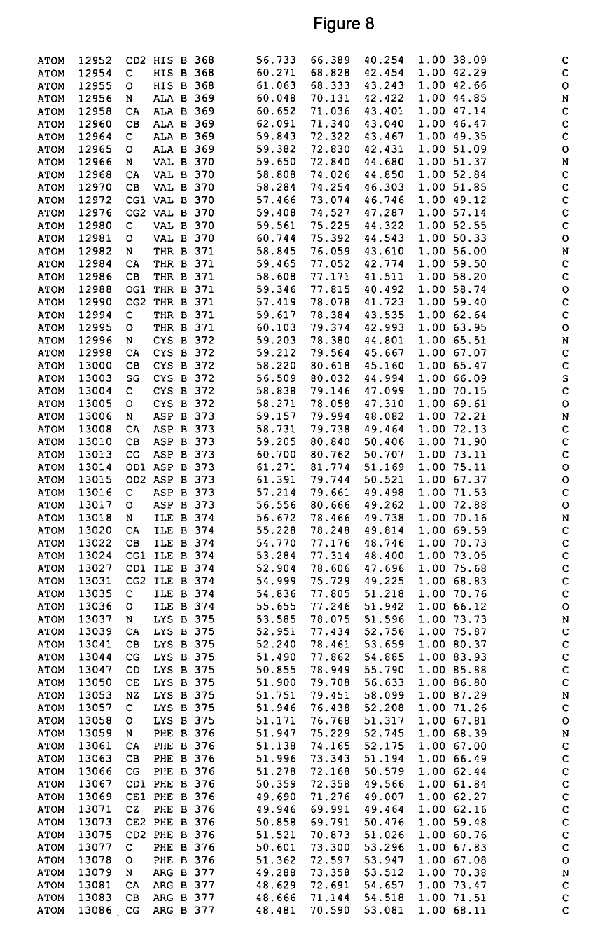

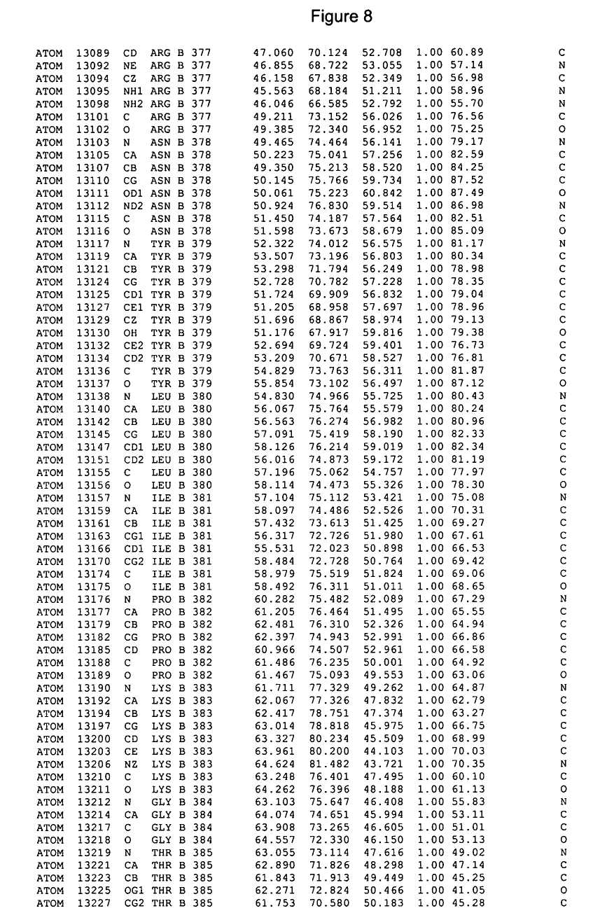

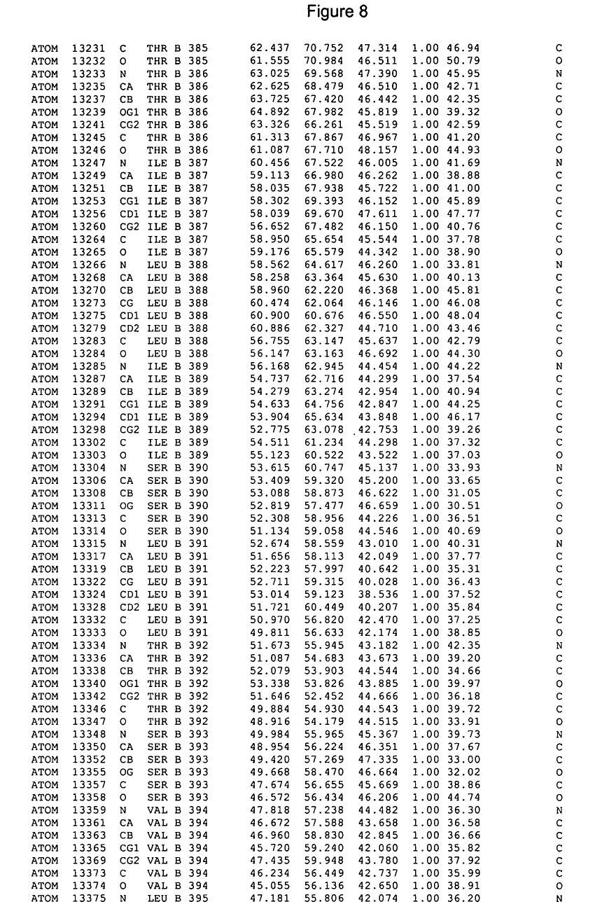

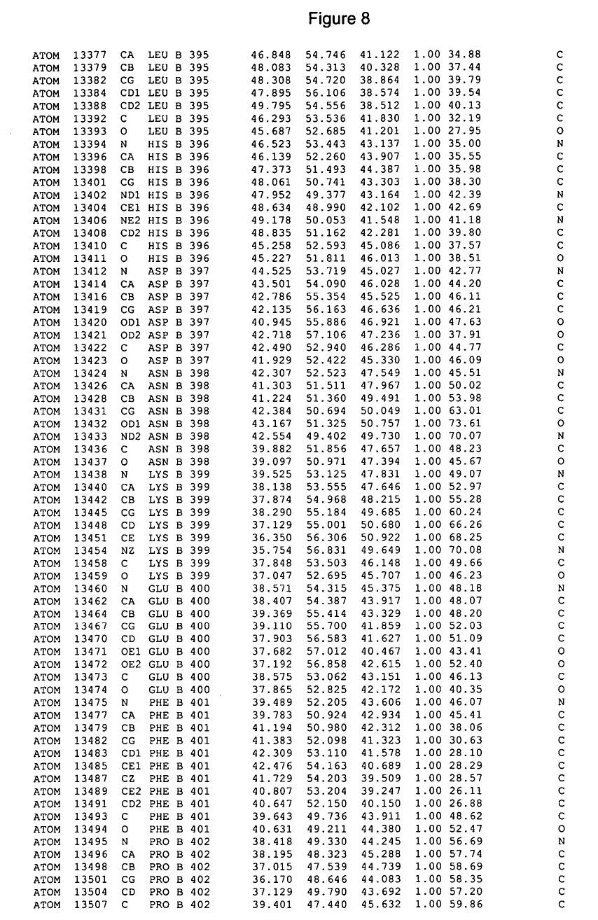

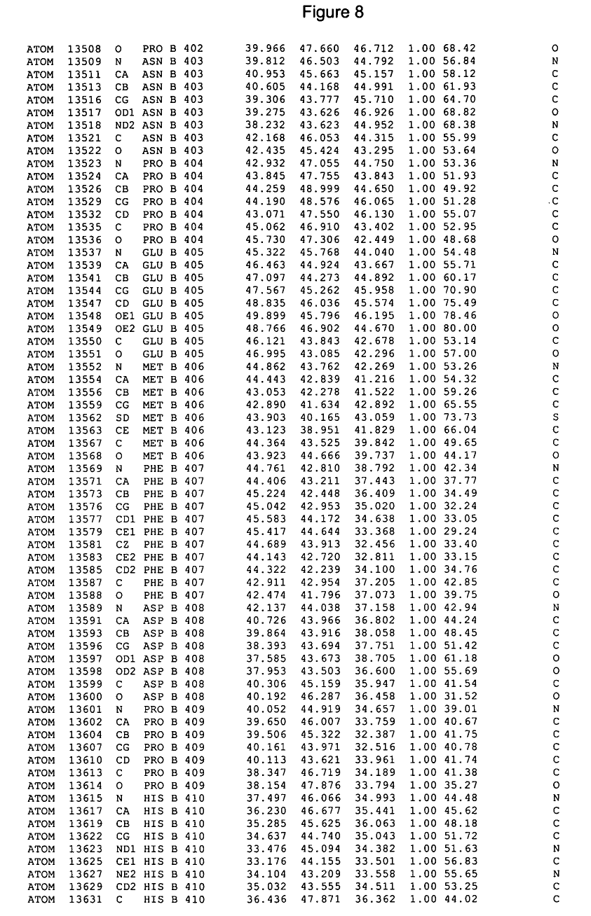

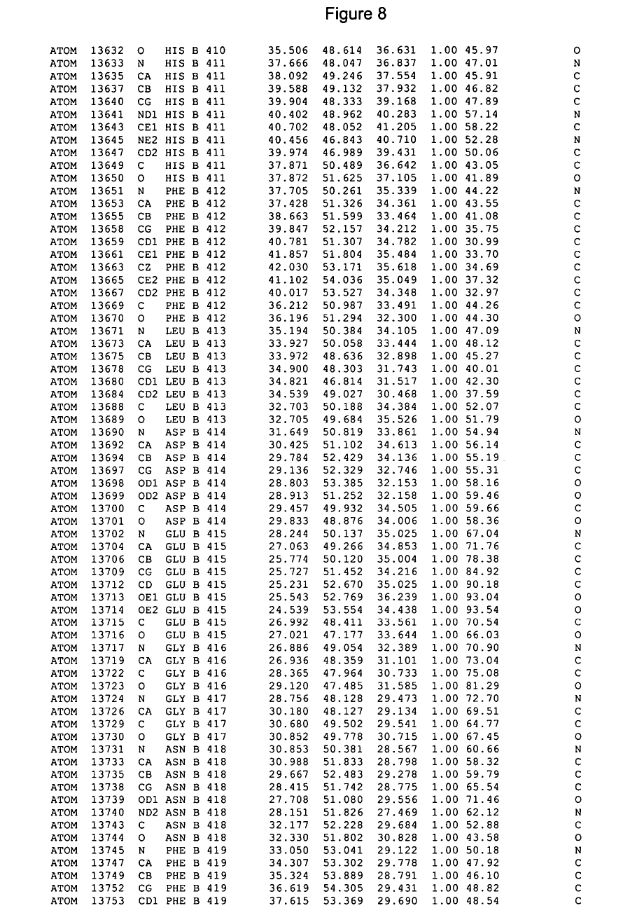

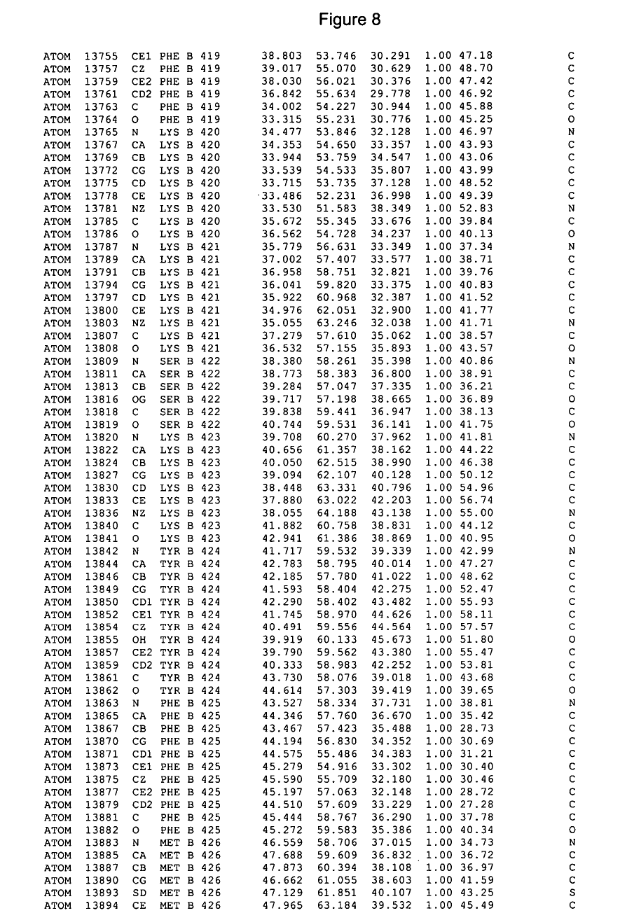

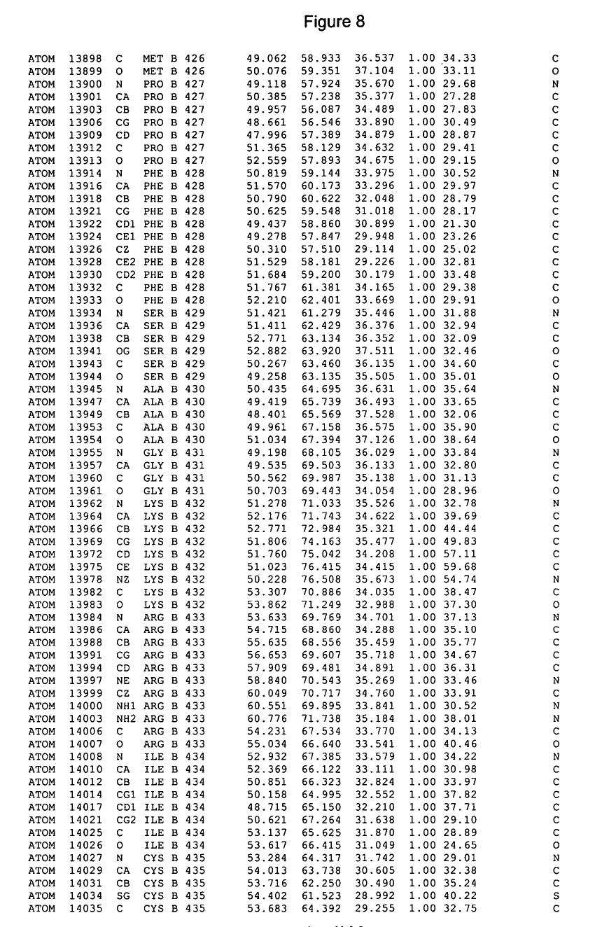

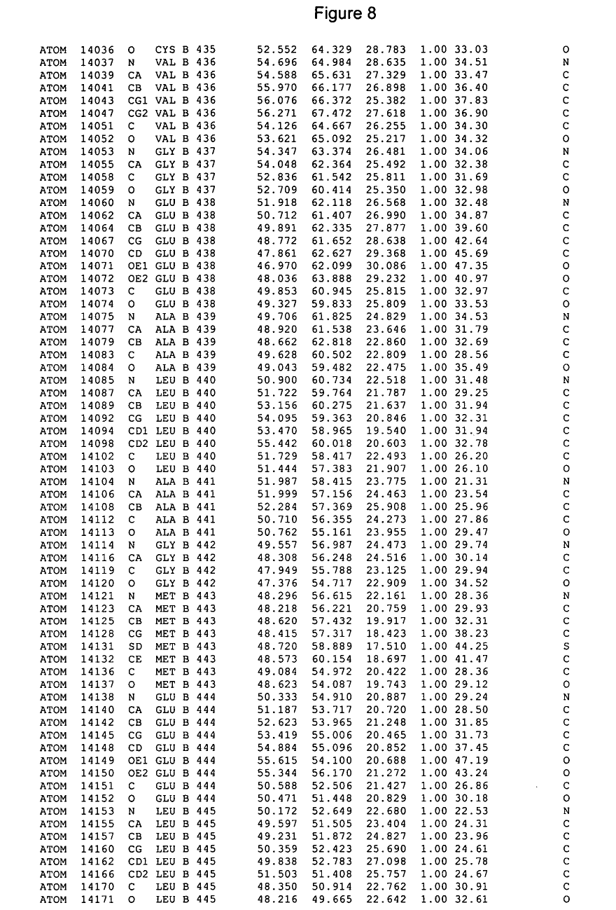

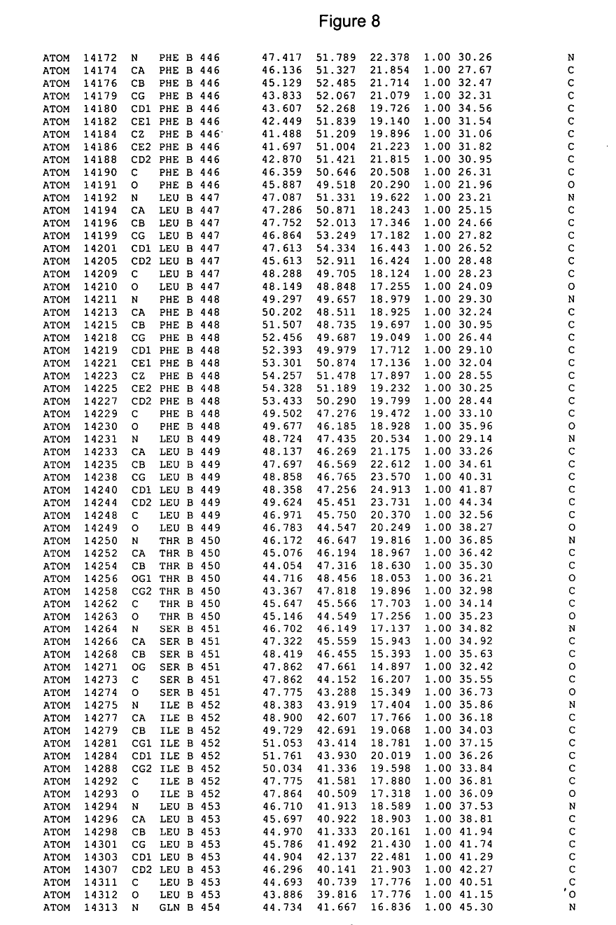

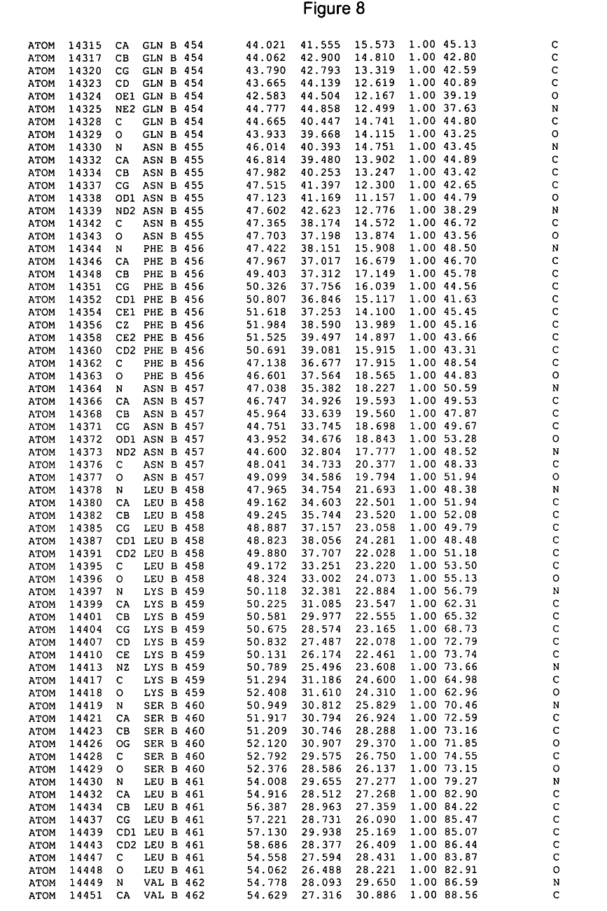

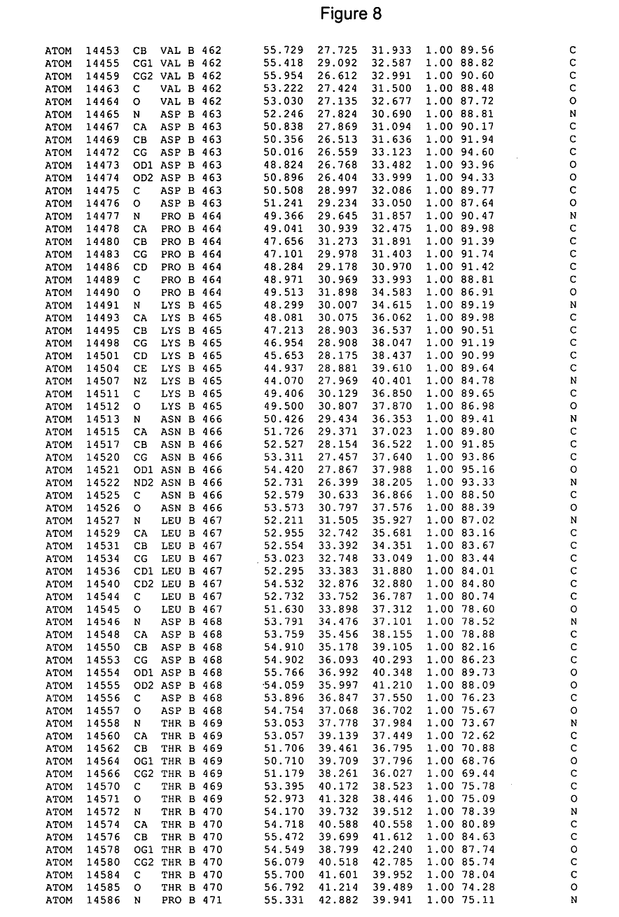

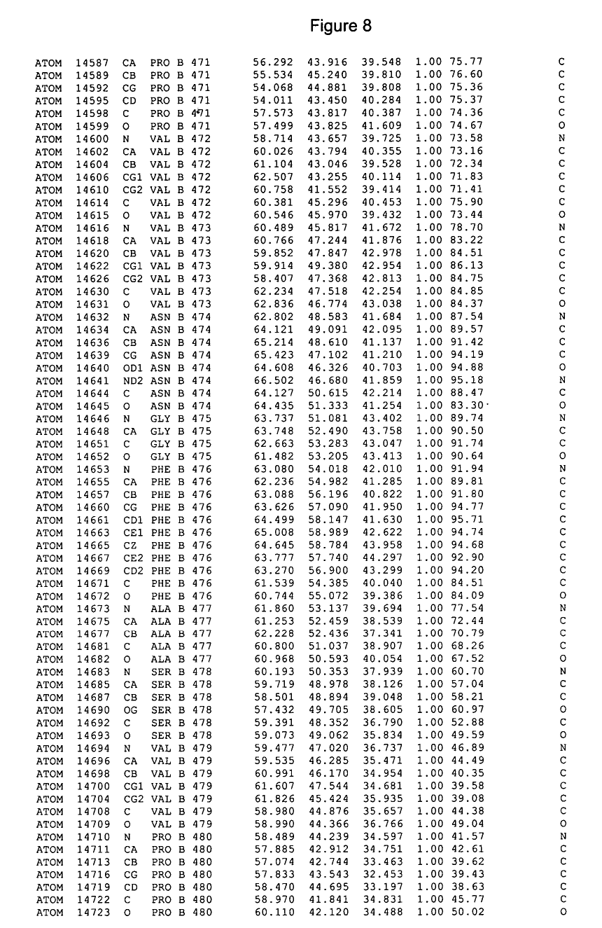

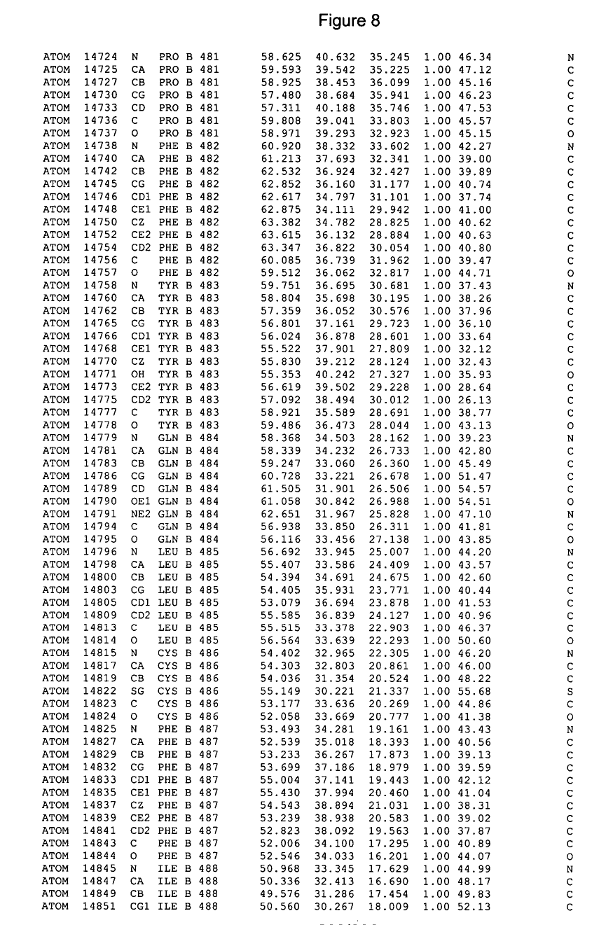

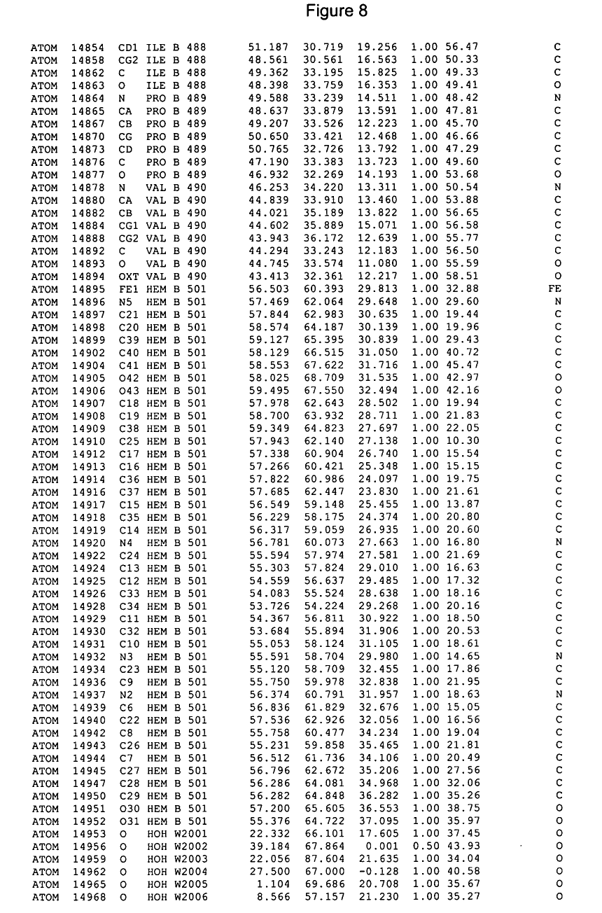

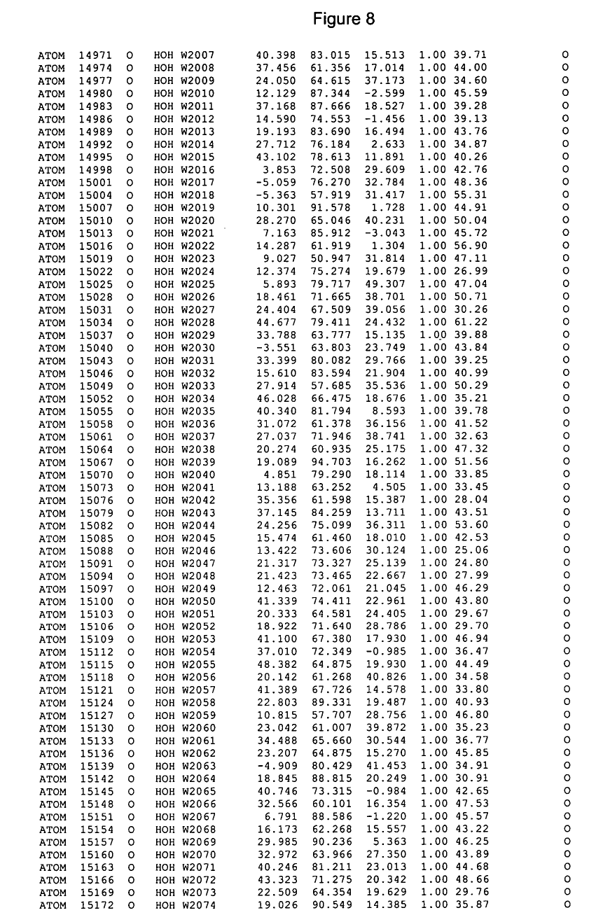

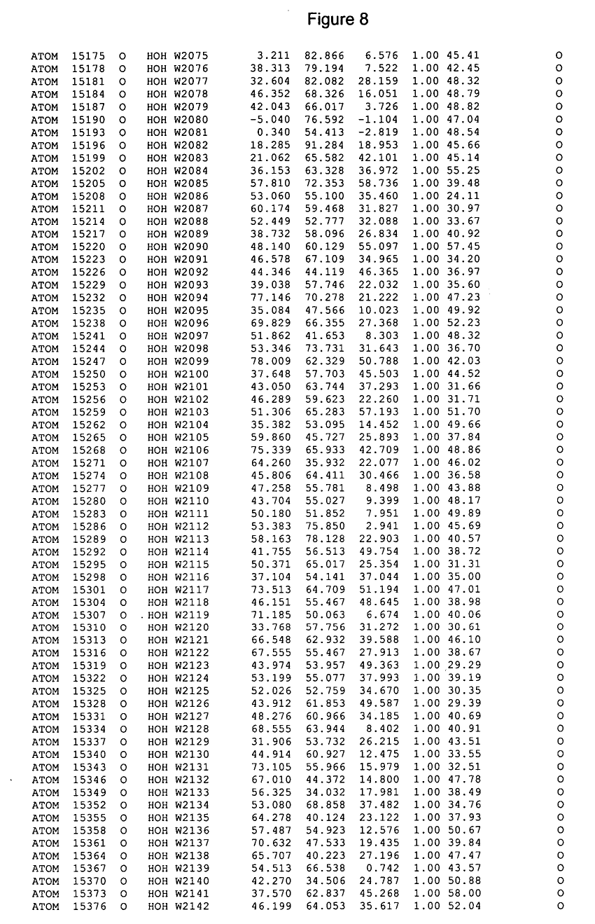

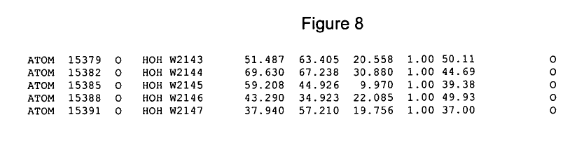

Our experiments have been based on the use of a particular N-terminal truncation of 2C9, as set out in SEQ ID NOs:2 and 4 and shown in FIG. 7. This protein also comprises a polyhistidine tag at the C-terminus. The N-terminal truncation and tag are both features which can be varied by those of skill in the art using routine skill. For example, alternative N-terminal sequence might be utilised, for example for production in host cells other than E. coli. Likewise, other tags may be used for purification of the protein as described below. These N— and C-terminals terminal modification may be made to a 2C9 protein which retains the core sequence of residues 31-490 of the wild type sequence illustrated in FIG. 7.

The present invention relates to a P450 2C9 protein which comprises the following changes:

position 220 or position 222 is proline; and

optionally up to 30, for example up to 25, for example up to 10, for example up to 5 other positions are altered,

the positions 220 and 222 being numbered according to wild type 2C9. This numbering is shown in FIG. 7.

Preferably the change is to position 220.

It will be appreciated from the discussion above that by 2C9 protein, it is meant a protein comprising residues 31 to 490 of the wild type sequence, optionally with N— and/or C-terminal sequences provided to facilitate expression and recovery of the protein.

Where present, the N-terminal sequence is preferably not the wild-type sequence. Preferably, it is shorter that the wild type sequence (which is 30 amino acids). Preferably, the N-terminal region joined to residue 31 is the truncation illustrated in the accompanying examples, i.e. SEQ ID NO:6 plus a proline residue between it and residue 31 (also proline). This type of N-terminal sequence reduces the tendency of 2C9 to anchor to membranes and to aggregate compared to the wild type sequence.

Where present, the C-terminal sequence is preferably no larger than 30, and preferably no larger than 10 amino acids in size.

In a preferred aspect, one of the up to 30 changes is to the position 221, such that it is not proline. However this is not essential as it has been shown that crystals can be obtained with proline at position 221 as long as one of the changes made above is also included.

A particular advantage of the proteins of the invention is that they are crystallisable. That is, we have found that we have been able to form crystals which diffract X-rays, and thus we have been able to analyse these crystals to provide structural coordinate data at a resolution of 3.1 Å or better, such as 2.55 Å.

It has also been shown in WO03/035693 that additional changes to the 2C9 wild type sequence in addition to the changes at any of 220-222 may be introduced. A number of specific changes are illustrated in WO03/035693 which include changes to the FG loop region and changes to the surface region of 2C9. More generally up to 20 changes in total on top of changes to positions 220 and 221 may be made.

B. Production of 2C9 Co-Crystals.

A number of methods are known as such in the art for obtaining protein crystals. 2C9 protein may be obtained as described in WO 03/035693, the contents of which are incorporated herein by reference.

Conveniently, the final protein is concentrated to 10-60, e.g. 20-40 mg/ml in 10-100 mM potassium phosphate with high salt (e.g. 500 mM NaCl or KCl) by using concentration devices that are commercially available. The protein may be concentrated in presence of 20% glycerol, 2.0 mM DTT and 1 mM EDTA.

The protein is crystallised by vapour diffusion at 5-25° C. against a range of buffer compositions. Crystals may be prepared using commercially available screening kits such as, Polyethylene glycol (PEG)/ion screens, PEG grid, Ammonium sulphate grid, PEG/ammonium sulphate grid or the like purchased from Hampton Research, Emerald Biostructure, Molecular Dimension and from others.

Typically the vapour diffusion buffer comprises 0-27.5%, preferably 2.5-27.5% PEG 1 K-20 K, preferably 1-8K or PEG 2000MME-5000MME, preferably PEG 2000 MME, or 0-10% Jeffamine M-600 and/or 5-20%, e.g. 10-20% propanol or 15-20% ethanol or about 15%-30%, e.g. about 15% 2-methyl-2,4-pentanediol (MPD), optionally with 0.01 M -1.6 M salt or salts and/or 0-0.15, e.g. 0-0.1, M of a solution buffer and/or 0-35%, such as 0-15%, glycerol and/or 0-35% PEG300-400; but preferably:

10-25% PEG 1K-8K or PEG 2000MME or 0-10% Jeffamine M-600 and/or 5-15%, e.g. 10-15%, propanol or ethanol, optionally with 0.1 M -0.2 M salt or salts and/or 0-0.15, e.g. 0-0.1 M solution buffer and/or PEG400, but more preferably:

15-20% PEG 3350 or PEG 4000 or PEG 2000MME or 0-10% Jeffamine M-600 or 5-15%, e.g. 10-15% propanol or ethanol, optionally with 0.1 M -0.2 M salt or salts and/or 0-0.15 M solution buffer.

Another preferred set of conditions are: 0.1M Tris pH 8.0-8.8, 2.5-25% PEG 400, 5-15% PEG 8000, 10-15% glycerol, 0-5% dioxane, preferably 0.1M Tris pH 8.4, 15-25% PEG 400, 5-12.5% PEG 8000, 10% glycerol.

Specifically preferred crystallisation conditions for the 2C9 proteins described herein are:

0.05-0.1 M Tris-HCl pH 8.0-8.8, 0.1-0.2 M Lithium sulphate, 10-15% PEG 4000;

0.1 M Tris pH 8.0-8.8, 15-30% PEG 400, 5% PEG 8000, 10% glycerol; and

0.1-0.4 M KH₂PO₄, 0-25% PEG 3350, 0-10% glycerol.

Specifically preferred conditions for co-crystallisation of a compound, such as warfarin and preferably S-warfarin are:

0.1M Tris 8.4, 25% PEG 400, 12.5% PEG 8000, 10% glycerol, 3% dioxane; or 0.1 M Tris pH 8-8.8, 15-30% PEG 400, 5% PEG 8000, 10% Glycerol.

The compound may be one or more compounds which are substrates or inhibitors or both.

The salt may be an alkali metal (particularly lithium, sodium and potassium), alkaline earth metal (e.g. magnesium or calcium), ammonium, ferric, ferrous or transition metal salt (e.g. zinc) of a halide (e.g. bromide, chloride or fluoride), acetate, formate, nitrate, sulphate, tartrate, citrate or phosphate. This includes sodium fluoride, potassium fluoride, ammonium fluoride, ammonium acetate, lithium acetate, magnesium acetate, sodium acetate, potassium acetate, calcium acetate, zinc acetate, ammonium chloride, lithium chloride, magnesium chloride, potassium chloride, sodium chloride, potassium bromide, magnesium formate, sodium formate, potassium formate, ammonium formate, ammonium nitrate, lithium nitrate, potassium nitrate, sodium nitrate, ammonium sulphate, potassium sulphate, lithium sulphate, sodium sulphate, di-sodium tartrate, potassium sodium tartrate, di-ammonium tartrate, potassium dihydrogen phosphate, tri-sodium citrate, tri-potassium citrate, zinc acetate, ferric chloride, calcium chloride, magnesium nitrate, magnesium sulphate, sodium dihydrogen phosphate, di-sodium hydrogen phosphate, di-potassium hydrogen phosphate, ammonium dihydrogen phosphate, di-ammonium hydrogen phosphate, tri-lithium citrate, nickel chloride, ammonium iodide, di-ammonium hydrogen citrate.

Solution buffers if present include, for example, Hepes, Tris, imidazole, cacodylate, tri-sodium citrate/citric acid, tri-sodium citrate/HCl, acetic acid/sodium acetate, phosphate-citrate, sodium potassium phosphate, 2-(N-morpholino)-ethane sulphonic acid/NaOH (MES), CHES, bis-trispropane, CAPS, potassium dihydrogen phosphate, sodium dihydrogen phosphate, dipotassium hydrogen phosphate or disodium hydrogen phosphate.

The pH range is desirably maintained at pH 4.2-10.5, preferably 4.2-8.5, more preferably 4.7-8.5 and most preferably 6.5-8.5.

Crystals may be prepared using a Hampton Research Screening kit, Poly-ethylene glycol (PEG)/ion screens, PEG grid, Ammonium sulphate grid, PEG/ammonium sulphate grid or the like.

Crystallisation may also be performed in the presence of an inhibitor or substrate of P450, e.g. fluvoxamine, fluconazole, 2-phenyl imidazole, warfarin, piroxicam or tenoxicam.

Additives can be added to a crystallisation condition identified to influence crystallisation. Additive Screens are to be used during the optimisation of preliminary crystallisation conditions where the presence of additives may assist in the crystallisation of the sample and the additives may improve the quality of the crystal e.g. Hampton additive Screens which use glycerol, polyols and other protein stabilizing agents in protein crystallisation (R. Sousa. Acta. Cryst. (1995) D51, 271-277) or divalent cations (Trakhanov, S. and Quiocho, F. A. Protein Science (1995) 4,9, 1914-1919).

In a further aspect, the invention provides a method for making a protein crystal of a P450 protein described herein, which method comprises growing a crystal by vapour diffusion using a reservoir buffer. The growing of the crystal is by vapour diffusion and is performed by placing an aliquot of the solution on a cover slip as a hanging drop above a well containing the reservoir buffer. The aliquot contains protein solution and reservoir buffer, typically in a ratio of 1 part protein solution to 1 part reservoir buffer. The protein solution was 0.7 mM. Preferably the reservoir buffer is 0.1 M Tris pH 8-8.8, 15-30% PEG 400, 5% PEG 8000, 10% Glycerol. Alternative crystallisation conditions comprise (i) 0-0.2 M Tris-HCl (pH 8-9.5, preferably pH 8.4-8.8), 0-20% PEG 400, 0-20% PEG 8000, 0-20% glycerol or (ii) 0-0.2 M Tris-HCl (pH 8-0.25 M Li₂SO₄, 0-20% PEG 4000; more particularly (iii) 0.1 M Tris-HCl (pH 8.8), 15% PEG 400, 5% PEG 8000, 10% glycerol, (iv) 0.1 M Tris-HCl (pH 8.5), 0.2 M Li₂SO₄, 15% PEG 4000 or (v) 0.1 M Tris-HCl (pH 8.4), 15% PEG 400, 5% PEG 8000, 10% glycerol. Conditions (iii) and (v) are particularly preferred.

In a co-crystallisation experiment, typically 2.5 mM to 5 mM of the compound is added to the reservoir solution to generate co-crystals.

In another aspect, co-crystals of 2C9 and a ligand may be obtained by back-soaking. This may be achieved by:

generating a 2C9-warfarin co-crystal of the invention;

removing warfarin from the co-crystal by soaking the crystal in a removal buffer;

soaking the crystal in a soaking solution comprising the ligand.

In an alternative aspect, co-crystals of 2C9 and a ligand may be obtained by co-crystallisation or soaking of a ligand into a 2C9 crystal.

Generation of the 2C9-S-Warfarin Complex Crystals.

Co-crystals of 2C9, such as construct 1155, with warfarin, preferably S-warfarin, are generated in a similar way to the generation of apo crystals. In order to obtain suitably large, well formed crystals it is necessary to set up a limited grid screen the following crystallization condition 0.1 M Tris pH 8-8.8, 15-30% PEG 400, 5% PEG 8000, 10% Glycerol. It may prove necessary to vary some of the crystallization variables (e.g. buffer pH, precipitant concentration) further than in the screen described above. Typically 5 mM of warfarin is added to the well solution but it may prove necessary to vary the ratio of S-warfarin stock to optimize the crystals. Crystals typically grow to their maximum dimensions over a period of 7 days at 25° C.

Removal of S-Warfarin from the Crystals

Crystals of S-warfarin grown by the above method are then soaked in a solution typically containing 12.5% PEG 400, 7% PEG 8000, 15% glycerol, 0.25 M KCl and 0.075 M buffers which can be Tris pH 8.4, or imidazole pH 8.5 to remove the warfarin. Further suitable conditions include 10-12.5 % PEG 400 for example 10% or 12.5% PEG 400, 7% PEG 8000, 15% glycerol, 0.25 M KCl and 0.075 M buffers which can be Tris pH 8.4, or Imidazole buffer pH 8.0-8.5 or Hepes pH 8.0. Preferably the buffer is Imidazole buffer pH 8.0-8.5 e.g. pH 8.0 or 8.5 or Hepes pH 8.0

Introduction of a New Compound into the Crystals

Once the crystals have had S-warfarin soaked out of them, they are transferred into a soaking solution. A suitable solution may contain 12.5% PEG 400, 7% PEG 8000, 15% glycerol, 0.25 M KCl and 0.075 M buffer which can be Tris pH 8.4, BisTris pH 6 or NaOAc pH 5.0. The soaking solution also contains the new compound. Typically the new compound may be at a concentration of 2.5-5 mM. The choice of buffer is dependent on the solubility of the compound at the different pHs.

C. Crystals

In a further aspect, the invention thus provides a co-crystal of human 2C9 P450 protein and S-warfarin. The crystal of P450 has the trigonal space group P321, and contains two copies of 2C9 in an asymmetric unit, denominated as A and B in Table 1 and Table 2.

Such a crystal may be obtained using the methods described in the accompanying examples.

The crystal may be of a 2C9 protein which comprises the sequence of SEQ ID NO:4 other than the following changes:

position 220 or position 222 is proline; and

optionally up to 21, for example up to 10, for example up to 5 other positions are altered,

the positions being numbered according to wild type 2C9. Such a 2C9 may be the sequence of SEQ ID NO:2.

The methodology used to provide a P450 crystal illustrated herein may be used generally to provide a human P450 co-crystal resolvable at a resolution of at least 3.1 Å and preferably at least 3 Å, more preferably at least 2.55 Å.

The invention thus further provides a co-crystal of a P450 protein described herein having a resolution of at least 3.1 Å and preferably at least 3 Å, more preferably at least 2.55 Å.

D. Description of Structure.

The analysis of the crystals obtained in the present invention has allowed a detailed analysis of the structure of a human P450 molecule. Cytochrome P450 2C9 can be considered to be a two domain protein, with a smaller, predominantly beta strand domain and a larger, predominantly alpha helical domain, forming an overall triangular arrangement. All P450 structures solved to date have the same overall topology, leading to a nomenclature adopted by the literature to describe the individual alpha helices and beta strands within P450 structures (see Ravichandran et al, Science, 1993, 261, 731-736 for definitions). The protein as purified consists of residues 19-494 (numbering from full length 2C9), and all but the first and last few of these residues are distinguishable in the electron density. The beta strand domain consists of beta sheets 1 and 2 and alpha helices A and B. These structural elements are formed by the N-terminal region of the polypeptide chain (residues 30-90) and residues between the helices K and K′. These residues, along with the loops between helices B and C, and helices F and G (herein referred to as the B-C and F-G loops), are implicated in the interaction of mammalian P450s with the membrane when the protein is in its native membranous form. These loops also confer some of the reaction specificity to individual P450s and are among the most divergent regions of sequence.

The alpha helical domain consists of helices C through L. The haem moiety is located between the alpha helical and the beta strand domains, and sits above helix I (residues 284-315). The single protein ligand to the haem, cysteine 435, is found in a loop prior to the last alpha helix. Given the range of compounds that P450s metabolise, the substrate binding pockets of these enzymes can accommodate a variety of shapes and sizes. Access to and from the haem group may be regulated by the position of the loops that form the substrate binding site, leading to open and closed conformations of the enzyme. Mutational and activity data has allowed the mapping of regions of sequence to function.

CYP2C9 is a two-domain protein with an overall fold characteristic of the CYP450 family. Studies have shown that the B-C loop contributes to substrate specificity, and in both the apo and complexed structures of CYP2C9 residues 101 to 106 in the B-C loop form helix B′. In addition, residues 212 to 222 in the F-G loop form helices F′ and G′, a feature previously not observed in any other CYP450 structure. The haem is located between helices I and L and is pentacoordinated with Cys435 as the single ligand. As in other CYP450 structures, a water molecule is hydrogen bonded to a highly conserved threonine, Thr301, and is located 7 Å above the haem, appropriate for its role in the proton-transfer path. In addition there is some residual electron density located 4Å above the haem, running up to and along side helix I; the features of this electron density make an interpretation ambiguous. The haem is stabilised by hydrogen bonds between the propionates and the side chains of residues Trp120, Arg124, His368 and Arg433. A key residue implicated by previous mutagenesis studies, Arg97, also forms hydrogen bonds to the propionates, as well as the carbonyl oxygens of Val113 and Pro367. Thus it would appear that the main role of Arg97 in CYP2C9 is haem stabilisation rather than substrate interaction as previously suggested.

Several reports indicate that CYP2C9 has a preference for small acidic lipophilic compounds as substrates, implying the presence of basic residues within the protein active site, and leading to postulation of an ‘anionic-binding site’. However, although a number of hydrophobic residues are clearly defined within the active site cavity there appear to be no basic residues with the potential to interact with substrates. The active site cavity extends up and away from the I helix, with Phe114 and Phe476 lying on opposite sides of the channel, and the very top of the channel being formed by the B′ helix, and the B-C and F-G loops. Phe114 points into the active site and is well positioned to form interactions with substrates as implicated by mutagenesis. Residues Phe69, Phe100, Leu102, Leu208, Leu362, Leu366 and Phe476, form a hydrophobic patch in the active site while Arg105 and Arg108, previously implicated in the formation of the putative anionic-binding site, both point away from the cavity. In contrast to basic residues, there are in fact two acidic residues present in the active site of apo CYP2C9. Asp293 is close to Phe110 and Phe114, and hydrogen bonds to the backbone nitrogen of Ile112 and consequently is well ordered, while Glu300 points into the active site but shows a degree of flexibility in the apo structure. In addition, Gln214 and Asn217 are both found close to Phe476 and could offer potential hydrogen bonding interactions with ligands.

The human isoforms CYP2C9 and CYP2C1 9 differ by 43 residues out of 490, and of these there is only one non-conservative substitution within the active site; residue 99 is an isoleucine in CYP2C9 and a histidine in CYP2C19. However unlike CYP2C9, CYP2C19 shows no apparent preference for compounds containing an acidic group. The widely held view is that this difference in the substrate selectivity for these two isoforms is due to the nature of the amino acids within their active sites. With the absence of basic residues in the active site of CYP2C9, the selectivity of these proteins may lie elsewhere. Most of the residues in the loops that are believed to form the substrate-access channel are conserved between CYP2C9 and CYP2C19, with the exception of residue 72 which a lysine in CYP2C9 and a glutamate in CYP2C19.

Comparison with 2C5.

An overlay of the 2C5 structure of PDB code 1 DT6 and 2C9 structure indicates that while the gross features of the protein are largely conserved between the two proteins, there are some interesting differences. The first resolvable residue in the electron density is residue 30 (all numbering is in relation to the full length protein), and the last residue is residue 490. Thus there are 10 residues without electron density at the N-terminus and the four histidine C-terminal tag is also not resolved.

Starting at the N-terminus, the two proteins adopt the same position at residue 48. Following the polypeptide chain back towards the N-terminus, the position of the two sequences is out of register by one, and towards the end, two residues, while the backbone trace of the two proteins is very close. The sequence identity in this region is particularly high, so such a difference seems somewhat surprising. It is probably attributable to the comparatively low resolution of the 2C5 structure which made accurately assigning the sequence at the N-terminus difficult. The higher resolution of the 2C9 structure has made assigning the sequence in this region less ambiguous. Thus this structure of 2C9 may be more representative of the true conformation of the N-termini of both 2C5 and 2C9.

The first region in which the two proteins differ substantially is the region between the B and C helices (residues 99 to 111). The temperature factors of the chain between residues 99 and 109 for the 2C5 structure of PDB Code 1 DT6 (Williams et al. 2000, Mol. Cell. 5, 121) are high (the average B factor for all atoms in this range is 99.1 Å²), implying much mobility in this region, and hence little confidence can be placed in their position. In contract, the average B-factors for all atoms for residues 99 to 111 is 55.5 Å² in 2C9.

In the 2C9 structure residues 101 to 106 have adopted a helical formation (helix B′) that has been observed in bacterial P450 structures. These residues form part of the first of six substrate recognition sites (SRSs), SRS 1, and thus contribute to the active site of the P450. The electron density has allowed unambiguous interpretation of all side chain positions in this region. A notable feature in this region is Arg97, which is proposed to be an important cation in the active site (2C9 substrate are predominantly acidic). The equivalent residue in 2C5 (Arg97) adopted a different conformation, and as a result did not form part of the active site. His99 has been implicated in omeprazole activity (Ibeanu et al., (1996), J Biol Chem, Vol. 271, 12496-12501); it is the only residue in SRS 1 not conserved between 2C9 and 2C19 (in 2C9 is it a Ile in 2C19 a His), and mutation of this residue alone in 2C19 confers omeprazole activity to the resulting mutant protein. The 2C9 structure confirms that this residue forms part of the active site.

The next region of divergence between the 2C5 and 2C9 structures is the region between the F and G helices. Residues 212 to 222 inclusive, which form part of the F-G loop, were absent in the published 2C5 structure. These residues are well resolved in the 2C9 structure, and form two turns of helix (all secondary structure assignment done using the program DSSP (Kabsch and Sander, Biopolymers 22 (1983) 2577-2637). Residues 220 and 221, while not contributing to the active site, clearly do have some impact on the accessibility of the active site, by mediating the position of the F-G loop. One of the disadvantages of mapping regions of sequence involved in substrate contact is the inability to distinguish between those regions which directly contact substrates (by lining the active site) and those that mediate the interaction the substrate has with the P450 by regulating structural elements within the enzyme.

The 2C9 structure will allow the distinction between direct and indirect impact of individual residues on substrate specificity and activity. The redesign of compounds to facilitate or remove interactions with 2C9 is clearly going to be simplified by this distinction.

Helices H and I adopt the same spatial conformation in the two proteins; the loop between the two helices is three residues longer and is clearly resolved in the electron density.

Substrate Recognition Sites

Several reports indicate that CYP2C9 has a preference for small acidic lipophilic compounds as substrates, implying the presence of basic residues within the protein active site, and leading to postulation of an ‘anionic-binding site’. However, although a number of hydrophobic residues are clearly defined within the active site cavity there appear to be no basic residues with the potential to interact with substrates. The active site cavity extends up and away from the I helix, with Phe114 and Phe476 lying on opposite sides of the channel, and the very top of the channel being formed by the B′ helix, and the B-C and F-G loops. Phe114 points into the active site and is well positioned to form interactions with substrates as implicated by mutagenesis. Residues Phe69, Phe100, Leu102, Leu208, Leu362, and Leu366 and Phe476, form a hydrophobic patch in the active site while Arg105 and Arg108, previously implicated in the formation of the putative anionic-binding site, both point away from the cavity. In contrast to basic residues, there are in fact two acidic residues present in the active site of apo CYP2C9. Asp293 is close to Phe110 and Phe114, and hydrogen bonds to the backbone nitrogen of Ile112 and consequently is well ordered, while Glu300 points into the active site but shows a degree of flexibility in the apo structure. In addition, Gln214 and Asn217 are both found close to Phe476 and could offer potential hydrogen bonding interactions with ligands.

A total of six substrate recognition sites (SRS) have been proposed by Gotoh (Gotoh, J. Biol. Chem., 267 (1992), 83-90). Some of the residues that line the binding pocket of the 2C9 structure include residues within these predicted SRS and include several residues that have been linked to changes in both specificity and reaction rates within mutant forms of the protein. The regiospecific hydroxylation of warfarin has been linked to polymorphism at residue 359; which lies above and to one side of the haem group, while residue 114 which has been shown to change Warfarin and diclofenac hydroxylation rates, lies above and to the other side of the haem group.

The structure of the present invention confirms that many of the residues inferred as potential SRS residues in the prior art by other methods (e.g. sequence alignment and mutagenesis) are found in the various SRSs seen in our structure. We have also identified many other residues which are likely to provide side chains capable of interacting with many P450 substrates. For example, our structure indicates a number of residues, particularly with hydrophobic side chains, are in the SRS regions.

However, a surprising feature of the active site pocket which has not previously been appreciated is that it is significantly larger than expected. The volume of the pocket is about 470 Å3. Given most substrate molecules are unlikely to be larger than 200 Å3, this raises the possibility that 2C9 may bind multiple compounds simultaneously. This may have implications for biological function as it raises the possibility of 2C9 using an allosteric mechanism during the reaction. For example, one compound bound in one part of the active site could increase the catalytic activity against other (or the same) substrate molecule. It may also provide a mechanism for the complex phenomena of drug-drug interactions. If one drug molecule is bound in a part of the active site, this could change the affinity of 2C9 for another drug molecule as the first molecule may offer direct molecular interactions for the second molecule to bind. This mechanism could provide an opportunity to alter/reduce the potential for drug-drug interactions by making specific chemical modifications to either of the drug molecules so that these inter-molecular interactions can no longer occur. This can be modeled in silico or determined crystallographically.

Ligand Binding Site

The active site pocket is lined with several amino acid residues which potentially can interact with a ligand. These amino acid residues include:

72, 74, 97, 98, 99, 100, 101, 102, 103, 104, 105, 106, 107, 110, 112, 113, 114, 116, 204, 205, 208, 213, 214, 216, 217, 233, 364, 365, 366, 367, 368, 369, 384, 385, 386, 387, 388, 476 and 477 of the 2C9 sequence as numbered in Table 1.

The amino acid residues which are of particular interest are:

97, 98, 99, 100, 101, 102, 103, 104, 105, 106, 107, 112, 113, 114, 233, 208, 204, 205, 213, 214, 216, 217, 364, 365, 366, 367, 476 are 477.

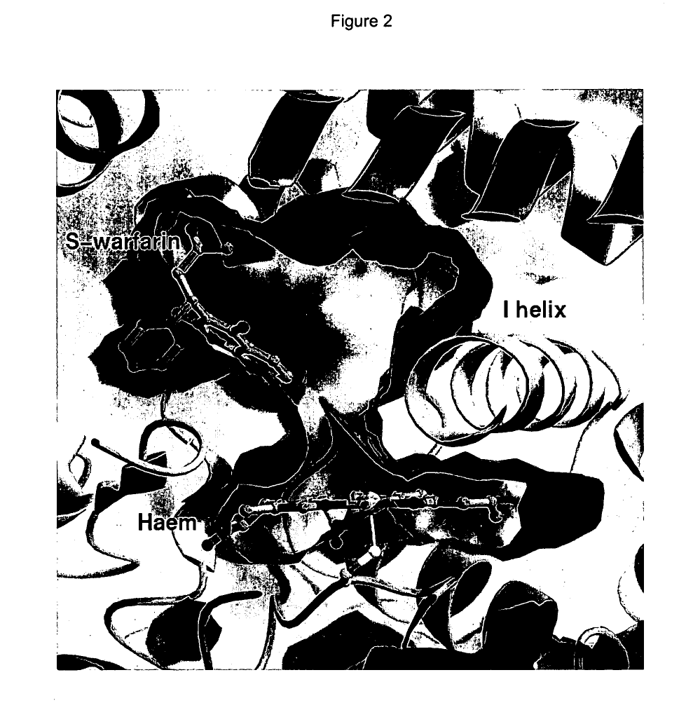

The structure of 2C9 with warfarin shows that the binding pocket in which warfarin binds is lined by predominantly hydrophobic residues Arg97, Gly98, Ile99, Phe100, Leu102, Ala103, Val113, Phe114, Asn217, Thr364, Ser365, Leu366, Pro367 and Phe476. The residue Leu 208 is also present in this binding pocket. As can be seen from FIG. 2, this ligand binding pocket occupies only one part of the total 2C9 binding pocket and is physically distinct from the region of the haem molecule also present in this pocket.

The discovery of the ligand binding site in which warfarin binds can be exploited in several ways for drug design. This is because the residues of this site will also interact with other compounds as well. In the simplest manifestation, if a drug is an inhibitor of 2C9 and this is undesirable, by altering the interactions with the site via medicinal chemistry, its interaction with the P450 can be modified. This would also be helpful if the drug binds at this site and does not inhibit but increases metabolism of a co-administered drug, in this instance new chemical modifications could be made to alter interactions with this site and also if the two molecules were seen to directly interact with each other (chemically modifying either molecule could be possible). This latter scenario is novel in the field of drug-drug interactions. The interactions with this ligand binding site can be increased or decreased depending on whether greater or lesser affinity for this pocket is desirable.

In the embodiments of the invention described herein where selected coordinates of the P450 structure may be used, the coordinates may include some or all of the residues of the binding pocket region discussed above and herein.

Haem Binding Pocket.

Some of the residues mentioned above, together with additional residues, form a region around the haem molecule in the 2C9 binding pocket. The main interaction for the compounds binding at the haem is with the iron atom of the haem itself. There are other residues around this region, although these may not be expected to form strong interactions themselves with the compounds.

The residues are of the haem pocket are:

97, 98, 111, 112, 113, 114, 115, 116, 178, 290, 293, 294, 295, 297, 298, 299, 300, 301, 302, 361, 362, 365, 366, 367, 368, 369, 389, 391 and 433.

The residues of particular interest are:

97, 112, 113, 114, 293, 294, 295, 296, 297, 298, 299, 300, 301, 302, 361, 362, 365 and 366.

The residues which we found to be of most significance are:

Leu294, Ala297, Gly298, Thr301, Thr302, Leu362 and Leu366.

Other Features

In the 2C9 structure the side chain position of Arg97 is clearly resolved, forming an interaction with the haem and Val113. Phe114 points into the active site and is well positioned to form pi-pi stacking interactions with substrates as has been suggested by a number of groups. Phe110 is in close proximity, but not as exposed at Phe114.

Arg105 and Arg108, which have also been suggested as potentially contributing to a cation site within the active site, both point away from the cavity.

The residues at positions 286 and 289 have been implicated in substrate specificity (Klose et al., (1998), Arch. Biochem. Biophys., Vol. 357, 240-248). Only residue 289 actually lines the active site, but both are in close proximity to Phe110 of the B-C loop, and hence their role in substrate specificity may be an indirect one via the packing of structural elements, rather than a direct one through substrate contact.

Phe476 forms a hydrophobic patch in the active site along with Phe100, Leu102, Leu208, Leu362, and Leu366.

There are 4 other alleles of 2C9 which have currently been identified, which have an amino acid substitution. 2C9*2 has R144C, 2C9*3 1359L, 2C9*4 1359T and 2C9*5 D360E. Ile359 does not lie in the active site, but is close to Thr305 and Thr361. It is not easy to envisage a direct effect of this residue on ability to catalyse compounds, but as has been noted for other residues, a mutation here may cause the shift of structural elements, which will impact on the active site. A similar effect may be true for Asp360. Arg 144 does not form part of the binding pocket of 2C9. It has however been widely believed that the variation in drug metabolism properties exhibited by those individuals possessing the 2C9 R144C allele variation is due to a modified interaction between the P450 and the reductase. The peripheral location of this residue in the structure of 2C9 would support this argument.

Dimer Interface

The rotation angle between the two copies in the asymmetric unit is not 180°, and as a result the interface between the two copies (here referred to as A and B) is non-symmetrical. The interface involves a number of hydrogen bonds between residues in helix D of molecule A and the G-H loop of molecule B, the G-H loop of molecule A and the C-terminus and helix D of molecule B, the C terminus of A and the G-H loop of molecule B.

E. Crystal Coordinates.

In a further aspect, the invention also provides a crystal of P450 having the three dimensional atomic coordinates of Table 1 and Table 2. An advantageous feature of the structures defined by the atomic coordinates is that they have a high resolution (about 2.55-2.6 Å).

Another advantageous feature of the invention in that it provides atomic coordinate data relating to the loop between helices F and G (the FG loop). The FG loop is one of the most divergent topological regions between the mammalian and bacterial P450 enzymes. As such, it is one of the more difficult parts of the mammalian enzymes to model when using a bacterial structure as a modelling template. The structure of P450BM3 (Ravichandran et al, 1993, ibid) has been widely used within the field as a structural template for modelling the human forms. P450BM3 has just twelve residues in the FG loop, as opposed to the 21 residues in the 2C isoforms. The only mammalian P450 structure in the public domain is that of the rabbit 2C5 isoform, solved by X-ray crystallography to a resolution of 3.0 Å (Williams et al, Mol Cell (2000), 5, 121-131). While the 2C5 structure does provide an improved modelling template when compared to the bacterial structures, the position of the FG loop was not resolvable in the crystal structure. In contrast, the 2C9 structure described here includes the FG loop. Residues within the FG loop have not been widely implicated in the substrate selectivity of P450s, and lie outside the substrate recognition sites (SRS's) identified by Gotoh (Gotoh, O, J. Biol. Chem, 267; 83-90 (1992)). Residues within the FG loop have been shown to modify the compound binding specificity of 2C9 (Tsao et al, Biochemistry (2001), 40, 1937-1944). It was not clear whether this eff ect was due to direct interaction of residues within the FG loop and the compound, or a secondary effect caused by the interaction of these residues with residues within the pocket that fall within the substrate recognition sites (SRS) of the enzymes. It is now evident from our structure that the residues of the FG loop do not contribute to the binding pocket. The structure of 2C9 will therefore more readily facilitate the identification of direct and indirect interactions between compounds and 2C9.

Another advantageous feature is that the average B-factor of the 2C9 structure is 43.9 Å² in contrast to the apo 2C5 structure of PDB Code 1 DT6 (Williams et al. 2000, Mol. Cell. 5, 121) which had an overall B-factor of 58.6 Å², resulting in a better definition for most of the side chains within the structure. This is advantageous for all uses of the coordinates, especially in silico work, molecular replacement, and homology modelling.

A further advantage of the 2C9 structure of Table 2 described herein is that it is an unliganded, apo structure. This makes it particularly suitable for soaking in ligands and hence determining co-complex structures and, are also ideal for homology modelling purposes as there is no conformational bias from a ligand.

The BC and FG loops are among the most varied features of cytochromes P450. Both loops contribute to the enzymes catalytic cycle; the BC loop directly by providing residues that form part of the active site, and mediate specificity and activity interactions, and the FG loop by movement allowing substrate entry and exit. In this high resolution 2C9 structure both of these loops are well resolved, in contrast to the 2C5 structure.

Tables 1 and 2 give atomic coordinate data for P450 2C9. In the Tables the third column denotes the atom, the fourth the residue type, the fifth the chain identification (either A or B), the sixth the residue number (the atom numbering is with respect to the full length wild type protein), the seventh, eighth and ninth columns are the X, Y, Z coordinates respectively of the atom in question, the tenth column the occupancy of the atom, the eleventh the temperature factor of the atom, the twelfth (where present) the chain identification, and the last the atom type.

The tables is presented in an internally consistent format. In Table 1 the coordinates of the atoms of each amino acid residue are listed such that the backbone nitrogen atom is first, followed by the C-alpha backbone carbon atom, designated CA, followed by the carbon and oxygen of the protein backbone and finally side chain residues (designated according to one standard convention). In Table 2 the carbon and oxygen backbone atoms follow the side-chain atoms. Thus alternative file formats (e.g. such as a format consistent with that of the EBI Macromolecular Structure Database (Hinxton, UK)) which may include a different ordering of these atoms, or a different designation of the side-chain residues, ligand or haem molecule atoms, may be used or preferred by others of skill in the art. However it will be apparent that the use of a different file format to present or manipulate the coordinates of the Tables is within the scope of the present invention.

The coordinates of Tables 1 and 2 provide a measure of atomic location in Angstroms, given to 3 decimal places. The coordinates are a relative set of positions that define a shape in three dimensions, but the skilled person would understand that an entirely different set of coordinates having a different origin and/or axes could define a similar or identical shape. Furthermore, the skilled person would understand that varying the relative atomic positions of the atoms of the structure so that the root mean square deviation of the residue backbone atoms (i.e. the nitrogen-carbon-carbon backbone atoms of the protein amino acid residues) is less than 2.0 Å, preferably less than 1.5 Å, more preferably less than 1.0 Å, more preferably less than 0.5 Å, more preferably less than 0.3 Å, such as less than 0.25 Å, or less than 0.2 Å, and most preferably less than 0.1 Å, when superimposed on the coordinates provided in Tables 1 and 2 for the residue backbone atoms, will generally result in a structure which is substantially the same as the structure of Tables 1 and 2 in terms of both its structural characteristics and usefulness for structure-based analysis of P450-interactivity molecular structures.

Likewise the skilled person would understand that changing the number and/or positions of the water molecules and/or substrate molecules of Tables 1 and 2 will not generally affect the usefulness of the structure for structure-based analysis of P450-interacting structure. Thus for the purposes described herein as being aspects of the present invention, it is within the scope of the invention if: the Tables 1 and 2 coordinates are transposed to a different origin and/or axes; the relative atomic positions of the atoms of the structure are varied so that the root mean square deviation of residue backbone atoms is less than 2.0 Å, preferably less than 1.5 Å, more preferably less than 1.0 Å, even more preferably less than 0.64 Å and most preferably less than 0.5 Å, more preferably less than 0.3 Å, such as less than 0.25 Å, or less than 0.2 Å, and most preferably less than 0.1 Å, when superimposed on the coordinates provided in Tables 1 and 2 for the residue backbone atoms; and/or the number and/or positions of water molecules and/or substrate molecules is varied.

Reference herein to the coordinate data of Tables 1 and 2 and the like thus includes the coordinate data in which one or more individual values of the Table are varied in this way. By “root mean square deviation” we mean the square root of the arithmetic mean of the squares of the deviations from the mean.

Those of skill in the art will appreciate that in many applications of the invention, it is not necessary to utilise all the coordinates of Tables 1 or 2, but merely apportion of them. Such a portion of co-ordinates is also referred herein as “selected co-ordinates”. For example, as described below, in methods of modelling candidate compounds with P450, selected coordinates of 2C9 may be used.

By “selected coordinates” it is meant for example at least 5, preferably at least 10, more preferably at least 50 and even more preferably at least 100, for example at least 500 or at least 1000 atoms of the 2C9 structure. Likewise, the other applications of the invention described herein, including homology modelling and structure solution, and data storage and computer assisted manipulation of the coordinates, may also utilise all or a portion of the coordinates (i.e. selected coordinates) of Tables 1 or 2. The selected coordinates may include or may consist of atoms found in the 2C9 P450 binding pocket, as described herein below, and particularly those of the ligand binding region such as those in Table 5, or those of Table 5 together with Leu208.

Also, modifications in the 2C9 crystal structure due to e.g. mutations, additions, substitutions, and/or deletions of amino acid residues (including the deletion of one or more 2C9 protomers) could account for variations in the 2C9 atomic coordinates. However, atomic coordinate data of 2C9 modified so that a ligand that bound to one or more binding sites of 2C9 would be expected to bind to the corresponding binding sites of the modified 2C9 are, for the purposes described herein as being aspects of the present invention, also within the scope of the invention. Preferably, the modified data define at least one 2C9 binding cavity.

Protein structure similarity is routinely expressed and measured by the root mean square deviation (r.m.s.d.), which measures the difference in positioning in space between two sets of atoms. The r.m.s.d. measures distance between equivalent atoms after their optimal superposition. The r.m.s.d. can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-alpha atoms only. For the purposes of this invention, the r.m.s.d. can be calculated over any of these, using any of the methods outlined below.

Methods of comparing protein structures are discussed in Methods of Enzymology, vol 115, pg 397-420. The necessary least-squares algebra to calculate r.m.s.d. has been given by Rossman and Argos (J. Biol. Chem. , vol 250, pp 7525 (1975)) although faster methods have been described by Kabsch (Acta Crystallogr., Section A, A92, 922 (1976); Acta Cryst. A34, 827-828 (1978)), Hendrickson (Acta Crystallogr., Section A, A35, 158 (1979)); McLachan (J. Mol. Biol., vol 128, pp 49 (1979)) and Kearsley (Acta Crystallogr., Section A, A45, 208 (1989)). Some algorithms use an iterative procedure in which the one molecule is moved relative to the other, such as that described by Ferro and Hermans (Ferro and Hermans, Acta Crystallographic, A33, 345-347 (1977)). Other methods e.g. Kabsch's algorithm locate the best fit directly.

Programs for determining r.m.s.d include MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript and on http://bioinfo1.mbfys.lu.se/TOP/maDs.html)).

It is usual to consider C-alpha atoms and the rmsd can then be calculated using programs such as LSQKAB (Collaborative Computational Project 4. The CCP4 Suite: Programs for Protein Crystallography, Acta Crystallographica, D50, (1994), 760-763), QUANTA (Jones et al., Acta Crystallography A47 (1991), 110-119 and commercially available from Accelerys, San Diego, Calif.), Insight (commercially available from Accelerys, San Diego, Calif.), Sybyl® (commercially available from Tripos, Inc., St Louis), O (Jones et al., Acta Crystallographica, A47, (1991), 110-119), and other coordinate fitting programs.

In, for example the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail in Section G. The atomic coordinates can then be superimposed according to this alignment and an r.m.s.d. value calculated. The program Sequoia (C. M. Bruns, I. Hubatsch, M. Ridderstrom, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Once aligned, the r.m.s.d. can be calculated using programs detailed above. For sequence identical, or highly identical, the structural alignment of proteins can be done manually or automatically as outlined above. Another approach would be to generate a superposition of protein atomic coordinates without considering the sequence.

It is more normal when comparing significantly different sets of coordinates to calculate the r.m.s.d. value over C-alpha atoms only. It is particularly useful when analysing side chain movement to calculate the r.m.s.d. over all atoms and this can be done using LSQKAB and other programs.

Thus, for example, varying the atomic positions of the atoms of the structure by up to about 0.5 Å, preferably up to about 0.3 Å, preferably up to about 0.25 Å, preferably up to about 0.2 Å and preferably up to about 0.1 Å in any direction will result in a structure which is substantially the same as the structure of Table 1 in terms of both its structural characteristics and utility e.g. for molecular structure-based analysis.

Those of skill in the art will appreciate that in many applications of the invention, it is not necessary to utilise all the coordinates of Table 1, but merely a portion of them. For example, as described below, in methods of modelling candidate compounds with P450, selected coordinates of 2C9 may be used.

By “selected coordinate” it is meant for example at least 5, preferably at least 10, more preferably at least 50 and even more preferably at least 100, for example at least 500 or at least 1000 atoms of the 2C9 structure. Likewise, the other applications of the invention described herein, including homology modelling and structure solution, and data storage and computer assisted manipulation of the coordinates, may also utilise all or a portion of the coordinates (i.e. selected coordinates) of Table 1. The selected coordinates may include or may consist of atoms found in the 2C9 P450 binding pocket, as described herein below.

F. Chimaeras

The use of chimaeric proteins to achieve desired properties is now common in the scientific literature. For example, Sieber et al (Nature Biotechnology (2001) 19, 456-460) produced hybrids between human cytochrome P450 isoform 1A2 and the bacterial P450 BM3, in order to make proteins with the specificity of 1A2, but which had desirable expression and solubility properties of BM3. Active site chimaeras are also described: for example, Swairjo et al (Biochemistry (1998) 37, 10928-10936) made loop chimaeras of HIV-1 and HIV-2 protease to try to understand determinants of inhibitor-binding specificity.

Of particular relevance are cases where the active site is modified so as to provide a surrogate system to obtain structural information. Thus Ikuta et al (J Biol Chem (2001) 276, 27548-27554) modified the active site of cdk2, for which they could obtain structural data, to resemble that of cdk4, for which no X-ray structure is currently available. In this way they were able to obtain protein/ligand structures from the chimaeric protein which were useful in cdk4 inhibitor design. In a similar way, based on comparison of primary sequences of highly related isoforms (such as 2C19 or even 2D6), the active site of the 2C9 protein could be modified to resemble those isoforms. Protein structures or protein/ligand structures of the chimaeric proteins could be used in structure-based alteration of the metabolism of compounds which are substrates of that related P450 isoform.

Even if the percentage of the amino acid sequence identity between mammalian P450 ranks from 20 to 80%, the overall folding of mammalian P450s is expected to be very similar, with the same spatial distribution of the structural elements. Furthermore, this class of enzymes exhibits distinct substrate specificities that rely on only a limited number of residues located in non-contiguous parts of the polypeptide chain. The substrate-binding pocket of P450 is generally constituted by residues that fall in the SRS regions (substrate recognition sites) defined by Gotoh (Gotoh, O, J. Biol. Chem, 267; 83-90 (1992)) and in loops of the molecule.

Aspects of the present invention therefore relate to modification of P450 proteins such that the active sites mimic those of related isoforms. For example, from a knowledge of the structure and residues of the active site of the human 2C9 protein described herein, and that of the rabbit 2C5 protein published previously, a person skilled in the art could modify the 2C5 protein such that the active site mimicked that of human 2C9. This protein could then be used to obtain information on compound binding through the determination of protein/ligand complex structures using the chimaeric 2C5 protein.

For example, in one aspect the present invention provides a chimaeric protein having a binding cavity which provides a substrate specificity substantially identical to that of P450 2C9 protein, wherein the chimaeric protein binding cavity is lined by a plurality of atoms which correspond to selected P450 2C9 atoms lining the P450 2C9 binding cavity, the relative positions of the plurality of atoms corresponding to the relative positions, as defined by Table 1 or Table 2, of the selected P450 2C9 atoms.

It is possible to postulate that only few changes would be required to inter-convert the substrate specificities of P450 isoforms that exhibit more than 70% of amino acid identity. For example, 2C9 and 2C19, although they differ at only 43 of 490 amino acids, exhibits clear substrate specificity differences. Using a panel of 2C9/2C19 chimaeric proteins, Jung et al. (Jung, F. Biochemistry, 37, 16270-16279 (1998)), have identified the sequences differences that confer to 2C19 a high affinity binding to sulfaphenazole, a very potent and specific inhibitor of 2C9. Site directed mutagenesis experiments have revealed that the conversion of 2C19 to a 2C9-like protein was possible by introducing a limited number of substitutions in the 2C19 amino acid sequence. These mutations are located in the SRS3 and SRS4 regions of the proteins. Similar studies performed by Klose etal. (Arch. Biochem. Biophys. 357, 240-248 (1998)) and Tsao etal. (Biochemistry, 40, 1937-1944, (2001)) have demonstrated the feasibility of the transfer of substrate specificities between 2C9 and 2C19 by mutating SRS regions.

The substrate specificity of an enzyme generally relies on only a limited number of residues located in non-contiguous parts of the polypeptide chain. The substrate specificities of these isoforms could be analysed by substituting these residues by site-directed mutagenesis. The minimal changes that would be required to convert another protein into a 2C9-like chimera could be at least two amino acids selected from Table 3. These mutations can be introduced by site-directed mutagenesis e.g. using a Stratagene QuikChange™ Site-Directed Mutagenesis Kit or cassette mutagenesis methods (Ausubel, F. M., Brent, R., Kingston, R. E. et al. editors. Current Protocols in Molecular Biology. John Wiley & Sons, Inc., New York, Sambrook, J., Fritsch, E. F., and Maniatis, T. (1989). Molecular Cloning: a Laboratory Manual. 2nd ed. Cold Spring Harbor Laboratory Press, Cold Spring Harbor, N.Y.). Thus the invention provides a chimaeric protein having one or more binding pockets defined by the residues of any one of Tables 3-5.

This strategy could clearly be applied for proteins that exhibit high sequence homology with or without overlapping substrate specificities and from different species. The rabbit 2C5 and the human 2C9 and 2C19 P450s have been reported to be involved in the metabolism of progesterone with different rates, the rabbit isoform being clearly the most efficient enzyme. The use of the crystal structures solved for 2C5 and 2C9 would allow the characterization of the binding mode of the progesterone molecule in the substrate pocket of these proteins. This in turn would allow the identification of residues to be modified in the human isoforms to convert them into efficient progesterone metabolising enzymes.

In one embodiment, a chimaeric 2C9 enzyme is produced which is isoformal with another enzyme of the 2C subfamily. For example, 2C9 could be turned into a 2C1 9-like isoform with a few amino acid changes. Based on the information available from the literature on the structure/activity studies performed on the 2C9 and 2C19 isoforms, and the analysis of the structure of the human 2C9, we postulate that the 2C9 protein could be converted to a 2C19-like protein with the substrate specificities attributed to 2C19.

The residues to be mutated are one or more of:

Substitute SRS 1 of 2C9 with SRS 1 of 2C19 (the amino acid change introduced is I99H); and/or

Substitute SRS 3 of 2C9 with SRS 3 of 2C19 (the amino acid changes introduced are V237L and K241 E); and/or

Substitute SRS 4 of 2C9 with SRS 4 of 2C19 (the amino acid changes introduced are S286N, E288V, N289I, V292A and F295L—the key changes could be S286N, N289I, V292A and F295L); and/or

Move SRS5 of 2C19 to 2C9 (the amino acid L362I is introduced).

The minimal changes that would be required to convert 2C9 to 2C19 could be 199H, K241 E, S286N, N289I, V292A, F295L and L362I and more likely to be I99H, S286N, N289I, V292A, and F295L. These mutations can be introduced by site-directed mutagenesis or cassette mutagenesis methods, as described herein.

A 2C19-like chimera can also be made by making the following changes: I99H, S286N, E288V, N289I, V292A, F295L. An alternative minimal change would be I99H, S286N, N289I.

The crystallisation of such chimeras and the determination of the three-dimensional structures relies on the ability of our 2C9 proteins to yield crystals that diffract at high resolution. The aim is to modify the inside part or 2C9 to produce a new substrate binding site of 2C19 without modifying the outside shell of the proteins that allow the protein to crystallise.

Examples 17-22 of WO03/035693, the contents of which are incorporated herein by reference, illustrate the production of 2C9-2C19 chimeric proteins.

G. Homology Modelling.

The invention also provides a means for homology modelling of other proteins (referred to below as target P450 proteins). By “homology modelling”, it is meant the prediction of related P450 structures based either on x-ray crystallographic data or computer-assisted de novo prediction of structure, based upon manipulation of the coordinate data of Table 1.

The P450 structure set out in Table 1 is, as explained in further detail herein, a dimer structure. The various in silico modelling techniques described in this section and in the other sections of this application may utilize either the dimer structure of Table 1 or either of the subunits A and B. To avoid unnecessary repetition, reference is made herein to the coordinate data of Table 1 but this will be understood to mean either the data for both subunits or just one of the subunits.

“Homology modelling” extends to target P450 proteins which are analogues or homologues of the 2C9 P450 protein whose structure has been determined in the accompanying examples. It also extends to P450 protein mutants of 2C9 protein itself.

In general, the method involves comparing the amino acid sequences of the 2C9 P450 protein of Table 1 with a target P450 protein by aligning the amino acid sequences. Amino acids in the sequences are then compared and groups of amino acids that are homologous (conveniently referred to as “corresponding regions”) are grouped together. This method detects conserved regions of the polypeptides and accounts for amino acid insertions or deletions.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

Homology between amino acid sequences can be determined using commercially available algorithms. The programs BLAST, gapped BLAST, BLASTN, PSI-BLAST and BLAST 2 sequences (provided by the National Center for Biotechnology Information) are widely used in the art for this purpose, and can align homologous regions of two amino acid sequences. These may be used with default parameters to determine the degree of homology between the amino acid sequence of the Table 1 protein and other target P450 proteins which are to be modelled.

Analogues are defined as proteins with similar three-dimensional structures and/or functions and little evidence of a common ancestor at a sequence level.

Homologues are defined as proteins with evidence of a common ancestor i.e. likely to be the result of evolutionary divergence and are divided into remote, medium and close sub-divisions based on the degree (usually expressed as a percentage) of sequence identity.

A homologue is defined here as a protein with at least 15% sequence identity or which has at least one functional domain, which is characteristic of 2C9. This includes polymorphic forms of 2C9.

There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene/chromosome/genome duplication, i.e. the common ancestor of the genes occurred since the last speciation event.

A mutant is a 2C9 characterized by replacement or deletion of at least one amino acid from the wild type 2C9. Such a mutant may be prepared for example by site-specific mutagenesis, or incorporation of natural or unnatural amino acids.

The present invention contemplates “mutants” wherein a “mutant” refers to a polypeptide which is obtained by replacing at least one amino acid residue in a native or synthetic 2C9 with a different amino acid residue and/or by adding and/or deleting amino acid residues within the native polypeptide or at the N— and/or C-terminus of a polypeptide corresponding to 2C9 and which has substantially the same three-dimensional structure as 2C9 from which it is derived. By having substantially the same three-dimensional structure is meant having a set of atomic structure coordinates that have a root mean square deviation (r.m.s.d.) of less than or equal to about 2.0 Å when superimposed with the atomic structure coordinates of the 2C9 from which the mutant is derived when at least about 50% to 100% of the C_{α} atoms of the 2C9 are included in the superposition. A mutant may have, but need not have, enzymatic or catalytic activity.

To produce homologues or mutants, amino acids present in the said protein can be replaced by other amino acids having similar properties, for example hydrophobicity, hydrophobic moment, antigenicity, propensity to form or break α-helical or β-sheet structures, and so. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags, MBP tag, and epitope tags.

Amino acid substitutions, deletions and additions which do not significantly interfere with the three-dimensional structure of the 2C9 will depend, in part, on the region of the 2C9 where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

Conservative amino acid substitutions are well-known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues to a 2C9 binding pocket or catalytic residue in order to provide convenient cloning sites in cDNA encoding the polypeptide, to aid in purification of the polypeptide, etc. Such substitutions, deletions and/or additions which do not substantially alter the three dimensional structure of 2C9 will be apparent to those having skills in the art.

It should be noted that the mutants contemplated herein need not exhibit enzymatic activity. Indeed, amino acid substitutions, additions or deletions that interfere with the catalytic activity of the 2C9 but which do not significantly alter the three-dimensional structure of the catalytic region are specifically contemplated by the invention. Such crystalline polypeptides, or the atomic structure coordinates obtained there from, can be used to identify compounds that bind to the protein. The homlogues could also be polymorphic forms of 2C9 such as alleles or mutants as described in section (A).

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Homology modelling as such is a technique that is well known to those skilled in the art (see e.g. Greer, Science, Vol. 228, (1985), 1055, and Blundell et al., Eur. J. Biochem, Vol. 172, (1988), 513). The techniques described in these references, as well as other homology modelling techniques generally available in the art, may be used in performing the present invention.

Thus the invention provides a method of homology modelling comprising the steps of:

(a) aligning a representation of an amino acid sequence of a target P450 protein of unknown three-dimensional structure with the amino acid sequence of the P450 of Table 1 to match homologous regions of the amino acid sequences;

(b) modelling the structure of the matched homologous regions of said target P450 of unknown structure on the corresponding regions of the P450 structure as defined by Table 1; and

(c) determining a conformation (e.g. so that favourable interactions are formed within the target P450 of unknown structure and/or so that a low energy conformation is formed) for said target P450 of unknown structure which substantially preserves the structure of said matched homologous regions.

Preferably one or all of steps (a) to (c) are performed by computer modelling.

The presence of the FG loop in our structure is particularly advantageous for modelling of other P450s especially mammalian P450s, which have longer FG loops than bacterial P450s as there is currently nothing known in the art about the conformation of the FG loop in mammalian structures. This is advantageous for modelling compounds into this structure or modelled structures.

The data of Table 1 will be particularly advantageous for homology modelling of other human P450 proteins, in particular human P450s such as 2C8, 2C18, 2C19, 2D6, 3A4, 1Al, 1A2, 2E1. These proteins may be the target P450 protein in the method of the invention described above.

In a particularly preferred aspect, the homology model is selected from the group consisting of 2C19, 2C18 and 2C8. The resulting homology models may be used in the methods described herein below in sections H, I and J.

The aspects of the invention described herein which utilise the P450 structure in silico may be equally applied to homologue models of P450 obtained by the above aspect of the invention, and this application forms a further aspect of the present invention. Thus having determined a conformation of a P450 by the method described above, such a conformation may be used in a computer-based method of rational drug design as described herein.

H. Structure Solution

The structure of the human 2C9 P450 can also be used to solve the crystal structure of other target P450 proteins including other crystal forms of 2C9, mutants, co-complexes of 2C9, where X-ray diffraction data or NMR spectroscopic data of these target P450 proteins has been generated and requires interpretation in order to provide a structure.

In the case of 2C9, this protein may crystallise in more than one crystal form. The structure coordinates of 2C9, or portions thereof, as provided by this invention are particularly useful to solve the structure of those other crystal forms of 2C9. They may also be used to solve the structure of 2C9 mutants, 2C9 co-complexes, or of the crystalline form of any other protein with significant amino acid sequence homology to any functional domain of 2C9.

In the case of other target P450 proteins, particularly the human P450 proteins referred to in Section D above, the present invention allows the structures of such targets to be obtained more readily where raw X-ray diffraction data is generated.

Thus, where X-ray crystallographic or NMR spectroscopic data is provided for a target P450 of unknown three-dimensional structure, the structure of P450 as defined by Table 1 may be used to interpret that data to provide a likely structure for the other P450 by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of 2C9, a 2C9 mutant, or a 2C9 co-complex, or the crystal of a target P450 protein with amino acid sequence homology to any functional domain of 2C9, may be determined using the 2C9 structure coordinates of this invention as provided herein. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelrys San Diego, Calif.), MOLREP (A. Vagin, A. Teplyakov, MOLREP: an automated program for molecular replacement, J. Appl. Cryst. (1997) 30, 1022-1025, part of the CCP4 suite) or AMoRe (Navaza, J. (1994). AMoRe: an automated package for molecular replacement. Acta Cryst. A50, 157-163).

Thus, in a further aspect of the invention provides a method for determining the structure of a protein, which method comprises;

providing the coordinates of Table 1, and

positioning the coordinates in the crystal unit cell of said protein so as to provide a structure for said protein.

In a preferred aspect of this invention the coordinates are used to solve the structure of target P450s particularly homologues of 2C9 for example 2C19, 2C8, 2C18.

The invention may also be used to assign peaks of NMR spectra of such proteins, by manipulation of the data of Table 1.

I. Computer Systems.

In another aspect, the present invention provides systems, particularly a computer system, the systems containing either (a) atomic coordinate data according to Table 1, said data defining the three-dimensional structure of P450 or at least selected coordinates thereof; (b) structure factor data (where a structure factor comprises the amplitude and phase of the diffracted wave) for P450, said structure factor data being derivable from the atomic coordinate data of Table 1; (c) atomic coordinate data of a target P450 protein generated by homology modelling of the target based on the data of Table 1; (d) atomic coordinate data of a target P450 protein generated by interpreting X-ray crystallographic data or NMR data by reference to the data of Table 1; or (e) structure factor data derivable from the atomic coordinate data of (c) or (d).

The atomic coordinate data may be the data of the entire Table or a selected portion thereof.

The invention also provides such systems containing atomic coordinate data of target P450 proteins wherein such data has been generated according to the methods of the invention described herein based on the starting data provided by Table 1.

Such data is useful for a number of purposes, including the generation of structures to analyse the mechanisms of action of P450 proteins and/or to perform rational drug design of compounds which interact with P450, such as compounds which are metabolised by P450s.

In a further aspect, the present invention provides computer readable storage medium with either (a) atomic coordinate data according to Table 1 recorded thereon, said data defining the three-dimensional structure of P450, or at least selected coordinates thereof; (b) structure factor data for P450 recorded thereon, the structure factor data being derivable from the atomic coordinate data of Table 1; (c) atomic coordinate data of a target P450 protein generated by homology modelling of the target based on the data of Table 1; (d) atomic coordinate data of a target P450 protein generated by interpreting X-ray crystallographic data or NMR data by reference to the data of Table 1; or (e) structure factor data derivable from the atomic coordinate data of (c) or (d).

The atomic coordinate data may be the data of the entire Table or a selected portion thereof.

As used herein, “computer-readable storage medium” refers to any medium or media which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such a storage medium, the atomic coordinate data can be routinely accessed to model P450 or selected coordinates thereof. For example, RASMOL (Sayle et al., TIBS, Vol. 20, (1995), 374) is a publicly available computer software package which allows access and analysis of atomic coordinate data for structure determination and/or rational drug design.

On the other hand, structure factor data, which are derivable from atomic coordinate data (see e.g. Blundell et al., in Protein Crystallography, Academic Press, New York, London and San Francisco, (1976)), are particularly useful for calculating e.g. difference Fourier electron density maps.

As used herein, “a computer system” refers to the hardware means, software means and data storage means used to analyse the atomic coordinate data of the present invention. The minimum hardware means of the computer-based systems of the present invention typically comprises a central processing unit (CPU), a working memory and data storage means, and e.g. input means, output means etc. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

In another aspect, the invention provides a computer-readable storage medium, comprising a data storage material encoded with computer readable data, wherein the data are defined by all or a portion (i.e. selected coordinates as defined herein) of the structure coordinates of 2C9 of Table 1, or a homologue of 2C9, wherein said homologue comprises backbone atoms that have a root mean square deviation from the backbone atoms (nitrogen-carbona-carbon) of Table 1 of not more than 2.0 Å (preferably not more than 1.5 Å).

The invention also provides a computer-readable data storage medium comprising a data storage material encoded with a first set of computer-readable data comprising a Fourier transform of at least a portion (i.e. selected coordinates as defined herein) of the structural coordinates for 2C9 according to Table 1; which, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

A further aspect of the invention provides a method of providing data for generating structures and/or performing drug design with 2C9, 2C9 homologues or analogues, complexes of 2C9 with a compound, or complexes of 2C9 homologues or analogues with compounds, the method comprising:

(i) establishing communication with a remote device containing computer-readable data comprising at least one of: (a) atomic coordinate data according to Table 1, said data defining the three-dimensional structure of 2C9, at least one sub-domain of the three-dimensional structure of 2C9, or the coordinates of a plurality of atoms of 2C9; (b) structure factor data for 2C9, said structure factor data being derivable from the atomic coordinate data of Table 1; (c) atomic coordinate data of a target 2C9 homologue or analogue generated by homology modelling of the target based on the data of Table 1, such as the data of Table 18; (d) atomic coordinate data of a protein generated by interpreting X-ray crystallographic data or NMR data by reference to the data of Table 1; and (e) structure factor data derivable from the atomic coordinate data of (c) or (d); and

(ii) receiving said computer-readable data from said remote device.

Thus the remote device may comprise e.g. a computer system or a computer-readable storage medium of one of the previous aspects of the invention. The device may be in a different country or jurisdiction from where the computer-readable data is received.

The communication may be via the internet, intranet, e-mail etc, transmitted through wires or by wireless means such as by terrestrial radio or by satellite. Typically the communication will be electronic in nature, but some or all of the communication pathway may be optical, for example, over optical fibers.

J. Uses of the Structures of the Invention.

The crystal structures obtained according to the present invention (including the structures of Table 1 as well the structures of target P450 proteins obtained in accordance with the methods described herein) may be used in several ways for drug design. For example, many drugs or drug candidates fail to be of clinical use due to the detrimental interactions with P450 proteins, resulting in a rapid clearance of the drugs from the body. The present invention will allow those of skill in the art to attempt to rescue such compounds from development by following these structure-based chemical strategies detailed below.

In the case where a drug molecule is being metabolised by a P450, information on the binding orientation by either co-crystallisation, soaking or computationally docking the binding orientation of the drug in the binding pocket can be determined. This will guide specific modifications to the chemical structure designed to mediate or control the interaction of the drug with the protein. Such modifications can be designed with an aim of reducing the metabolism of the drug by P450 and so of improving its therapeutic action.

The crystal structure could also be useful to understand drug-drug interactions. Many examples exist where adverse reactions to drugs are recorded if administered while the patient is already taking other medicines. The mechanism behind this detrimental and often dangerous drug-drug interaction scenario may be when one drug behaves as an inhibitor of a P450 resulting in toxic levels of the other drug building-up due to less or no metabolism occurring. The crystal structure of the present invention complexed to such an inhibitor (either in vitro or in silico) may also allow rational modifications either to modify the inhibitor such that it no longer inhibits or inhibits less, or to modify the second drug such that it could bind better to the P450 (so becoming metabolised) and so displace the inhibitor.

P450s display significant polymorphic variations dependent on the age, gender or ethnic origin of the patient. This can manifest itself in adverse reactions from some segments of patient populations to some drugs. By using the crystal structures of the present invention to map the relevant mutation with respect to the binding mode of the drug, chemical modifications could also be made to the drug to avoid interactions with the variable region of the protein. This could ensure more consistent therapeutic value from the drug for such segments of the population and avoid dangerous side-effects.

Some pharmaceutical compounds are converted by P450s into active metabolites. In the case of such compounds, a greater understanding of how such compounds are converted by a P450 will allow modification of the compound so that it can be converted at a different rate. For example, increasing the rate of conversion may allow a more rapid delivery of a desired therapeutic effect, whereas decreasing the rate of conversion may allow for higher doses to be administered or the development of sustained release pharmaceutical preparations, for example comprising a mixture of compounds which are metabolised at different rates to form the same active metabolite.

Thus, the determination of the three-dimensional structure of P450 provides a basis for the design of new compounds which interact with P450 in novel ways. For example, knowing the three-dimensional structure of P450, computer modelling programs may be used to design different molecules expected to interact with possible or confirmed active sites, such as binding sites or other structural or functional features of P450.

In general, the invention may be used to perform a method of assessing the ability of a compound to interact with P450 2C9 protein which comprises:

obtaining or synthesising said compound;

forming a crystallised complex of a P450 2C9 protein and said compound, said complex diffracting X-rays for the determination of atomic coordinates of said complex to a resolution of better than 3.1 Å, preferably 2.55 Å; and

analysing said complex by X-ray crystallography to determine the ability of said compound to interact with the P450 2C9 protein.

Such analysis may utilise the coordinate data of Tables 1 or Table 2, or selected coordinates of Table 1 or Table 2. In the case of the latter, the selected coordinates may include those of an iron ion.

(i) Obtaining and Analysing Crystal Complexes.

In one approach, the structure of a compound bound to a P450 may be determined by experiment. This will provide a starting point in the analysis of the compound bound to P450, thus providing those of skill in the art with a detailed insight as to how that particular compound interacts with P450 and the mechanism by which it is metabolised.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al., in Protein Crystallography, Academic Press, New York, London and San Francisco, (1976)), it is necessary to know beforehand the protein 3D structure (or at least the protein structure factors). Therefore, determination of the P450 structure also allows production of difference Fourier electron density maps of P450-compound complexes to be produced, determination of the binding position of a drug, and hence may greatly assist the process of rational drug design.

Accordingly, the invention provides a method for determining the structure of a compound bound to P450, said method comprising:

providing a crystal of 2C9 P450 according to the invention;

soaking the crystal with said compounds; and

determining the structure of said 2C9 P450 compound complex by employing the data of Table 1.

Alternatively, the P450 and compound may be co-crystallised. Thus the invention provides a method for determining the structure of a compound bound to P450, said method comprising; mixing the protein with the compound(s), crystallising the protein-compound(s) complex; and determining the structure of said P450-compound(s) complex by reference to the data of Table 1.

The analysis of such structures may employ (i) X-ray crystallographic diffraction data from the complex and (ii) a three-dimensional structure of P450, or at least selected coordinates thereof, to generate a difference Fourier electron density map of the complex, the three-dimensional structure being defined by atomic coordinate data according to Table 1. The difference Fourier electron density map may then be analysed.

Therefore, such complexes can be crystallised and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallised P450 and the solved structure of uncomplexed P450. These maps can then be analysed e.g. to determine whether and where a particular compound binds to P450 and/or changes the conformation of P450.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “O” (Jones et al., Acta Crystallographica, A47, (1991), 110-119) can be used.

In addition, in accordance with this invention, 2C9 mutants may be crystallised in co-complex with known 2C9 substrates or inhibitors or novel compounds. The crystal structures of a series of such complexes may then be solved by molecular replacement and compared with that of the 2C9 of Table 1. Potential sites for modification within the various binding sites of the enzyme may thus be identified. This information provides an additional tool for determining the most efficient binding interactions, for example, increased hydrophobic interactions, between 2C9 and a chemical entity or compound.

For example there are alleles of 2C9, which differ from the native 2C9 by only 1 or 2 amino acid substitutions, and yet individuals who express these allelic variants may exhibit very different drug metabolism profiles. Polymorphisms in the human CYP2C9 genes can influence the outcome of a treatment for a range of diseases including cancer. The metabolism of chemotherapeutic agents used in the treatment of cancer can be investigated using the structure provided here and the agents then altered using the methods described herein.

All of the complexes referred to above may be studied using well-known X-ray diffraction techniques and may be refined against 1.5 to 3.5 Å resolution X-ray data to an R value of about 0.30 or less using computer software, such as CNX (Brunger et al., Current Opinion in Structural Biology, Vol. 8, Issue 5, October 1998, 606-611, and commercially available from Accelrys, San Diego, Calif.), and as described by Blundell et al, (1976) and Methods in Enzymology, vol.114 & 115, H. W. Wyckoff et al., eds., Academic Press (1985).

This information may thus be used to optimise known classes of 2C9 substrates or inhibitors, and more importantly, to design and synthesize novel classes of 2C9 inhibitors and design drugs with modified P450 metabolism.

(ii) In Silico Analysis and Design.

Although the invention will facilitate the determination of actual crystal structures comprising a P450 and a compound which interacts with the P450, current computational techniques provide a powerful alternative to the need to generate such crystals and generate and analyse diffraction date. Accordingly, a particularly preferred aspect of the invention relates to in silico methods directed to the analysis and development of compounds which interact with P450 structures of the present invention.

Determination of the three-dimensional structure of 2C9 provides important information about the binding sites of 2C9, particularly when comparisons are made with similar enzymes. This information may then be used for rational design and modification of 2C9 substrates and inhibitors, e.g. by computational techniques which identify possible binding ligands for the binding sites, by enabling linked-fragment approaches to drug design, and by enabling the identification and location of bound ligands using X-ray crystallographic analysis. These techniques are discussed in more detail below.

Thus as a result of the determination of the P450 three-dimensional structure, more purely computational techniques for rational drug design may also be used to design structures whose interaction with P450 is better understood (for an overview of these techniques see e.g. Walters et al (Drug Discovery Today, Vol.3, No.4, (1998), 160-178; Abagyan, R.; Totrov, M. Curr. Opin. Chem. Biol. 2001, 5, 375-382). For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47, 409-443), which require accurate information on the atomic coordinates of target receptors may be used.

The aspects of the invention described herein which utilize the P450 structure in silico may be equally applied to both the 2C9 structure of Table 1 and the models of target P450 proteins obtained by other aspects of the invention. Thus having determined a conformation of a P450 by the method described above, such a conformation may be used in a computer-based method of rational drug design as described herein. In addition the availability of the structure of the P450 2C9 will allow the generation of highly predictive pharmacophore models for virtual library screening or compound design.

Accordingly, the invention provides a computer-based method for the analysis of the interaction of a molecular structure with a P450 structure of the invention, which comprises:

providing the structure of a P450 of the invention;

providing a molecular structure to be fitted to said P450 structure; and

fitting the molecular structure to the P450 structure.

The P450 structure of the invention may be the structure of Table 1 or selected coordinates thereof.

In an alternative aspect, the method of the invention may utilize the coordinates of atoms of interest of the P450 binding region which are in the vicinity of a putative molecular structure, for example within 10-25 Å of the catalytic regions or within 5-10 Å of a compound bound, in order to model the pocket in which the structure binds. These coordinates may be used to define a space which is then analysed “in silico”. Thus the invention provides a computer-based method for the analysis of molecular structures which comprises:

providing the coordinates of at least two atoms of a P450 structure of the invention (“selected coordinates”);

providing a molecular structure to be fitted to said coordinates; and

fitting the structure to the selected coordinates of the P450.

In practice, it will be desirable to model a sufficient number of atoms of the P450 as defined by the coordinates of Table 1 which represent a binding pocket. Binding pockets and other features of the interaction of P450 with co-factor are described in the accompanying example. Thus, in this embodiment of the invention, there will preferably be provided the coordinates of at least 5, preferably at least 10, more preferably at least 50 and even more preferably at least 100 selected atoms such as at least 500 or at least 1000 atoms of the P450 structure.

Although every different compound metabolised by P450 may interact with different parts of the binding pocket of the protein, the structure of this P450 allows the identification of a number of particular sites which are likely to be involved in many of the interactions of P450 with a drug candidate. The residues are set out in the accompanying example. Thus in this aspect of the invention, the selected coordinates may comprise coordinates of some or all of these residues.

In order to provide a three-dimensional structure of compounds to be fitted to a P450 structure of the invention, the compound structure may be modelled in three dimensions using commercially available software for this purpose or, if its crystal structure is available, the coordinates of the structure may be used to provide a representation of the compound for fitting to a P450 structure of the invention.

The binding pockets of cytochrome P450 molecules are of a size which can accommodate more than one ligand. Indeed, some drug-drug interactions may occur as a result of interaction of the compounds within the binding pocket of the same P450. In any event, the findings of the present invention may be used to examine or predict the interaction of two or more separate molecular structures within the P450 2C9 binding pocket of the invention.

By “fitting”, it is meant determining by automatic, or semi-automatic means, interactions between at least one atom of a molecular structure and at least one atom of a P450 structure of the invention, and calculating the extent to which such an interaction is stable. Interactions include attraction and repulsion, brought about by charge, steric considerations and the like. Various computer-based methods for fitting are described further herein.

More specifically, the interaction of a compound or compounds with P450 can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, J. Mol. Biol. 1982, 161, 269-288, Makino et al, J. Comput. Chem. 1997, 18, 1812-1825), AUTODOCK (Goodsell et al, Proteins 1990, 8, 195-202, Morris et al, J. Comput. Chem. 1998, 19, 1639-1662.), FlexX, (Rarey et al, J. Mol. Biol. 1996, 261, 470-489) or ICM (Abagyan et al, J. Comput. Chem. 1994, 15, 488-506). This procedure can include computer fitting of compounds to P450 to ascertain how well the shape and the chemical structure of the compound will bind to the P450.

Also computer-assisted, manual examination of the active site structure of P450 may be performed. The use of programs such as GRID (Goodford, J. Med. Chem., 28, (1985), 849-857)—a program that determines probable interaction sites between molecules with various functional groups and an enzyme surface—may also be used to analyse the active site to predict, for example, the types of modifications which will alter the rate of metabolism of a compound.

Computer programs can be employed to estimate the attraction, repulsion, and steric hindrance of the two binding partners (i.e. the P450 and a compound).

If more than one P450 active site is characterized and a plurality of respective smaller compounds are designed or selected, a compound may be formed by linking the respective small compounds into a larger compound which maintains the relative positions and orientations of the respective compounds at the active sites. The larger compound may be formed as a real molecule or by computer modelling.

Detailed structural information can then be obtained about the binding of the compound to P450, and in the light of this information adjustments can be made to the structure or functionality of the compound, e.g. to alter its interaction with P450. The above steps may be repeated and re-repeated as necessary.

As indicated above, molecular structures which may be fitted to the P450 structure of the invention include compounds under development as potential pharmaceutical agents. The agents may be fitted in order to determine how the action of P450 modifies the agent and to provide a basis for modelling candidate agents which are metabolised at a different rate by a P450.

Molecular structures which may be used in the present invention will usually be compounds under development for pharmaceutical use. Generally such compounds will be organic molecules which are typically from about 100 to 2000 Da, more preferably from about 100 to 1000 Da in molecular weight. Such compounds include peptides and derivatives thereof, steroids, anti-inflammatory drugs, anti-cancer agents, anti-bacterial or antiviral agents, neurological agents and the like. In principle, any compound under development in the field of pharmacy can be used in the present invention in order to facilitate its development or to allow further rational drug design to improve its properties.

A single reductase provides several different isoforms of P450 with the electrons required in the catalytical cycle. As such, knowledge of the cytochrome P450 reductase (CPR) binding site on P450 and its characteristics present a means of altering the rate of catalysis, by mediating the P450 CPR interactions. The structure of 2C9 will allow the in silico identification of residues important in the P450-CPR interface.

(iii) Analysis and Modification of Compounds and Metabolites

Where the primary metabolite of a potential or actual pharmaceutical compound is known, and this metabolite is generated by the action of P450, the structure of the agent and its metabolite may both be modelled and compared to each other in order to better determine residues of P450 which interact with the agent. In any event, the present invention provides a process for predicting potential pharmaceutical compounds with a desired activity which are metabolised by P450 at a rate different from a starting compound having the same desired activity, which method comprises:

fitting a starting compound to a P450 structure of the invention or selected coordinates thereof;

determining or predicting how said compound is metabolised by said P450 structure; and

modifying the compound structure so as to alter the interaction between it and the P450.

It would be understood by those of skill in the art that modification of the structure will usually occur in silico, allowing predictions to be made as to how the modified structure interacts with the P450.

Greer et al. (J. of Medicinal Chemistry, Vol. 37, (1994), 1035-1054) describes an iterative approach to ligand design based on repeated sequences of computer modelling, protein-ligand complex formation and X-ray crystallographic or NMR spectroscopic analysis. Thus novel thymidylate synthase inhibitor series were designed de novo by Greer et al., and P450 ligands may also be designed or modified in the this way. More specifically, using e.g. GRID on the solved structure of P450, a ligand for P450 may be designed that complements the functionalities of the P450 binding sites. Alternatively a ligand for P450 may be modified such that it complements the functionalities of the P450 binding sites better or less well. The ligand can then be synthesised, formed into a complex with P450, and the complex then analysed by X-ray crystallography to identify the actual position of the bound ligand. The structure and/or functional groups of the ligand can then be adjusted, if necessary, in view of the results of the X-ray analysis, and the synthesis and analysis sequence repeated until an optimised ligand is obtained. Related approaches to structure-based drug design are also discussed in Bohacek etal., Medicinal Research Reviews, Vol. 16, (1996), 3-50. Design of a compound with alternative P450 properties using structure based drug design may also take into account the requirements for high affinity to a second, target protein. Gschwend et al., (Bioorganic & Medicinal Chemistry Letters, Vol 9, (1999), 307-312) and Bayley et al., (Proteins: Structure, Function and Genetics, Vol 29, (1997) 29-67) describe approaches where structure based drug design is used to reduce affinity to one protein whilst maintaining affinity for a target protein.

Modifications will also be those conventional in the art known to the skilled medicinal chemist, and will include, for example, substitutions or removal of groups containing residues which interact with the amino acid side chain groups of a P450 structure of the invention. For example, the replacements may include the addition or removal of groups in order to decrease or increase the charge of a group in a test compound, the replacement of a group to increase or decrease the size of the group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Although it is usually desired to alter a compound to prevent its metabolism by P450, or at least to reduce the rate at which P450 metabolises the compound, the present invention also includes developing compounds which are metabolised more rapidly than a starting compound. Additionally the present invention includes developing compounds with high affinity for a P450, where such a compound blocks metabolism of another drug.

Where a potential modified compound has been developed by fitting a starting compound to the P450 structure of the invention and predicting from this a modified compound with an altered rate of metabolism, the invention further includes the step of synthesizing the modified compound and testing it in a in vivo or in vitro biological system in order to determine its activity and/or the rate at which it is metabolised.

The above-described processes of the invention may be iterated in that the modified compound may itself be the basis for further compound design. The above-described processes may also be used to modify a compound which interacts with a second compound within the 2C9 binding pocket.

(iv) Analysis of Compounds in Binding Pocket Regions.

Our finding of distinct regions in the large 2C9 binding pocket for the binding of warfarin and that of the haem allows the analysis and design methods described in the preceding subsections to be focused on these regions.

Warfarin and other compounds are metabolised by 2C9 by hydroxylation. The iron residue of the haem is considered crucial to the reaction. However, in our structure, the iron ion of the haem is located about 10 Å away from the carbon atom of warfarin which is subject to hydroxylation. This may be too far away for the reaction to occur. While not wishing to be bound by any one particular theory, it is believed that the region of the binding pocket in which the warfarin is found may represent a holding position for this and other compounds in the ligand-binding region. The ligand may have to move from this region towards the haem-binding region e.g. for the hydroxylation reaction to occur or to inhibit the reaction. The movement of the ligand between sites may be due to an allosteric or conformationally driven switch e.g. upon reductase binding, or a change in affinity for pockets possibly due to changes in redox state of the iron ion.

Such a mechanism provides a means to modify ligands of 2C9 in order to alter their metabolism. By altering (i.e. increasing or decreasing) their affinity to the ligand-binding region compared to the haem binding region it may alter (i.e. increase or decrease) their ability to move towards the haem-binding region. For example by increasing their affinity to the ligand-binding region over the haem binding region may decrease their ability to move towards the haem-binding region. Alternatively, decreasing their affinity to the ligand-binding region may be desired to decrease their affinity to this region compared to the haem binding region and hence increase their ability to move towards the haem binding region. If compound binding to the ligand-binding pocket is a necessary prerequisite of compound binding in the haem-binding region and its subsequent metabolism by or inhibition of 2C9, elimination of binding to the ligand-binding region may eliminate all compound metabolism by 2C9 or inhibition of 2C9. An alternative or additional approach is to modify such substrates to increase or decrease their affinity for residues of the haem-binding region. Changes of this type may be introduced in order to increase or decrease the turnover of the substrates.

Other compounds have also been shown to bind to the ligand binding site (LBS) which binds warfarin. X-ray crystallographic studies by the inventors have shown that piroxicam and tenoxicam, two other substrates of CYP2C9 also bind to CYP2C9 at this ligand binding site distant from the haem as described herein for S-warfarin. The ligand binding site is therefore a binding site for warfarin, piroxicam and tenoxicam and thus the residues of this site interact with other compounds as well. The similarity between piroxicam or tenoxicam and S-warfarin is striking given that all three substrates are poorly metabolized by CYP2C9. This suggests that the metabolism mechanism hypothesized for S-warfarin could also be the same for piroxicam and tenoxicam.

The LBS may also be useful in a strategy for improving the pharmacokinetics of existing drugs by inhibiting their metabolism by 2C9. Poor or variable pharmacokinetics is a key problem for a number of drugs, especially those that have a narrow therapeutic window. An example is the anticoagulant warfarin, which requires significant monitoring to achieve the right therapeutic dose in patients.

The main current approach to improving pharmacokinetics of drugs is to redesign the drug molecule. This is sometimes difficult to do. An alternative but less utilised approach is to co-administer the drug with an inhibitor of the P450 that metabolises the drug molecule. Such an inhibitor will modulate the metabolism of the drug molecule and thus improve its pharmacokinetics. The methods described herein may be utilised to design such an inhibitor, which preferably in this case is a selective 2C9 inhibitor as a non-selective inhibitor may interfere with normal and important functions of P450s.

The discovery and characterisation of an alternative pocket (also called herein the ligand binding pocket or region) for warfarin, some distance away from the haem group in 2C9, shows that warfarin metabolism may be more complex than currently thought. It is conceived that a molecule that binds to the warfarin pocket may interfere with warfarin metabolism without necessarily interfering with the metabolism of other 2C9 substrates and more importantly without interfering with the normal operation of other P450 isoforms. It is therefore conceived that inhibitors of this pocket in 2C9 may be usefully co-administered with warfarin to provide a more predictable, stable and uniform pharmacokinetic profile amongst patients who currently use warfarin as an anticoagulant. It is also conceived that other P450's may have pockets (different form each other and from 2C9) which are distant from the haem group and which may be important in the metabolism of those drug molecules. Binding to these pockets may offer a general, but potentially more selective way of modulating pharmacokinetic properties of drug molecules. This may also be useful for drug molecules where the pharmacokinetic profile is not satisfactory e.g. piroxicam and tenoxicam.

Thus the invention also provides a method of administering a pharmaceutical compound metabolized by 2C9 to a patient (e.g. warfarin particularly S-warfarin, piroxicam or tenoxicam) wherein said compound is administered simultaneously or sequentially with a second compound which binds at the ligand binding pocket of 2C9. Such a second compound may be obtained using the methods of the present invention.

Thus in one embodiment, the present invention provides a method for modifying the structure of a compound in order to alter its metabolism by a P450, which method comprises:

fitting a starting compound to one or more coordinates of at least one amino acid residue of the ligand-binding region of the P450;

modifying the starting compound structure so as to increase or decrease its interaction with the ligand-binding region;

wherein said ligand-binding region is defined as the P450 residues numbered as: 72, 74, 97, 98, 99, 100, 101, 102, 103, 104, 105, 106, 107, 110, 112, 113, 114, 116, 204, 205, 208, 213, 214, 216, 217, 233, 364, 365, 366, 367, 368, 369, 384, 385, 386, 387, 388, 476 and 477; though preferably: 97, 98, 99, 100, 101, 102, 103, 104, 105, 106, 107, 112, 113, 114, 233, 208, 204, 205, 213, 214, 216, 217, 364, 365, 366, 367, 476 and 477; and most preferably as: Arg97, Gly98, Ile99, Phe100, Leu102, Ala103, Val113, Phe114, Asn217, Thr364, Ser3665, Leu366, Pro367 and Phe476; or alternatively Arg97, Gly98, Ile99, Phe100, Leu102, Ala103, Val113, Phe114, Leu208, Asn217, Thr364, Ser365, Leu366, Pro367 and Phe476.

In another embodiment, the invention provides a method for modifying the structure of a compound in order to alter its metabolism by a P450, which method comprises:

fitting a starting compound to one or more coordinates of at least one amino acid residue of the haem-binding region of the P450;

modifying the starting compound structure so as to increase or decrease its interaction with the haem-binding region;

wherein said haem-binding region is defined as the P450 residues numbered as: 97, 98, 111, 112, 113, 114, 115, 116, 178, 290, 293, 294, 295, 297, 298, 299, 300, 301, 302, 361, 362, 365, 366, 367, 368, 369, 389, 391 and 433; though preferably: 97, 112, 113, 114, 293, 294, 295, 296, 297, 298, 299, 300, 301, 302, 361, 362, 365 and 366; and most preferably: Leu294, Ala297, Gly298, Thr301, Thr302, Leu362, Leu366.

In one embodiment, when a starting compound is fitted to at least one amino acid of the haem-binding region, a second compound structure may be fitted to the ligand-binding region. This will allow that the interaction of the starting compound structure or modified structures thereof with the second compound structure may be determined.

In another embodiment, when a starting compound structure is fitted to the ligand-binding region, a second compound structure may be fitted to the haem binding region. This will allow the interaction of the starting compound structure or modified structures thereof with the second compound structure to be determined.

In these embodiments of the invention, the compound fitted to the haem-binding region may be a first compound structure—for example warfarin, piroxicam or tenoxicam—whose metabolism may differ between individuals. By fitting second compound structures to the ligand-binding region it may be possible to design compounds which alter the metabolism of the first compound by for example directing the first compound to the haem binding region preferentially over occupation of the ligand binding region.

The haem binding region also optionally includes the iron ion bound to the haem molecule, and if desired, one or more of the other atoms of the haem molecule itself. In a preferred aspect of the invention, the iron ion is also included in the haem-binding region.

In aspects of the invention in which the iron ion of the haem binding region is included in analysis, design, modification or fragment linking of structures, the coordinates of Table 2 may be used in place of Table 1. Thus all references herein to the use of the structure or coordinates of Table 1, wherein such uses are performed with an iron ion of the Table, shall apply mutatis mutandis to Table 2.

Desirably, in the above aspects of the invention, coordinates from at least two, preferably at least five, and more preferably at least ten amino acid residues of the P450 (including where desired the iron ion) will be used.

For the avoidance of doubt, the term “modifying” is used as defined in the preceding subsection, and once such a compound has been developed it may be synthesised and tested also as described above.

(v) Fragment Linking and Growing.

The provision of the crystal structures of the invention will also allow the development of compounds which interact with the binding pocket regions of P450s (for example to act as inhibitors of a P450) based on a fragment linking or fragment growing approach.

For example, the binding of one or more molecular fragments can be determined in the protein binding pocket by X-ray crystallography. Molecular fragments are typically compounds with a molecular weight between 100 and 200 Da (Carr et al, 2002). This can then provide a starting point for medicinal chemistry to optimise the interactions using a structure-based approach. The fragments can be combined onto a template or used as the starting point for ‘growing out’ an inhibitor into other pockets of the protein (Blundell et al, 2002). The fragments can be positioned in the binding pocket of the P450 and then ‘grown’ to fill the space available, exploring the electrostatic, van der Waals or hydrogen-bonding interactions that are involved in molecular recognition. The potency of the original weakly binding fragment thus can be rapidly improved using iterative structure-based chemical synthesis.

At one or more stages in the fragment growing approach, the compound may be synthesized and tested in a biological system for its activity. This can be used to guide the further growing out of the fragment.

Where two fragment-binding regions are identified, a linked fragment approach may be based upon attempting to link the two fragments directly, or growing one or both fragments in the manner described above in order to obtain a larger, linked structure, which may have the desired properties.

Where the binding site of two or more ligands are determined they may be connected to form a potential lead compound that can be further refined using e.g. the iterative technique of Greer et al. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design P450 inhibitors is made possible by the determination of the P450 structure.

(vi) Compounds of the Invention.

Where a potential modified compound has been developed by fitting a starting compound to the P450 structure of the invention and predicting from this a modified compound with an altered rate of metabolism (including a slower, faster or zero rate), the invention further includes the step of synthesizing the modified compound and testing it in a in vivo or in vitro biological system in order to determine its activity and/or the rate at which it is metabolised.

The method comprises: (a) providing 2C9 under conditions where, in the absence of modulator, the 2C9 is able to metabolise known substrates; (b) providing the compound; and (c) determining the extent to which the compound is metabolised in the presence of 2C9 or (d) determining the extent to which the compound inhibits metabolism of a known substrate of 2C9.

More preferably, in the latter steps the compound is contacted with P450 under conditions to determine its function.

For example, in the contacting step above the compound is contacted with P450 in the presence of the compound, and typically a buffer and substrate, to determine the ability of said compound to inhibit P450 or to be metabolised by P450. The substrate may be e.g. methoxy-4-(trifluoromethyl)-coumarin. So, for example, an assay mixture for P450 may be produced which comprises the compound, substrate and buffer.

In another aspect, the invention includes a compound which is identified by the methods of the invention described above.

Following identification of such a compound, it may be manufactured and/or used in the preparation, i.e. manufacture or formulation, of a composition such as a medicament, pharmaceutical composition or drug. These may be administered to individuals.

Thus, the present invention extends in various aspects not only to a compound as provided by the invention, but also a pharmaceutical composition, medicament, drug or other composition comprising such a compound. The compositions may be used for treatment (which may include preventative treatment) of diseases such as cancer or myocardial ischemia/reperfusion injury.

The cytochrome P450 enzymes play a critical role in the oxidative metabolism of a variety of endogenous and exogenous compounds, including drugs. Some intermediate CYP metabolites are believed to play a role in carcinogenesis. CYPs involved in estrogen metabolism are expressed in both tumor and non-tumor breast tissue. CYP2C9 is detected in breast tumours and is involved in the conversion of estrone sulfate to the 16-hydroxy sulfate metabolite. (Modugno, F; Knoll, C; Kanbour-Shakir, A; Romkes, M; Breast Cancer Research and Treatment (2003), 82(3), 191-197). Thus CYP inhibitors designed using the methods herein could be useful in the treatment of cancer.

Examples of cancers include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

CYPs are also implicated in myocardial ischemia/reperfusion injury and reduction of ischemia and reperfusion-induced myocardial damage has been observed by cytochrome P450 inhibitors, thus the methods described herein could be used to cytochrome P450 inhibitors for reduction of ischemia and reperfusion-induced myocardial damage.

Accordingly, compounds identified according to the present invention and compositions thereof may be used in the treatment of conditions mentioned above. Such treatment may comprise administration of such a composition to a patient, e.g. for treatment of disease; the use of such an inhibitor in the manufacture of a composition for administration, e.g. for treatment of disease; and a method of making a pharmaceutical composition comprising admixing such an inhibitor with a pharmaceutically acceptable excipient, vehicle or carrier, and optionally other ingredients.

Thus a further aspect of the present invention provides a method for preparing a medicament, pharmaceutical composition or drug, the method comprising:

(a) identifying or modifying a compound by a method of any one of the other aspects of the invention disclosed herein; (b) optimising the structure of the molecule; and (c) preparing a medicament, pharmaceutical composition or drug containing the optimised compound.

The above-described processes of the invention may be iterated in that the modified compound may itself be the basis for further compound design.

By “optimising the structure” we mean e.g. adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimisation is regularly undertaken during drug development programmes to e.g. enhance potency, promote pharmacological acceptability, increase chemical stability etc. of lead compounds.

Modification will be those conventional in the art known to the skilled medicinal chemist, and will include, for example, substitutions or removal of groups containing residues which interact with the amino acid side chain groups of a P450 structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see “Remington: The Science and Practice of Pharmacy”, 20th Edition, 2000, pub. Lippincott, Williams & Wilkins.

### EXAMPLES

The invention is illustrated by the following examples.

### Example 1

### Co-Crystallisation of 2C9-FGloop K206E with S-Warfarin

CYP 2C9 catalyses the 6- and 7-hydroyxiation of the active enantiomer of warfarin, S-warfarin, to inactive metabolites. To explore the molecular basis of drug recognition we have determined the crystal structure of CYP 2C9 complexed with S-warfarin.

2C9-FGloop K206E was produced in E. coli as described in Annex 1, the contents of which correspond to Example 1, 2, and 8 of WO03/035693. To establish the effect of the truncation and mutagenesis, a comparison of the activity and specificity of the protein was performed, and it was confirmed that the 6′ and 7′ hydroxylation of warfarin remained unchanged compared to wild-type 2C9.

Crystals were obtained by the hanging drop vapour diffusion method, using a 1:1 ratio of protein at 40 mg/ml in a solution of 10 mM potassium phosphate, pH 7.4, 0.5M potassium chloride, 20% (v/v) glycerol, 1 mM EDTA, 2 mM DTT against a crystallisation well solution of 0.1M Tris, pH 8.4, 15-25% (v/v) PEG 400, 5-12.5% (w/v) PEG 8000, 10% (v/v) glycerol supplemented with 5 mM S-warfarin. Crystals formed over a period of 1-4 days at 25° C., and were frozen directly from the crystallisation solution.

The crystals were of space group P321 of unit cell dimensions of a=164.76 Å, b=164.76 Å, c=110.76 Å. Crystals of this unit cell dimension, ±5% for each dimension, form a further aspect of the invention.

X-ray data were collected at beam line ID14.2 at the ESR, processed using MOSFLM, scaled and further reduced using the CCP4 suite of programs. The apo structure of 2C9 described in Examples 9 and 11 of WO03/035693 (set out in Annexes 1 D and 2B below) was used in the refinement of the warfarin structure. The warfarin ligand was positioned in the electron density maps using AUTOSOLVE and refined using CNX and REFMAC.

The residues lining the binding pocket of 2C9 are set out in Table 3 as follows:

TABLE 3
All residues lining the 2C9 binding pocket

| | | | | | | | | | | | |
|---|---|---|---|---|---|---|---|---|---|---|---|
| ARG | 97 | GLY | 98 | ILE | 99 | PHE | 100 | LEU | 102 | ALA | 103 |
| ALA | 106 | ASN | 107 | GLY | 109 | PHE | 110 | GLY | 111 | ILE | 112 |
| VAL | 113 | PHE | 114 | THR | 167 | PHE | 168 | ILE | 178 | CYS | 179 |
| ILE | 181 | ILE | 182 | MET | 198 | LEU | 201 | ASN | 202 | ASN | 204 |
| ILE | 205 | LEU | 208 | SER | 209 | SER | 210 | PRO | 211 | ILE | 213 |
| GLN | 214 | ASN | 217 | LEU | 233 | VAL | 237 | MET | 240 | LYS | 241 |
| ASN | 289 | VAL | 292 | ASP | 293 | LEU | 294 | PHE | 295 | GLY | 296 |
| ALA | 297 | GLY | 298 | THR | 299 | GLU | 300 | THR | 301 | THR | 302 |
| SER | 303 | THR | 304 | THR | 305 | ARG | 307 | ASP | 360 | LEU | 361 |
| LEU | 362 | PRO | 363 | THR | 364 | SER | 365 | LEU | 366 | PRO | 367 |
| ASN | 474 | GLY | 475 | PHE | 476 | ALA | 477 | SER | 478 | VAL | 479 |
| LYS | 72 | ILE | 74 | PRO | 101 | GLU | 104 | ARG | 105 | SER | 115 |
| ASN | 116 | TYR | 216 | THR | 290 | HIS | 368 | ALA | 369 | GLY | 384 |
| THR | 385 | THR | 386 | ILE | 387 | LEU | 388 | ILE | 389 | LEU | 391 |
| ARG | 433 | | | | | | | | | | |

Using the data to build models of the binding pocket (FIG. 2), we found that warfarin lies in a predominantly hydrophobic pocket lined by the residues set out in Table 4 below:

Some residues found in the binding pocket have never before been identified as binding site residues. These are listed in Table 5, and also include Leu208. Identification of these will greatly facilitate the modelling of compound binding.

TABLE 4
Residues newly identified as lining the 2C9 binding pocket

| | | | | | | | | | | | |
|---|---|---|---|---|---|---|---|---|---|---|---|
| THR | 167 | PHE | 168 | ILE | 178 | CYS | 179 | ILE | 181 | ILE | 182 |
| MET | 198 | PRO | 211 | ILE | 213 | ASN | 217 | VAL | 479 | | |

We have also identified residues of the warfarin binding pocket. The interaction of compounds with these residues are of particular interest, as is the design of modified compounds which are altered to interact to a greater or lesser extent with these residues.

TABLE 5
Residues of the 2C9 warfarin binding pocket

| | | | | | | | | | | | |
|---|---|---|---|---|---|---|---|---|---|---|---|
| ARG | 97 | GLY | 98 | ILE | 99 | PHE | 100 | LEU | 102 | ALA | 103 |
| VAL | 113 | PHE | 114 | ASN | 217 | THR | 364 | SER | 365 | LEU | 366 |
| PRO | 367 | PHE | 476 | | | | | | | | |

The warfarin binding pocket additionally may include Leu208.

More specifically, the phenyl group of warfarin packs against the side chains of Phe476 and Phe100 and also contacts Pro367. Although the binding of warfarin to CYP2C9 appears not to have induced major conformational changes within the protein, some local rearrangements are observed. For example, the presence of the compound has slightly displaced the loop containing residues 474-478 by 0.5-1.5 Å. The side chain of Phe476, which showed conformational mobility in the apo structure, forms a pi-pi stacking interaction with the warfarin phenyl group. The bicylic scaffold of warfarin also makes van der Waals contact with the side chains of Ala103, Phe114 and Pro367. Hydrogen bonding interactions are also observed between carbonyl oxygen atoms of the warfarin and backbone amide nitrogen atoms of Phe100 and Ala103.

Although many of the residues lining the binding pocket where the warfarin bound previously have been shown by site directed mutagenesis to alter the catalytic properties of CYP2C9, this region had not been previously identified as a ligand binding site. Furthermore, the lack of significant conformational change within the protein upon compound binding is reflected in the active site volume remaining constant at ˜470 Å³. As the volume occupied by the warfarin molecule is ˜160 Å³, this raises the intriguing possibility for additional small molecules to simultaneously bind within the active site. When bound in this orientation and location, the site of hydroxylation of warfarin is about 10 Å away from the haem iron (FIG. 2), which is believed to be too distant for the hydroxylation to occur, suggesting that additional movement of the compound from this primary recognition site towards the haem is required to facilitate catalysis. We speculate that such a two step conformational movement may be triggered by an electron-transfer driven conformational change within CYP2C9, perhaps on reduction of the haem iron or interaction with the electron-transfer partner, cytochrome P450 reductase. It is also possible that some ligands are unable to move closer to the haem and so can behave as competitive inhibitors by occupying this binding site.

The discovery of this novel binding site may have implications for understanding the complex mechanisms employed by the human CYP450 proteins during their biological function. There are many reports that the human CYP450 proteins catalyse reactions exhibiting atypical kinetics such as activation, auto-activation and substrate inhibition. The vast majority of observations citing this cooperativity have been made with human CYP3A4, which routinely exhibits a capacity for multiple ligand binding during its function. The crystal structure indicates that human CYP2C9 may also have the capacity to bind multiple substrates/ligands simultaneously during its function and is consistent with reports which implicate a ‘two-site model’ for CYP2C9. The warfarin binding site could be one of these sites which, when occupied, ‘activates’ CYP2C9 through an allosteric mechanism, as there is sufficient space for other substrate molecules to bind at the haem (FIG. 2). This is consistent with data that shows CYP2C9 increases its catalytic activity against other substrates in the presence of warfarin. Furthermore, although the phenomenon of CYP450-mediated drug-drug interactions is widely reported for many drugs, the molecular basis remains unclear. A drug molecule bound at the binding site would be ideally placed to make direct molecular interactions with another drug molecule interacting with the haem group.

These new finding thus provide a further aspect to the invention, namely the use of some or all of the binding pocket residues of the site where warfarin binds in examining the interaction of compounds with CYP 2C9 and/or modelling or modifying the structures of compounds to alter their interaction with CYP 2C9.

Thus, in the methods of the invention described above in which selected coordinates of a structure of the invention is used to design, modify or otherwise analyse the interaction of a compound with CYP 2C9, such selected coordinates may include coordinates from at least one, preferably at least 2, for example at least 4, such as at least 7, more preferably at least 10 and in one embodiment all 14, residues of Table 5, or Table 5 together with Leu208.

In another aspect, the modelling of a compound with selected residues of Table 5—or Table 5 together with Leu208 may be performed in conjunction with the modelling of a further compound in the CYP 2C9 binding pocket. Thus the invention provides a computer-based method for the analysis of the interaction of two molecular structures within a P450 binding pocket structure, which comprises:

providing the P450 structure of Table 1 or selected coordinates thereof which include coordinates from at least one (and preferably at least 2, for example at least 4, such as at least 7, more preferably at least 10 and in one embodiment all 14) of the residues of the ligand-binding region as defined herein;

providing a first molecular structure to be fitted to said selected coordinates of residues of said region;

fitting the first molecular structure to said P450 structure including at least one of the selected coordinates thereof

providing a second molecular structure; and

fitting the second molecular structure to said P450 structure.

Optionally the method of analysis further comprises providing a third molecular structure and also fitting that structure to the P450 structure. Indeed, further molecular structures may be provided and fitted in the same way.

The second and where applicable third molecular structure may be fitted to coordinates of amino acids from another part of the P450 binding pocket, such as another part of the ligand-binding region or to the haem-binding region as defined herein. In one embodiment, the second and/or third molecular structure may be fitted, in addition to or instead of, to the haem structure in the P450 binding pocket.

Following the fitting of the molecular structures, a person of skill in the art may seek to use molecular modelling to determine to what extent the structures interact with each other (e.g. by hydrogen bonding, other non-covalent interactions, or by reaction to provide a covalent bond between parts of the structures) or the interaction of one structure with 2C9 is altered by the presence of another structure. The person of skill in the art may use in silico modelling methods to alter one or both structures in order to design new structures which interact in different ways with CYP 2C9, so as to speed up or slow down their metabolism, as the case may be.

Newly designed structures may be synthesised and their interaction with CYP 2C9 may be determined or predicted as to how the newly designed structure is metabolised by said P450 structure. This process may be iterated so as to further alter the interaction between it and the CYP 2C9.

### Example 2

### Relevance of the S-Warfarin Remote Binding Site for Drug Metabolism in Human Cytochrome P450 2C9

In this example the relevance of the remote warfarin biding site identified above is illustrated.

Cytochrome P450s 2C9trunc (1003, SEQ ID N04) and 2C9-FGloop K206E (1155, SEQ ID NO:2) metabolize the biologically active enantiomer of warfarin, S-warfarin one of the most widely prescribed oral anticoagulant, to the 6- and 7-hydroxyl metabolites to terminate the action of the drug. The structure of 2C9-FGloop K206E complexed with S-warfarin determined at 2.6 A resolution has revealed a new binding mode of warfarin distant from the heam, at the entry of the substrate channel, near the B′-C and F-G regions. To validate this remote binding pocket and to address its physiological relevance in drug metabolism in 2C9, residues L102, A103, L208 and N217, located in this remote binding pocket at ˜3.5 A from the S-warfarin molecule, have been mutated by site directed mutagenesis and substituted by larger residues like tyrosine and tryptophan in the 2C9trunc and 2C9-FGloop K206E backgrounds. Effects of the amino acid substitution on the functionality of the mutated enzymes were assessed in a reconstituted assay performed with the purified 2C9 mutants, NADPH cytochrome P450 reductase and using S-warfarin and diclofenac as prototypical substrates of 2C9.

Validation of the 2C9 Crystal Structure

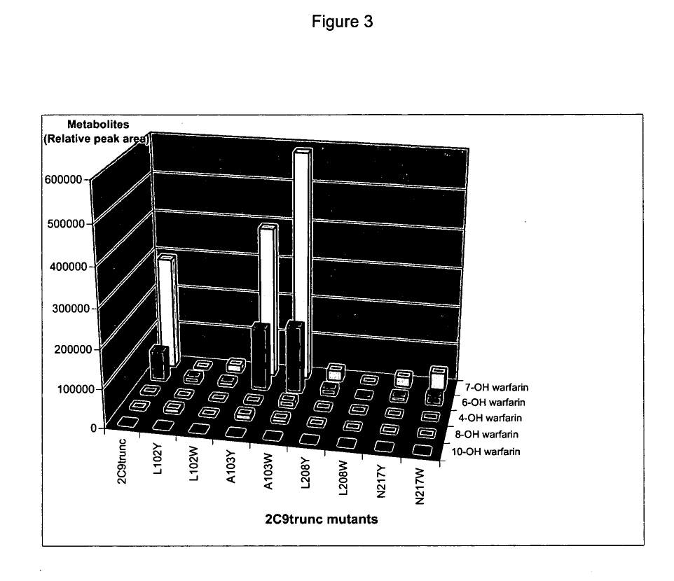

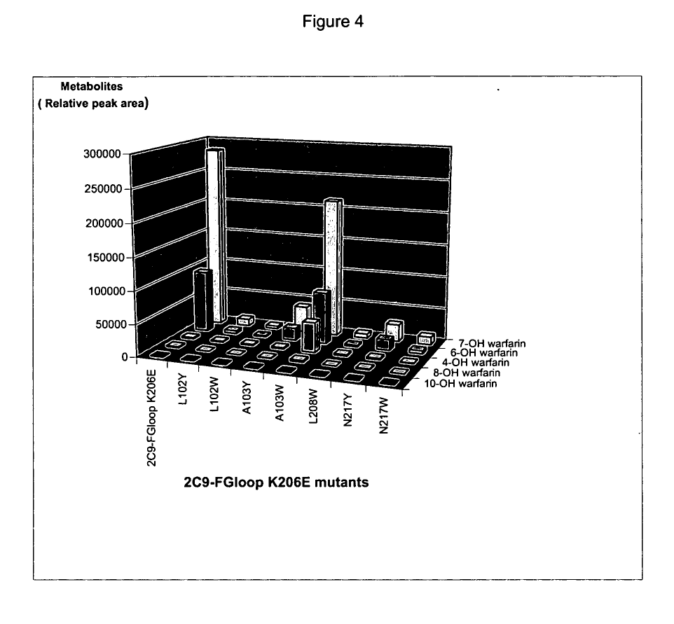

2C9trunc and 2C9-FGloop K206E metabolize S-warfarin to 6-hydroxyl S-warfarin and 7-hydroxyl S-warfarin at metabolic ratio of 7-hydroxyl S-warfarin/6-hydroxyl S-warfarin of 5.4 and 4.3 respectively. Only minor traces of 4-hydroxyl S-warfarin metabolites were observed and no 8-hydroxyl- or 10 hydroxyl metabolites were detectable. This metabolic profile and the kinetic parameters for S-warfarin (FIGS. 3 and 4, Table 7) are consistent with previous reports in the literature (Haining et al. Arch Biochem. Biophys. 1996;3 33(2):447-58, Haining et al. Biochemistry. 1999; 38(11): 3285-92, Thijssen et al., Drug Metab Dispos. 2000 (11): 1284-90). Analysis of the diclofenac metabolism also indicates that 2C9-FGloop K206E converts diclofenac exclusively to 4-hydroxy diclofenac with K_{M} and k_{cat} values that are comparable to those exhibited by 2C9trunc. These data have therefore confirmed that the mutations in the FG region which promote the crystallisation of 2C9 have not affected the regio selectivity in 2C9-FGloop K206E. They also allow to extrapolate that similar distant binding mode of S-warfarin also exists in 2C9trunc, the native N-terminal-truncated 2C9, Table 7.

Relevance of the Remote S-Warfarin Binding Pocket for Drug Metabolism

Substitution of residues L102, A103, L208 and N217 by larger residues like tyrosine and tryptophan in the S-warfarin binding pocket have affected the kinetics and/or the regio specificity of 2C9 enzymes for S-warfarin (FIG. 3). All the mutations, with the exception of A103Y and A103W have dramatically decreased the S-warfarin metabolism. Highly variable metabolic ratios of 7-hydoxyl versus 6-hydroxyl were also observed, with values ranking between 1.8 and 3.4 and much lower than the 5.4 value observed for 2C9trunc, indicating that the mutations may have also affected the positioning of the S-warfarin molecule in the active site during metabolism. For examples, substitutions of residues L102, L208 and N217 by tyrosine or tryptophan have almost totally suppressed the S-warfarin metabolism in 2C9. The maximal decrease in activity was produced by the L102Y and L208W substitutions, the mutated enzymes catalysing less than 1% of the 7-hydroxylase activity supported by the parent enzyme (FIG. 3). Substitutions L102W, L208Y, N217Y and N217W in 2C9trunc have produced enzymes with 6-hydroxylation and 7-hydroxylation activities that represent less than 25% and 15% respectively of the activities supported by 2C9trunc. Mutation L102Y, beside to the dramatic decrease in activity, has also induced a change in substrate specificity with the production of 6-hydroxyl and 8-hydroxyl metabolites of warfarin and only traces of 7-hydroxyl metabolites. At the opposite, substitution A103W and A103Y have produced enzymes with enhanced the S-warfarin activity that may reflect a better stabilization of S-warfarin molecule in a more favourable orientation for metabolism, via pi-pi staking between the phenyl rings of the substituted residues and those of the substrate molecule (FIG. 3). The same set of mutations, when made in the 2C9-FGloop K206E background, has globally confirmed the results observed in 2C9trunc with minor discrepancies (FIG. 4):

a) 2C9-FGloop K206E L102Y enzyme, as observed for 2C9-FGloop K206E L102Y, has the activity dramatically decreased but without any change in the substrate regio specificity;

b) the mutant A103W despite being fully functional for the 6- and 7-hydroxylation activity, has also produced large amounts of 4-hydroxyl metabolites;

c) the A103Y mutation has a 7 fold decrease in S-warfarin activity, without having any effect of substrate regio specificity.

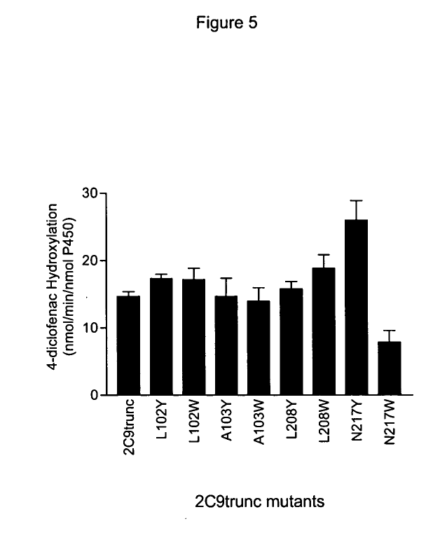

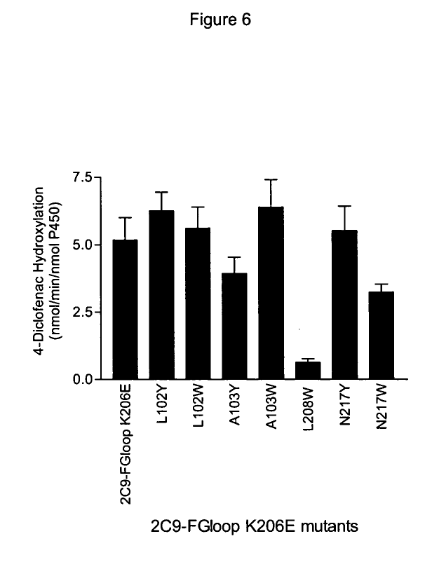

The 4-hydroxylation diclofenac assay, when used to address the effect of the amino acid changes made in the S-warfarin pocket on the metabolism of another substrate of 2C9, indicates that none of the mutations has significantly affected the metabolism of diclofenac by 2C9trunc (FIG. 5). Similar results were obtained for the mutants produced in the 2C9-FGloop K206E background with the exception mutation L208W (FIG. 6). Our results clearly demonstrate the involvement of residues L102, A103, L208 and N217 that line this distant binding pocket to the metabolism of S-warfarin in both 2C9trunc and 2C9-FGlooop K206EI enzymes. Our data also indicate that the remote binding pocket is specific to S-warfarin as the mutations made in this region had only minor effects on the metabolism of diclofenac.

We therefore postulate that S-warfarin and diclofenac could follow distinct routes for metabolism. To the opposite of S-warfarin that requires the binding to the remote binding pocket before moving to a position close to the heam, diclofenac is able to bypass the binding to this remote pocket and bind directly near the heam.

The analysis of metabolism of S-warfarin by 2C9 at low substrate concentrations indicates that metabolism of S-warfarin has followed Michaelis-Menten kinetics and therefore has excluded the simultaneous binding of two molecules of S-warfarin into the active site during the metabolism. Our results fit a model of two binding sites for S-warfarin that could be independently and sequentially occupied by S-warfarin during metabolism. In our 2C9 model, the S-warfarin molecule is first binding to the remote substrate binding pocket or “selecting site” in the active site before moving to a second binding site, at a position closer to the heam where the S-warfarin molecule is then metabolised. The displacement of S-warfarin from the distal (selecting site) to a proximal site (metabolic site) in the active site of 2C9 could be triggered by a conformation change in 2C9 upon reduction of the enzyme or by interactions with the NADPH cytochrome P450 reductase. The remote binding site appears to be the recognition site for S-warfarin that controls the entry of S-warfarin under the right orientation into the active site for metabolism as emphasized by the effects of the amino acid changes made to this binding pocket on the altered substrate regio specificity and the dramatic decrease in the S-warfarin metabolism.

Methods

Site Directed Mutagenesis

Replacement of residues L102, A103, L208 and N217 by tyrosine or tryptophan was conducted using the Quickchange mutagenesis method kit, as per the supplier's protocol (Stratagene, UK) and using pCW-2C9trunc and pCW-2C9-FGloop K206E as templates, the E. coli XL1 Blue strain and the mutating oligonucleotides listed on Table 6. The presence of the desired mutation was confirmed by automated DNA sequencing.

S-Warfarin Hydroxylation

The reactions were carried out in 50 mM KPi, pH 7.4, 1 mM EDTA, 0-200 μM S-warfarin (dissolved in 100% DMSO) for the Km determination or 100 μM S-warfarin for the regioselectivity study, 100 pmol of purified cytochrome P450, 0.3 units of purified human cytochrome P450 reductase in a total volume of 250 μL. The maximal percentage of solvent in the reaction was 1%. After 3 minutes pre-incubation at 37° C., the reaction was started by addition of 1 mM NADPH. The reaction was incubated for further 60 minutes before stopping with 50 μl of an acetonitrile/formic acid mixture (98:2). Sample were incubated on ice for 10 minutes before centrifugation at 13 000 rpm for 10 minutes to remove the precipitated proteins. Under these conditions the reaction remained linear over 90 minutes. Warfarin and its monohydroxylated metabolites were quantified by a LC-MSMS method. Routinely, 40 μl of supernatant were directly analyzed by reverse phase HPLC on a 150×4.6 mm 5 μm C8 Zorbax XDB column (Agilent, Stockport, UK) with C8 Security Guard cartridge (Phenomenex, Macclesfield, UK) at 40° C. at a flow rate of 0.8 ml/min via an Agilent 1100 binary LC pump (Agilent, Stockport, UK) and a CTC HTC-PAL autosampler (CTC, Zwingen, Switzerland) using the following set up. The mobile phase was 0.1% formic acid in water: 0.1% formic acid in acetonitrile (60:40 v/v) applied isocratically from 0-8 min to resolve the 6-, 7-, and 8-hydroxy metabolites. The percentage of organic phase was increased to 95% from 8.1-11 min to elute warfarin and the internal standard. The column was re-equilibrated under the starting conditions from 11.1-14 min. The total run time was 14 min. Metabolites were detected by tandem mass spectrometry. In order to avoid excessive high concentrations of warfarin (>4 μM substrate concentrations in kinetic studies) entering MS detector, the mobile phase was switched to waste from approximately 10-12 min. Typical retention times were; 5.3, 6.0, 6.2, 7.0, 7.5 and 10 minutes for 4′-, 10-, 6-, 7-, 8-hydroxywarfarin and warfarin respectively. The detector was a Waters Ultima Platinum MSMS mass analyzer (Waters/Micromass, Manchester UK) operating in positive ion electrospray mode. The following compounds and mass transitions were monitored: S-warfarin m/z 309.1→163.1 amu, 4′-hydroxywarfarin m/z 325.1→163.1 amu, 6-, 7- and 8-hydroxywarfarin m/z 328.15→179.1 and 10-hydroxywafarin m/z 325.1→251.1 amu.

Calibration standards were prepared in at least duplicate using authentic reference compounds introduced into control incubation media (without NADPH) over the range 0.5-1000 ng/ml (corresponding to 1.6-3200 nM warfarin or 1.5-3000 nM for hydroxyl metabolites), and aliquots were treated identically to test samples. Calibration curves, based on peak area or area, were linear over the stated range. Samples requiring only semi-quantitative metabolite profiling were analyzed in identical conditions excepted that the calibration standards were prepared in at least duplicate at a single concentration (500 ng/ml) to ascertain the relative intensity of response to warfarin and its metabolites whereby the ratio of metabolites formed in test incubations could be accurately determined.

### Diclofenac 4′-hydroxylation

Reactions were carried out in 50 mM KPi, pH 7.4, 1.5 mM MgCl₂, 0.1 mM EDTA,0-100 μM diclofenac for the Km determination or 100 μM for the regioselectivity determination, 20 pmol P450 and 0.3 units of purified human cytochrome P450 reductase in a total volume of 250 μL. After 3 minutes pre-incubation at 37° C., the reaction was started by addition of 1 mM NADPH. The reaction was incubated for further 12 minutes before stopping with 50 μl of an acetonitrile/acetic acid mixture (9:1). Sample were incubated on ice for 10 minutes before centrifugation at 13 000 rpm for 10 minutes to remove the precipitated proteins. Metabolites were separated by reverse phase HPLC using a C18 column (3.5 cm×2.1 mm) ACE5 C18 column (Hichrom, UK), using a GP50 gradient pump coupled to an AS50 autosampler (Dionex Inc., Sunnyvale, USA) at a flow rate of 0.35 ml/min. Metabolites were separated with a step gradient using 20 mM KPi, pH 7.4, 10% acetonitrile (buffer A) and 20 mM KPi, pH 7.4, 50% acetonitrile (buffer B). The gradient profile was: 0-4 min, 100% buffer A; 4-12 min 0-100% buffer B, 12-16 min, 100% buffer B, 16-22 min 100% buffer A. The formation of metabolite was monitored at 280 nm and quantified with reference to an authentic 4-hydoxyl diclofenac standard

TABLE 6
Oligonucleotides used to generate the indicated mutations in the 2C9trunc and
2C9-FGloop K206E background.

| | | | |
|---|---|---|---|
| Mutated | | | |
| Residue | Template | Oligonucleotides^{a} | SEQ ID NO: |

| | | | |
|---|---|---|---|
| L102Y | 2C9trunc | 5′ GGAAGAGGCATTTTCCCATATGCTGAAAGAGCTAACAG3′ | 7 |
| | 2C9-FGloop | 5′ CTGTTAGCTCTTTCAGCATATGGGAAAATGCCTCTTCC3′ | 8 |
| | K206E | | |
| L102W | 2C9trunc | 5′ GGAAGAGGCATTTTCCCATGGGCTGAAAGAGCTAACAG3′ | 9 |
| | 2C9-FGloop | 5′ CTGTTAGCTCTTTCAGCCCATGGGAAAATGCCTCTTCC3′ | 10 |
| | K206E | | |
| A103Y | 2C9trunc | 5′ GGAAGAGGCATTTTCCCACTGTATGAAAGAGCTAACAGAG3′ | 11 |
| | 2C9-FGloop | 5′ CTCTGTTAGCTCTTTCATACAGTGGGAAAATGCCTCTTCC3′ | 12 |
| | K206E | | |
| A103W | 2C9trunc | 5′ GGAAGAGGCATTTTCCCACTTGGTGAAAGAGCTAACAGAG3′ | 13 |
| | 2C9-FGloop | 5′ CTCTGTTAGCTCTTTCACCAAGTGGGAAAATGCCTCTTCC3′ | 14 |
| | K206E | | |
| L208Y | 2C9trunc | 5′ GAATGAAAACATCAAGATTTACAGCAGCCCCTGGATCCAG3′ | 15 |
| | | 5′ CTGGATCCAGGGGCTGCTGTAAATCTTGATGTTTTCATTC3′ | 16 |
| L208W | 2C9trunc | 5′ GAATGAAAACATCAAGATTTGGAGCAGCCCCTGGATCCAG3′ | 17 |
| | | 5′ CTGGATCCAGGGGCTGCTCCAAATCTTGATGTTTTCATTC3′ | 18 |
| L208W | 2C9-FGloop | 5′ GAAAACATCGAGATTTGGAGCAGCCCCTGGATCCAGG3′ | 19 |
| | K206E | 5′ CCTGGATCCAGGGGCTGCTCCAAATCTCGATGTTTTC3′ | 20 |
| N217Y | 2C9trunc | 5′ GCCCCTGGATCCAGATCTGCTATAATTTTTCTCCTATC3′ | 21 |
| | | 5′ GATAGGAGAAAAATTATAGCAGATCTGGATCCAGGGGC3′ | 22 |
| N217Y | 2C9-FGloop | 5′ CCCTGGATCCAGGTCTACTATAATTTCCCTGCTCTCC3′ | 23 |
| | K206E | 5′ GGAGAGCAGGGAAATTATAGTAGACCTGGATCCAGGG3′ | 24 |
| N217W | 2C9trunc | 5′ GCCCCTGGATCCAGATCTGCTGGAATTTTTCTCCTATC3′ | 25 |
| | | 5′ GATAGGAGAAAAATTCCAGCAGATCTGGATCCAGGGGC3′ | 26 |
| N217W | 2C9-FGloop | 5′ CCTGGATCCAGGTCTACTGGAATTTCCCTGCTCTCC3′ | 27 |
| | K206E | 5′ GGAGAGCAGGGAAATTCCAGTAGACCTGGATCCAGG3′ | 28 |

^{a}The mutated codons are underlined. The mutations were made in the 2C9trunc and 2C9-FGloop K206E backgrounds by site directed mutagenesis.

TABLE 7
Kinetic parameters of 2C9trunc and 2C9-FGloop
K206E for typical 2C9 substrates^{a}.

| | K_{M} (μM) | k_{cat} (min⁻¹) | k_{cat}/K_{M} | Fold |
|---|---|---|---|---|
| S-warfarin | | | | |
| (6-hydroxylation) | | | | |
| 2C9trunc | 10.33 ± 0.65 | 0.007 ± 0.0001 | 0.0007 | 1 |
| 2C9-FGloop K206E | 15 ± 3.75 | 0.009 ± 0.0007 | 0.0006 | 0.86 |
| S-warfarin | | | | |
| (7-hydroxylation) | | | | |
| 2C9trunc | 8.43 ± 0.55 | 0.039 ± 0.0008 | 4.54 | 1 |
| 2C9-FGloop K206E | 13.39 ± 1.54 | 0.036 ± 0.0011 | 2.66 | 0.59 |
| Diclofenac | | | | |
| (4′-hydroxylation) | | | | |
| 2C9trunc | 5.56 ± 1.03 | 14.63 ± 0.64 | 2.63 | 1 |
| 2C9-FGloop K206E | 8.68 ± 1.25 | 9.12 ± 0.32 | 1.05 | 0.40 |

^{a}Assays were performed in triplicates as described in the Methods section. The error values shown are the standard deviations calculated from fitting the Michaelis-Menten equation to the data. The last column gives the fold change in k_{cat}/K_{M} relative to the wild type truncated 2C9trunc.

### Example 3

### Back-Soaking of 2C9FGloop K206E -Warfarin Crystals

Generation of the 2C9-S-Warfarin Complex Crystals.

Co-crystals of 2C9 construct 1155 with S-warfarin are generated in a similar way to the generation of apo crystals. To order to obtain suitably large, well formed crystals it remains necessary to set up a limited grid screen around a known crystallization condition. This is typically achieved by setting up crystallizations using the conditions 0.1 M Tris pH 8-8.8, 15-30% PEG 400, 5% PEG 8000, 10% Glycerol

It may prove necessary to vary some of the crystallization variables (e.g. buffer pH, precipitant concentration) further than in the screen described above. A crystallization tray is pipetted out, with each crystallization well containing 1 ml of the above solutions. A stock solution of 0.2M S-warfarin is generated by dissolving S-warfarin in 40% DMSO, 60% ethanol. 19 μl of the first well solution is then removed, placed in an eppendorf and 1 μl of the 0.2M stock of S-warfarin is added to it. This is mixed well and the crystallization hanging drop is set up using 1 μl of protein and 1 μl of this S-warfarin/well mix. This is repeated in turn for each of the wells in the plate. It may prove necessary to vary the ratio of S-warfarin stock to optimize the crystals (e.g. using a 19.5:0.5 ratio of well to S-warfarin). Crystals typically grow to their maximum dimensions over a period of 7 days at 25° C.

Removal of S-Warfarin from the Crystals

Crystals of S-warfarin grown by the above method are then soaked in a solution typically containing 12.5% PEG 400, 7% PEG 8000, 15% glycerol, 0.25 M KCl and 0.075 M buffers which can be Tris pH 8.4, or imidazole pH 8.5. When a Tris buffer is used, the resulting electron density maps show density for an unknown ligand bound to the haem that we believe comes from the crystallization solutions. When an imidazole buffer is used, imidazole is bound to the haem.

Introduction of a New Compound into the Crystals

Once the crystals have had S-warfarin soaked out of them, they are transferred into a soaking solution containing 12.5% PEG 400, 7% PEG 8000, 15% glycerol, 0.25 M KCl and 0.075 M buffer which can be Tris pH 8.4, BisTris pH 6 or NaOAc pH 5.0. The soaking solution also contains the new compound at a concentration of 2.5-5 mM. The choice of buffer is dependent on the solubility of the compound at the different pHs. The crystals tolerate a drop in pH from 8.4 to pH 6 or 5 but diffraction power of these crystals is slightly worse than those soaked in solution at pH 8.4. The soaking time is typically 4-6 hours. The crystals are then frozen using a solution of 12.5% PEG 400, 10% PEG 8000, 21% glycerol, 0.3 M KCl and 0.075 M buffer (using the same buffer as in the resoaking solutions).

### Example 4

### Refinement of 2C9-FGloop K206E Structure

The structure of the apo 2C9FGloop K206E was produced according to Examples 4, 11 and 16 of WO03/035693, which are set out below as Annex 2. In this structure (Table 8 of WO03/035693) the position of the iron ions (atoms 7419 and 14895 of the Table) of the haem group were as follows:

| | | | | | | | | | |
|---|---|---|---|---|---|---|---|---|---|
| ATOM | 7419 | FE1 | HEM A 501 | 13.254 | 69.399 | 20.011 | 1.00 | 36.38 | FE |
| ATOM | 14895 | FE1 | HEM B 501 | 56.685 | 60.810 | 29.745 | 1.00 | 32.88 | FE |

The coordinates of these iron ions were refined to locate the iron ion centrally in the haem molecule. The resulting structure is as set out in Table 2 herein, which corresponds to Table 8 of WO03/035693 apart from the coordinates for atoms 7419 and 1485.

### Example 5

### Docking Experiment

The crystal structure of 2C9 was used to computationally dock a drug molecule into the binding pocket. The drug diclofenac, a known substrate for human 2C9, was generated and placed into the 2C9 binding pocket using interactive computer graphics. The observed interactions can now be used to chemically modify diclofenac via a structure-based design strategy to mediate its interaction with human 2C9 and improve its therapeutic potential.

Annex 1

Annex 1A: Production of DNA encoding 2C9 Proteins.

Summary

Cytochrome P450 2C9 was targeted for crystallisation. Conversion of this intrinsic membranous protein to a more water-soluble form, by removal of the N-terminus trans-membrane domain was performed prior to crystallisation.

Several N-terminus truncations, largely described in the literature, have been used to produce N-truncated cytochrome P450s (including 2E1, 2D6, 2B1 and others). However, most of these N-terminal truncations failed to produce fully soluble proteins and in most cases, the truncated P450s still remained associated with membranes.

The membrane anchor domain MDSLWLVLCLSCLLLLSLWRQSSGRGKL (SEQ ID NO:29) present in 2C9 (residues 2 to 29) was substituted by a short hydrophilic peptide MAKKTSSKGR (SEQ ID NO:6). The introduction of a highly charged polypeptide at the N-terminus of this protein was found to greatly decrease the membrane association of these proteins. It has also been found that the nature of the second codon in a lacZ expression system influences the level of expression (Looman et al, EMBO J., 6;2489-24992, 1987) and here alanine at position 2 provided good expression in E. coli.

Cytochrome P450 exhibits a high tendency to form large aggregates. The N-terminal deletion of cytochrome P450 has prevented aggregation and reduced polydispersity. This, in turn, facilitates the crystallisation of these proteins.

A four histidine tag was inserted at the C-terminus of 2C9 to help purification in high salt buffers.

Our preliminary results, using conditions from commercially available screening kits, indicated that the apo and native N-terminus truncated 2C9, 2C9trunc, did not produce any useful crystals. Thus the protein requires further modifications to promote crystallisation, and more importantly to promote production of useful crystals. Accordingly, the FG loop of the protein was considered for modification.

The design of the modification in the F-G loop was based on the published results on the crystallisation of the rabbit cytochrome P450 that indicated that the F and G helices were involved in the formation of a crystal contact. We predicted that the relative position of the F-G loop in the protein 2C9trunc could interfere with the ability of the F and G helices to constitute crystal contacts. It was proposed that the F-G loop, longer and more mobile than the counterpart found in the bacterial P450 BM3, may be stabilized or conformationally changed by six amino acid substitutions: Ile215Val, Cys216Tyr, Ser220Pro, Pro221Ala, Ile222Leu and Ile223Leu. In the resultant construct, 2C9-FGloop, the position of proline 220 is moved by one residue. The proline residue, often reported as initiating changes in secondary structure, may induce a conformational change in the F-G loop and facilitate the formation of crystal contacts. In the generation of the protein 2C9-P220, the proline is moved from position 221, as seen in 2C9 wild type to position 220 as seen in 2C19 wild type. Thus the serine 220 was mutated to proline and proline 221 was mutated to threonine. The introduction of these two changes alone was sufficient to promote crystallisation. A single mutation of S220P, retaining the proline at 221 was also sufficient to get crystallisation.

In the generation of the protein 1424, the proline is moved from position 221, as seen in 2C9 wild type to position 222. This shows that the proline can be moved one amino acid either side of 221 to promote successful crystallisation.

We believe having a proline at 220 or 222, preferably proline 220 is a critical determinant for crystallisation of 2C9. In particular it is a critical determinant for obtaining apo crystals of 2C9. It is also important for obtaining diffraction quality crystals of 2C9. Residue 221 can be alanine, or threonine. It can also be proline or serine.

The mutagenesis of human 2C9 cytochrome P450 was performed by a variety of standard recombinant DNA techniques including cassette mutagenesis, site-directed mutagenesis or specific cloning protocols. For cassette mutagenesis, complementary oligonucleotides bearing the mutations were annealed and cloned, using natural restriction sites or sites that have been introduced by PCR mutagenesis into the P450 cDNA. The constructs were verified by restriction mapping followed by full sequencing. Other techniques are described herein or are well known as such to those of skill in the art.

N-Terminal Truncation of P450

The expression vector pCWOri+, provided by Prof. F. W. Dahlquist, University of Oregon, Eugene, Oreg., USA, was used to express the truncated human cytochrome P450s in the E. coli strain XL1 Blue (Stratagene). A full-length cDNAs encoding cytochrome P450 2C9 was used as a template for PCR amplification, engineering the 5′ terminus deletion, insertion of silent restriction sites and insertion of a four Histidine tag at the C-terminus.

A NotI restriction site (underlined) was introduced in 2C9 at position 87 by PCR amplification using the following 5′ oligonucleotide:

(SEQ ID NO:30)

5′-ATAAGAATGCGGCCGCCTGGCCCCACTCCTCTCCCAGTGATTGGA
AATATC-3′.

The 3′ oligonucleotides:

5′-TGCGGTCGACTCAGTGGTGGTGGTGGACAGGAATGAAGCAGAGCTGGTAG-3′ (SEQ ID NO: 31) with a SaII cloning site (underlined) and the four Histidine tag (italics) was used. A total of 30 cycles at 94° C. for 1 min, 52° C. for 1 min, and 72° C. for 2 min were followed by an extension of 10 min at 72° C. The 1420-bp PCR fragment was double digested with NotI/SaII and purified by gel agarose elution and extraction.

The complementary oligonucleotides

| | |
|---|---|
| 5′-TATGGCTAAGAAAACGAGCTCTAAAGGGC-3′ | (SEQ ID NO:32) |
| and | |
| 5′-GGCCGCCCTTTAGAGCTCGTTTTCTTAGCCA-3′ | (SEQ ID NO:33) |

with the NdeI and NotI overhang restriction sites (underlined) were designed to substitute the residues 2-29 of the native N terminus of human cytochrome P450 2C9 by the short AKKTSSKGR polypeptide. The oligonucleotides were annealed by mixing 10 μg of each Oligonucleotide in 100 μl of water, heating at 100° C. for 5 min and slow cooling at room temperature.

The 1420-bp PCR fragment was mixed to the double stranded oligonucleotide and ligated in the vector pCWori+, previously digested with NdeI and SaII. An aliquot of the ligation product was used to transform E. coli XL1 Blue strain to yield the plasmid pCW-2C9trunc that encodes for the amino-terminal truncated 2C9.

The truncated 2C9 was used to make the proteins for further crystallisation experiments.

Construction of 2C9-FGloop

The plasmid pCW-2C9trunc was used as template for the insertion of six amino acids substitutions, Ile215Val, Cys216Tyr, Ser220Pro, Pro221Ala, Ile222Leu, Ile223Leu in the FG loop. pCW-2C9trunc was digested by NdeI and BamHI restriction enzyme and the 579-bp corresponding to the 5′ terminus of the P450 gene was purified by gel agarose extraction and elution. A double strand oligonucleotide designed to introduce the six amino acids substitution in the FG loop, was generated by annealing the following complementary oligonucleotides 5′-GATCCAGGTCTACAATAATTTCCCTGCTCTCCTTGATTATTTC-3′ (SEQ ID NO:34) and 5′-CCGGGAAATAATCAAGGAGAGCAGGGAAATTATTGTAGACCTG-3′ (SEQ ID NO:35) with the overhang BamHI and XmaI restriction sites (underlined) and the six mutated codons (italics). The 579-bp fragment and the double strand oligonucleotide were ligated in the vector pCW-2C9trunc, previously digested by NdeI and XmaI. An aliquot of the ligation was used to transform XI1 Blue E. coli and yield the plasmid pCW-2C9-FGloop.

Construction of 2C9-P220

2C9-P220 is a 2C9trunc mutant carrying the mutations S220P and P221T. This mutant was made using the Stratagene Quikchange™ mutagenesis kit (catalogue number #200518), according to manufacturers instructions. The Quikchange™ mutagenesis method generates a mutated plasmid with staggered nicks and uses DpnI digestion to remove all parental DNA. Reactions were made incorporating 5.0 μL×10 reaction buffer, 5-50 ng pCW-2C9trunc plasmid DNA, 1.0 μL dNTP and 125 ng oligonucleotide primers as follows, with mutated bases shown in lowercase and the two amino acid change underlined:

(SEQ ID NO:36)

5′ CCAGATCTGCAATAATTTTcCgaCcACATTGATTTACTTCCC 3′

(SEQ ID NO:37)

5′ GGGAAGTAATCAATGATgGtcGgAAAATTATTGCAGATCTGG 3′

Reactions were made to 50 μL with sterile water, 2.5 U Pfu Turbo was then added and the reaction overlayed with 30 μL mineral oil. Thermocycling was then carried out as follows: 95° C., 30 sec (1 cycle), 95° C., 30 sec, 55° C., 1 min, 68° C. 13.5 min (18 cycles) and finally a holding period at 4° C. A control reaction was also included with water in place of oligonucleotide primers.

Following thermocycling 10 U DpnI was added, under the level of the mineral oil, to each reaction. The reactions were then gently mixed followed by centrifugation in a bench top microcentrifuge, 1 min, 13,000 rpm and incubated at 37° C. for 3 hr. Digested product (1 μL) was then used to transform 50 μL competent E. coli XL1 -Blue cells. The whole transformation as then plated onto Luria agar plates containing 100 μg/ml carbenicillin, inverted, and incubated overnight at 37° C. Colonies were isolated and the plasmid DNA pCW-2C9-P220 isolated and sequenced to check for the insertion of the correct mutation.

Construction of 2C9-FGloop-K206E

The plasmid pCW-2C9-FGloop was used as a template for the substitution Lys206Glu (where the numbering is of the full length wild type 2C9, SwissProt: P11712, not that of SEQ ID NO:2 or 4). Primers were designed to lie across the region to be mutated;

(SEQ ID NO:38)

5′-GGAAAAGTTGAATGAAAACATCGAGATTTTGAGCAGCCCCTGG-3′

(SEQ ID NO:39)

5′-CCAGGGGCTGCTCAAAATCTCGATGTTTTCATTCAACTTTTCC-3′

where the mutated codon is shown in bold. These primers were then used in the protocol for Quikchange™ mutagenesis (Stratagene) which is briefly summarised.

Primers were resuspended to 125 ng/μl and used in a PCR reaction which elongated around the plasmid from the mutagenic primer. The template DNA was then digested using Dpnl, a methylation specific restriction endonuclease which preferentially degrades the template due to its methylation. After DpnI treatment 1 μl of the resultant sample was transformed into E. coli XL1 Blue strain. Colonies were picked and sequenced. Plasmids containing the mutation were chosen and digested with the restriction endonucleases NdeI and SaII. The NdeI SaII DNA fragment corresponding to the coding sequence of the 2C9-FGloop K206E mutant was then sub-cloned into a pCW vector digested with Ndel and Sall. This served to remove any errors incorporated during the PCR phase of the Quickchange mutagenesis.

Annex 1B: Expression of 2C9P220 and 2C9-FGloop.

Bacteria Expression

A single ampicillin resistant colony of XL1 blue cells was grown overnight at 37° C. in Terrific Broth (TB) with shaking to near saturation and used to inoculate fresh TB media. Bacteria were grown to an OD600 nm=0.4 in 1 litre of TB broth containing 100 μg/ml of ampicillin at 37° C. at 185 rpm in 2 litre flask. The haem precursor delta aminolevulinic acid (80 mg/l) was added 30 min prior to induction with 1 mM isopropyl-β-D-thiogalactopyranoside (IPTG) and the temperature lowered to 30° C. The bacterial culture was continued under agitation at 30° C. for 48 to 72 hours.

(a) Protein Purification

The cells were pelleted at 10000 g for 10 min and resuspended in a buffer containing 500 mM KPi, pH 7.4, 20 % glycerol, 10 mM mercaptoethanol, 0.1% (v/v) of protease inhibitor cocktail (Calbiochem), 10 mM imidazole, 0.01 mg/ml DNase 1 and 5 mM MgSO₄.

The cells were lysed by passing twice through a Constant Systems Cell Homogeniser at 12000 psi. The cell debris was then removed by centrifugation at 70000 g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The protein bound-NiNTA resin was pelleted by centrifugation at 2000 g for 2 min at 4° C. The resin was washed with 20 resin volumes of 500 mM KPi, pH 7.4,20% glycerol, 10 mM mercaptoethanol, 10 mM imidazole, 1:1000 dilution of protease inhibitor cocktail, 0.3% (v/v) IGEPAL CA630 and the resin pelleted by centrifugation at 2000×g for 2 min at 4° C. The resin was then washed with 10 resin volumes of 500 mM KPi, pH 7.4, 20% glycerol, 10 mM mercaptoethanol, 20 mM imidazole, 0.1% (v/v) protease inhibitors, 0.3% IGEPAL CA630 and the resin recovered by centrifugation as described above. The washing step was repeated as described above with buffer containing 50 mM imidazole. The resin was packed into a column at 4° C. and the cytochrome P450 eluted with 500 mM KPi, pH 7.4, 20% glycerol, 10 mM mercaptoethanol, 300 mM imidazole, 0.1% (v/v) of protease inhibitor cocktail, 0.3% (v/v) IGEPAL CA630.

(b) An Alternative Method for Protein Purification is as Follows:

The cells were pelleted at 10000 g for 10 min and resuspended in a buffer containing 500 mM KPi, pH 7.4, 20% glycerol, 10 mM mercaptoethanol, 0.1% (v/v) of protease inhibitor cocktail (Calbiochem), 0.01 mg/ml DNase 1 and 5 mM MgSO₄.

The cells were lysed by passing twice through a Constant Systems Cell Homogeniser at 12000 psi. The cell debris was then removed by centrifugation at 70000 g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The NiNTA resin was pelleted by centrifugation at 2000 g for 2 min at 4° C. and washed, as described above, with 20 resin volumes of 500 mM KPi, pH 7.4, 20% glycerol, 10 mM mercaptoethanol, 50 mM glycine 0.1% (v/v) protease inhibitors, 0.3% IGEPAL CA630, followed by washing with 10 resin volumes of 500 mM KPi, pH 7.4, 20% glycerol, 10 mM mercaptoethanol, 7.5 mM Histidine, 0.1% (v/v) protease inhibitors, 0.3% IGEPAL CA630. The resin was recovered by centrifugation between washing steps and then the resin was packed into a column at 4° C. The protein was eluted with 500 mM KPi, pH 7.4, 20% glycerol, 10 mM mercaptoethanol, 100 mM histidine, 0.1% (v/v) of protease inhibitor cocktail, 0.3% (v/v) IGEPAL CA630.

The cytochrome P450 obtained from the NiNTA column by either elution protocol was quickly desalted (<10 min) into 10 mM KPi, pH 7.4, 20% glycerol, 0.2 mM DTT, 1 mM EDTA using a HiPrep 26/10 desalting column (Pharmacia), at a flow rate of 5 ml/min and collecting 16 ml fractions. The desalted cytochrome P450 was directly applied to a CM Sepharose column (Pharmacia), previously equilibrated with 10 mM KPi, pH 7.4, 20% glycerol, 0.2 mM DTT, 1 mM EDTA. The following step elution was applied: wash with 10 column volumes of 10 mM KPi, pH 7.4, 20% glycerol, 0.2 mM DTT, 1 mM EDTA, wash with the above buffer with 75 mM KCl in order to remove any trace of detergent, then eluted with the above buffer with KCl concentration increased to 500 mM. The protein was concentrated up to 40 mg/ml using a microconcentrator for crystallisation assays.

At this stage, the protein can be optionally further purified by running a gel filtration column. The concentrated P450 sample was applied on the top of a Superose 6 HR10/30 gel filtration column (Pharmacia) and eluted at 0.2 ml/min with buffer containing 100 mM KPi, pH 7.4, 300 mM KCl, 20% glycerol, 0.2 mM DTT. The protein was collected and concentrated up to 40 mg/ml, as described above, for crystallisation and quality assays.

Annex 1C: Crystallisation and Structure Analysis of 2C9-FGloop K206E.

E. coli transformed with the 2C9-FGloop K206E vector described above were grown and described in Annex 1B.

Protein Purification

The cells were pelleted at 10000 g for 10 min and resuspended in a buffer containing 500 mM KPi, pH 7.4, 20 % glycerol, 10 mM mercaptoethanol, 0.1% (v/v) of protease inhibitor cocktail (Calbiochem), 10 mM imidazole, 40 U/ml DNase 1 and 5 mM MgSO₄.

The cells were lysed by passing twice through a Constant Systems Cell Homogeniser at 12000 psi. The cell debris was then removed by centrifugation at 70000 g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The protein bound-NiNTA resin was pelleted by centrifugation at 2000 g for 2 min at 4° C. The resin was washed with 20 resin volumes of 500 mM KPi, pH 7.4, 20% glycerol, 10 mM mercaptoethanol, 10 mM imidazole, 1:1000 dilution of protease inhibitor cocktail, 0.3% (v/v) IGEPAL CA630 and the resin pelleted by centrifugation at 2000×g for 2 min at 4° C. The resin was then washed with 10 resin volumes of 500 mM KPi, pH 7.4, 20% glycerol, 10 mM mercaptoethanol, 20 mM imidazole, 0.1% (v/v) protease inhibitors, 0.3% IGEPAL CA630 and the resin recovered by centrifugation as described above.

The resin was packed into a column at 4° C and the cytochrome P450 eluted with 500 mM KPi, pH 7.4, 20 % glycerol, 10 mM mercaptoethanol, 300 mM imidazole, 0.1% (v/v) of protease inhibitor cocktail, 0.3% (v/v) IGEPAL CA630.

The cytochrome P450 obtained from the NiNTA column by either elution protocol was quickly desalted into 10 mM KPi, pH 7.4, 20% glycerol, 2.0 mM DTT, 1 mM EDTA using a HiPrep 26/10 desalting column (Pharmacia), at a flow rate of 5 ml/min and collecting 17 ml fractions.

The desalted cytochrome P450 was directly applied to a CM Sepharose column (Pharmacia), previously equilibrated with 10 mM KPi, pH 7.4, 20% glycerol, 2.0 mM DTT, 1 mM EDTA. The following step elution was applied: wash with 10 column volumes of 10 mM KPi, pH 7.4, 20% glycerol, 2.0 mM DTT, 1 mM EDTA, wash with the above buffer with 75 mM KCl in order to remove any trace of detergent, then eluted with the above buffer with KCl concentration increased to 500 mM.

The protein was concentrated up to 40 mg/ml using a microconcentrator for crystallisation assays. To characterize the protein, the quality of the final preparation was evaluated by:

(a) SDS Polyacrylamide Gel Electrophoresis

This was performed using commercial gels (Nugen) followed by CBB staining according to the manufacturer's instructions. The purity as estimated by scanning a digital image of a gel was estimated to be at least 95%.

(b) Mass Spectroscopy

Mass spectroscopy was performed using a Bruker “BioTOF” electrospray time of flight instrument. Samples were either diluted by a factor of 1000 straight from storage buffer into methanol/water/formic acid (50:48:2 v/v/v), or subjected to reverse phase HPLC separation using a C4 column. Calibration was achieved using Bombesin and angiotensin I using the 2+ and 1+ charge state. Data were acquired between 200 and 2000 m/z range and were subsequently processed using Bruker's X-mass program. Mass accuracy was typically below 1 in 10 000.
Mass spec of 2C9-FGloop-K206E: 53966 Da (observed) 53964.67 Da (predicted)

(c) Functionality Assays

Activity assays on P450 2C9 were performed in a 96-well plate assay format with a Fluoroscan Ascent FL Instruments (Labsystem), using the methoxy-4-(trifluoromethyl)-coumarin as a fluorescent substrate.

Fifteen pmoles of P450 were reconstituted with 0.1 unit of purified human oxidoreductase, in presence of 140 μM of substrate methoxy-4-(trifluoromethyl)-coumarin, a NADPH regenerating system that includes 0.15 mM NADP⁺, 0.38 mM Glucose-6-phosphate and 2.9 unit/ml glucose-6-phosphate dehydrogenase in 170 μl final volume of 25 mM KPi, pH 7.4, 0.38 mM MgCl₂. Incubations were performed at 37° C. for several minutes and 7-hydroxy-4-(trifluoromethyl)-coumarin was used as metabolite standard to determinate the metabolic rate. The excitation and emission wavelengths used were respectively 409 and 530 nm. The activity of the 2C9-FGloop-K206E was 0.083 pmol/min/pmol P450 with 2C9 substrate.

Crystallisation of 2C9-FGLoop-K206E

Crystals of the 2C9-FGloop-K206E were grown using the hanging drop vapour diffusion method. Protein at 40 mg/ml in 10 mM Kpi pH 7.4, 0.5 M KCl, 2 mM DTT, 1 mM EDTA, 20% glycerol, was mixed in a 1:1 ratio, using 0.5 μl drops, with a reservoir solution. The crystals of 2C9-FGloop-K206E grew over a reservoir solution containing 0.2 M dibasic potassium phosphate and 20% PEG 3350 (Alternative conditions were also used, which were 0.1 M Tris-HCl, pH 8.5; 0.2 M LiSO4; 15% PEG 4000). Crystals formed within 1-7 days at 25° C., and had morphologies of hexagonal needles and rods. The approximate cell dimensions of the crystals were 165 Å, 165 Å, 112 Å, 90°, 90°, 120°. The crystals were flash frozen in liquid nitrogen, using 80% reservoir solution, 10% PEG 400 and 10% glycerol as a cryoprotectant.

Annex 1D: Structure of 2C9-FGloop K206E.

Data was collected from a 2C9-FGloop-K206E crystal (prepared as described in Annex 1C) to 3.0 Å resolution at beamline ID14.1 (wavelength 0.933 Å) at the European Synchrotron Radiation Source using a Quantum4 CCD detector from a single crystal at 100K. A total of 90 one degree oscillation images were collected and processed using MOSFLM 6.11 (Leslie, A. G. W. (1992). Jnt CCP4/ESF-EACMB Newslett. Protein Crystallogr. 26), scaled using SCALA 4.1, and reduced using the CCP4 suite of programs (Collaborative Computational Project, Number 4, (1994). The CCP4 suite: programs for protein crystallography. Acta Cryst. D50, 760-763).

Table of data statistics

Resolution

| | 15-3.0 Å | 3.16-3.0 Å |
|---|---|---|
| Completeness (%) | 99.4 | 98.7 |
| Multiplicity | 5.2 | 4.8 |
| I/Sigma(I) | 3.5 | 1.3 |
| Rmerge (%) | 12.7 | 54.2 |

The crystals belong to spacegroup P321 and have cell dimensions 165.46 Å, 165.46 Å, 111.70 Å, 90°, 90°, 120°. There are two copies in the asymmetric unit, and the crystals have a solvent content of 68%. The structure was solved by molecular replacement using the 2C5 structure (pdbid 1 DT6) (Williams, P A; Cosme, J; Sridhar, V; Johnson, E F; McRee, D E, Molecular Cell, Volume 5, Issue 1, January 2000,Pages 121-131) and the program AMORE (Navaza, J. (1994). AMoRe: an automated package for molecular replacement. Acta Cryst. A50, 157-163), giving a correlation coefficient of 67.8% and an R-factor of 38.9%. The coordinates of the structure are set out in Table 1 of WO03/035693. The two copies in the asymmetric unit are related by a rotation of 145° about the Z-axis. The initial maps (both averaged and unaveraged) were relatively clean, and containing unmistakable electron density for the heme group which was omitted from the search model. This solution was using as a starting point for refinement using the program CNX

Annex 2

Annex 2A: Crystallisation of 2C9-FGloop K206E.

Bacteria Expression

A single ampicillin resistant colony of XL1 blue cells transformed with the 2C9-FGloop K206E-expressing plasmid described above in Annex 1A was grown overnight at 37° C. in Terrific Broth (TB) with shaking to near saturation and used to inoculate fresh TB media. Bacteria were grown to an OD600 nm=0.4 in 1 litre of TB broth containing 100 μg/ml of ampicillin at 37° C. at 185 rpm in 2 litre flask. The heme precursor delta aminolevulinic acid (80 mg/l) was added 30 min prior to induction with 1 mM isopropyl-β-D-thiogalactopyranoside (IPTG) and the temperature lowered to 25° C. The bacterial culture was continued under agitation at 25° C. for 72 hours.

Protein Purification

The cells were pelleted at 10000 g for 10 min and resuspended in a buffer containing 500 mM KPi, pH 7.4, 20% glycerol, 10 mM mercaptoethanol, 0.1% (v/v) of protease inhibitor cocktail (Calbiochem), 10 mM imidazole, 40 U/ml DNase 1 and 5 mM MgSO₄.

The cells were lysed by passing twice through a Constant Systems Cell Homogeniser at 10000 psi. The cell debris was then removed by centrifugation at 22000×g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The protein bound-NiNTA resin was pelleted by centrifugation at 2000 g for 2 min at 4° C. The resin was washed with 30 resin volumes of 500 mM KPi, pH 7.4, 20% glycerol, 10 mM mercaptoethanol, 10 mM imidazole, 1:1000 dilution of protease inhibitor cocktail, 0.3% (v/v) IGEPAL CA630 and the resin pelleted by centrifugation at 2000×g for 2 min at 4° C. The resin was then washed with 15 resin volumes of 500 mM KPi, pH 7.4, 20% glycerol, 10 mM mercaptoethanol, 20 mM imidazole, 0.1% (v/v) protease inhibitors, 0.3% IGEPAL CA630 and the resin recovered by centrifugation as described above.

The resin was packed into a column at 4° C. and the cytochrome P450 eluted with 500 mM KPi, pH 7.4, 20 % glycerol, 10 mM mercaptoethanol, 300 mM imidazole, 0.1% (v/v) of protease inhibitor cocktail, 0.3%(v/v) IGEPAL CA630.

The cytochrome P450 obtained from the NiNTA column was quickly desalted into 10 mM KPi, pH 7.4, 20% glycerol, 2.0 mM DTT, 1 mM EDTA using a HiPrep 26/10 desalting column (Pharmacia), at a flow rate of 5 ml/min.

The desalted cytochrome P450 was directly applied to a CM Sepharose column (Pharmacia), previously equilibrated with 10 mM KPi, pH 7.0, 20% glycerol, 2.0 mM DTT, 1 mM EDTA. The following step elution was applied: wash with 20 column volumes of 10 mM KPi, pH 7.0, 20% glycerol, 2.0 mM DTT, 1 mM EDTA, wash with the above buffer with 75 mM KCl in order to remove any trace of detergent, then eluted with the above buffer with KCl concentration increased to 500 mM.

The protein was concentrated up to 40 mg/ml using a microconcentrator for crystallisation assays.

Crystallisation of 2C9-FGloop K206E

Crystals of the 2C9-FGloop-K206E were grown using the hanging drop vapour diffusion method. Protein at 40 mg/ml in 10 mM Kpi pH 7.0, 0.5 M KCl, 2 mM DTT, 1 mM EDTA, 20% glycerol, was mixed in a 1:1 ratio, using 0.5 μl drops, with a reservoir solution. The crystals of 2C9-FGloop-K206E were grown over a reservoir solution containing: 0.1 M Tris-HCl pH 8.4, 15% PEG 400, 5% PEG 8000, 10% glycerol.

Rod shaped crystals formed within 1 day at 25° C. The crystals were flash frozen in liquid nitrogen, using the reservoir solution as a cryoprotectant. The approximate cell dimensions of the crystals were 164.9 Å, 164.9 Å, 111.1 Å, α=90°, β=3=90°, γ=120°.

Annex 2B: Production of a 2.6 Å resolution structure of 2C9-FGloop K206E

Data was collected to 2.6 Å resolution from a crystal of 2C9-FGloop-K206E crystal (prepared as described in Annex 2A) at beam line 14.1 at the European Synchrotron Radiation Facility, using a Quantum4 CCD detector from a single crystal at 100 K. The crystal was grown against a reservoir solution of 0.1M Tris pH 8.4, 15% PEG 400, 5% PEG 8000, 10% Glycerol, and was frozen directly from the reservoir solution. A total of 50 images were collected and processed using MOSFLM (Leslie, A. G. W. (1992). Jnt CCP4/ESF-EACMB Newslett. Protein Crystallogr. 26), scaled using SCALA and reduced using the CCP4 suite of programs (Collaborative Computational Project, Number 4, (1994). The CCP4 suite: programs for protein crystallography. Acta Cryst D50, 760-763).

Table of data statistics

Resolution

| | 50-2.6 Å | 2.74-2.60 Å |
|---|---|---|
| Completeness | 96.5% | 84.3% |
| Multiplicity | 2.6 | 2.0 |
| I/Sigma I | 6.8 | 1.2 |
| R merge | 8.7 | 57.0 |

This data was used in refinement, using the model generated by the refinement against the initial 3.0 Å data, to generate a set of coordinates for the 2C9FGloop structure. A consistent set of 5% of the reflections was flagged for Free R calculation, and extended to the higher resolution. The refinement was continued using the programs CNX (Brunger et al., Current Opinion in Structural Biology, Vol. 8, Issue 5, October 1998, 606-611, and commercially available from Accelerys, San Diego, Calif.) and REFMAC (Collaborative Computational Project, Number 4, (1994). The CCP4 suite: programs for protein crystallography. Acta Cryst. D50, 760-763), to an R factor of 21.9% and an R free factor 25.0%.

Annex 2C: Refinement of 2C9-FGloop K206E Structure.

Data generated in Annex 2B was further refined to generate a table of coordinates of the 2C9 structure. A total of 147 water molecules have been added (manually and automatically) and included in the refinement. This resulted in an Rfactor of 20.7% and a R free factor of 25.9%.

Summary

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention.

1. A co-crystal of cytochrome P450 2C9 and warfarin with unit cell dimensions:
a=b=164.76 Å±5%, and c=110.76 Å±5%.

2. A co-crystal of P450 protein and warfarin having the structure defined by the co-ordinates of Table 1.

3. A method of making P450 2C9 protein co-crystals with a compound, which method comprises the hanging drop vapour-diffusion technique, using a precipitant solution comprising: 0.1 M Tris, pH 8.4, 15-25% (v/v) PEG 400, 5-12.5% (w/v) PEG 8000,10% (v/v) glycerol supplemented with 1-10 mM substrate.

4. A method of making P450 2C9 protein co-crystals with a compound, which method comprises the hanging drop vapour-diffusion technique, using a precipitant solution comprising: 0.1 M Tris pH 8-8.8, 15-30% PEG 400, 5% PEG 8000, 10% Glycerol supplemented with 1-10 mM substrate.

5. The method of claim 3 wherein said compound is S-warfarin.

6. A method of obtaining a co-crystals of P450 2C9 and a ligand by:
generating a 2C9-warfarin co-crystal;
removing warfarin from the co-crystal by soaking the crystal in a removal buffer;
soaking the crystal in a soaking solution comprising the ligand.

7. A computer-based method for the analysis of the interaction of a molecular structure with a P450 structure, which comprises:
providing the P450 structure of Table 1 or Table 2 or selected coordinates thereof;
providing a molecular structure to be fitted to said P450 structure or selected coordinates thereof; and
fitting the molecular structure to said P450 structure.

8. The method of claim 7 wherein said selected coordinates include atoms from one or more of the residues of the ligand-binding region, said region being defined as residues:
72, 74, 97, 98, 99, 100, 101, 102, 103, 104, 105, 106, 107, 110, 112, 113, 114, 116, 204, 205, 208, 213, 214, 216, 217, 233, 364, 365, 366, 367, 368, 369, 384, 385, 386, 387, 388, 476 and 477.

9. The method of claim 7 wherein said selected coordinates include atoms from one or more of the residues of the haem-binding region, said regions being defined as residues:
97, 98, 111, 112, 113, 114, 115, 116, 178, 290, 293, 294, 295, 297, 298, 299, 300, 301, 302, 361, 362, 365, 366, 367, 368, 369, 389, 391 and 433.

10. The method of claim 7 wherein the selected coordinates include atoms from one or more of the residues Arg97, Gly98, Ile99, Phe100, Leu102, Ala103, Val113, Phe114, Asn217, Thr364, Ser365, Leu366, Pro367 and Phe476

11. The method of claim 7 wherein the selected coordinates include atoms from one or more of the residues Arg97, Gly98, Ile99, Phe100, Leu102, Ala103, Val113, Phe114, Leu218, Asn217, Thr364, Ser365, Leu366, Pro367 and Phe476.

12. The method of claim 7 wherein said selected coordinates further include those of the iron ion bound to the haem molecule.

13. The method of claim 12 wherein said selected coordinates are of Table 2.

14. The method of claim 7 which further comprises modifying the molecular structure to change its interaction with one or more of the selected coordinates.

15. The method of claim 7 which further comprises the steps of:
obtaining or synthesising a compound which has said molecular structure; and
contacting said compound with P450 protein to determine the ability of said compound to interact with the P450.

16. The method of claim 7 which further comprises the steps of:
obtaining or synthesising a compound which has said molecular structure;
forming a complex of a 2C9 P450 protein and said compound; and
analysing said complex by X-ray crystallography to determine the ability of said compound to interact with the P450.

17. The method of claim 7 which further comprises the steps of:
obtaining or synthesising a compound which has said molecular structure; and
determining or predicting how said compound is metabolised by said P450 structure; and
modifying the compound structure so as to alter the interaction between it and the P450.

18. A compound having the modified structure identified using the method of claim 17.

19. A method of predicting three dimensional structures of P450 homologues or analogues of unknown structure, the method comprises the steps of:
aligning a representation of an amino acid sequence of a target P450 protein of unknown three-dimensional structure with the amino acid sequence of the P450 of Table 1 to match homologous regions of the amino acid sequences;
modelling the structure of the matched homologous regions of said target P450 of unknown structure on the corresponding regions of the P450 structure as defined by Table 1; and
determining a conformation for said target P450 of unknown structure which substantially preserves the structure of said matched homologous regions.

20. The method of claim 19 wherein said target P450 protein is selected from the group consisting of 2C8, 2C1 8 and 2C19.

21. A chimaeric protein having a binding cavity which provides a substrate specificity substantially identical to that of P450 2C9 protein,
wherein the chimaeric protein binding cavity is lined by a plurality of atoms which correspond to selected P450 2C9 atoms lining the P450 2C9 binding cavity, the relative positions of said plurality of atoms corresponding to the relative positions, as defined by Table 1, of said selected P450 2C9 atoms.

22. A method for determining the structure of a protein, which method comprises;
providing the co-ordinates of Table 1 or selected coordinates thereof, and
either (a) positioning said co-ordinates in the crystal unit cell of said protein so as to provide a structure for said protein, or (b) assigning NMR spectra peaks of said protein by manipulating said co-ordinates.

23. A method for determining the structure of a compound bound to P450 protein, said method comprising:
providing a crystal of P450 protein;
soaking the crystal with the compound to form a complex; and
determining the structure of the complex by employing the data of Table 1 or a portion thereof.

24. A method for determining the structure of a compound bound to P450 protein, said method comprising:
mixing P450 protein with the compound;
crystallising a P450 protein-compound complex; and
determining the structure of the complex by employing the data of Table 1 or a portion thereof.

25. A computer-based method for the analysis of the interaction of two molecular structures within a P450 binding pocket structure, which comprises:
providing the P450 structure of Table 1 or selected coordinates thereof which include coordinates of at least one of the residues of the ligand-binding region;
providing a first molecular structure to be fitted to said selected coordinates of residues of said region;
fitting the first molecular structure to said P450 structure including at least one of the selected coordinates thereof;
providing a second molecular structure; and
fitting the second molecular structure to said P450 structure.

26. The method of claim 25 wherein said ligand binding region includes at least one residue selected from the group consisting of Arg97, Gly98, 11e99, Phe100, Leu102, Ala103, Val113, Phe114, Asn217, Thr364, Ser365, Leu366, Pro367 and Phe476.

27. The method of claim 25 wherein said ligand binding region includes at least one residue selected from the group consisting of Arg97, Gly98, Ile99, Phe100, Leu102, Ala103, Val113, Phe114, Leu 208, Asn217, Thr364, Ser365, Leu366, Pro367 and Phe476.

28. The method of claim 25 wherein said molecular structure fitted to the ligand-binding region is warfarin, piroxicam or tenoxicam.

29. The method of claim 25 which further comprises modifying the structure fitted to the ligand binding region.

30. The method of claim 25 wherein said second molecular structure is fitted to the haem-binding region.

31. A computer-based method for the analysis of the interaction of two molecular structures within a P450 binding pocket structure, which comprises:
providing the P450 structure of Table 1 or selected coordinates thereof which include coordinates of at least one of the residues of the haem-binding region;
providing a first molecular structure to be fitted to said selected coordinates of residues of said region;
fitting the first molecular structure to said P450 structure including at least one of the selected coordinates thereof;
providing a second molecular structure; and
fitting the second molecular structure to said P450 structure.

32. The method of claim 30 wherein the the haem binding region includes at least one residue selected from the group consisting of Leu294, Ala297, Gly298, Thr301, Thr302, Leu362, Leu366.

33. The method of claim 30 wherein said molecular structure fitted to the haem-binding region is warfarin, piroxicam or tenoxicam.

34. The method of claim 30 which further comprises modifying the structure fitted to the haem binding region.

35. The method of claim 31 wherein said second molecular structure is fitted to the ligand-binding region.

36. A method of administering a pharmaceutical compound metabolized by 2C9 to a patient wherein said compound is administered simultaneously or sequentially with a second compound which binds at the ligand binding pocket of 2C9.

37. A computer system, intended to generate structures and/or perform optimisation of compounds which interact with P450, P450 homologues or analogues, complexes of P450 with compounds, or complexes of P450 homologues or analogues with compounds, the system containing computer-readable data comprising one or more of:
(a) atomic coordinate data according to Table 1, said data defining the three-dimensional structure of P450 or at least selected coordinates thereof;
(b) structure factor data for P450, said structure factor data being derivable from the atomic coordinate data of Table 1;
(c) atomic coordinate data of a target P450 protein generated by homology modelling of the target based on the data of Table 1;
(d) atomic coordinate data of a target P450 protein generated by interpreting X-ray crystallographic data or NMR data by reference to the data of Table 1; and
(e) structure factor data derivable from the atomic coordinate data of (c) or (d).

38. The computer system of claim 37, wherein said atomic coordinate data is for at least one of the atoms provided by the residues of Table 3.

39. The computer system of claim 37, wherein said atomic coordinate data is for at least one of the atoms of the ligand-binding region, said region being defined as residues:
72, 74, 97, 98, 99, 100, 101, 102, 103, 104, 105, 106, 107, 110, 112, 113, 114, 116, 204, 205, 208, 213, 214, 216, 217, 233, 364, 365, 366, 367, 368, 369, 384, 385, 386, 387, 388, 476 and 477.

40. The computer system of claim 37, wherein said atomic coordinate data is for at least one of the atoms of the haem-binding region, said region being defined as residues:
97, 98, 111, 112, 113, 114, 115, 116, 178, 290, 293, 294, 295, 297, 298, 299, 300, 301, 302, 361, 362, 365, 366, 367, 368, 369, 389, 391 and 433.

41. The computer system of claim 37 comprising:
(i) a computer-readable data storage medium comprising data storage material encoded with said computer-readable data;
(ii) a working memory for storing instructions for processing said computer-readable data; and
(iii) a central-processing unit coupled to said working memory and to said computer-readable data storage medium for processing said computer-readable data and thereby generating structures and/or performing rational drug design.

42. The computer system of claim 41 further comprising a display coupled to said central-processing unit for displaying said structures.

43. A method of providing data for generating structures and/or performing optimisation of compounds which interact with P450, P450 homologues or analogues, complexes of P450 with compounds, or complexes of P450 homologues or analogues with compounds, the method comprising:
(i) establishing communication with a remote device containing computer-readable data comprising at least one of: (a) atomic coordinate data according to Table 1, said data defining the three-dimensional structure of P450, or the coordinates of a plurality of atoms of P450; (b) structure factor data for P450, said structure factor data being derivable from the atomic coordinate data of Table 1; (c) atomic coordinate data of a target P450 homologue or analogue generated by homology modelling of the target based on the data of Table 1; (d) atomic coordinate data of a protein generated by interpreting X-ray crystallographic data or NMR data by reference to the data of Table 1; and (e) structure factor data derivable from the atomic coordinate data of (c) or (d); and
(ii) receiving said computer-readable data from said remote device.

44. A computer-readable storage medium comprising a data storage material encoded with computer-readable data, wherein the data are defined by:
(a) atomic coordinate data according to Table 1, said data defining the three-dimensional structure of P450 or at least selected coordinates thereof;
(b) structure factor data for P450, said structure factor data being derivable from the atomic coordinate data of Table 1;
(c) atomic coordinate data of a target P450 protein generated by homology modeling of the target based on the data of Table 1;
(d) atomic coordinate data of a target P450 protein generated by interpreting X-ray crystallographic data or NMR data by reference to the data of Table 1; and
(e) structure factor data derivable from the atomic coordinate data of (c) or (d).

45. The computer-readable storage medium of claim 44, wherein said atomic coordinate data is for at least one of the atoms provided by the residues of:
Table 3;
the ligand-binding region ligand-binding region, said region being defined as residues: 72, 74, 97, 98, 99, 100, 101, 102, 103, 104, 105, 106, 107, 110, 112, 113, 114, 116, 204, 205, 208, 213, 214, 216, 217, 233, 364, 365, 366, 367, 368, 369, 384, 385, 386, 387, 388, 476 and 477;
Table 5;
Table 5 together with Leu208; or
the haem-binding region, said region being defined as residues: 97, 98, 111, 112, 113, 114, 115, 116, 178, 290, 293, 294, 295, 297, 298, 299, 300, 301, 302, 361, 362, 365, 366, 367, 368, 369, 389, 391 and 433.

46. A computer-readable storage medium, comprising a data storage material encoded with computer readable data, wherein the data are defined by all or a portion of the structure coordinates of the P450 protein of Table 1, or a homologue of P450, wherein said homologue comprises backbone atoms that have a root mean square deviation from the backbone atoms of Table 1 of not more than 2.0 Å.

47. A computer-readable storage medium comprising a data storage material encoded with a first set of computer-readable data comprising a Fourier transform of at least a portion of the structural coordinates for the P450 protein according to Table 1; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.


SEQUENCE LISTING (41 sequences)

SEQ ID NO: 1 - DNA, 1428 bp, Artificial sequence
  DNA sequence encoding 2C9-FGloop K206E (also referred to as 1155)
atggctaagaaaacgagctctaaagggcggccgcctggccccactcctctcccagtgattggaaatatcctacagataggtattaaggacatcagcaaatccttaaccaatctctcaaaggtctatggccctgtgttcactctgtattttggcctgaaacccatagtggtgctgcatggatatgaagcagtgaaggaagccctgattgatcttggagaggagttttctggaagaggcattttcccactggctgaaagagctaacagaggatttggaattgttttcagcaatggaaagaaatggaaggagatccggcgtttctccctcatgacgctgcggaattttgggatggggaagaggagcattgaggaccgtgttcaagaggaagcccgctgccttgtggaggagttgagaaaaaccaaggcctcaccctgtgatcccactttcatcctgggctgtgctccctgcaatgtgatctgctccattattttccataaacgttttgattataaagatcagcaatttcttaacttaatggaaaagttgaatgaaaacatcgagattttgagcagcccctggatccaggtctacaataatttccctgctctccttgattatttcccgggaactcacaacaaattacttaaaaacgttgcttttatgaaaagttatattttggaaaaagtaaaagaacaccaagaatcaatggacatgaacaaccctcaggactttattgattgcttcctgatgaaaatggagaaggaaaagcacaaccaaccatctgaatttactattgaaagcttggaaaacactgcagttgacttgtttggagctgggacagagacgacaagcacaaccctgagatatgctctccttctcctgctgaagcacccagaggtcacagctaaagtccaggaagagattgaacgtgtgattggcagaaaccggagcccctgcatgcaagacaggagccacatgccctacacagatgctgtggtgcacgaggtccagagatacattgaccttctccccaccagcctgccccatgcagtgacctgtgacattaaattcagaaactatctcattcccaagggcacaaccatattaatttccctgacttctgtgctacatgacaacaaagaatttcccaacccagagatgtttgaccctcatcactttctggatgaaggtggcaattttaagaaaagtaaatacttcatgcctttctcagcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctctgcttcattcctgtccaccaccaccactga

SEQ ID NO: 2 - protein, 475 aa, Artificial sequence
  2C9-FGloop K206E
MAKKTSSKGRPPGPTPLPVIGNILQIGIKDISKSLTNLSKVYGPVFTLYFGLKPIVVLHGYEAVKEALIDLGEEFSGRGIFPLAERANRGFGIVFSNGKKWKEIRRFSLMTLRNFGMGKRSIEDRVQEEARCLVEELRKTKASPCDPTFILGCAPCNVICSIIFHKRFDYKDQQFLNLMEKLNENIEILSSPWIQVYNNFPALLDYFPGTHNKLLKNVAFMKSYILEKVKEHQESMDMNNPQDFIDCFLMKMEKEKHNQPSEFTIESLENTAVDLFGAGTETTSTTLRYALLLLLKHPEVTAKVQEEIERVIGRNRSPCMQDRSHMPYTDAVVHEVQRYIDLLPTSLPHAVTCDIKFRNYLIPKGTTILISLTSVLHDNKEFPNPEMFDPHHFLDEGGNFKKSKYFMPFSAGKRICVGEALAGMELFLFLTSILQNFNLKSLVDPKNLDTTPVVNGFASVPPFYQLCFIPVHHHH

SEQ ID NO: 3 - DNA, 1428 bp, Artificial sequence
  DNA sequence encoding 2C9trunc (also referred to as 1003)
atggctaagaaaacgagctctaaagggcggccgcctggccccactcctctcccagtgattggaaatatcctacagataggtattaaggacatcagcaaatccttaaccaatctctcaaaggtctatggccctgtgttcactctgtattttggcctgaaacccatagtggtgctgcatggatatgaagcagtgaaggaagccctgattgatcttggagaggagttttctggaagaggcattttcccactggctgaaagagctaacagaggatttggaattgttttcagcaatggaaagaaatggaaggagatccggcgtttctccctcatgacgctgcggaattttgggatggggaagaggagcattgaggaccgtgttcaagaggaagcccgctgccttgtggaggagttgagaaaaaccaaggcctcaccctgtgatcccactttcatcctgggctgtgctccctgcaatgtgatctgctccattattttccataaacgttttgattataaagatcagcaatttcttaacttaatggaaaagttgaatgaaaacatcaagattttgagcagcccctggatccagatctgcaataatttttctcctatcattgattacttcccgggaactcacaacaaattacttaaaaacgttgcttttatgaaaagttatattttggaaaaagtaaaagaacaccaagaatcaatggacatgaacaaccctcaggactttattgattgcttcctgatgaaaatggagaaggaaaagcacaaccaaccatctgaatttactattgaaagcttggaaaacactgcagttgacttgtttggagctgggacagagacgacaagcacaaccctgagatatgctctccttctcctgctgaagcacccagaggtcacagctaaagtccaggaagagattgaacgtgtgattggcagaaaccggagcccctgcatgcaagacaggagccacatgccctacacagatgctgtggtgcacgaggtccagagatacattgaccttctccccaccagcctgccccatgcagtgacctgtgacattaaattcagaaactatctcattcccaagggcacaaccatattaatttccctgacttctgtgctacatgacaacaaagaatttcccaacccagagatgtttgaccctcatcactttctggatgaaggtggcaattttaagaaaagtaaatacttcatgcctttctcagcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctctgcttcattcctgtccaccaccaccactga

SEQ ID NO: 4 - protein, 475 aa, Artificial sequence
  2C9trunc
MAKKTSSKGRPPGPTPLPVIGNILQIGIKDISKSLTNLSKVYGPVFTLYFGLKPIVVLHGYEAVKEALIDLGEEFSGRGIFPLAERANRGFGIVFSNGKKWKEIRRFSLMTLRNFGMGKRSIEDRVQEEARCLVEELRKTKASPCDPTFILGCAPCNVICSIIFHKRFDYKDQQFLNLMEKLNENIKILSSPWIQICNNFSPIIDYFPGTHNKLLKNVAFMKSYILEKVKEHQESMDMNNPQDFIDCFLMKMEKEKHNQPSEFTIESLENTAVDLFGAGTETTSTTLRYALLLLLKHPEVTAKVQEEIERVIGRNRSPCMQDRSHMPYTDAVVHEVQRYIDLLPTSLPHAVTCDIKFRNYLIPKGTTILISLTSVLHDNKEFPNPEMFDPHHFLDEGGNFKKSKYFMPFSAGKRICVGEALAGMELFLFLTSILQNFNLKSLVDPKNLDTTPVVNGFASVPPFYQLCFIPVHHHH

SEQ ID NO: 5 - protein, 490 aa, Homo sapiens [sequence omitted]

SEQ ID NO: 6 - protein, 10 aa, Artificial sequence
  N-terminal sequence of SEQ ID NO 2 and SEQ ID NO 4
MAKKTSSKGR

SEQ ID NO: 7 - DNA, 38 bp, Artificial sequence
  Oligonucleotide
ggaagaggcattttcccatatgctgaaagagctaacag

SEQ ID NO: 8 - DNA, 38 bp, Artificial sequence
  Oligonucleotide
ctgttagctctttcagcatatgggaaaatgcctcttcc

SEQ ID NO: 9 - DNA, 38 bp, Artificial sequence
  Oligonucleotide
ggaagaggcattttcccatgggctgaaagagctaacag

SEQ ID NO: 10 - DNA, 38 bp, Artificial sequence
  Oligonucleotide
ctgttagctctttcagcccatgggaaaatgcctcttcc

SEQ ID NO: 11 - DNA, 40 bp, Artificial sequence
  Oligonucleotide
ggaagaggcattttcccactgtatgaaagagctaacagag

SEQ ID NO: 12 - DNA, 40 bp, Artificial sequence
  Oligonucleotide
ctctgttagctctttcatacagtgggaaaatgcctcttcc

SEQ ID NO: 13 - DNA, 40 bp, Artificial sequence
  Oligonucleotide
ggaagaggcattttcccacttggtgaaagagctaacagag

SEQ ID NO: 14 - DNA, 40 bp, Artificial sequence
  Oligonucleotide
ctctgttagctctttcaccaagtgggaaaatgcctcttcc

SEQ ID NO: 15 - DNA, 40 bp, Artificial sequence
  Oligonucleotide
gaatgaaaacatcaagatttacagcagcccctggatccag

SEQ ID NO: 16 - DNA, 40 bp, Artificial sequence
  Oligonucleotide
ctggatccaggggctgctgtaaatcttgatgttttcattc

SEQ ID NO: 17 - DNA, 40 bp, Artificial sequence
  Oligonucleotide
gaatgaaaacatcaagatttggagcagcccctggatccag

SEQ ID NO: 18 - DNA, 40 bp, Artificial sequence
  Oligonucleotide
ctggatccaggggctgctccaaatcttgatgttttcattc

SEQ ID NO: 19 - DNA, 37 bp, Artificial sequence
  Oligonucleotide
gaaaacatcgagatttggagcagcccctggatccagg

SEQ ID NO: 20 - DNA, 37 bp, Artificial sequence
  Oligonucleotide
cctggatccaggggctgctccaaatctcgatgttttc

SEQ ID NO: 21 - DNA, 38 bp, Artificial sequence
  Oligonucleotide
gcccctggatccagatctgctataatttttctcctatc

SEQ ID NO: 22 - DNA, 38 bp, Artificial sequence
  Oligonucleotide
gataggagaaaaattatagcagatctggatccaggggc

SEQ ID NO: 23 - DNA, 37 bp, Artificial sequence
  Oligonucleotide
ccctggatccaggtctactataatttccctgctctcc

SEQ ID NO: 24 - DNA, 37 bp, Artificial sequence
  Oligonucleotide
ggagagcagggaaattatagtagacctggatccaggg

SEQ ID NO: 25 - DNA, 38 bp, Artificial sequence
  Oligonucleotide
gcccctggatccagatctgctggaatttttctcctatc

SEQ ID NO: 26 - DNA, 38 bp, Artificial sequence
  Oligonucleotide
gataggagaaaaattccagcagatctggatccaggggc

SEQ ID NO: 27 - DNA, 36 bp, Artificial sequence
  Oligonucleotide
cctggatccaggtctactggaatttccctgctctcc

SEQ ID NO: 28 - DNA, 36 bp, Artificial sequence
  Oligonucleotide
ggagagcagggaaattccagtagacctggatccagg

SEQ ID NO: 29 - protein, 29 aa, Homo sapiens
MDSLVVLVLCLSCLLLLSLWRQSSGRGKL

SEQ ID NO: 30 - DNA, 51 bp, Artificial sequence
  Oligonucleotide
ataagaatgcggccgcctggccccactcctctcccagtgattggaaatatc

SEQ ID NO: 31 - DNA, 50 bp, Artificial sequence
  Oligonucleotide
tgcggtcgactcagtggtggtggtggacaggaatgaagcagagctggtag

SEQ ID NO: 32 - DNA, 29 bp, Artificial sequence
  Oligonucleotide
tatggctaagaaaacgagctctaaagggc

SEQ ID NO: 33 - DNA, 31 bp, Artificial sequence
  Oligonucleotide
ggccgccctttagagctcgttttcttagcca

SEQ ID NO: 34 - DNA, 43 bp, Artificial sequence
  Oligonucleotide
gatccaggtctacaataatttccctgctctccttgattatttc

SEQ ID NO: 35 - DNA, 43 bp, Artificial sequence
  Oligonucleotide
ccgggaaataatcaaggagagcagggaaattattgtagacctg

SEQ ID NO: 36 - DNA, 41 bp, Artificial sequence
  Primer
ccagatctgcaataattttccgaccacattgattacttccc

SEQ ID NO: 37 - DNA, 42 bp, Artificial sequence
  Primer
gggaagtaatcaatgatggtcggaaaattattgcagatctgg

SEQ ID NO: 38 - DNA, 43 bp, Artificial sequence
  Primer
ggaaaagttgaatgaaaacatcgagattttgagcagcccctgg

SEQ ID NO: 39 - DNA, 43 bp, Artificial sequence
  Primer
ccaggggctgctcaaaatctcgatgttttcattcaacttttcc

SEQ ID NO: 40 - protein, 9 aa, Artificial sequence
  2C9-FGloop K206E
AKKTSSKGR

SEQ ID NO: 41 - protein, 475 aa, Artificial sequence
  2C9-FGloop
MAKKTSSKGRPPGPTPLPVIGNILQIGIKDISKSLTNLSKVYGPVFTLYFGLKPIVVLHGYEAVKEALIDLGEEFSGRGIFPLAERANRGFGIVFSNGKKWKEIRRFSLMTLRNFGMGKRSIEDRVQEEARCLVEELRKTKASPCDPTFILGCAPCNVICSIIFHKRFDYKDQQFLNLMEKLNENIKILSSPWIQVYNNFPALLDYFPGTHNKLLKNVAFMKSYILEKVKEHQESMDMNNPQDFIDCFLMKMEKEKHNQPSEFTIESLENTAVDLFGAGTETTSTTLRYALLLLLKHPEVTAKVQEEIERVIGRNRSPCMQDRSHMPYTDAVVHEVQRYIDLLPTSLPHAVTCDIKFRNYLIPKGTTILISLTSVLHDNKEFPNPEMFDPHHFLDEGGNFKKSKYFMPFSAGKRICVGEALAGMELFLFLTSILQNFNLKSLVDPKNLDTTPVVNGFASVPPFYQLCFIPVHHHH